(19) United States
(12) Patent Application Publication
(10) Pub. No.: US 2020/0048183 A1
(43) Pub. Date: Feb. 13, 2020

(54) ANTIMICROBIAL AGENTS

(71) Applicant: THE UNIVERSITY OF WARWICK, Coventry (GB)

(72) Inventors: Gregory Leonard CHALLIS, Warwickshire (GB); Xinyun JIAN, Warwickshire (GB); Christian HOBSON, Warwickshire (GB); Joleen Solange Liesbet MASSCHELEIN, Warwickshire (GB)

(21) Appl. No.: 16/606,845

(22) PCT Filed: Apr. 23, 2018
(86) PCT No.: PCT/GB2018/051058

§ 371 Date: Oct. 21, 2019

(30) Foreign Application Priority Data
Apr. 21, 2017 (GB) PCT/GB2017/051124
Oct. 25, 2017 (GB) 1717551.4

(51) Int. Cl. C07C 69/757; C07D 309/10; C07C 271/12

(52) U.S. Cl. CPC C07C 69/757; A61K 45/06; C07C 271/12; C07D 309/10

(57) ABSTRACT

The invention provides novel analogues of enacyloxin Ha and their pharmaceutically acceptable salts, metabolites, isomers (e.g. stereoisomers) and prodrugs. Such compounds are effective in the treatment of infections caused by Gram-negative bacteria such as Acinetobacter baumannii. Compounds in accordance with the invention include those of formula (A), and their pharmaceutically acceptable salts, metabolites, isomers (e.g. stereoisomers) and prodrugs: In formula (A): X is 0 or NR^{x} (where R* is either H or C₁₋₃ alkyl, e.g. CH₃); R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group); R² is H, F, Cl, Br, I or CH₃; R³ is H or OH; R⁸ is a straight-chained or branched C₁₋₈ alkyl group (e.g. a C₁₋₆ alkyl group); Y is one of the following groups: (wherein each * denotes the point of attachment of the group to the remainder of the molecule; R⁹ is H, F, Cl, Br or I; R⁴ and R⁵ are independently selected from H and OH, or R⁴ and R⁵ together are =0, preferably R⁴ is H and R⁵ is OH; R⁶ is H, F, Cl, Br, I or CH₃; R⁷ is H and R^{7′} is OH, or R⁷ and R^{7′} together are =0, preferably R7 is H and R7′ is OH); and each — independently represents an optional bond (i.e. each of C₂-C₃, C₄-C₅, C₆-C₇, C₈-C₉ and C₁₀-C₁₁ are independently either C—C (single) or C═C (double) bonds).

The present invention relates to novel polyketide compounds, their preparation, and their use as antimicrobial agents. The invention further relates to antimicrobial agents obtained from a Burkholderia ambifaria strain, or from a variant and/or mutant thereof.

Bacterial pathogens are prominent in many diseases and the treatment of bacterial infections has become increasingly difficult over recent years with the emergence of a number of antibiotic resistant bacterial strains. Examples include methicillin-resistant Staphylococcus aureus (MRSA), vancomycin-resistant Enterococci (VRE), and multidrug-resistant Gram-negative bacteria such as Acinetobacter baumannii. In addition to the emergence of antibiotic resistant strains, there are many bacterial infections that remain difficult to treat, for example, infections in immuno-compromised patients (e.g. those with AIDS).

There is therefore an ongoing need to identify new antimicrobial agents that can be used to treat microbial infections effectively, including those caused by drug resistant microbes, for example, infections caused by drug-resistant bacteria.

WO 2011/101631 describes the compound enacyloxin IIa having anti-bacterial activity. Enacyloxin IIa is produced by strains of Burkholderia and Frateuria species and has the following structural formula:

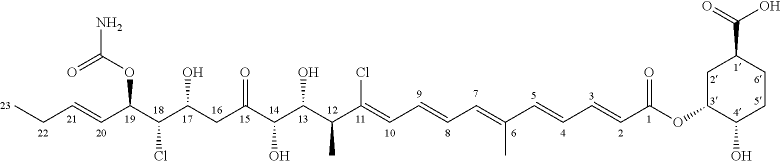 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O]

However, this compound is unstable. Notably, the ester group is prone to rearrangement and hydrolysis. This chemical instability limits its potential for clinical use. Although formula (I) in WO 2011/101631 encompasses a wide range of potential compounds, no actual derivatives of enacyloxin IIa are made or tested in this earlier application.

The inventors have now identified novel polyketide compounds having anti-microbial activity and which may be used as alternative anti-bacterial agents to enacyloxin IIa. Compared to enacyloxin IIa, they are structurally simpler and, in some cases, more chemically stable. This structural simplification and improvement in stability represents a significant advance over the earlier known compound. In comparison to enacyloxin IIa, many of the compounds identified by the inventors have at least equal potency against multidrug-resistant pathogenic bacteria such as Acinetobacter baumannii. Based on what is known about the structure-activity relationship for enacyloxin IIa, it could not have been predicted that biological activity would be retained following the modifications described herein.

The present invention provides derivatives of enacyloxin IIa having anti-bacterial activity. These may be produced using methods known in the art, for example techniques capable of modifying the genes responsible for the biosynthesis of enacyloxin IIa in order to produce recombinant microbes that biosynthesise the derivatives. Such derivatives are as herein described and differ from enacyloxin IIa in at least one respect, for example these may differ at one or more key positions on the polyketide chain and/or in respect of modifications made to the dihydroxycyclohexane carboxylic acid (DHCCA) moiety. Specific methods which may be used to produce the derivatives may involve the use of a heterologous host for the expression of enacyloxin biosynthetic genes, knock-out mutagenesis, mutasynthesis, semi-synthetic modification and/or total chemical synthesis.

Accordingly, the present invention provides novel polyketide compounds that are effective against a range of microbes, including bacteria and resistant bacteria, and in particular against multidrug-resistant Gram-negative bacteria such as Acinetobacter baumannii. The invention also provides recombinant microorganisms capable of producing such compounds.

The compounds of the invention, including but not limited to those specified in the examples herein, possess the ability to inhibit and/or prevent the growth of microbes. Such compounds may be useful in the treatment of a wide variety of microbial infections.

The present invention further provides pharmaceutical compositions comprising one or more compounds according to the invention. In addition, compounds of the invention may be useful in the treatment of microbial infections described herein either when used alone or in combination with other therapeutic agents.

Further aspects of the present invention include: processes for the preparation of the compounds according to the invention; methods for the treatment of infections by microbes, including drug-resistant strains thereof, comprising administering a compound according to the present invention; and uses of the compounds according to the present invention.

First Aspect

In a first aspect the invention provides compounds in which the carbamoyl group (—OCONH₂) at the C19 position of enacyloxin IIa is replaced by another group or moiety.

In one embodiment of this aspect the invention provides compounds in which the C15-C19 positions in the polyketide chain of enacyloxin IIa are modified to form an interrupting tetrahydropyran group. Such compounds may also differ from enacyloxin IIa at other key positions on the polyketide chain (for example, by modification of the substituent groups at one or more of positions C11, C14 and C21 of enacyloxin IIa, and/or by replacement of the ester linkage by an amide linkage) and/or in respect of modifications made to the terminal dihydroxycyclohexane carboxylic acid (DHCCA) moiety.

In another embodiment of this aspect the invention provides compounds in which the —OCONH₂ group at the C19 position in the polyketide chain of enacyloxin IIa is either replaced by an OH group or by an oxo group, and in which the oxo group at the C15 position of enacyloxin IIa is optionally replaced by H and OH. Such compounds may also differ from enacyloxin IIa at other key positions on the polyketide chain (for example, by modification of the substituent groups at one or more of positions C11, C14, C18 and C21) and/or in respect of modifications made to the terminal dihydroxycyclohexane carboxylic acid (DHCCA) moiety.

Viewed from a first aspect the invention thus provides a compound of formula (A), or a pharmaceutically acceptable salt, metabolite, isomer (e.g. stereoisomer) or prodrug thereof:

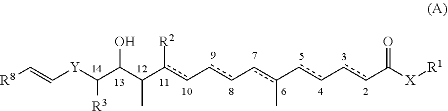

wherein:
X is O or NR^{x} (where R^{x} is either H or C₁₋₃ alkyl, e.g. CH₃);
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁸ is a straight-chained or branched C₁₋₈ alkyl group (e.g. a C₁₋₆ alkyl group);
Y is one of the following groups:

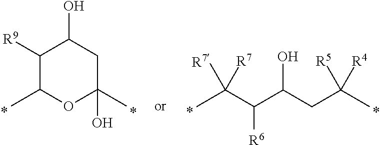

(where each * denotes the point of attachment of the group to the remainder of the molecule);
R⁹ is H, F, Cl, Br or I;
R⁴ and R⁵ are independently selected from H and OH, or R⁴ and R⁵ together are ═O, preferably R⁴ is H and R⁵ is OH;
R⁶ is H, F, Cl, Br, I or CH₃;
R⁷ is H and R^{7′} is OH, or R⁷ and R^{7′} together are ═O, preferably R⁷ is H and R^{7′} is OH; and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment of formula (A), the invention provides a compound of formula (I), or a pharmaceutically acceptable salt, metabolite, isomer (e.g. stereoisomer) or prodrug thereof:

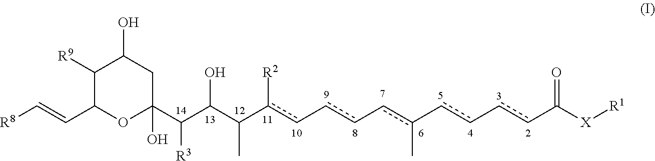

wherein:
X is O or NR^{x} (where R^{x} is either H or C₁₋₃ alkyl, e.g. CH₃);
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁸ is a straight-chained or branched C₁₋₈ alkyl group (e.g. a C₁₋₆ alkyl group);
R⁹ is H, F, Cl, Br or I; and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment of formula (I), X is O.

In one embodiment of formula (I), R¹ is a cyclohexyl or cyclopentyl ring which is optionally substituted by one or more substituents. In another embodiment of formula (I), R¹ is a cyclohexenyl ring which is optionally substituted by one or more substituents.

In another embodiment of formula (I), R¹ is a straight-chained or branched C₁₋₆ alkyl group (e.g. C₁₋₃ alkyl group) which may be substituted by one or more substituents. Preferably it is a straight-chained alkyl group.

Optional substituents which may be present in group R¹ include one or more of the following: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H, (or an ester thereof). Suitable ester-forming groups include optionally substituted alkyl, alkenyl, cycloalkyl, cycloalkenyl, aryl and heteroaryl groups. Examples of such groups include optionally substituted C₁₋₁₂-alkyl, C₁₋₁₂-alkenyl, C₃₋₁₀-cycloalkyl, aryl and heteroaryl groups, wherein the aryl and heteroaryl groups may contain from 5 to 10 carbon atoms and the heteroaryl groups further contain one or more (e.g. 1, 2, 3 or 4) heteroatoms selected from N, O and S. In one embodiment, the substituents which may be present in group R¹ may be selected from any of the following: OH, NH₂, SH, F, Cl, Br, I, CH₃, CO₂H, PO₃H₂ and SO₃H₂.

In one embodiment, one, two or three (preferably one or two) substituents may be present in group R¹. Where more than one substituent is present, these may be the same or different. Preferably, at least one of the substituents will be CO₂H or an ester thereof as herein defined.

In one embodiment, where R¹ is substituted by more than one substituent (e.g. two or three substituents), the substituents may be selected from the group consisting of CO₂H (or an ester thereof), and OH. Preferably R¹ may be substituted by one CO₂H group (or an ester thereof), and/or by one OH group, e.g. by one CO₂H group (or an ester thereof), and by one OH group.

In one embodiment, R¹ is an optionally substituted cyclohexyl or cyclopentyl group. Substituents on these rings may be any of those herein described. Preferably, the substituents may be selected from CO₂H (or an ester thereof), and OH. In one embodiment, R¹ is a cyclohexyl ring substituted by one CO₂H group (or an ester thereof), and by one OH group. These substituents may be present at any ring positions, but in one embodiment these may be para to one another.

Where R¹ is a straight-chained or branched C₁₋₆ alkyl group (e.g. C₁₋₃ alkyl group), this is preferably substituted. Preferred substituents are selected from CO₂H (or an ester thereof), and OH. In one embodiment, R¹ is a straight-chained or branched (preferably straight-chained) C₁₋₆ alkyl group (e.g. C₁₋₃ alkyl group) substituted by one CO₂H group (or an ester thereof), and/or by one OH group. Where present, any CO₂H group (or an ester thereof) will typically be provided at the terminal position of the alkyl group.

Examples of R¹ groups include any of the following (in which * denotes the point of attachment of the substituent to the remainder of the molecule):

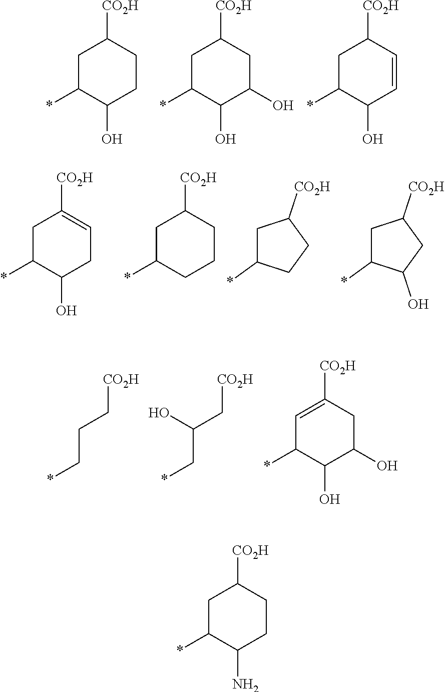

In one embodiment of formula (I), R² is F, Cl, Br or I. In a preferred embodiment, R² is Cl.

In another embodiment of formula (I), R² is Br.

In one embodiment of formula (I), R³ is OH. In another embodiment of formula (I), R³ is H.

In one embodiment of formula (I), R⁸ is a straight-chained or branched C₁₋₅ alkyl, preferably a straight-chained or branched C₁₋₄ alkyl. Examples of such groups include methyl, ethyl, isopropyl, and tert. butyl. In a preferred embodiment, R⁸ is ethyl.

In one embodiment of formula (I), R⁹ is H or Cl. In a preferred embodiment, R⁹ is Cl.

In another embodiment of formula (I), R⁹ is Br.

In one embodiment of formula (I), R² and R⁹ are both Br. In another embodiment, R² is Br and R⁹ is Cl.

In one embodiment of formula (I), each of C₂-C₃, C₄-C₅, C₆-C₇, C₈-C₉ and C₁₀-C₁₁ are C═C (double) bonds.

In one embodiment of formula (I), R¹ is a substituted cyclohexyl group and R⁸ is ethyl.

In one embodiment, the invention provides a compound of formula (Ia) or a pharmaceutically acceptable, salt, metabolite, isomer (e.g. stereoisomer) or prodrug thereof:

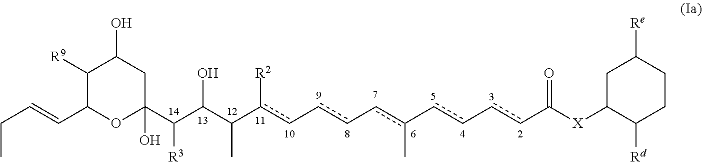

wherein:
X is as herein defined;
R^{d} is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R², R³ and R⁹ are as herein defined; and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment, the invention provides compounds of formula (Ia) and their pharmaceutically acceptable salts, metabolites, isomers (e.g. stereoisomers) and prodrugs, wherein:
X=O or NH;
R^{d} is H, OH, NH₂, SH, F, Cl, Br, I, or CH₃, preferably OH;
R^{e} is H, CO₂H, PO₃H₂, or SO₃H₂, preferably CO₂H;
R² is H, F, Cl, Br, I or CH₃;
R³ is H or OH;
R⁹ is H, Cl or Br, e.g. R⁹ is H or Cl; and
each of C₂-C₃, C₄-C₅, C₆-C₇, C₈-C₉ and C₁₀-C₁₁ are independently either C—C (single) or C═C (double) bonds.

In one embodiment of formula (Ia), X is O.

In one embodiment of formula (Ia), R² is Cl.

In one embodiment of formula (Ia), R² is Br.

In one embodiment of formula (Ia), R³ is OH.

In one embodiment of formula (Ia), each of C₂-C₃, C₄-C₅, C₆-C₇, C₈-C₉ and C₁₀-C₁₁ are C═C (double) bonds.

In one embodiment, the invention provides a compound of formula (Ib), or a pharmaceutically acceptable salt, metabolite, isomer (e.g. stereoisomer) or prodrug thereof:

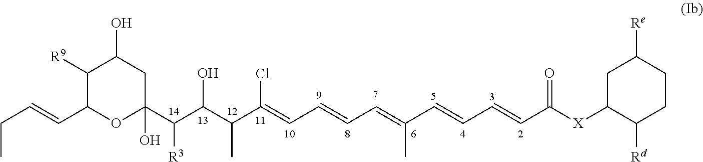

wherein X, R^{d}, R^{e}, R³ and R⁹ are as herein defined.

In another embodiment, the invention provides a compound of formula (Ic), or a pharmaceutically acceptable salt, metabolite, or prodrug thereof:

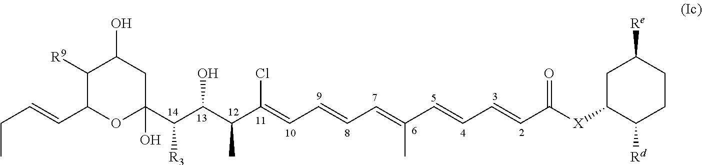

wherein X, R^{d}, R^{e}, R³ and R⁹ are as herein defined.

In one embodiment of formula (Ib) or formula (Ic), R^{d} is OH and R^{e} is CO₂H.

Examples of compounds of formula (I) according to the invention include the following, and their pharmaceutically acceptable salts, metabolites, isomers (e.g. stereoisomers) and prodrugs:

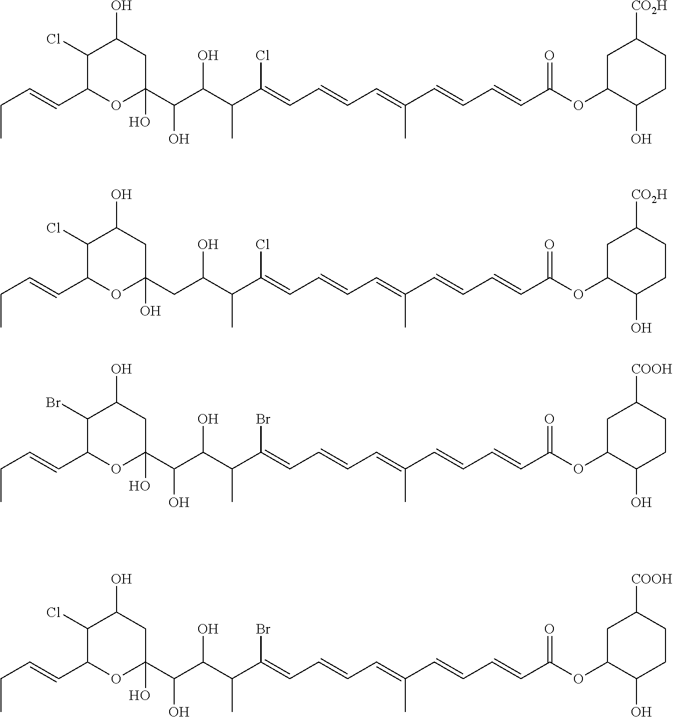

Further examples of compounds of formula (l) according to the invention include the following, and their pharmaceutically acceptable salts, metabolites, and prodrugs:

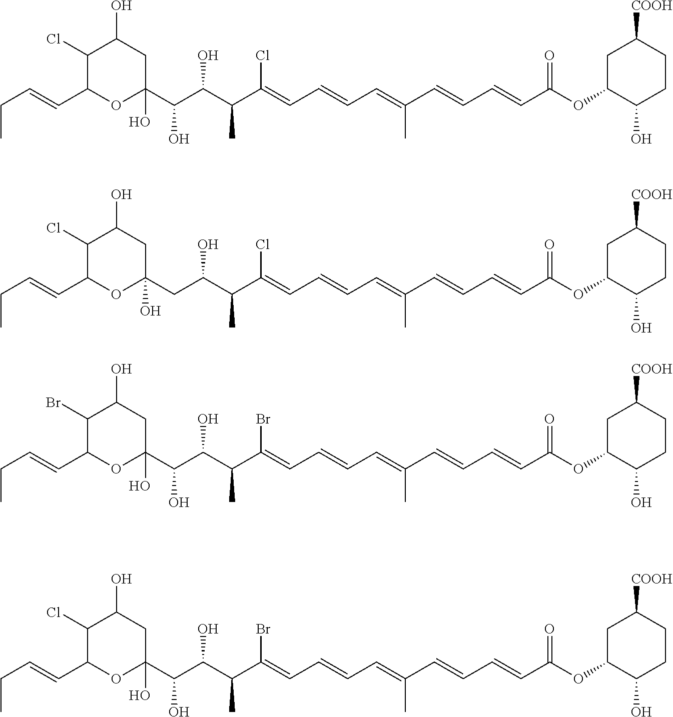

In another embodiment of formula (A), the invention provides a compound of formula (IV), or a pharmaceutically acceptable salt, metabolite, isomer (e.g. stereoisomer) or prodrug thereof:

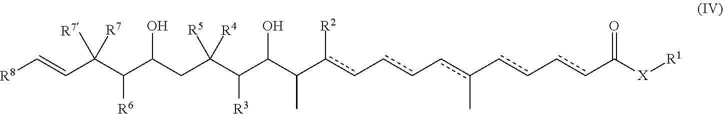

wherein:
X is O or NR^{x}(where R^{x} is either H or C₁₋₃ alkyl, e.g. CH₃);
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁴ and R⁵ are independently selected from H and OH, or R⁴ and R⁵ together are ═O, preferably
R⁴ is H and R⁵ is OH;
R⁶ is H, F, Cl, Br, I or CH₃;
R⁷ is H and R^{7′} is OH, or R⁷ and R^{7′} together are ═O, preferably R⁷ is H and R^{7′} is OH;
R⁸ is a straight-chained or branched C₁₋₈ alkyl group (e.g. C₁₋₈ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment of formula (IV), X is O.

In one embodiment of formula (IV), R¹ is a cyclohexyl or cyclopentyl ring which is optionally substituted by one or more substituents. In another embodiment of formula (IV), R¹ is a cyclohexenyl ring which is optionally substituted by one or more substituents.

In another embodiment of formula (IV), R¹ is a straight-chained or branched C₁₋₆ alkyl group (e.g. C₁₋₃ alkyl group) which may be substituted by one or more substituents. Preferably it is a straight-chained alkyl group.

Optional substituents which may be present in group R¹ include one or more of the following: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof). Suitable ester-forming groups include optionally substituted alkyl, alkenyl, cycloalkyl, cycloalkenyl, aryl and heteroaryl groups. Examples of such groups include optionally substituted C₁₋₁₂-alkyl, C₁₋₁₂-alkenyl, C₃₋₁₀-cycloalkyl, aryl and heteroaryl groups, wherein the aryl and heteroaryl groups may contain from 5 to 10 carbon atoms and the heteroaryl groups further contain one or more (e.g. 1, 2, 3 or 4) heteroatoms selected from N, O and S. In one embodiment, the substituents which may be present in group R¹ may be selected from any of the following: OH, NH₂, SH, F, Cl, Br, I, CH₃, CO₂H, PO₃H₂ and SO₃H₂.

In one embodiment, one, two or three (preferably one or two) substituents may be present in group R¹. Where more than one substituent is present, these may be the same or different. Preferably, at least one of the substituents will be CO₂H or an ester thereof as herein defined.

In one embodiment, where R¹ is substituted by more than one substituent (e.g. two or three substituents), the substituents may be selected from the group consisting of CO₂H (or an ester thereof), and OH. Preferably R¹ may be substituted by one CO₂H group (or an ester thereof), and/or by one OH group, e.g. by one CO₂H group (or an ester thereof), and by one OH group.

In one embodiment, R¹ is an optionally substituted cyclohexyl or cyclopentyl group. Substituents on these rings may be any of those herein described. Preferably, the substituents may be selected from CO₂H (or an ester thereof), and OH. In one embodiment, R¹ is a cyclohexyl ring substituted by one CO₂H group (or an ester thereof), and by one OH group. These substituents may be present at any ring positions, but in one embodiment these may be para to one another.

Where R¹ is a straight-chained or branched C₁₋₆ alkyl group (e.g. C₁₋₃ alkyl group), this is preferably substituted. Preferred substituents are selected from CO₂H (or an ester thereof), and OH. In one embodiment, R¹ is a straight-chained or branched (preferably straight-chained) C₁₋₆ alkyl group (e.g. C₁₋₃ alkyl group) substituted by one CO₂H group (or an ester thereof), and/or by one OH group. Where present, any CO₂H group (or an ester thereof) will typically be provided at the terminal position of the alkyl group.

Examples of R¹ groups include any of the following (in which * denotes the point of attachment of the substituent to the remainder of the molecule):

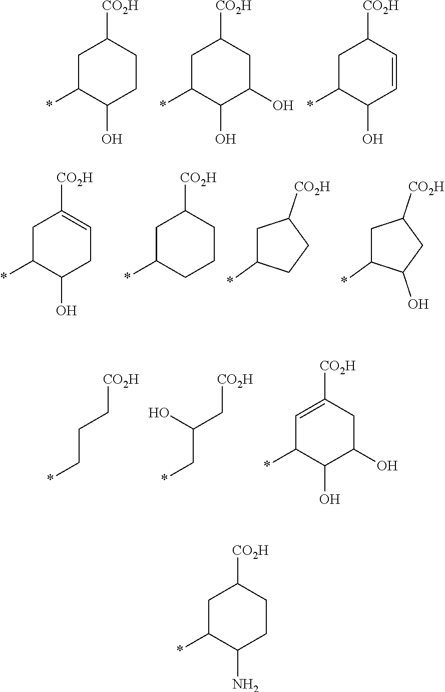

In one embodiment of formula (IV), R² is H, F, Cl, Br or I. In a preferred embodiment, R² is Cl or H. Preferably, R² is Cl. In another embodiment of formula (IV), R² is Br.

In one embodiment of formula (IV), R³ is OH. In another embodiment of formula (IV), R³ is H.

In one embodiment of formula (IV), R⁴ is H and R⁵ is OH.

In one embodiment of formula (IV), R⁶ is H or Cl, preferably H.

In one embodiment of formula (IV), R⁶ is Br.

In one embodiment of formula (IV), R² and R⁶ are both Br. In another embodiment, R² is Br and R⁶ is either H or Cl.

In one embodiment of formula (IV), R⁷ is H and R^{7′} is OH.

In one embodiment of formula (IV), R⁸ is a straight-chained or branched C₁₋₅ alkyl, preferably a straight-chained or branched C₁₋₄ alkyl. Examples of such groups include methyl, ethyl, isopropyl, and tert. butyl. In a preferred embodiment, R⁸ is ethyl.

In one embodiment of formula (IV), each of C₂-C₃, C₄-C₅, C₆-C₇, C₈-C₉ and C₁₀-C₁₁ are C═C (double) bonds.

In one embodiment of formula (IV), R⁴ is H and R⁵ is OH, and R⁷ is H and R^{7′} is OH. In one embodiment, the invention thus provides a compound of formula (IVa), or a pharmaceutically acceptable salt, metabolite, isomer (e.g. stereoisomer) or prodrug thereof:

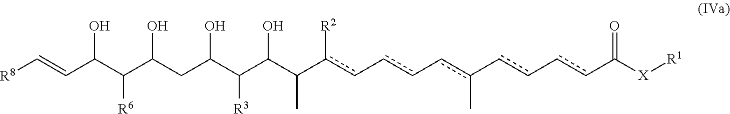

wherein R¹, R², R³, R⁶ and R⁸ are as herein defined.

In one embodiment of formula (IVa), R¹ is a substituted cyclohexyl group and R⁸ is ethyl.

In one embodiment, the invention provides a compound of formula (IVb) or a pharmaceutically acceptable salt, metabolite, isomer (e.g. stereoisomer) or prodrug thereof:

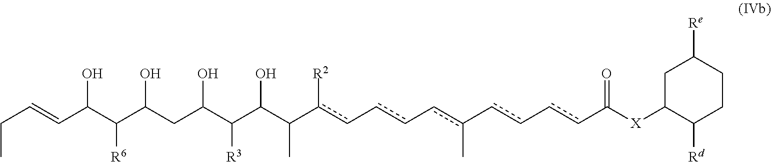

wherein:
X is as herein defined;
R^{d} is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R², R³ and R⁶ are as herein defined; and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment, the invention provides compounds of formula (IVb) and their pharmaceutically acceptable salts, metabolites, isomers (e.g. stereoisomers) and prodrugs, wherein:
X is O or NH, preferably O;
R^{d} is H, OH, NH₂, SH, F, Cl, Br, I, or CH₃, preferably OH;
R^{e} is H, CO₂H, PO₃H₂, or SO₃H₂, preferably CO₂H;
R² is H, F, Cl, Br, I or CH₃, preferably H, Cl or Br, e.g. H or Cl;
R³ is H or OH;
R⁶ is H, Cl or Br, e.g. R⁶ is H or Cl; and
each of C₂-C₃, C₄-C₅, C₆-C₇, C₈-C₉ and C₁₀-C₁₁ are independently either C—C (single) or C═C (double) bonds.

In one embodiment of formula (IVb), X is O.

In one embodiment of formula (IVb), R² is Cl. In another embodiment, R² is Br.

In one embodiment of formula (IVb), R³ is OH.

In one embodiment of formula (IVb), each of C₂-C₃, C₄-C₅, C₆-C₇, C₈-C₉ and C₁₀-C₁₁ are C═C (double) bonds.

In one embodiment, the invention provides a compound of formula (IVc), or a pharmaceutically acceptable salt, metabolite, isomer (e.g. stereoisomer) or prodrug thereof:

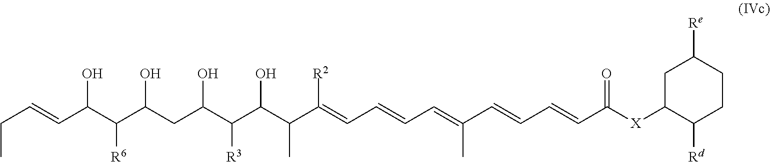

wherein X, R^{d}, R^{e}, R², R³ and R⁶ are as herein defined.

In another embodiment, the invention provides a compound of formula (IVd), or a pharmaceutically acceptable salt, metabolite, or prodrug thereof:

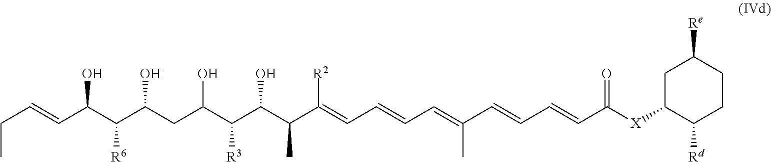

wherein X, R^{d}, R^{e}, R², R³ and R⁶ are as herein defined.

In one embodiment of formula (IVc) or formula (IVd), R^{d} is OH and R^{e} is CO₂H.

Examples of compounds of formula (IV) according to the invention include the following, and their pharmaceutically acceptable salts, metabolites, isomers (e.g. stereoisomers) and prodrugs:

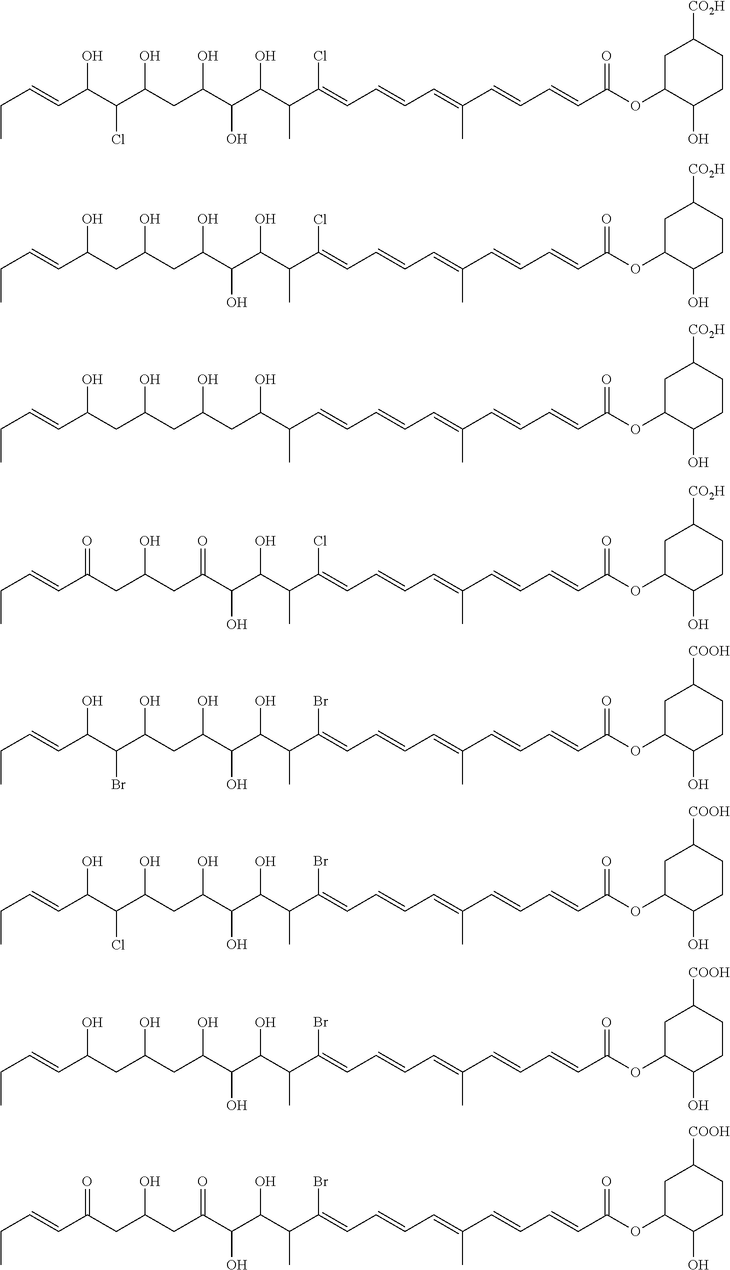

Further examples of compounds of formula (IIIa) and (IIIb) according to the invention include the following, and their pharmaceutically acceptable salts, metabolites, and prodrugs:

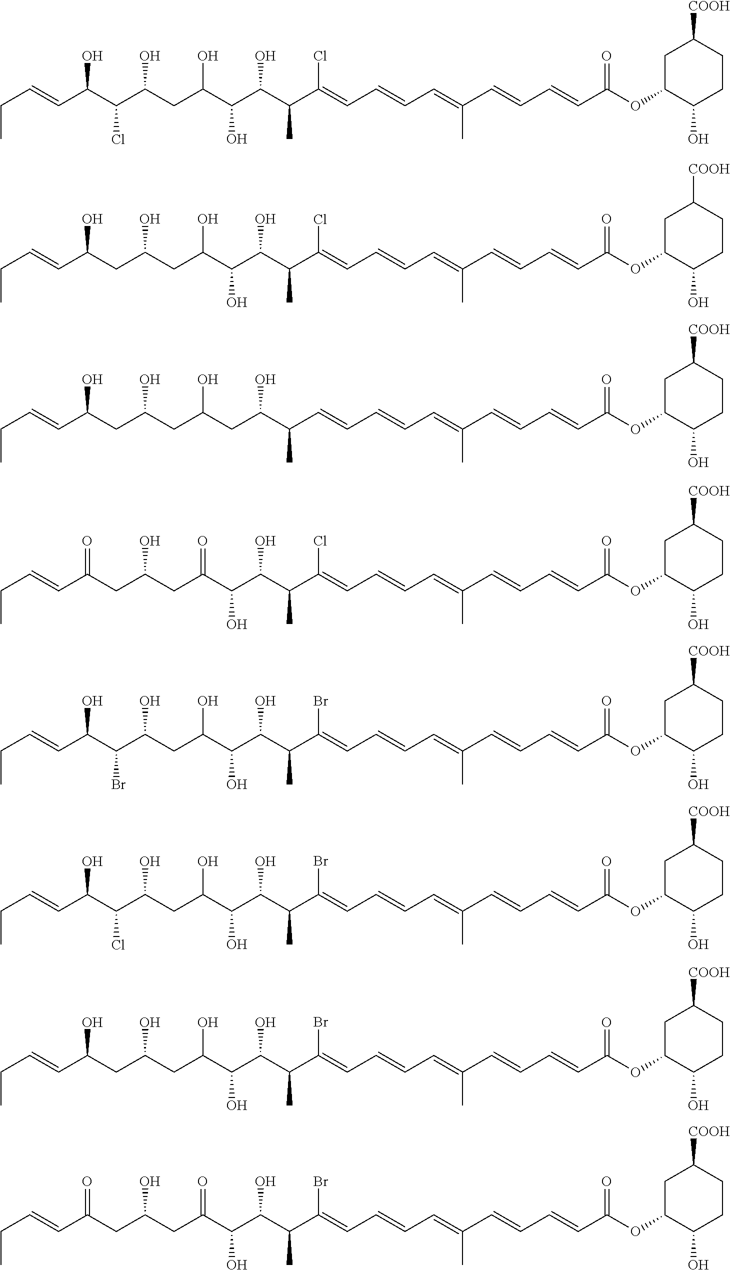

### Second Aspect

In a second aspect the invention provides compounds in which the ester linkage in the polyketide chain of enacyloxin IIa is replaced by an amide linkage. Such compounds may also differ from enacyloxin IIa at other key positions on the polyketide chain (for example, by modification of the substituent groups at one or more of positions C11, C14, C15, C18, C19 and C21) and/or in respect of modifications made to the terminal dihydroxycyclohexane carboxylic acid (DHCCA) moiety.

Viewed from a second aspect the invention provides a compound of formula (II), or a pharmaceutically acceptable salt, metabolite, isomer (e.g. stereoisomer) or prodrug thereof:

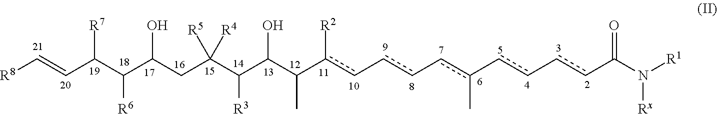

wherein:
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁷ is H, OH, or —OC(O)NR′₂ (where each R′ is independently H or C₁₋₃alkyl, e.g. CH₃), preferably
R⁷ is H, OH or —OC(O)NH₂;
R⁸ is a straight-chained or branched C₁₋₈ alkyl group (e.g. C₁₋₆ alkyl group);
R^{x} is either H or C₁₋₃ alkyl, e.g. CH₃; and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment of formula (II), R¹ is a cyclohexyl or cyclopentyl ring which is optionally substituted by one or more substituents. In another embodiment of formula (II), R¹ is a cyclohexenyl ring which is optionally substituted by one or more substituents.

In another embodiment of formula (II), R¹ is a straight-chained or branched C₁₋₆ alkyl group (e.g. C₁₋₃ alkyl group) which may be substituted by one or more substituents. Preferably it is a straight-chained alkyl group.

Optional substituents which may be present in group R¹ include one or more of the following: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof). Suitable ester-forming groups include optionally substituted alkyl, alkenyl, cycloalkyl, cycloalkenyl, aryl and heteroaryl groups. Examples of such groups include optionally substituted C₁₋₁₂-alkyl, C₁₋₁₂-alkenyl, C₃₋₁₀-cycloalkyl, aryl and heteroaryl groups, wherein the aryl and heteroaryl groups may contain from 5 to 10 carbon atoms and the heteroaryl groups further contain one or more (e.g. 1, 2, 3 or 4) heteroatoms selected from N, O and S. In one embodiment, the substituents which may be present in group R¹ may be selected from any of the following: OH, NH₂, SH, F, Cl, Br, I, CH₃, CO₂H, PO₃H₂ and SO₃H₂.

In one embodiment, one, two or three (preferably one or two) substituents may be present in group R¹. Where more than one substituent is present, these may be the same or different. Preferably, at least one of the substituents will be CO₂H or an ester thereof as herein defined.

In one embodiment, where R¹ is substituted by more than one substituent (e.g. two or three substituents), the substituents may be selected from the group consisting of CO₂H (or an ester thereof), and OH. Preferably R¹ may be substituted by one CO₂H group (or an ester thereof), and/or by one OH group, e.g. by one CO₂H group (or an ester thereof), and by one OH group.

In one embodiment, R¹ is an optionally substituted cyclohexyl or cyclopentyl group. Substituents on these rings may be any of those herein described. Preferably, the substituents may be selected from CO₂H (or an ester thereof), and OH. In one embodiment, R¹ is a cyclohexyl ring substituted by one CO₂H group (or an ester thereof), and by one OH group. These substituents may be present at any ring positions, but in one embodiment these may be para to one another.

Where R¹ is a straight-chained or branched C₁₋₆ alkyl group (e.g. C₁₋₃ alkyl group), this is preferably substituted. Preferred substituents are selected from CO₂H (or an ester thereof), and OH. In one embodiment, R¹ is a straight-chained or branched (preferably straight-chained) C₁₋₆ alkyl group (e.g. C₁₋₃ alkyl group) substituted by one CO₂H group (or an ester thereof), and/or by one OH group. Where present, any CO₂H group (or an ester thereof) will typically be provided at the terminal position of the alkyl group.

Examples of R¹ groups include any of the following (in which * denotes the point of attachment of the substituent to the remainder of the molecule):

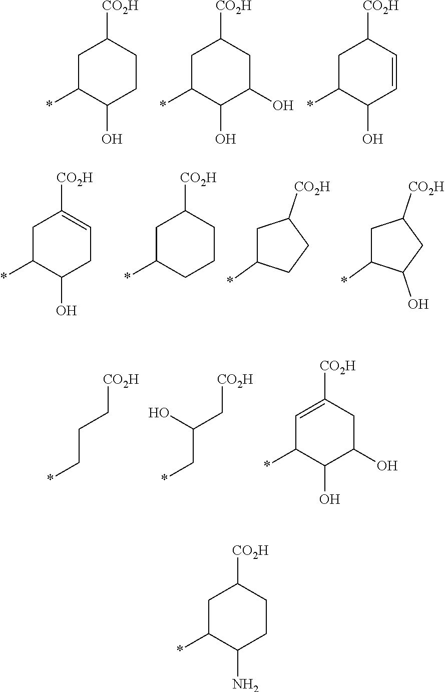

In one embodiment of formula (II), R² is F, Cl, Br or I. In a preferred embodiment, R² is Cl.

In one embodiment of formula (II), R³ is OH.

In one embodiment of formula (II), R⁴ and R⁵ together are ═O.

In one embodiment of formula (II), R⁶ is Cl.

In one embodiment of formula (II), R⁷ is —OC(O)NH₂.

In one embodiment of formula (II), R⁸ is a straight-chained or branched C₁₋₅ alkyl, preferably a straight-chained or branched C₁₋₄ alkyl. Examples of such groups include methyl, ethyl, isopropyl, and tert. butyl. In a preferred embodiment, R⁸ is ethyl.

In one embodiment of formula (II), each of C₂-C₃, C₄-C₅, C₆-C₇, C₈-C₉ and C₁₀-C₁₁ are C═C (double) bonds.

In one embodiment of formula (II), R¹ is a substituted cyclohexyl group and R⁸ is ethyl.

In one embodiment, the invention provides a compound of formula (IIa) or a pharmaceutically acceptable salt, metabolite, isomer (e.g. stereoisomer) or prodrug thereof:

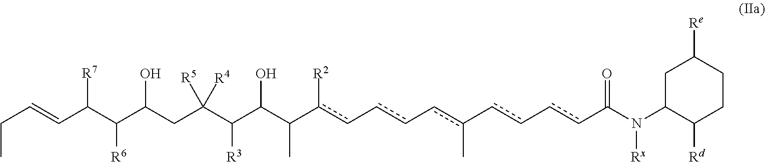

wherein:
R^{d} is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or
C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R² to R⁷, and R^{X} are as herein defined; and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment, the invention provides compounds of formula (IIa) and their pharmaceutically acceptable salts, metabolites, isomers (e.g. stereoisomers) and prodrugs, wherein:
R^{d} is H, OH, NH₂, SH, F, Cl, Br, I, or CH₃, preferably H, OH or NH₂;
R^{e} is H, CO₂H, PO₃H₂, or SO₃H₂, preferably CO₂H;
R² is F, Cl, Br or I, preferably Cl;
R³ is OH;
R⁴ and R⁵ together are ═O;
R⁶ is F, Cl, Br or I, preferably Cl;
R⁷ is —OC(O)NH₂;
R^{x} is H or CH₃, preferably H; and
C₂-C₃, C₄-C₅, C₆-C₇, C₈-C₉ and C₁₀-C₁₁ are independently either C—C (single) or C═C (double) bonds;

In one embodiment of formula (IIa), each of C₂-C₃, C₄-C₅, C₆-C₇, C₈-C₉ and C₁₀-C₁₁ are C═C (double) bonds.

In one embodiment, the invention provides a compound of formula (IIIb), or a pharmaceutically acceptable salt, metabolite, isomer (e.g. stereoisomer) or prodrug thereof:

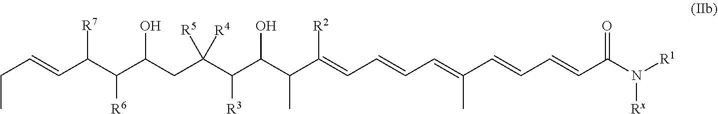

wherein R¹ to R⁷, and R^{x} are as herein defined.

In another embodiment, the invention provides a compound of formula (IIc), or a pharmaceutically acceptable salt, metabolite, or prodrug thereof:

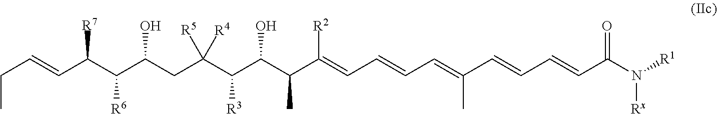

wherein R¹ to R⁷, and R^{x} are as herein defined.

In one embodiment of formula (IIb) and (IIc), R^{x} is H, R² is Cl, R³ is OH, R⁴ and R⁵ together are ═O, R⁶ is Cl and R⁷ is —OC(O)NH₂. R¹ is as herein defined, preferably a substituted cyclohexyl or substituted cyclopentyl group.

Examples of compounds of formula (II) according to the invention include the following, and their pharmaceutically acceptable salts, metabolites, isomers (e.g. stereoisomers) and prodrugs:

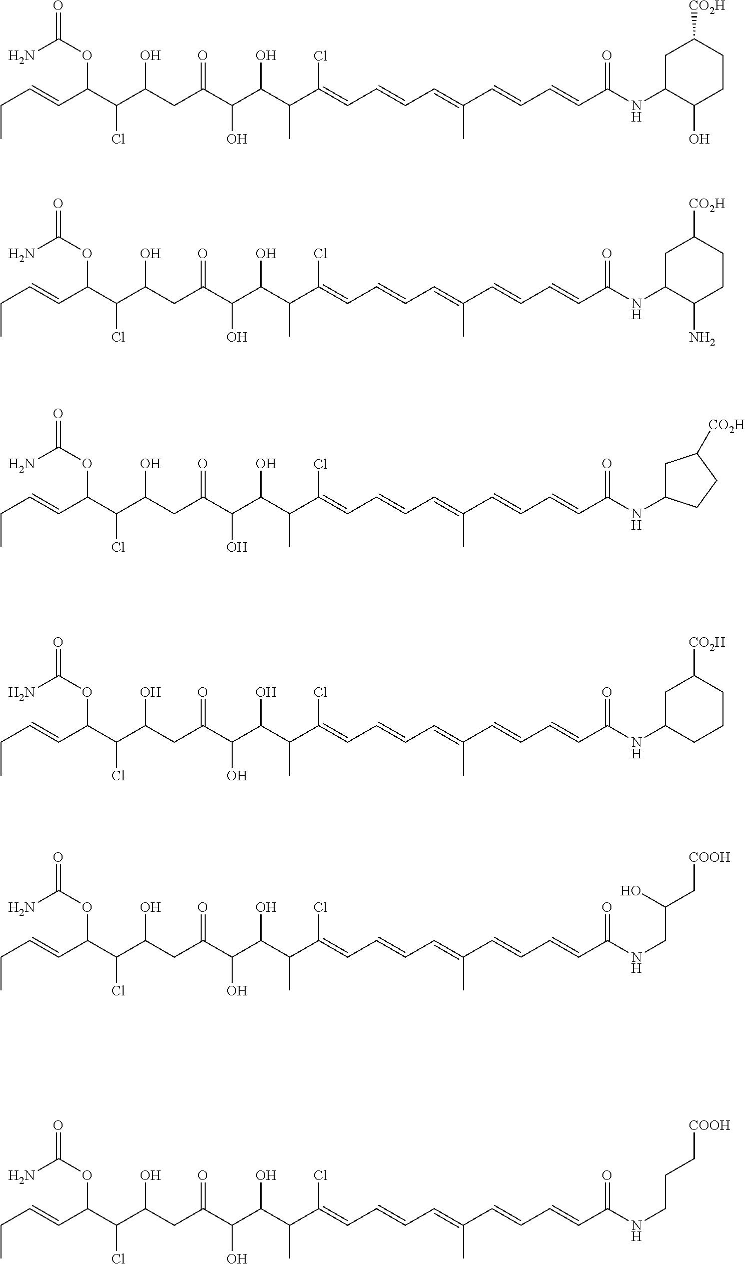

Further examples of compounds of formula (II) according to the invention include the following, and their pharmaceutically acceptable salts, metabolites, and prodrugs:

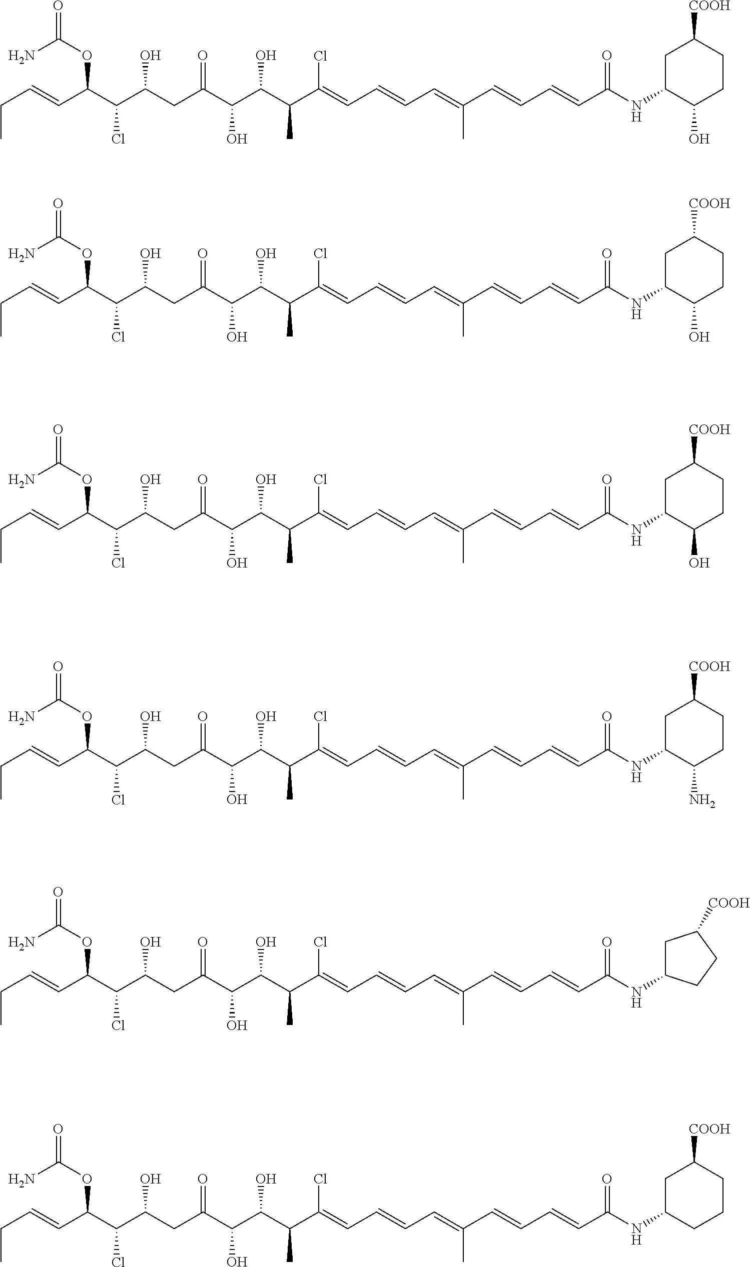

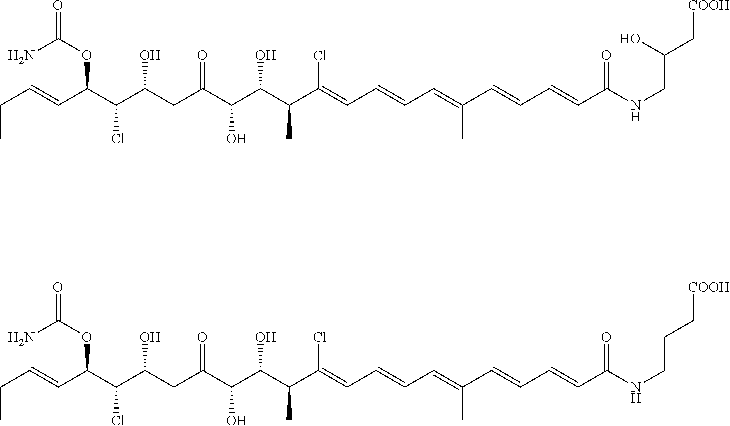 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)NCC(O)CC(=O)O ; CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)NCCCC(=O)O]

### Third Aspect

In a third aspect the invention provides compounds in which the OH and CO₂H substituents on the dihydroxycyclohexane carboxylic acid (DHCCA) moiety of enacyloxin IIa have different absolute or relative stereochemistries compared to enacyloxin IIa. Such compounds may also differ from enacyloxin IIa at other key positions on the polyketide chain (for example, by modification of the substituent groups at one or more of positions C11, C14, C15, C18, C19 and C21).

Viewed from a third aspect the invention provides compounds of formula (IIIa) and (IIIb), pharmaceutically acceptable salts, metabolites, or prodrugs thereof:

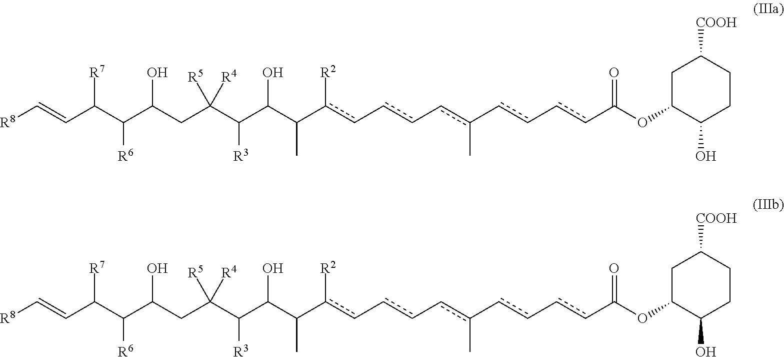

wherein:
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁷ is H, OH, or —OC(O)NR′₂ (where each R′ is independently H or C₁₋₃ alkyl, e.g. CH₃), preferably
R⁷ is H, OH or —OC(O)NH₂;
R⁸ is a straight-chained or branched C₁₋₈ alkyl group (e.g. C₁₋₆ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds).

In the compounds of formula (IIIa) and (IIIb) the stereochemistry at any of positions C6, C11 to C15 and C17 to C19 in the polyketide chain may vary depending on the nature of the particular substituent groups at these positions. All such stereoisomers are considered to form part of the invention.

In one embodiment of formula (IIIa) and (IIIb), R² is F, Cl, Br or I. In a preferred embodiment, R² is Cl.

In one embodiment of formula (IIIa) and (IIIb), R³ is OH.

In one embodiment of formula (IIIa) and (IIIb), R⁴ and R⁵ together are ═O.

In one embodiment of formula (IIIa) and (IIIb), R⁶ is Cl;

In one embodiment of formula (IIIa) and (IIIb), R⁷ is —OC(O)NH₂.

In one embodiment of formula (IIIa) and (IIIb), R⁸ is a straight-chained or branched C₁₋₅ alkyl, preferably a straight-chained or branched C₁₋₄ alkyl. Examples of such groups include methyl, ethyl, isopropyl, and tert. butyl. In a preferred embodiment, R⁸ is ethyl.

In one embodiment of formula (IIIa) and (IIIb), each of C₂-C₃, C₄-C₅, C₆-C₇, C₈-C₉ and C₁₀-C₁₁ are C═C (double) bonds.

In one embodiment, the invention provides compounds of formula (IIIc) and (IIId), or a pharmaceutically acceptable salt, metabolite, isomer (e.g. stereoisomer) or prodrug thereof:

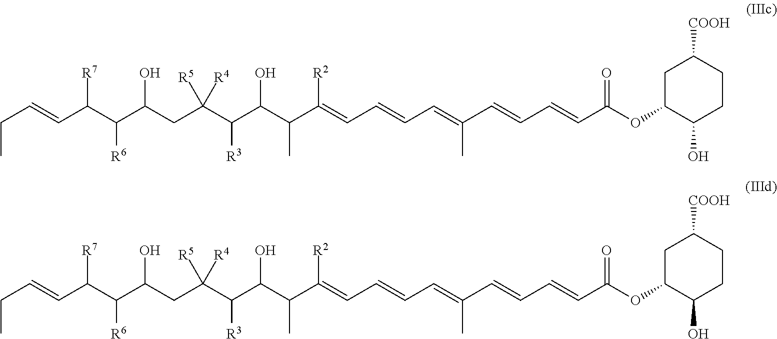

wherein R² to R⁷ are as herein defined.

In another embodiment, the invention provides compounds of formula (IIIe) and (IIIf), or a pharmaceutically acceptable salt, metabolite or prodrug thereof:

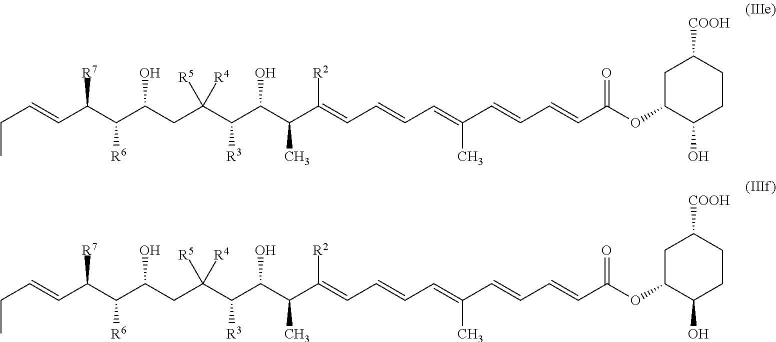

wherein R² to R⁷ are as herein defined.

Examples of compounds of formula (IIIa) and (IIIb) according to the invention include the following, and their pharmaceutically acceptable salts, metabolites, isomers (e.g. stereoisomers) and prodrugs:

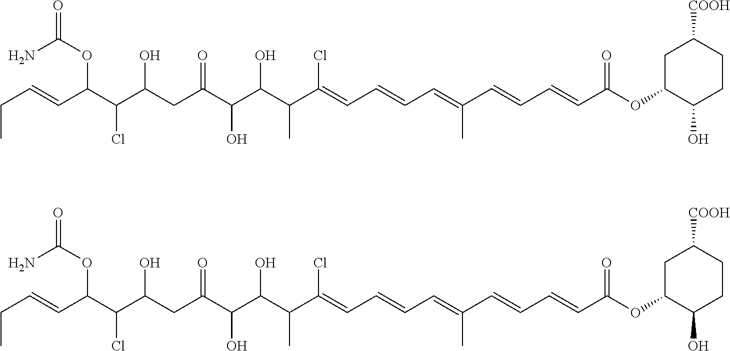 [SMILES: CC/C=C/C(OC(N)=O)C(Cl)C(O)CC(=O)C(O)C(O)C(C)/C(Cl)=C/C=C/C=C(C)/C=C/C=C/C(=O)O[C@@H]1C[C@H](C(=O)O)CC[C@@H]1O ; CC/C=C/C(OC(N)=O)C(Cl)C(O)CC(=O)C(O)C(O)C(C)/C(Cl)=C/C=C/C=C(C)/C=C/C=C/C(=O)O[C@@H]1C[C@H](C(=O)O)CC[C@H]1O]

Further examples of compounds of formula (IIIa) and (IIIb) according to the invention include the following, and their pharmaceutically acceptable salts, metabolites, and prodrugs:

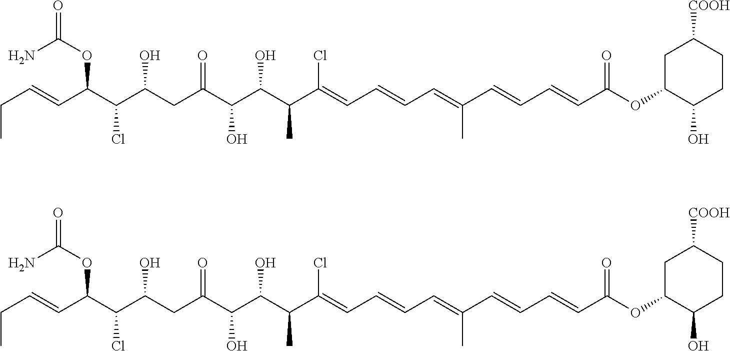 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@H](C(=O)O)CC[C@@H]1O ; CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@H](C(=O)O)CC[C@H]1O]

### Fourth Aspect

In a fourth aspect the invention provides compounds in which the terminal dihydroxycyclohexane carboxylic acid (DHCCA) moiety in enacyloxin IIa is modified, for example by the presence of additional substituents on the cyclohexane ring, or by replacement of the cyclohexane ring by a 5-membered carbocyclic ring or an unsaturated 6-membered carbocylic ring. Such compounds may also differ from enacyloxin IIa at other key positions on the polyketide chain (for example, by modification of the substituent groups at one or more of positions C11, C14, C15, C18, C19 and C21).

Viewed from a fourth aspect the invention thus provides a compound of formula (V), or a pharmaceutically acceptable salt, metabolite, isomer (e.g. stereoisomer) or prodrug thereof:

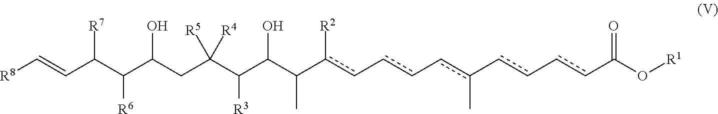

wherein:
R¹ is a 5-membered, saturated or unsaturated, carbocyclic ring substituted by one or more substituents, or
R¹ is a 6-membered, unsaturated, carbocyclic ring substituted by one or more substituents, or
R¹ is a 6-membered, saturated, carbocyclic ring substituted by one or three substituents;
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁷ is H, OH, or —OC(O)NR′₂ (where each R′ is independently H or C₁₋₃alkyl, e.g. CH₃), preferably
R⁷ is H, OH or —OC(O)NH₂;
R⁸ is a straight-chained or branched C₁₋₈ alkyl group (e.g. C₁₋₆ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment of formula (V), R¹ is a substituted cyclohexyl or cyclopentyl ring. Where R¹ is a cyclohexyl ring it is substituted by one or three substituents. Where R¹ is a cyclopentyl ring it is substituted by one or more substituents, for example one or two substituents.

In another embodiment of formula (V), R¹ is a cyclohexenyl ring which is optionally substituted by one or more substituents.

Optional substituents which may be present in group R¹ include one or more of the following: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof). Suitable ester-forming groups include optionally substituted alkyl, alkenyl, cycloalkyl, cycloalkenyl, aryl and heteroaryl groups. Examples of such groups include optionally substituted C₁₋₁₂-alkyl, C₁₋₁₂-alkenyl, C₃₋₁₀-cycloalkyl, aryl and heteroaryl groups, wherein the aryl and heteroaryl groups may contain from 5 to 10 carbon atoms and the heteroaryl groups further contain one or more (e.g. 1, 2, 3 or 4) heteroatoms selected from N, O and S. In one embodiment, the substituents which may be present in group R¹ may be selected from any of the following: OH, NH₂, SH, F, Cl, Br, I, CH₃, CO₂H, PO₃H₂ and SO₃H₂.

Where R¹ is a 5-membered, saturated or unsaturated, carbocyclic ring or a 6-membered, unsaturated, carbocyclic ring, it may be substituted by one, two or three (preferably one or two) substituents. Where more than one substituent is present, these may be the same or different.

Preferably, at least one of the substituents will be CO₂H or an ester thereof as herein defined.

Where R¹ is a 6-membered, saturated, carbocyclic ring it may be substituted by one or three substituents. Where three substituents are present, these may be the same or different. Preferably, at least one of the substituents will be CO₂H or an ester thereof as herein defined.

In one embodiment, where R¹ is substituted by more than one substituent (e.g. two or three substituents), the substituents may be selected from the group consisting of CO₂H (or an ester thereof), and OH. In one embodiment, R¹ may be substituted by one CO₂H group (or an ester thereof), and/or by one OH group, e.g. by one CO₂H group (or an ester thereof), and by one OH group. In another embodiment, R¹ may be substituted by one CO₂H group (or an ester thereof), and/or by two OH groups, e.g. by one CO₂H group (or an ester thereof) and by two OH groups.

In one embodiment, R¹ is a substituted cyclopentyl group. Substituents on this ring may be any of those herein described. Preferably, the substituents may be selected from CO₂H (or an ester thereof), and OH. In one embodiment, R¹ is a cyclopentyl ring substituted by one CO₂H group (or an ester thereof), and by one OH group. These substituents may be present at any ring positions.

In another embodiment, R¹ is a substituted cyclohexyl group. Substituents on this ring may be any of those herein described. Preferably, the substituents may be selected from CO₂H (or an ester thereof), and OH. In one embodiment, R¹ is a cyclohexyl ring substituted by one CO₂H group (or an ester thereof). In another embodiment, R¹ is a cyclohexyl ring substituted by one CO₂H group (or an ester thereof), and by two OH groups. These substituents may be present at any ring positions.

In another embodiment, R¹ is a substituted cyclohexenyl group. Substituents on this ring may be any of those herein described. Preferably, the substituents may be selected from CO₂H (or an ester thereof), and OH. In one embodiment, R¹ is a cyclohexenyl ring substituted by one CO₂H group (or an ester thereof), and by one OH group. These substituents may be present at any ring positions. In another embodiment, R¹ is a cyclohexenyl ring substituted by one CO₂H group (or an ester thereof), and by two OH groups. These substituents may be present at any ring positions.

Examples of R¹ groups in formula (V) include any of the following (in which * denotes the point of attachment of the substituent to the remainder of the molecule):

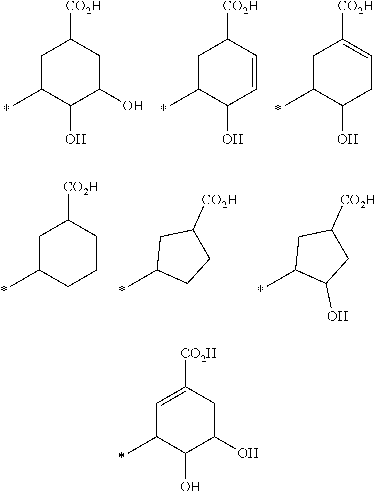

In one embodiment of formula (V), R² is F, Cl, Br or I. In a preferred embodiment, R² is Cl;

In one embodiment of formula (V), R³ is OH.

In one embodiment of formula (V), R⁴ and R⁵ together are ═O. In another embodiment, R⁴ is H and R⁵ is OH.

In one embodiment of formula (V), R⁶ is Cl.

In one embodiment of formula (V), R⁷ is —OC(O)NH₂. In another embodiment, R⁷ is OH.

In one embodiment of formula (V), R⁸ is a straight-chained or branched C₁₋₅ alkyl, preferably a straight-chained or branched C₁₋₄ alkyl. Examples of such groups include methyl, ethyl, isopropyl, and tert. butyl. In a preferred embodiment, R⁸ is ethyl.

In one embodiment of formula (V), each of C₂-C₃, C₄-C₅, C₆-C₇, C₈-C₉ and C₁₀-C₁₁ are C═C (double) bonds.

In one embodiment, the invention provides a compound of formula (Va) or a pharmaceutically acceptable, salt, metabolite, isomer (e.g. stereoisomer) or prodrug thereof:

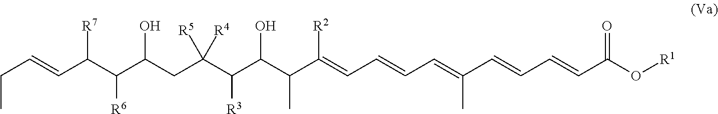

wherein R¹ to R⁷ are as herein defined.

In another embodiment, the invention provides a compound of formula (Vb), or a pharmaceutically acceptable salt, metabolite, or prodrug thereof:

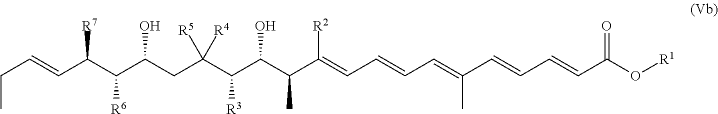

wherein R¹ to R⁷ are as herein defined.

In one embodiment of formula (Va) or formula (Vb), R² is Cl.

In one embodiment of formula (Va) or formula (Vb), R³ is OH.

In one embodiment of formula (Va) or formula (Vb), R⁴ and R⁵ together are ═O. In another embodiment, R⁴ is H and R⁵ is OH.

In one embodiment of formula (Va) or formula (Vb), R⁶ is Cl.

In one embodiment of formula (Va) or formula (Vb), R⁷ is —OC(O)NH₂.

Examples of compounds of formula (V) according to the invention include the following, and their pharmaceutically acceptable salts, metabolites, isomers (e.g. stereoisomers) and prodrugs:

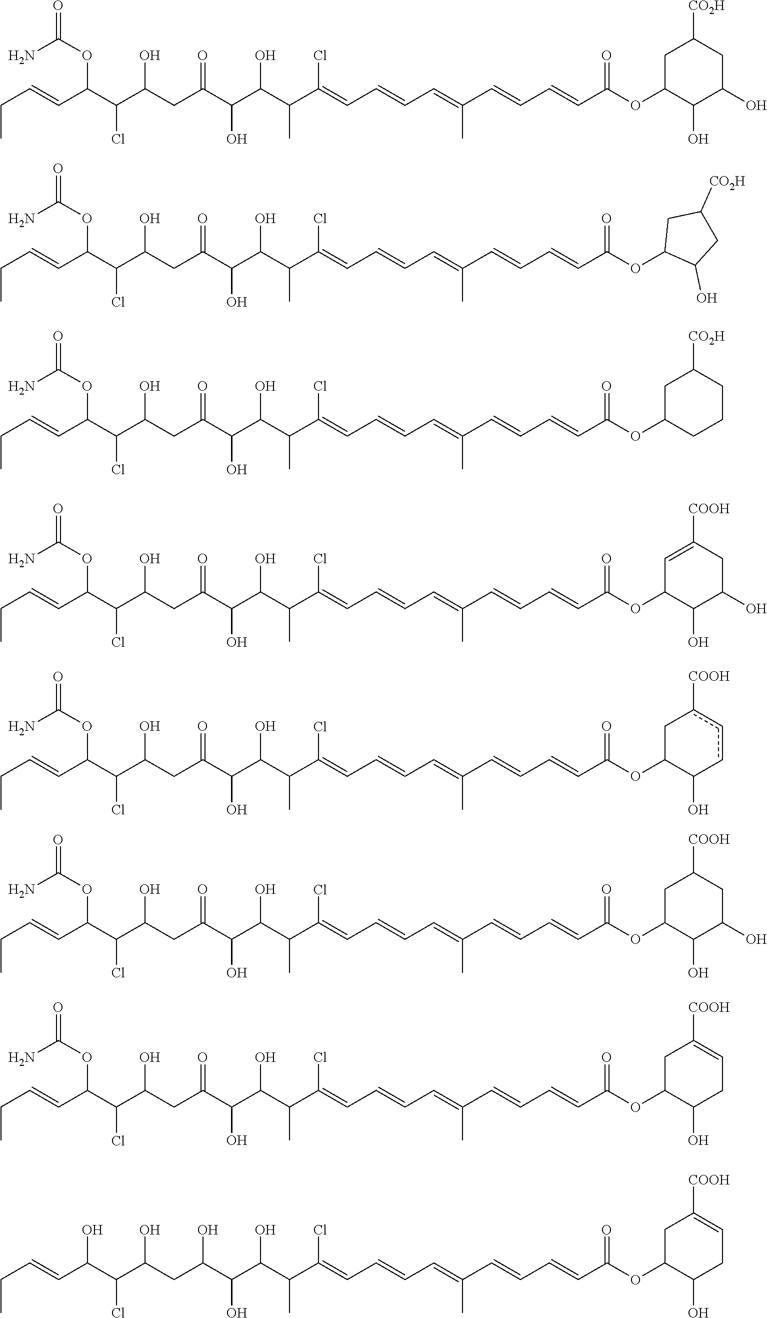

Further examples of compounds of formula (V) according to the invention include the following, and their pharmaceutically acceptable salts, metabolites, and prodrugs:

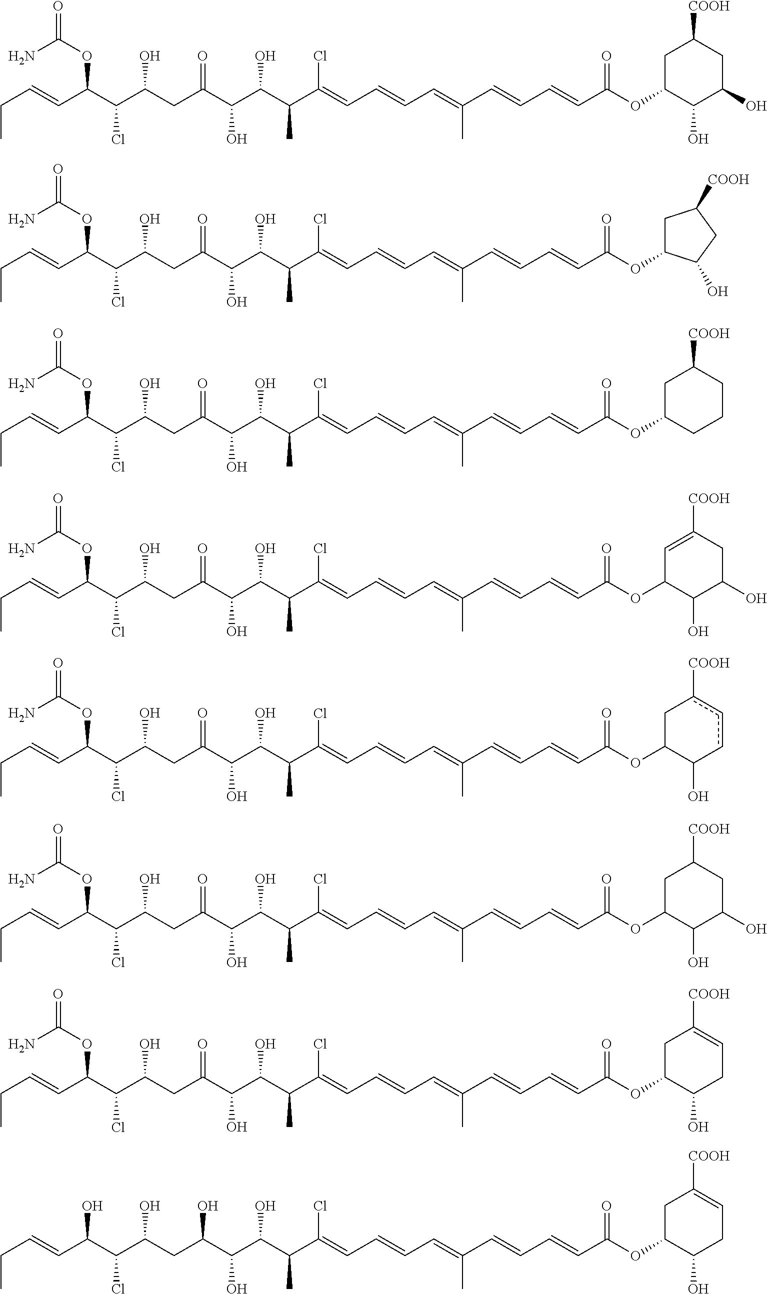

### Fifth Aspect

In a fifth aspect the invention provides compounds in which one or more groups at positions C11, C14, C15 or C18 of enacyloxin IIa are modified compared to the parent molecule by replacement of the substituents at these positions by H. Such compounds may also differ from enacyloxin IIa at other key positions on the polyketide chain (for example, by modification of the substituent groups at C21) and/or in respect of modifications made to the terminal dihydroxycyclohexane carboxylic acid (DHCCA) moiety.

Viewed from a fifth aspect the invention thus provides a compound of formula (VI), or a pharmaceutically acceptable salt, metabolite, isomer (e.g. stereoisomer) or prodrug thereof:

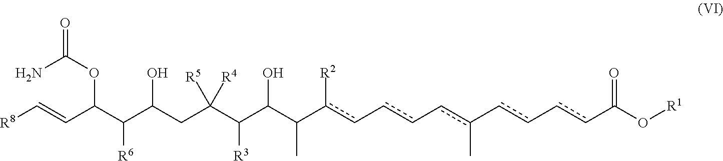

wherein:
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁸ is a straight-chained or branched C₁₋₈ alkyl group (e.g. C₁₋₆ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds;
with the proviso that either at least one of R², R³ and R⁶ is H, or R⁴ is H and R⁵ is OH).

In one embodiment of formula (VI), R¹ is a cyclohexyl or cyclopentyl ring which is optionally substituted by one or more substituents. In another embodiment of formula (VI), R¹ is a cyclohexenyl ring which is optionally substituted by one or more substituents.

In another embodiment of formula (VI), R¹ is a straight-chained or branched C₁₋₆ alkyl group (e.g. C₁₋₃ alkyl group) which may be substituted by one or more substituents. Preferably it is a straight-chained alkyl group.

Optional substituents which may be present in group R¹ include one or more of the following: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof). Suitable ester-forming groups include optionally substituted alkyl, alkenyl, cycloalkyl, cycloalkenyl, aryl and heteroaryl groups. Examples of such groups include optionally substituted C₁₋₁₂-alkyl, C₁₋₁₂-alkenyl, C₃₋₁₀-cycloalkyl, aryl and heteroaryl groups, wherein the aryl and heteroaryl groups may contain from 5 to 10 carbon atoms and the heteroaryl groups further contain one or more (e.g. 1, 2, 3 or 4) heteroatoms selected from N, O and S. In one embodiment, the substituents which may be present in group R¹ may be selected from any of the following: OH, NH₂, SH, F, Cl, Br, I, CH₃, CO₂H, PO₃H₂ and SO₃H₂.

In one embodiment, one, two or three (preferably one or two) substituents may be present in group R¹. Where more than one substituent is present, these may be the same or different. Preferably, at least one of the substituents will be CO₂H or an ester thereof as herein defined.

In one embodiment, where R¹ is substituted by more than one substituent (e.g. two or three substituents), the substituents may be selected from the group consisting of CO₂H (or an ester thereof), and OH. Preferably R¹ may be substituted by one CO₂H group (or an ester thereof), and/or by one OH group, e.g. by one CO₂H group (or an ester thereof), and by one OH group.

In one embodiment, R¹ is an optionally substituted cyclohexyl or cyclopentyl group. Substituents on these rings may be any of those herein described. Preferably, the substituents may be selected from CO₂H (or an ester thereof), and OH. In one embodiment, R¹ is a cyclohexyl ring substituted by one CO₂H group (or an ester thereof), and by one OH group. These substituents may be present at any ring positions, but in one embodiment these may be para to one another.

Where R¹ is a straight-chained or branched C₁₋₆ alkyl group (e.g. C₁₋₃ alkyl group), this is preferably substituted. Preferred substituents are selected from CO₂H (or an ester thereof), and OH. In one embodiment, R¹ is a straight-chained or branched (preferably straight-chained) C₁₋₆ alkyl group (e.g. C₁₋₃ alkyl group) substituted by one CO₂H group (or an ester thereof), and/or by one OH group. Where present, any CO₂H group (or an ester thereof) will typically be provided at the terminal position of the alkyl group.

Examples of R¹ groups include any of the following (in which * denotes the point of attachment of the substituent to the remainder of the molecule):

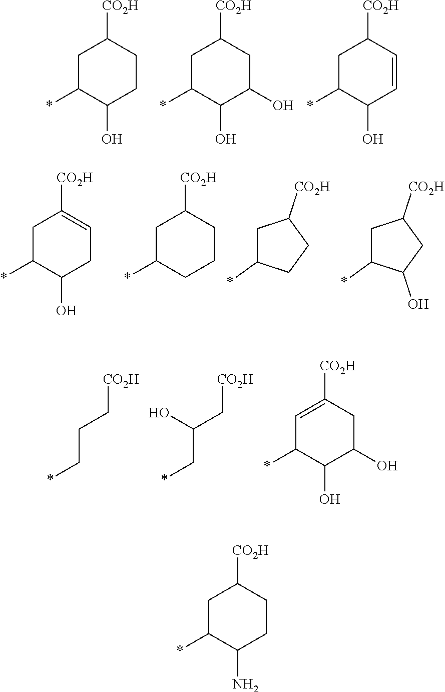

In one embodiment of formula (VI), R² is H, F, Cl, Br or I. In a preferred embodiment, R² is H or Cl. In another embodiment, R² is Br.

In one embodiment of formula (VI), R³ is OH. In another embodiment of formula (VI), R³ is H.

In one embodiment of formula (VI), R⁴ and R⁵ together are ═O. In another embodiment of formula (VI), R⁴ is H and R⁵ is OH.

In one embodiment of formula (VI), R⁶ is H or Cl.

In one embodiment of formula (VI), R⁶ is Br.

In one embodiment of formula (VI), R² and R⁶ are both Br. In another embodiment, R² is Br and R⁶ is either H or Cl.

In one embodiment of formula (VI), R⁸ is a straight-chained or branched C₁₋₅ alkyl, preferably a straight-chained or branched C₁₋₄ alkyl. Examples of such groups include methyl, ethyl, isopropyl, and tert. butyl. In a preferred embodiment, R⁸ is ethyl.

In one embodiment of formula (VI), each of C₂-C₃, C₄-C₅, C₆-C₇, C₈-C₉ and C₁₀-C₁₁ are C═C (double) bonds.

In one embodiment of formula (VI), R² is H.

In one embodiment of formula (VI), R³ is H.

In one embodiment of formula (VI), R⁶ is H.

In one embodiment of formula (VI), R⁴ is H and R⁵ is OH.

In one embodiment of formula (VI), R¹ is a substituted cyclohexyl group and R⁸ is ethyl.

In one embodiment, the invention provides a compound of formula (VIa) or a pharmaceutically acceptable, salt, metabolite, isomer (e.g. stereoisomer) or prodrug thereof:

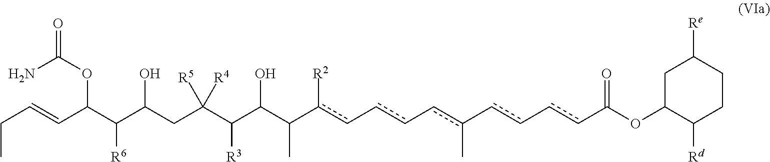

wherein:
R^{d} is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or
C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R² to R⁶ are as herein defined; and
each — independently represents an optional bond (i.e. each of C₂-C₃, C₄-C₅, C₆-C₇, C₈-C₉ and C₁₀-C₁₁ are independently either C—C (single) or C═C (double) bonds).

In one embodiment, the invention provides compounds of formula (Via) and their pharmaceutically acceptable salts, metabolites, isomers (e.g. stereoisomers) and prodrugs, wherein:
R^{d} is H, OH, NH₂, SH, F, Cl, Br, I, or CH₃, preferably OH;
R^{e} is H, CO₂H, PO₃H₂, or SO₃H₂, preferably CO₂H;
each of C₂-C₃, C₄-C₅, C₆-C₇, C₈-C₉, and C₁₀-C₁₁ are independently either C—C (single) or C═C (double) bonds;
R² is H, Cl or Br, e.g. R² is H or Cl;
R³ is H or OH;
R⁴ is H and R⁵ is OH; and
R⁶ is H, Cl or Br, e.g. R⁶ is H or Cl.

In one embodiment of formula (Via), each of C₂-C₃, C₄-C₅, C₆-C₇, C₈-C₉ and C₁₀-C₁₁ are C═C (double) bonds.

In one embodiment, the invention provides a compound of formula (VIb), or a pharmaceutically acceptable salt, metabolite, isomer (e.g. stereoisomer) or prodrug thereof:

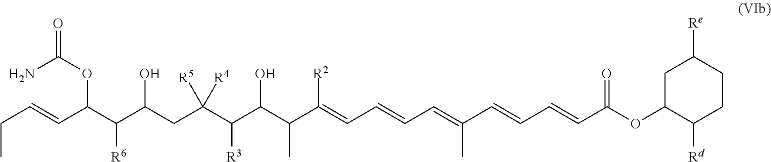

wherein R^{d}, R^{e}, and R² to R⁶ are as herein defined.

In another embodiment, the invention provides a compound of formula (VIc), or a pharmaceutically acceptable salt, metabolite, or prodrug thereof:

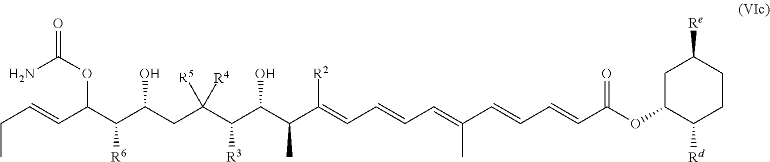

wherein R^{d}, R^{e}, and R² to R⁶ are as herein defined.

In one embodiment of formula (VIa), formula (VIb) or formula (VIc), R^{d} is OH and R^{e} is CO₂H.

Examples of compounds of formula (VI) according to the invention include the following, and their pharmaceutically acceptable salts, metabolites, isomers (e.g. stereoisomers) and prodrugs:

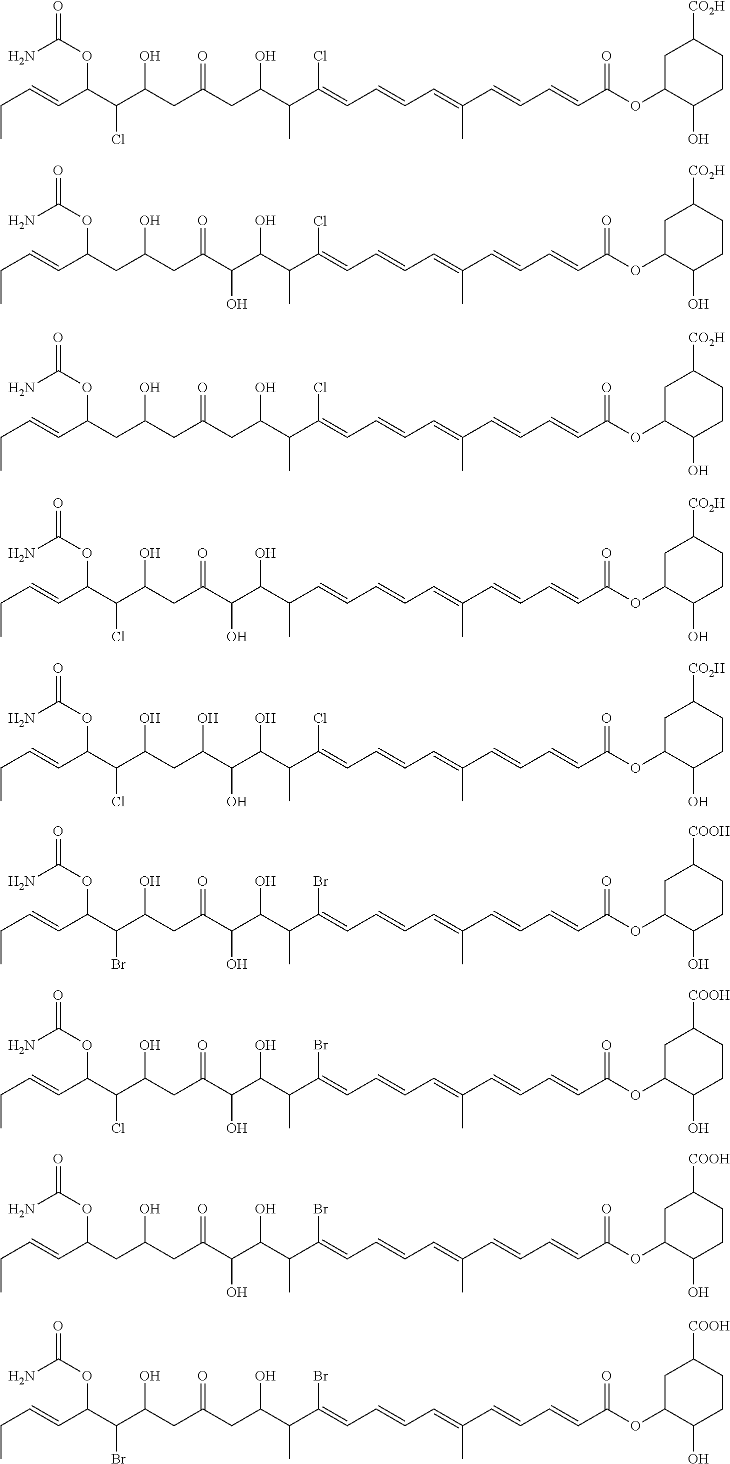

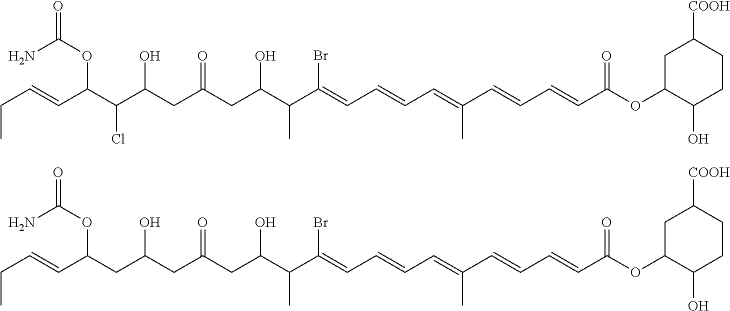

Further examples of compounds of formula (VI) according to the invention include the following, and their pharmaceutically acceptable salts, metabolites, and prodrugs:

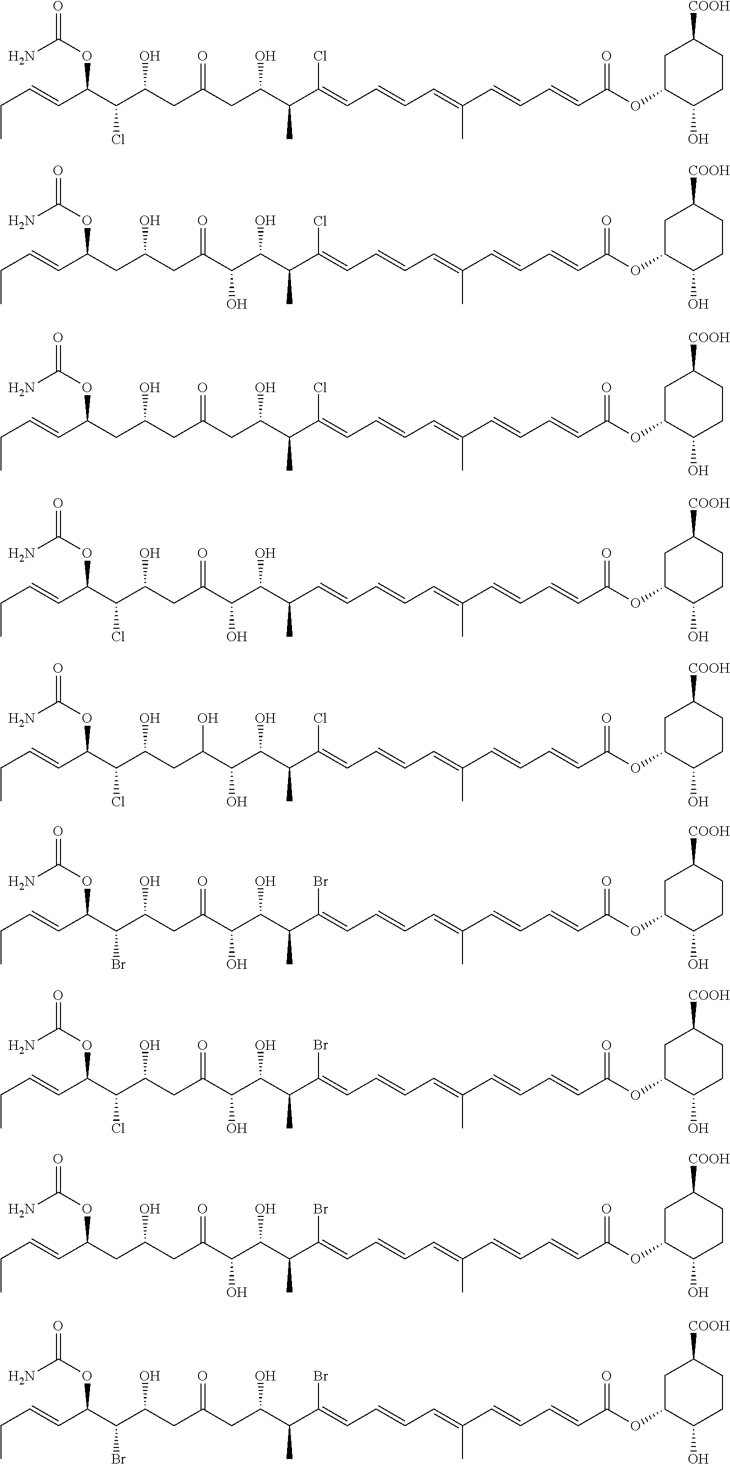

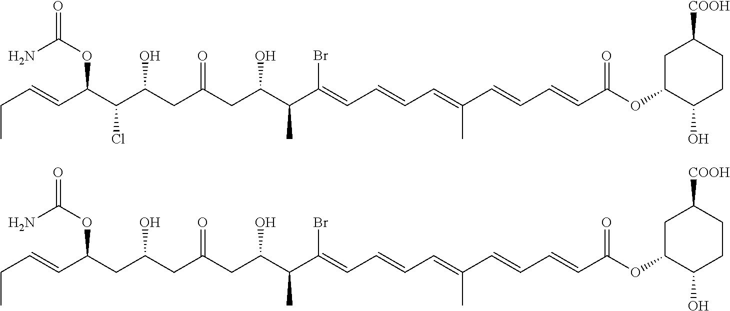

The compounds of the invention are suitable for pharmaceutical and medical use, in particular they are useful as antimicrobial agents. More specifically, the compounds of the present invention provide new agents for application against bacteria, multidrug-resistant bacteria and combinations thereof thus offering both separate and combination treatment potential. The compounds of the present invention have application for the treatment of various infections, for example including infections of the skin and skin structure, infections of the respiratory system, endocarditis, hospital acquired infections, infections of the digestive system, urinary system, nervous system, blood infection, soft tissue infection, nasal canal infections and infection associated with cystic fibrosis. The compounds of the present invention also find application in relation to or for animal/veterinary illnesses.

Thus, in another aspect, the invention provides a pharmaceutical composition comprising a compound according to the invention or a pharmaceutically acceptable salt, metabolite, isomer (e.g. stereoisomer) or prodrug thereof along with one or more physiologically acceptable carriers, excipients or diluents.

Also provided are methods of treating infections (such as those listed above) comprising administration of one or more compounds of the invention, optionally in combination with one or more further active agents.

In a related aspect, the invention provides a compound as defined herein for use as a medicament or in therapy, e.g. for use in the treatment of infections such as those listed above. In one embodiment, the compounds of the invention may be used to treat infections caused by a microbe which is resistant to known antimicrobial agents.

Where compounds of the invention are used in the treatment of an infection caused by a microbe, the microbe may be a Gram-negative bacterium. Such infectious Gram-negative bacteria are preferably selected from Acinetobacter species, Burkholderia species, Ralstonia species and Stenotrophomonas species. The bacterium may, for example, be Acinetobacter baumannii, Enterococcus faecium, Staphylococcus aureus, Klebsiella pneumoniae, Pseudomonas aeruginosa, or Enterobacter cloacae.

Preferably, the compounds according to the present invention are for use in the treatment of an infection caused by more than one type of microbe, for example, two or more different bacterial species.

Preferably, the compounds according to the present invention are for use in the treatment of an infection caused by a microbe that is resistant to at least one antimicrobial drug, for example an antimicrobial drug known in the art. The infection may be caused by one or more bacteria that show resistance to common antimicrobial drugs. The bacterium may be multidrug-resistant. For example, the infection may be caused by carbapenem-resistant Acinetobacter baumannii, or by MRSA or VRE.

The antimicrobial drug against which the microbe has become resistant may be an antibacterial drug. The antibacterial drug may be selected from, but is not limited to: drugs of the carbapenem family, drugs of the penicillin family, drugs of the vancomycin family, drugs of the aminoglycoside family, drugs of the quinolone family, drugs of the daptomycin family, drugs of the cephalosporin family, drugs of the macrolide family and combinations thereof. Examples of such antibacterial drugs include carbapenems, penicillin, ampicillin, methicillin, vancomycin, gentamycin, ofloxacin, ciprofloxacin, daptomycin, cefdimir, erythromycin, equivalents thereof, and combinations thereof.

Preferably, the compounds according to the present invention are for use in the treatment of an infection in an animal, preferably a mammal, more preferably a human. Preferably, the compounds according to the present invention are for use in the treatment of an infection in a non-human mammal, such as a dog, cat, horse, etc. The compounds according to the present invention therefore have application in both human and veterinary medicine.

Preferably, the compounds according to the present invention are for use in the treatment of an infection of the respiratory system, digestive system, urinary system, nervous system, a blood infection, a soft tissue infection, a skin infection, a nasal canal infection, or combinations thereof.

Preferably, the compounds according to the present invention are for use in the treatment of a bacterial infection of the respiratory system or a portion thereof, for example, the upper respiratory system.

Preferably, the compounds according to the present invention are for use in the treatment of an infection associated with immuno-compromised individuals, for example in the treatment of elderly or paediatric patients.

Preferably, the compounds and methods of the present invention are for use in treating a variety of infections that comprise different types of Gram-negative bacteria, including aerobic or anaerobic bacteria. These types of infections include intra-abdominal infections, pneumonia, bone and joint infections, and obstetrical/gynaecological infections and urinary tract infections.

The compounds and methods of the invention may also be used to treat an infection including, without limitation, endocarditis, nephritis, septic arthritis and osteomyelitis.

According to a further aspect of the present invention, there is provided a pharmaceutical composition comprising a compound according to the present invention, or a pharmaceutically acceptable salt, metabolite, or prodrug thereof, in combination with a pharmaceutically acceptable carrier. The pharmaceutical composition may further comprise one or more other therapeutic agents, for example selected from an anti-inflammatory agent, anti-cancer agent or immuno-modulatory agent, or different types of antibacterial and/or antifungal agents.

Preferably, a therapeutic agent, other than a compound of the present invention, may be administered concurrently with a compound of the present invention. In a preferred embodiment, an antibacterial and/or antifungal agent may be administered concurrently with a compound of the present invention. Co-administration of an antifungal agent and/or an antibacterial agent, other than a compound of the present invention, may be useful for mixed infections such as those caused by different types of bacteria, or those caused by both bacteria and fungi. The different therapeutic agents may be administered sequentially, separately or simultaneously.

Antibacterial agents and classes thereof that may be co-administered with a compound of the present invention preferably include, without limitation, penicillins and related drugs, carbapenems, cephalosporins and related drugs, aminoglycosides, ceftriaxone, daptomycins and macrolides.

Antifungal agents that may be co administered with a compound according to the present invention preferably include, without limitation, caspofungen, polyenes, such as amphotericin, nystatin and pimaricin; azoles, such as fluconazole, itraconazole, ketoconazole, voriconazole and sertaconazole; and allylamines, such as naftifine and terbinafine.

Another aspect of the present invention relates to the use of a compound according to the present invention for inhibiting the growth or survival of a microbe. The microbe may be resistant to at least one antimicrobial agent. The microbe is preferably a bacterium, for example at least one bacterium selected from Acinetobacter species, Mycobacterium species, Burkholderia species, Pseudomonas species, Ralstonia species, and Stenotrophomonas species.

According to another aspect of the present invention, there is provided a process for the preparation of a compound according to the present invention. Preferably, the process comprises cultivating a microorganism capable of producing a compound as herein described, such as Burkholderia ambifaria, e.g. one or more strains selected from BCCC0203 (also known as LMG P-24640; BCF), BCC0118 (also known as LMG P-24636; JLO), BCC 1248 (also known as LMG P-24641; KWO-1), BCC0250 (also known as LMG P-24637; WM2), BCC1241 (also known as LMG P-24639; KC311-6), BCC0207 (also known as LMG P-19182; AMMD; ATCC BAA-244; CCUG 44356; KCTC 12943; FC768; J2742 Vandamme R-696FC0768), and BCC0267 (also known as LMG P-19467; CEP0996; Coenye R-9935), or a mutant or variant thereof, optionally in the presence of any appropriate precursor compound such as those which are herein described.

In this aspect the term “variant” includes, but is not limited to, a bacterial strain that differs from the specified bacterial strain but which is able to produce at least one of compounds as described herein, e.g. according to the methods described herein. This term can also mean a bacterial strain that differs from the specified bacterial strain but which retains sufficient genotypic or phenotypic characteristics to maintain a taxonomic similarity.

In this aspect the term “mutant” includes, but is not limited to, a bacterial strain that has arisen as a result of mutation in, or gene editing of, the specified bacterial strain provided said mutant strain is able to produce at least one of the compounds as described herein, e.g. according to the methods described herein. This term can also mean a bacterial strain that differs from the specified bacterial strain as a result of mutation, or gene editing, which for example results in an altered gene, DNA sequence, enzyme, cell structure, etc.

Such mutants can be produced in a manner known in the art, for example by physical means such as irradiation (for example UV), by exposure to chemical mutagens or by genetic manipulation of DNA of the bacterium, e.g. to inactivate (e.g. to delete) certain biosynthetic genes. Methods for screening for mutants and isolating mutants will be known to a person skilled in the art.

Cultivation of the microorganism may be carried out in a culture or nutrient medium comprising a source of assimilable carbon, nitrogen, and inorganic salts, thereby producing a cultivation medium comprising the desired compound. Preferred nutrient media are agar-based (e.g. a BSM-agar supplemented with NaCl and glycerol). Where a precursor compound is supplied to the microorganism, e.g. a biosynthetic pathway blocked mutant, in order to produce certain compounds of the invention as herein described, this compound will typically be added to the nutrient medium. Where agar is used, for example, the precursor compound may be applied to the agar (e.g. at a concentration of about 10 mM) before spreading the chosen microorganism on top.

Optionally, the desired compound of the invention may be recovered from the cultivation medium or fermentation broth. The process may further comprise converting any compound obtained into an alternative compound according to the invention by known chemical syntheses. The process may also comprise converting the compound obtained into a pharmaceutically acceptable salt.

Conversion of any —COOH group to an ester derivative may be effected using methods which are known in the art (see, for example, March, J., Advanced Organic Chemistry, John Wiley & Sons, 4th edition, 1992). For example, a compound of the invention may be reacted with an optionally activated alkyl compound, such as a diazoalkane, to form the respective alkyl ester.

The compounds of the invention can be isolated and purified from the culture medium using known methods and taking account of the chemical, physical and biological properties of the natural substances. For the isolation, the compounds may be extracted from an agar culture or liquid culture using an organic solvent, such as methanol or ethyl acetate, and may be subjected to further purification.

The further purification of the compounds may be effected by chromatography on suitable materials, for example on reverse phase HPLC resins.

Insofar as the compounds herein described are present as stereoisomers, they can be separated using known methods, for example by means of separation using a chiral column.

Preferably, the producer microorganism is Burkholderia ambifaria, e.g. one or more strains selected from BCCC0203 (also known as LMG P-24640; BCF), BCC0118 (also known as LMG P-24636; JLO), BCC 1248 (also known as LMG P-24641; KWO-1), BCC0250 (also known as LMG P-24637; WM2), BCC1241 (also known as LMG P-24639; KC311-6), BCC0207 (also known as LMG P-19182; AMMD; ATCC BAA-244; CCUG 44356; KCTC 12943; FC768; J2742 Vandamme R-696FC0768), and BCC0267 (also known as LMG P-19467; CEP0996; Coenye R-9935), or a mutant or variant thereof as herein described. Other microorganisms, in particular bacteria, engineered to carry the appropriate biosynthetic genes may also be used.

Preferably, the nutrient medium in the process for the preparation of the compounds according to the present invention comprises glycerol as the sole carbon source. The glycerol may be present in an amount of between about 2 g/L and about 12 g/L, or between about 4 g/L and about 10 g/L, such as about 5 g/L.

Preferably, the method comprises incubating the bacterium on nutrient or minimal media up to and including at least part of the stationary phase. In preferred embodiments, the method comprises incubating the bacterium on minimal media for between about 16 hours and about 120 hours, or for between about 48 hours and about 96 hours, or for between about 48 hours and about 72 hours. In further preferred embodiments, the method comprises incubating the bacterium on minimal media for at least about 16 hours, or at least about 48 hours, or about 48 hours.

Preferably, the nutrient or minimal medium comprises a basal salts medium (BSM). Preferably, the basal salts medium comprises the formulation originally described by Hareland at al. (“Metabolic function and properties of 4-hydroxyphenylacetic acid 1-hydroxylase from Pseudomonas acidovorans”, J. Bacteriol. (1975) 121: 272-285).

The nutrient or minimal media may further comprise yeast extract. In a preferred embodiment, the yeast extract is present in an amount of between about 0.01% w/v and about 0.1% w/v, such as between about 0.025% w/v and about 0.075% w/v, or about 0.05% w/v.

The nutrient or minimal media may further comprise casamino acids. In a preferred embodiment, the casamino acids are present in an amount of between about 0.01% w/v and about 0.1% w/v, such as between about 0.025% w/v and about 0.075% w/v, or about 0.05% w/v.

Preferably, the bacterium is incubated at a temperature of between about 20° C. and about 37° C., such as between about 28° C. and about 32° C., or about 30° C. In some embodiments, the bacterium is incubated at a temperature of less than about 30° C.

Preferably, the production of the antimicrobial agents and the extraction thereof is carried out using a solid surface growth medium such as BSM (basal salts medium) agar. Preferably, the recovery of a compound according to the present invention from the growth medium comprises extraction of the compound with a solvent, preferably an organic solvent such as an alcohol (e.g. methanol) or ethyl acetate.

Preferably, the step of recovering the antimicrobial agent from agar-grown cultures comprises breaking up the nutrient or minimal media, preferably by cutting up the agar, prior to extraction of the antimicrobial agent using ethyl acetate. The microorganisms are grown on the agar surface, and the agar cut into blocks after growth. The antimicrobial agents are then extracted from the agar blocks using a solvent, preferably an organic solvent such as ethyl acetate.

The compounds in accordance with the invention may also be prepared from recombinant (genetically modified) or hybrid microbial systems, conveniently bacterial systems.

The biosynthetic gene cluster of Burkholderia ambifaria understood to be primarily responsible for the synthesis of the polyketide compounds of the present invention is the enacyloxin biosynthetic gene cluster. This gene cluster has been described in detail in Mahenthiralingam et al., Enacyloxins are products of an unusual hybrid modular polyketide synthase encoded by a cryptic Burkholderia ambifaria Genomic Island. Chem Biol. 18, 665, 2011.

As described in Mahenthiralingam et al., the enacyloxin gene cluster has 24 predicted genes (designated bamb_5910 to 5933). Table 1 below discloses the proposed function of each bamb gene

TABLE 1
Proposed functions of bamb genes in the enacyloxin biosynthetic
gene cluster of B. ambifaria AMMD

| Gene | Proposed function |
|---|---|
| bamb_5928 | FAD-dependent halogenase |
| bamb_5927 | α-Ketoglutarate and non-haem iron-dependent |
| | hydroxylase |
| bamb_5926 | Type II thioesterase |
| bamb_5925 | Polyketide synthase |
| bamb_5924 | Polyketide synthase |
| bamb_5923 | Polyketide synthase |
| bamb_5922 | Polyketide synthase |
| bamb_5921 | Polyketide synthase |
| bamb_5920 | Polyketide synthase |
| bamb_5919 | Polyketide synthase |
| bamb_5918 | Enoyl reductase involved in dihydroxycyclohexane |
| | carboxylic acid biosynthesis |
| bamb_5912 | Dehydratase involved in dihydroxycyclohexane |
| | carboxylic acid biosynthesis |
| bamb_5913 | Shikimate-5-dehydrogenase involved in |
| | dihydroxycyclohexane carboxylic acid biosynthesis |
| bamb_5914 | Enoyl reductase involved in dihydroxycyclohexane |
| | carboxylic acid biosynthesis |
| bamb_5915 | Nonribsomal peptide synthetase condensation |
| | domain |
| bamb_5916 | Acyl-CoA transferase involved in |
| | dihydroxycyclohexane carboxylic acid biosynthesis |
| bamb_5917 | Peptidyl Carrier Protein |
| bamb_5911 | LuxR family transcriptional regulator |
| bamb_5929 | Hypothetical protein |
| bamb_5933 | MATE family efflux protein |
| bamb_5910 | LuxR family transcriptional regulator |
| bamb_5930 | Carbamoyl transferase |
| bamb_5931 | α-Ketoglutarate and non-haem iron-dependent |
| | chlorinase |
| bamb_5932 | Pyrroloquinoline quinone-dependent oxidase |

The entire cluster, or any of the component genes thereof, including any of bamb_5910 to bamb_5933 may be used with recombinant techniques to prepare genetically modified (“recombinant”) microorganisms capable of producing the polyketide compounds according to the invention. Such microorganisms may be bacteria, in particular those which have, or are engineered to have, some or all components of another polyketide biosynthetic system (e.g. the vibroxin biosynthetic system). Selective and/or over expression of the individual gene components of the enacyloxin gene cluster, i.e. bamb_5910 to bamb_5933, and/or the mutation (sequence modification/editing) thereof, allows the design of polyketide compounds in accordance with the invention, and/or increased production of the target polyketide compound(s). Hybrid systems in which functionally complementary genes from other polyketide biosynthetic systems are expressed together with some, or all, of the enacyloxin biosynthetic gene cluster provides for further control of the design of polyketide compounds in accordance with the invention.

More specifically, it has been found that the polyketide compounds recited herein, in particular the specific compounds of the Examples, may be prepared by inactivating (e.g. by in frame deletion or disruption) of one or more of certain genes in an enacyloxin biosynthetic cluster, e.g. that of Burkholderia ambifaria (in particular strain BCCC0203), and culturing the genetically modified microorganism under conditions conducive to the production of polyketide compounds. In this regard, Table 2 sets out the polyketide structure obtained by mutants in particular enacyloxin biosynthetic cluster genes and also indicates which groups of enacyloxin may be modified by targeting particular enacyloxin biosynthetic cluster genes for inactivation. In other embodiments, microorganisms which do not have an intrinsic polyketide biosynthetic pathway may be recombinantly engineered to express a functional set of the enacyloxin biosynthetic genes which lacks the one or more genes which, when inactivated in Burkholderia ambifaria, result in the production of polyketide compounds in accordance with the invention. In still further embodiments microorganisms which do have an intrinsic polyketide biosynthetic pathway may be recombinantly engineered to express one or more of the enacyloxin biosynthetic genes and to have inactivated the intrinsic functional equivalent(s) of the one or more genes which, when inactivated in Burkholderia ambifaria, result in the production of polyketide compounds in accordance with the invention.

More specifically still, the inventors have found that

(i) polyketide compounds of the invention which have a hydrogen in place of a hydroxy group at the C14 position of enacyloxin IIa may be prepared from Burkholderia ambifaria in which bamb_5927 been inactivated, e.g. by in frame deletion,

(ii) polyketide compounds of the invention which have a hydrogen in place of a chlorine at the C18 position of enacyloxin IIa may be prepared from Burkholderia ambifaria in which bamb_5931 been inactivated, e.g. by in frame deletion,

(iii) polyketide compounds of the invention which have positions C₁₅-C₁₉ of enacyloxin IIa cyclised may be prepared from Burkholderia ambifaria in which bamb_5930 been inactivated, e.g. by in frame deletion,

(iv) polyketide compounds of the invention which have an additional hydroxyl position at position 5′ of the DHCCA group may be prepared from Burkholderia ambifaria in which bamb_5912 been inactivated, e.g. by in frame deletion,

(v) polyketide compounds of the invention which have a hydrogen in place of a chlorine at the C11 position of enacyloxin IIa may be prepared from Burkholderia ambifaria in which bamb_5928 been inactivated, e.g. by in frame deletion,

(vi) polyketide compounds of the invention which have a hydroxyl group in place of a carbonyl group at the C15 position of enacyloxin IIa may be prepared from Burkholderia ambifaria in which bamb_5932 been inactivated, e.g. by in frame deletion,

(vi) polyketide compounds of the invention which have a hydroxyl group in place of a carbonyl group at the C15 position of enacyloxin IIa and a hydroxyl group on place of a —OC(O)NH₂ group at the C19 position of enacyloxin IIa may be prepared from Burkholderia ambifaria in which bamb_5930 and bamb_5932 have been inactivated, e.g. by in frame deletion Combinations of such mutations may be made to provide polyketide compounds of the invention having a combination of the above described features.

The inventors have also found that polyketide compounds in which the moderately labile ester linkage is replaced by a more stable amide bond may be prepared by a mutasynthesis approach in which 3-amino-4-hydroxycyclohexane carboxylic acid (AHCCA) is fed to Burkholderia ambifaria mutants blocked in DHCCA biosynthesis by inactivation of any one, two, or all of bamb_5912 to bamb_5914. (Cis, cis) and (trans, trans)-3-amino-4-hydroxycyclohexane carboxylic acid can be produced in racemic form by high-pressure hydrogenation of commercially available 3-amino-4-hydroxybenzene carboxylic acid with rhodium on alumina as described, for example, by Wang et al., in Bioorganic and Medicinal Chemistry 14: 2242-2252, 2006, the entire contents of which are incorporated herein by reference.

The inventors have also found that polyketide compounds which have alternative functional groups to the DHCCA-derived moiety can be produced via a mutasynthesis strategy in which appropriate precursors are fed to Burkholderia ambifaria mutants blocked in DHCCA biosynthesis by inactivation of bamb_5912 to bamb_5914. Suitable precursors for the alternative moiety include, but are not limited to, 3,4-dihydroxycyclohexane carboxylic acid, 3-hydroxycyclopentane carboxylic acid, 3,4-diaminocyclohexane carboxylic acid, syn-3-aminocyclopentane carboxylic acid, 3-aminocyclohexane carboxylic acid, 4-amino-3-hydroxy butyric acid, 4-amino butyric acid, shikimate, 4-hydroxy butyric acid, 3,4-dihydroxybutyric acid, and other similar such compounds including any of the DHCCA derivatives herein described. Such precursor compounds are either commercially available or may readily be prepared using known chemical synthetic methods.

The inventors have also found that polyketide compounds which carry bromine groups at certain positions, e.g. in place of one or more of the chlorine groups of enacyloxin IIa, can be produced by including a source of bromide ions, e.g. bromide salts such as ammonium bromide, in the culture media of the microorganisms for use in the invention. These may be used in place of, or in addition to, any source of chloride ions such as ammonium chloride. By adjusting the relative proportions of bromide and chloride ions in the culture media, polyketide molecules with varying chlorine and/or bromine substitution patterns may be produced. It will be readily apparent that this approach to introducing bromine groups into polyketide compounds may be combined with any of the genetic engineering approaches described herein to create a variety of bromine modified polyketides in accordance with the invention. The preparation of polyketide compounds having bromine at C11 and/or C18 represents one embodiment of the invention.

In a further aspect the invention thus provides a brominated analogue of enacyloxin IIa, in particular an analogue of enacyloxin IIa in which one or both of the chlorine groups present in enacyloxin IIa are replaced by bromine groups. Methods for the preparation of such compounds, and their use as antimicrobial agents as herein described form further aspects of the invention.

The specific polyketide compounds which may be produced by the modified Burkholderia ambifaria described herein, or appropriately treated wild type or mutant Burkholderia ambifaria, are listed in Table 2.

The inactivation of one or more genes in a polyketide biosynthetic cluster (e.g. by in frame deletion or disruption) may be achieved by any convenient means, e.g. means which are able to modify, e.g. mutate or edit, nucleic acids carrying said genes. Mutation of host cells/nucleic acids can be achieved by routine means, e.g. exposure to radiation (e.g. UV) and/or chemical mutagens, or through recombinant techniques, e.g. transposon mutagenesis or homologous recombination mutagenesis. Gene editing technologies, e.g. CRISPR/Cas 9 may also be used.

Thus, in another aspect of the invention there is provided a genetically modified Burkholderia ambifaria, e.g. strain BCC0203, in which one or more of bamb_5912, bamb_5913, bamb_5914, bamb_5927, bamb_5928, bamb_5929, bamb_5930, bamb_5931 or bamb_5932, are inactivated.

By inactivated it is meant that the protein expression product of the gene, if any, is non-functional to the extent it cannot contribute materially to the synthesis of polyketides in the genetically modified Burkholderia ambifaria. More specifically, the protein expression product will have negligible levels of the relevant catalytic activity recited in Table 1.

In certain embodiments, the genetically modified Burkholderia ambifaria has inactivated forms of

(i) bamb_5927,

(ii) bamb_5931,

(iii) bamb_5930,

(iv) bamb_5912,

(v) bamb_5927 and bamb_5930,

(vi) bamb_5927 and bamb_5931,

(vii) bamb_5932,

(viii) bamb_5930 and bamb_5932,

(ix) bamb_5930 and bamb_5931,

(x) bamb_5930 to bamb_5932,

(xi) bamb_5927 to bamb_5932,

(xii) bamb_5912 to bamb_5914,

(xiii) bamb_5930 and bamb_5932,

(xiv) bamb_5912 to bamb_5914 and bamb_5930 and bamb_5932, or

(xv) bamb_5928.

In another aspect the invention provides a genetically modified Burkholderia ambifaria, e.g. strain BCC0203, in which all, or a portion, of one or more of bamb_5910 to bamb_5933 has been deleted in frame, e.g. by homologous recombination mutagenesis.

In certain embodiments the genetically modified Burkholderia ambifaria has all, or a portion, of one or more of bamb_5912, bamb_5913, bamb_5914, bamb_5927, bamb_5928, bamb_5929, bamb_5930, bamb_5931 or bamb_5932, deleted in frame, e.g. by homologous recombination mutagenesis.

In certain embodiments, the genetically modified Burkholderia ambifaria has all, or a portion, of

(i) bamb_5927,

(ii) bamb_5931,

(iii) bamb_5930,

(iv) bamb_5912,

(v) bamb_5927 and bamb_5930,

(vi) bamb_5927 and bamb_5931,

(vii) bamb_5932,

(viii) bamb_5930 and bamb_5932,

(ix) bamb_5930 and bamb_5931,

(x) bamb_5930 to bamb_5932,

(xi) bamb_5927 to bamb_5932,

(xii) bamb_5912 to bamb_5914,

(xiii) bamb_5930 and bamb_5932,

(xiv) bamb_5912 to bamb_5914 and bamb_5930 and bamb_5932, or

(xv) bamb_5928,

deleted in frame, e.g. by homologous recombination mutagenesis.

In these aspects the genetically modified Burkholderia ambifaria produces a polyketide compound of the invention upon culturing in conducive conditions, e.g. those described above.

The recombinantly engineered microorganisms described herein may be prepared by introducing one or more nucleic acids carrying the appropriate gene sequences into a microorganism under the control of suitable expression elements (and optionally other operably associated regulatory element(s)). Conveniently, the nucleic acid molecule will be in the form of a nucleic acid vector, particularly an expression vector, or a plasmid. The vector or plasmid may comprise one or more selectable markers and/or other genetic elements. The vector or plasmid may also comprise an origin of replication, for example a Gram positive and/or a Gram negative bacterial origin of replication. The vector or plasmid may also comprise one or more insertion sequences, e.g. Tn10, Tn5, Tn1545, Tn916 and/or ISCb.

Methods of introducing nucleic acid molecules, plasmids and vectors into microbial host cells are well known in the art. These include transformation, transfection and electroporation techniques. The nucleic acid molecule, vector or plasmid may become located in the cytoplasm of the microbial host cell. The nucleic acid, vector or plasmid may become located in (e.g. stably integrated into) the genome of the microbial host cell.

The microorganism being engineered may be a bacterium, for example, a Gram-positive or Gram-negative bacterium. In some embodiments the bacterium will be a bacterium that does not have some or all components of a polyketide biosynthetic system. In some embodiments, the bacterium will be a bacterium that has some or all components of a polyketide biosynthetic system (e.g. the enacyloxin or vibroxin biosynthetic system). In other embodiments the bacterium may be from the genus Vibrio, Burkholderia, Frateuria, Sorangium (e.g. Sorangium cellulosum) or Pseudomonas. In other embodiments standard experimental bacteria may be used as host, e.g. E. coli or Streptomyces. In some embodiments of the invention, the host cell is from the genus Burkholderia, e.g. Burkholderia gladioli, in particular Burkholderia gladioli strain LMG-P 26202 or Burkholderia gladioli pv. cocovenenans. In other embodiments the host cell is from the genus Vibrio, more particularly Vibrio rhizosphaerae (e.g. Vibrio rhizosphaerae MSSRF3). In other embodiments the host cell is not from the genus Burkholderia, more particularly Burkholderia ambifaria, more particularly Burkholderia ambifaria BCC0203 As used herein, the term “nucleic acid molecule” refers to a DNA or RNA molecule, which might be single- or double-stranded. Preferably, the nucleic acid molecule is a DNA molecule, most preferably a double-stranded DNA molecule.

In a further aspect of the invention there is provided a method for the preparation of a polyketide compound in accordance with the invention, e.g. those described herein, said method comprising culturing a genetically modified (e.g. recombinant) host microbial cell of the invention as defined above under conditions conducive to the production of a polyketide compound of the invention. Culture conditions may be those as described above. Recovery of the polyketide compound(s) or a portion thereof may conveniently be achieved as described above. Polyketide compounds obtained from such methods form a further aspect of the invention.

The compounds of the present invention may be used in therapy. As such, according to another aspect of the present invention, there is provided a method for the treatment of an infection, the method comprising administering to a subject in need thereof, a compound according to the present invention, or a pharmaceutically acceptable salt, metabolite, or prodrug thereof, wherein the infection is caused by a microbe, optionally wherein the microbe is resistant to an antimicrobial drug.

According to another aspect of the present invention, there is provided a method for the treatment of an infection, the method comprising administering to a subject in need thereof, a compound according to the present invention, or a pharmaceutically acceptable salt, metabolite, or prodrug thereof, wherein the infection is caused by at least one pathogenic bacterium that is susceptible to vibroxin or an analogue thereof as herein described, for example at least one bacterium selected from Acinetobacter species, Burkholderia species, Ralstonia species, and Stenotrophomonas species.

According to another aspect of the present invention, there is provided the use of a compound according to the present invention, or a pharmaceutically acceptable salt, metabolite, or prodrug thereof, preferably a therapeutically acceptable amount thereof, in the manufacture of a medicament for the treatment of a microbial infection.

Also provided is a method for inhibiting the growth of a microbe, the method comprising contacting the microbe with a compound according to the present invention, or a pharmaceutically acceptable salt, metabolite, or prodrug thereof, or with a bacterium capable of producing the compound. The method may be performed in vitro or in vivo. In the case of contact with a bacterium capable of producing the compound, suitable conditions, such as those identified above, may be provided in order that the antimicrobial compound is produced.

It is preferred that a therapeutically effective amount of a compound as herein described, in any stereochemical form, or a mixture of any stereochemical forms in any ratios, or a pharmaceutically acceptable salt, metabolite, or prodrug thereof, is present or is used in the above aspects of the invention.

The following definitions shall apply throughout the specification and the appended claims.

Within the context of the present application, the terms “comprises” and “comprising” are interpreted to mean “includes, among other things”. These terms are not intended to be construed as “consists of only”.

Unless otherwise stated or indicated, the term “alkyl” means a monovalent saturated, linear or branched, carbon chain, such as C₁₋₈, C₁₋₆ or C₁₋₄, which may be unsubstituted or substituted. The group may be partially or completely substituted with substituents independently selected from one or more of halogen (F, Cl, Br or I), hydroxy, nitro and amino. Non-limiting examples of alkyl groups methyl, ethyl, n-propyl, iso-propyl, n-butyl, iso-butyl, sec-butyl, tert-butyl, n-pentyl, iso-pentyl, n-pentyl, n-hexyl, etc. An alkyl group preferably contains from 1-6 carbon atoms, e.g. 1-4 carbon atoms.

Unless otherwise stated or indicated, the term “cycloalkyl” refers to a monovalent, saturated cyclic carbon system. Unless otherwise specified, any cycloalkyl group may be substituted in one or more positions with a suitable substituent. Where more than one substituent group is present, these may be the same or different. Suitable substituents include halogen (F, Cl, Br or I), hydroxy, nitro and amino.

Unless otherwise stated or indicated, the term “alkenyl” means a straight-chained or branched unsaturated hydrocarbon chain of 2-20 carbon atoms, such as C₂₋₁₀, C₂₋₈, C₂₋₆ or C₂₋₄, which may be unsubstituted or substituted, and containing at least one double bond. The group may be partially or completely substituted with substituents independently selected from one or more of halogen (F, Cl, Br or I), hydroxy, nitro and amino. Non-limiting examples of alkenyl groups include ethenyl, 1-propenyl, 2-propenyl, 2-methyl-1-propenyl, 1-butenyl, 2-butenyl-pentenyl, 3-pentenyl, 3-methyl-2-butenyl, 3-methyl-but-2-enyl, 3-hexenyl, 1,1-dimethyl-but-2-enyl, and the like.

Unless otherwise stated or indicated, the term “aryl” is intended to cover aromatic ring systems. Such ring systems may be monocyclic or polycyclic (e.g. bicyclic) and contain at least one unsaturated aromatic ring. Where these contain polycyclic rings, these may be fused. Preferably such systems contain from 6-20 carbon atoms, e.g. either 6 or 10 carbon atoms. Examples of such groups include phenyl, 1-napthyl, 2-napthyl and indenyl. A preferred aryl group is phenyl. Unless stated otherwise, any “aryl” group may be substituted by one or more substituents, which may be identical or different, for example halogen (F, Cl, Br or I), hydroxy, nitro and amino.

As used herein, the term “heteroaryl” is intended to cover heterocyclic aromatic groups. Such groups may be monocyclic or bicyclic and contain at least one unsaturated heteroaromatic ring system. Where these are monocyclic, these comprise 5- or 6-membered rings which contain at least one heteroatom selected from nitrogen, oxygen and sulphur and contain sufficient conjugated bonds to form an aromatic system. Where these are bicyclic, these may contain from 9-11 ring atoms. Examples of heteroaryl groups include thiophene, thienyl, pyridyl, thiazolyl, furyl, pyrrolyl, triazolyl, imidazolyl, oxadiazolyl, oxazolyl, pyrazolyl, imidazolonyl, oxazolonyl, thiazolonyl, tetrazolyl, thiadiazolyl, benzimidazolyl, benzooxazolyl, benzofuryl, indolyl, isoindolyl, pyridonyl, pyridazinyl, pyrimidinyl, imidazopyridyl, oxazopyridyl, thiazolopyridyl, imidazopyridazinyl, oxazolopyridazinyl, thiazolopyridazinyl and purinyl. Preferred heteroaryl groups include pyrrole, indole, thiazole, triazole or pyridine. Unless stated otherwise, any “heteroaryl” may be substituted by one or more substituents, which may be identical or different, for example halogen (F, Cl, Br or I), hydroxy, nitro and amino.

The term “antimicrobial” includes antibiotics and chemicals capable of inhibiting or preventing the growth of, or capable of killing, microbes, especially bacteria. An example of an antimicrobial chemical is a disinfectant.

The term “antibiotic” means an agent produced by a living organism, such as a bacterium, that is capable of inhibiting the growth of another living organism, for example another bacterium, or is capable of killing another living organism, for example another bacterium.

The term “therapeutically effective amount” means an amount of an agent or compound which provides a therapeutic benefit in the treatment of a microbial infection.

The term “treatment” includes prevention, reduction, amelioration or elimination or the disorder or condition.

The term “pharmaceutically acceptable” means being useful in preparing a pharmaceutical composition that is generally safe, non-toxic and neither biologically nor otherwise undesirable and includes being useful for veterinary use as well as human pharmaceutical use.

Suitable pharmaceutically acceptable salts may include acid addition salts which may, for example, be formed by mixing a solution of the antimicrobial agent with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the antimicrobial agents of the invention carry an acidic moiety, suitable pharmaceutically acceptable salts thereof may include alkali metal salts (e.g. sodium or potassium salts); alkaline earth metal salts (e.g. calcium or magnesium salts); and salts formed with suitable organic ligands (e.g. ammonium, quaternary ammonium and amine cations formed using counter-anions such as halide, hydroxide, carboxylate, sulfate, phosphate, nitrate, alkyl sulfonate and aryl sulfonate). Illustrative examples of pharmaceutically acceptable salts include but are not limited to acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), pahnitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like.

The term “metabolite” means any intermediate or product resulting from metabolism of a compound according to the present invention.

The term “prodrug” means a functional derivative of a compound according to the present invention, such as an ester or an amide, that is biotransformed in the body to form the active drug. Reference is made to Goodman and Gilman's, The Pharmacological basis of Therapeutics, 8th ed., McGraw-Hill, Int. Ed. 1992, “Biotransformation of Drugs”, p. 13-15.

The term “isomer” used herein refers to all forms of structural and spatial isomers. In particular, the term “isomer” is intended to encompass stereoisomers. Unless otherwise indicated, the structures presented herein are not intended as an accurate representation of the cis-trans stereochemistry of any of the double bonds in the molecule. All potential combinations of cis and trans stereochemistries are considered to be encompassed by the invention.

With regard to stereoisomers, a number of the compounds herein described may have one or more asymmetric carbon atoms and may occur as racemates, racemic mixtures and as individual enantiomers or diastereomers. All such isomeric forms are included within the present invention, including mixtures thereof. Cis (E) and trans (Z) isomerism may also occur. The present invention includes the individual stereoisomers of the compounds of the invention, together with mixtures thereof. Separation of diastereoisomers or cis and trans isomers may be achieved by conventional techniques, e.g. by fractional crystallisation, chromatography or HPLC. A stereoisomeric mixture of the compounds may also be prepared from a corresponding optically pure intermediate or by resolution, such as by HPLC of the corresponding racemate using a suitable chiral support or by fractional crystallisation of the diastereoisomeric salts formed by reaction of the corresponding racemate with a suitable optically active acid or base, as appropriate.

The term “potentially susceptible bacteria” or means bacteria which have the potential for their growth to be inhibited or destroyed by an antimicrobial agent produced by an antimicrobial producing bacterium. Examples include those listed herein whose growth may be inhibited by the antimicrobial agents of the present invention.

Antimicrobial agents of the invention can be incorporated into pharmaceutical compositions suitable for administration. Such compositions typically comprise at least one antimicrobial of the invention and at least one pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that syringability exists. It must be stable under the conditions of manufacture, transfer and storage. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminium mono stearate and gelatin.

Sterile injectable solutions can be prepared by incorporating the active compound (e.g., an antimicrobial according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavouring.

For administration by inhalation, the compounds can be delivered in the form of an aerosol spray of liquid, or powdered or formulated antibiotic (e.g. within liposomes as stated below) from pressured container or dispenser which contains a suitable propellant, e.g. a gas such as carbon dioxide, or a nebulizer.

For topical administration, the compounds may be delivered in the form of gels, creams, ointments, sprays, lotions, salves, powders, aerosols, drops, solutions and any of the other conventional pharmaceutical forms in the art. Ointments, gels and creams may, for example, be formulated with an aqueous or oily base with the addition of suitable thickening and/or gelling agents. Any thickening or gelling agents used should be non-toxic and non-irritant. Formulations for topical treatment, e.g. treatment of bacterial infected wounds, may be based on gel formulations, e.g. hydrogels. The compounds of the invention may be incorporated into such hydrogel formulations. Lotions may be formulated with an aqueous or oily base and will, in general, also contain one or more emulsifying, dispersing, suspending, thickening or colouring agents. Powders may be formed with the aid of any suitable powder base. Drops (e.g. eye drops), sprays (e.g. nasal sprays) and solutions may be formulated with an aqueous or non-aqueous base also comprising one or more dispersing, solubilising or suspending agents. Aerosol sprays are conveniently delivered from pressurised packs, with the use of a suitable propellant.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, pessaries or suppositories. For transdermal administration, the active compounds can be formulated into ointments, salves, gels, or creams as generally known in the art.

The compounds can also be prepared in the form of suppositories (e.g. with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration. According to this aspect, the invention provides a kit comprising at least one compound according to the invention or a pharmaceutical composition of the invention, optionally in addition to one or more further active agents as defined herein, preferably with instructions for the administration thereof in the therapeutic treatment of the human or animal body, e.g. the treatment of infection by one or more infectious organisms as hereinbefore defined.

When using the compounds according to the present invention, the dose can vary within wide limits and, as is customary and is known to the physician, is to be suited to the individual conditions in each individual case. It depends, for example, on the nature and severity of the disease to be treated, on the mode of administration, or on whether an acute or chronic condition is treated or whether prophylaxis is carried out. An appropriate dosage can be established using clinical approaches well known in the medical art. In general, the daily dosage for achieving the desired results in an adult weighing about 75 kg is from about 0.01 to about 100 mg/kg, preferably from about 0.1 to about 50 mg/kg, in particular from about 0.1 to about 10 mg/kg.

Within this specification embodiments have been described in a way which enables a clear and concise specification to be written, but it is intended and will be appreciated that embodiments may be variously combined or separated without departing from the invention.

The invention will now be further illustrated by the following non-limiting examples and the accompanying figures, in which:

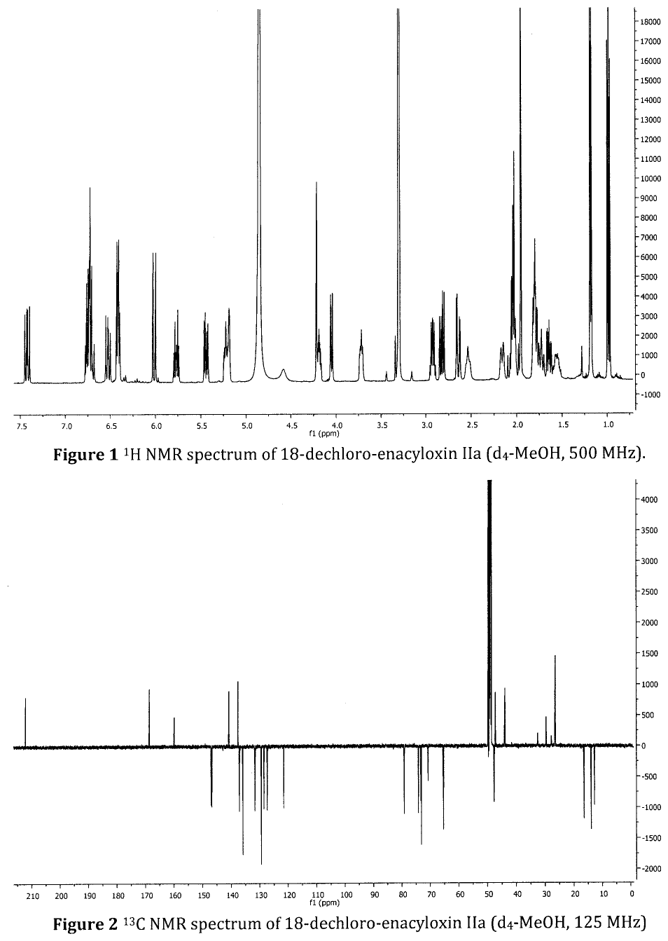

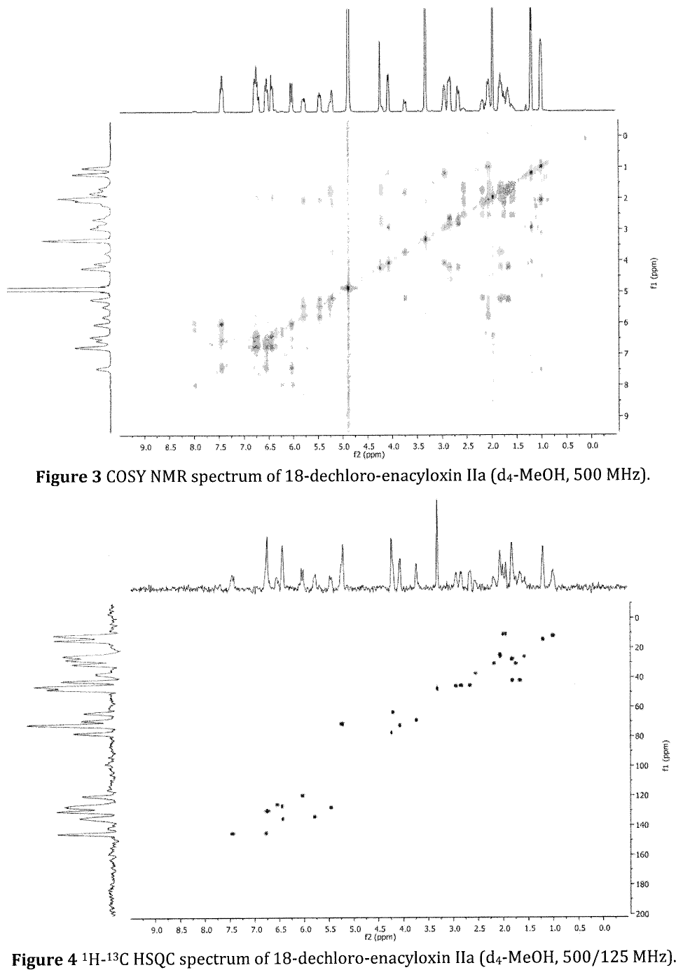

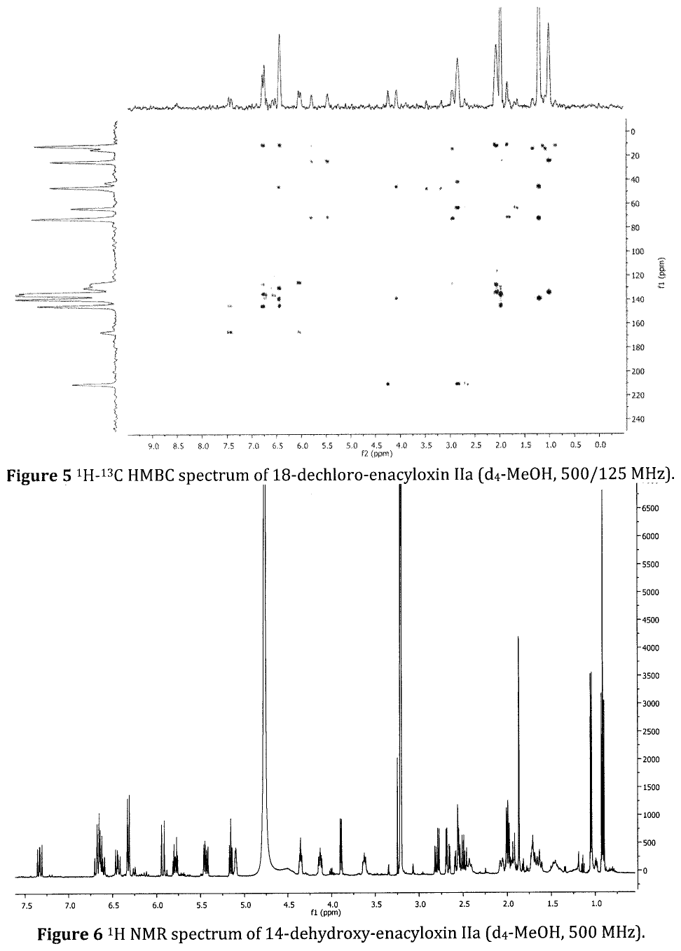

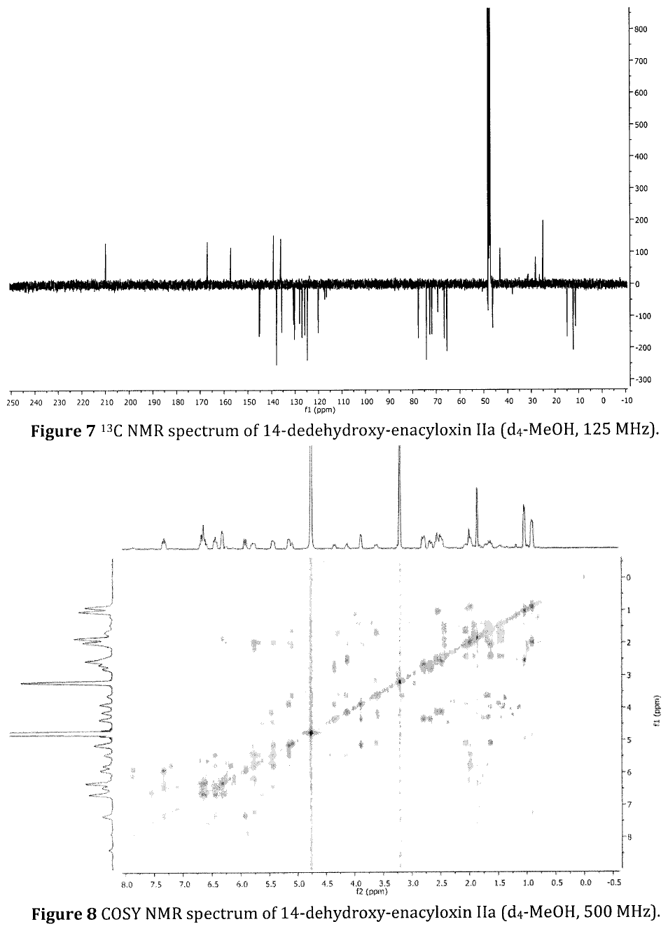

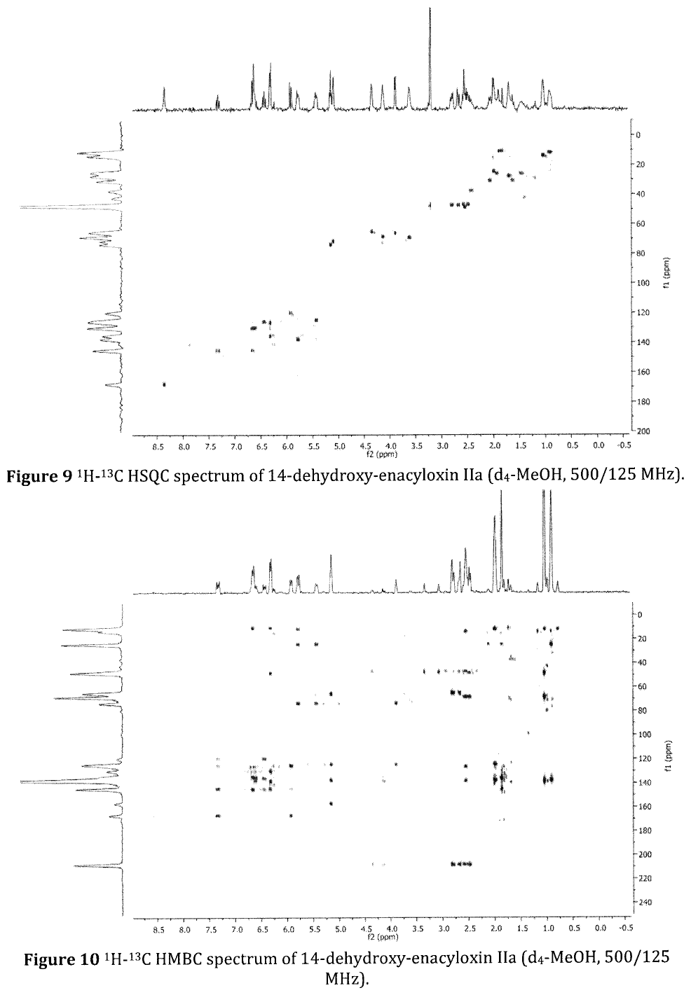

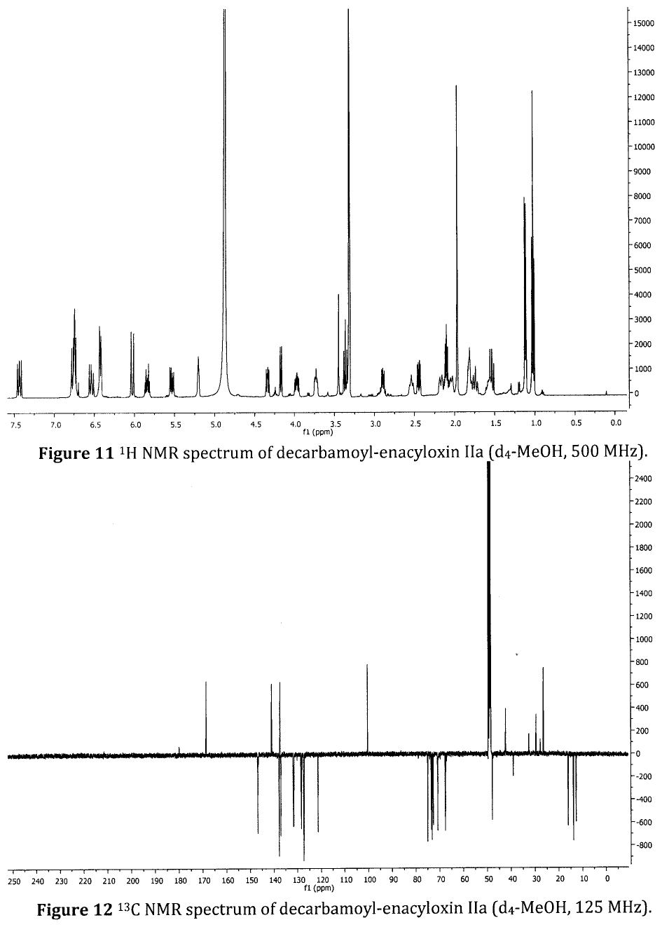

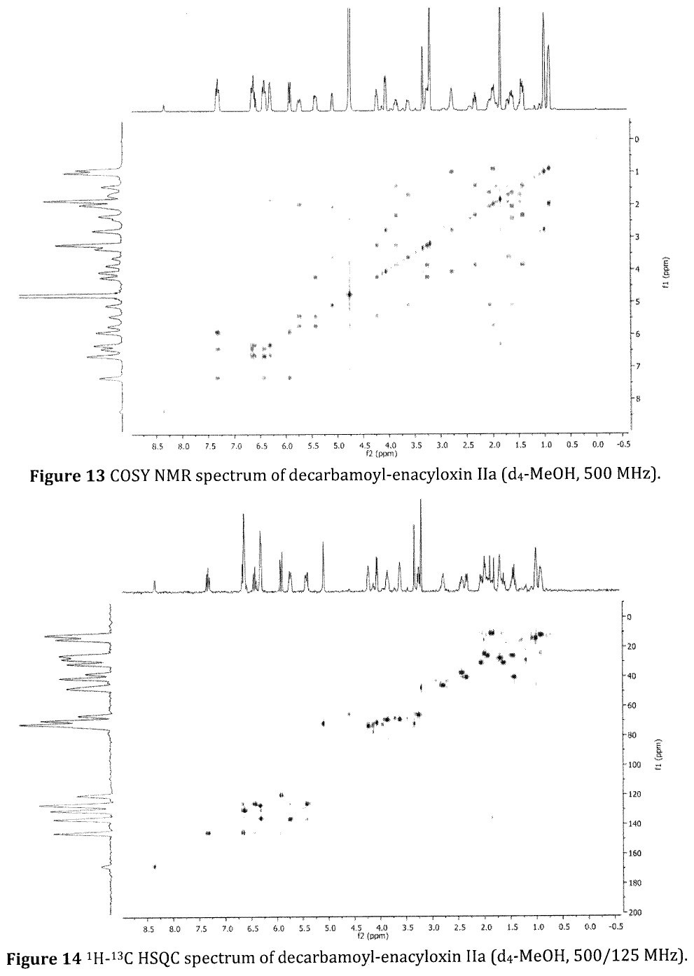

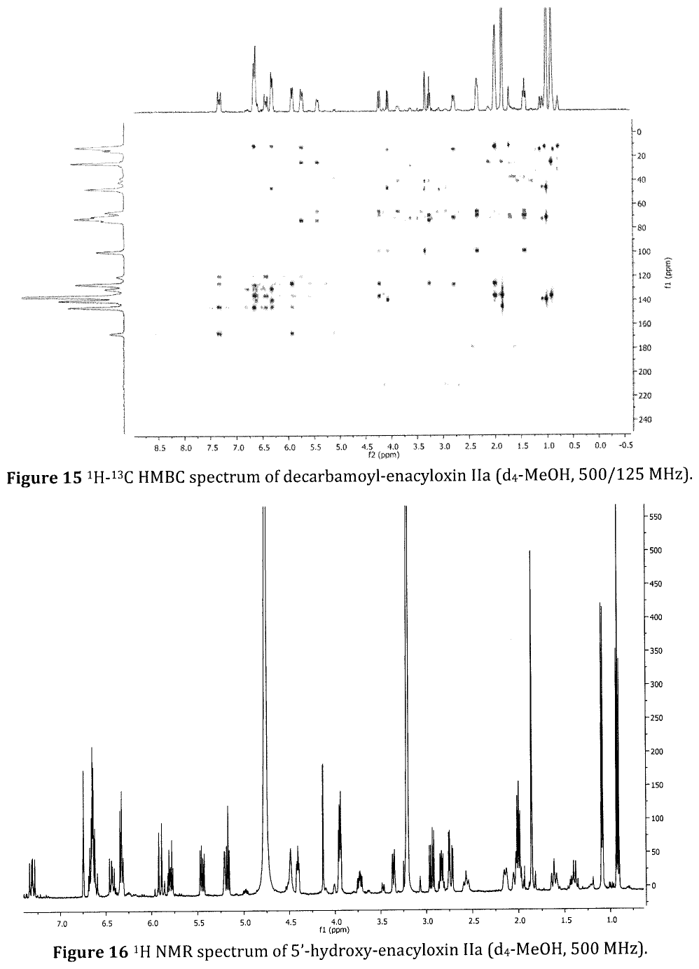

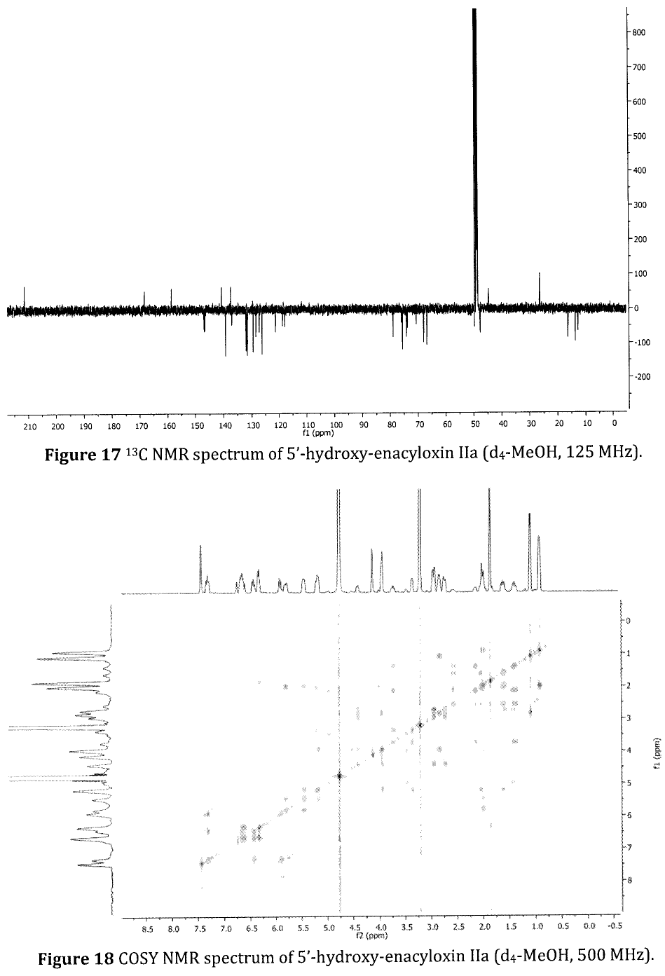

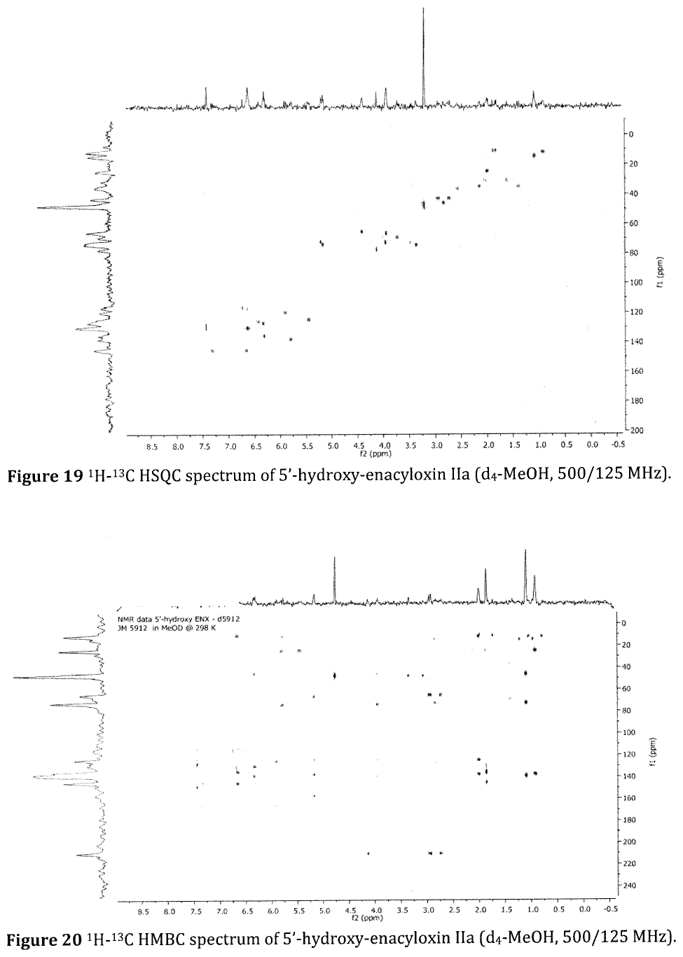

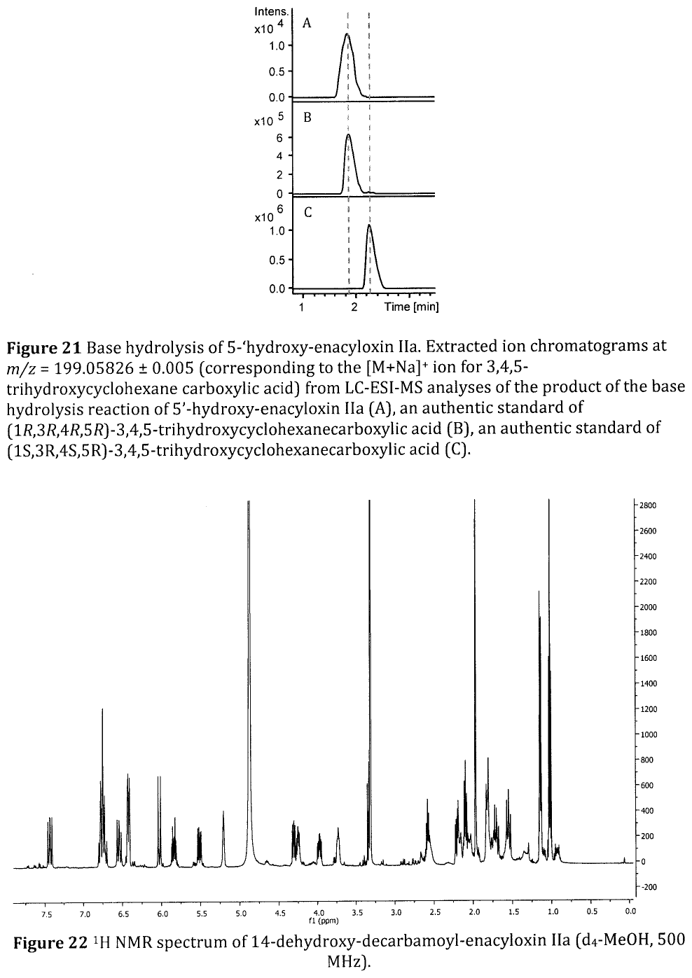

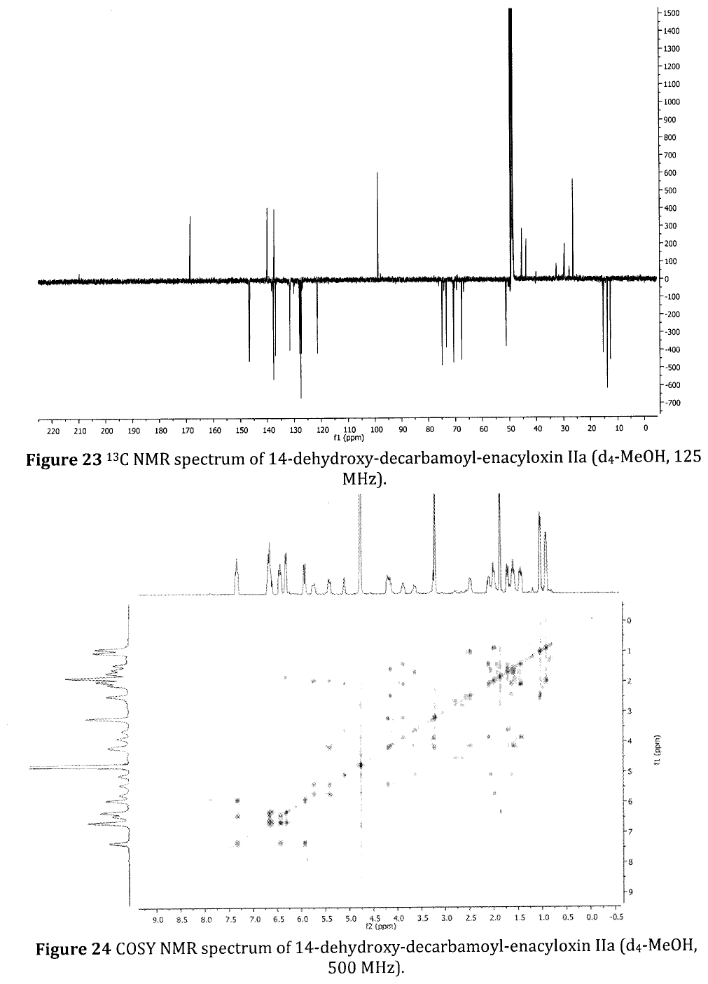

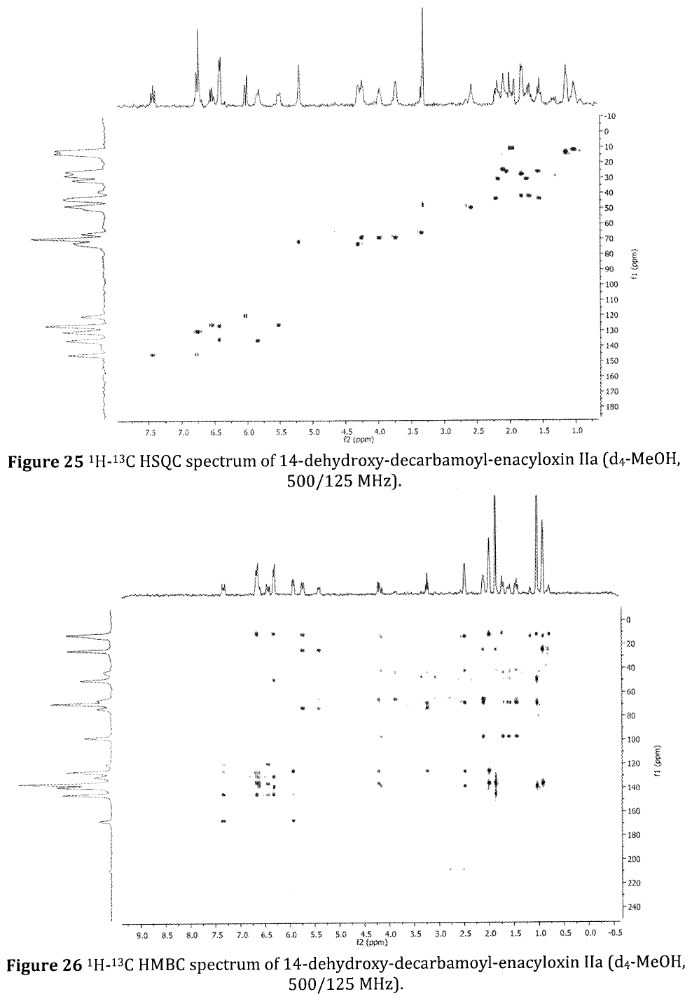

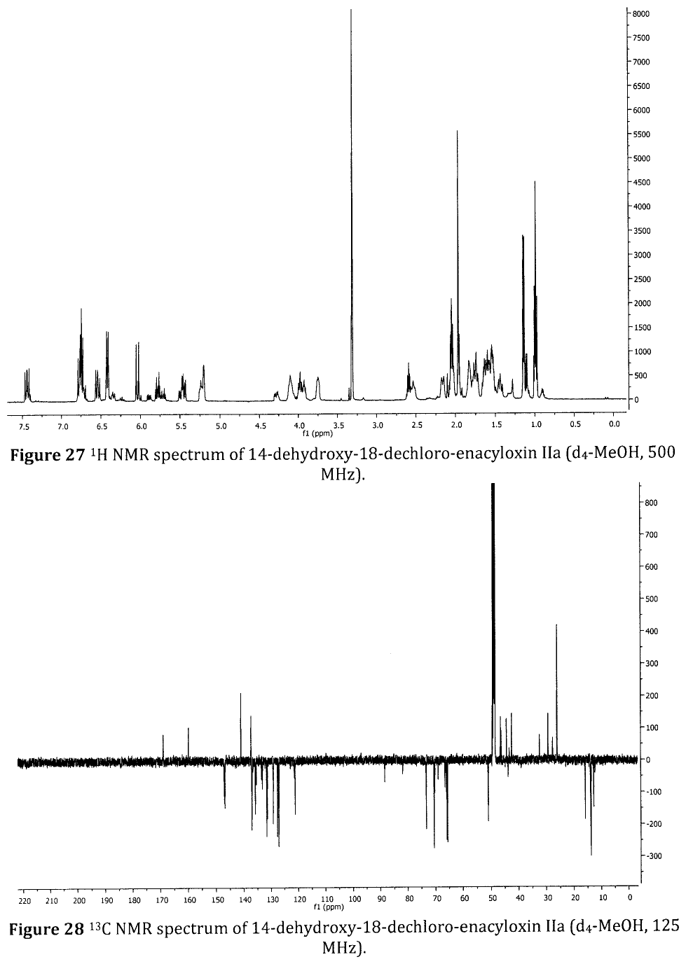

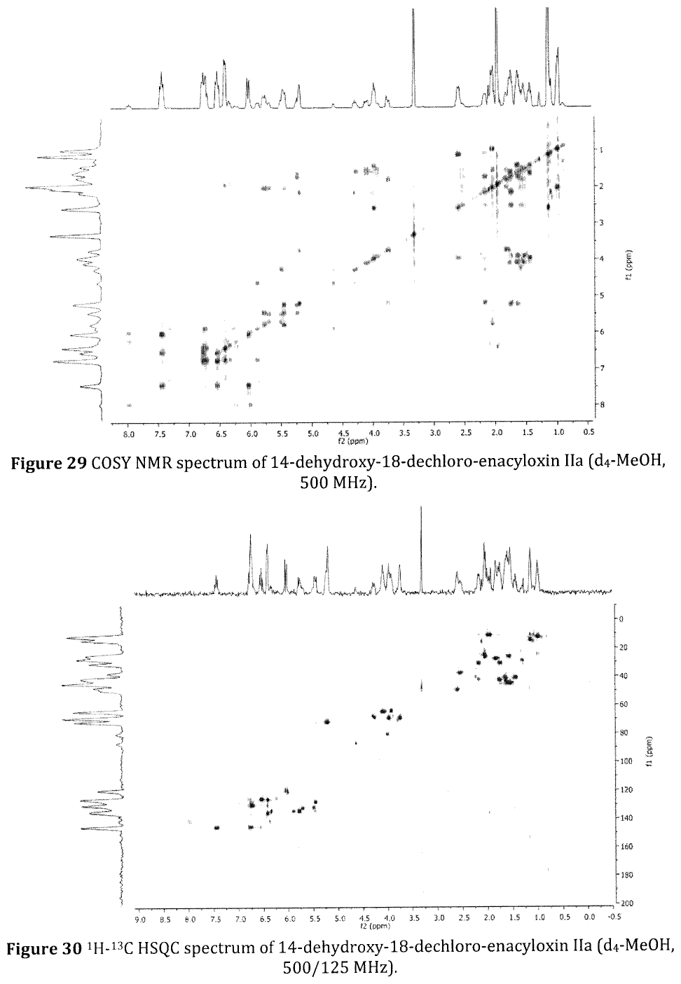

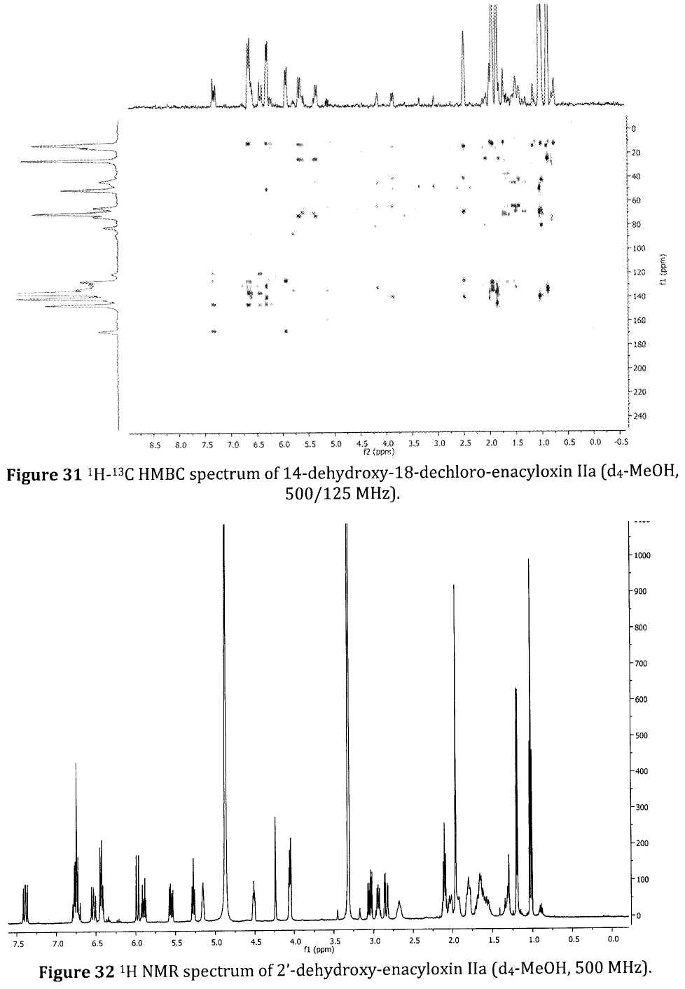

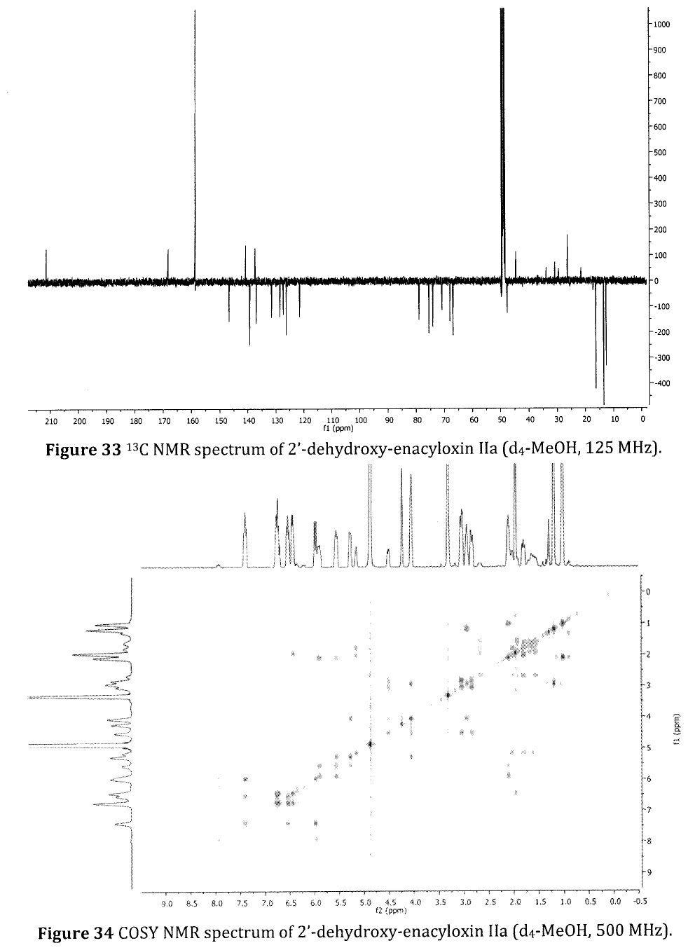

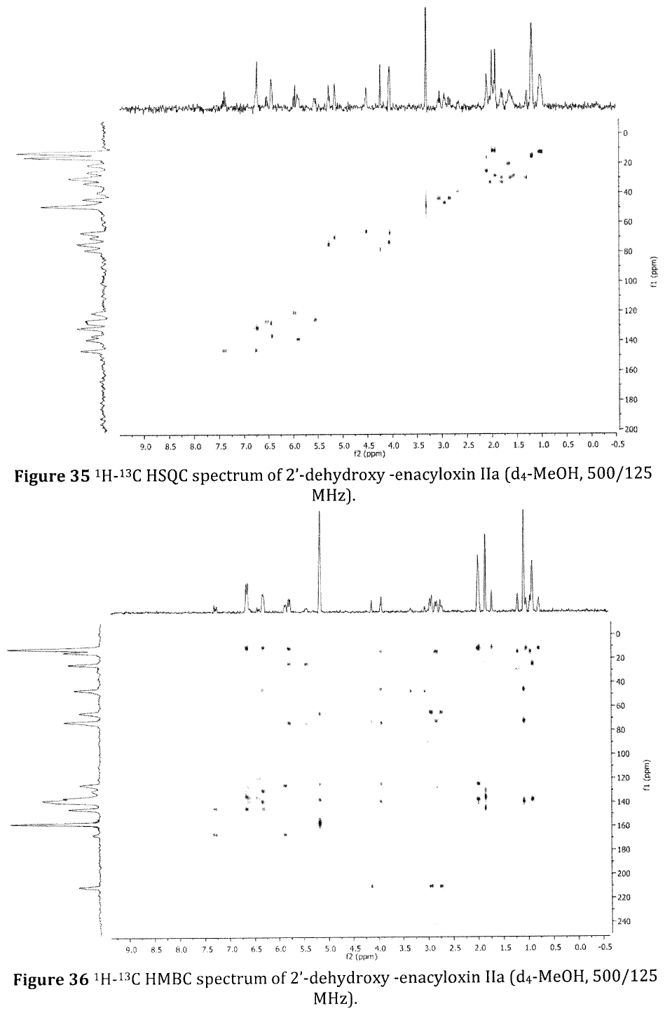

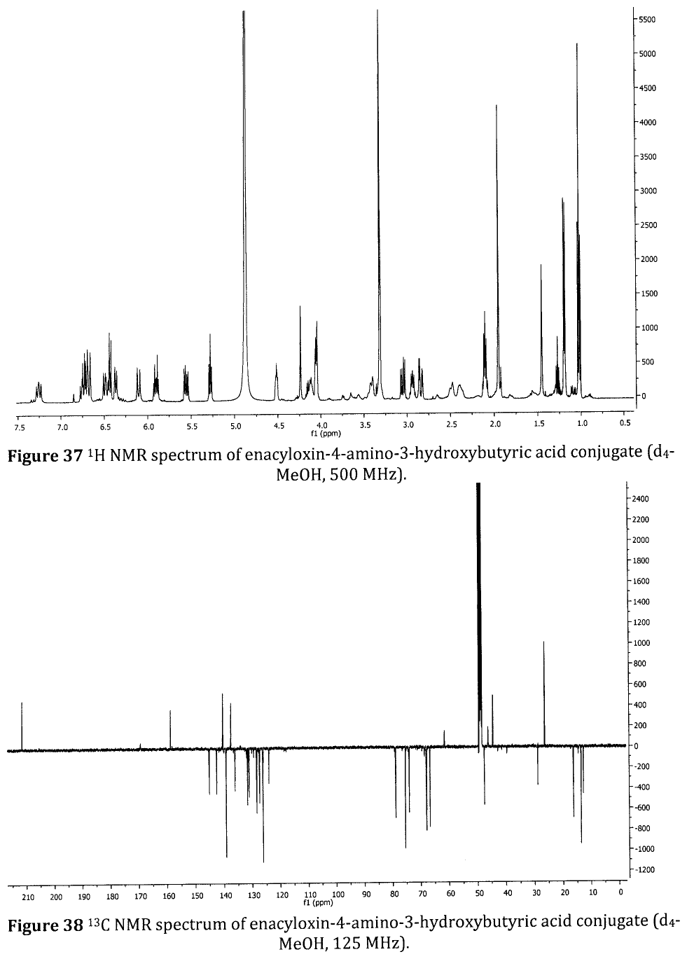

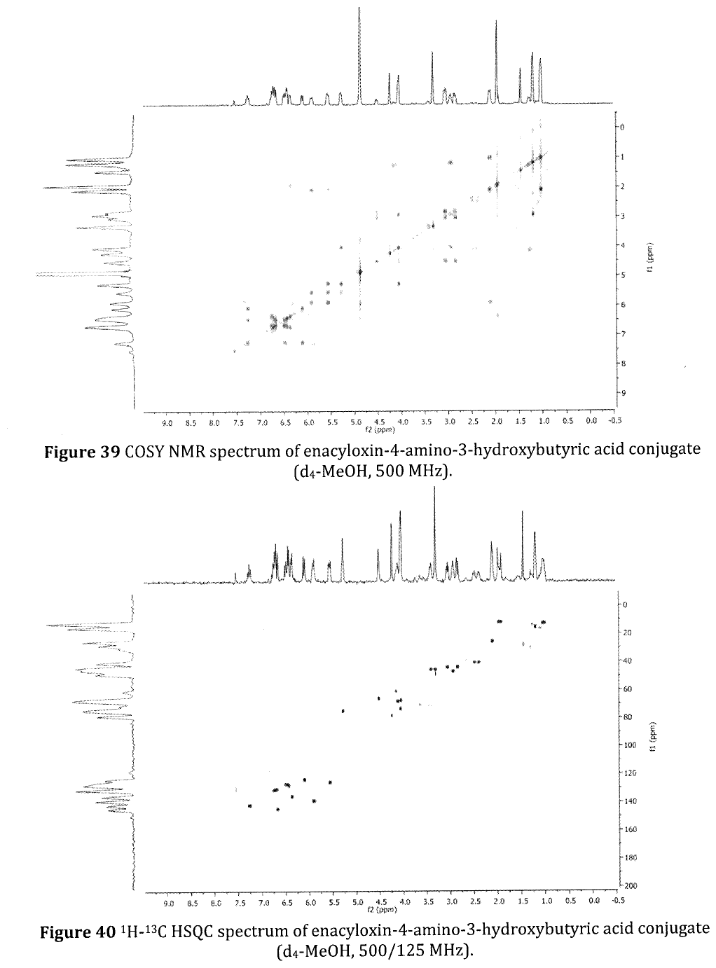

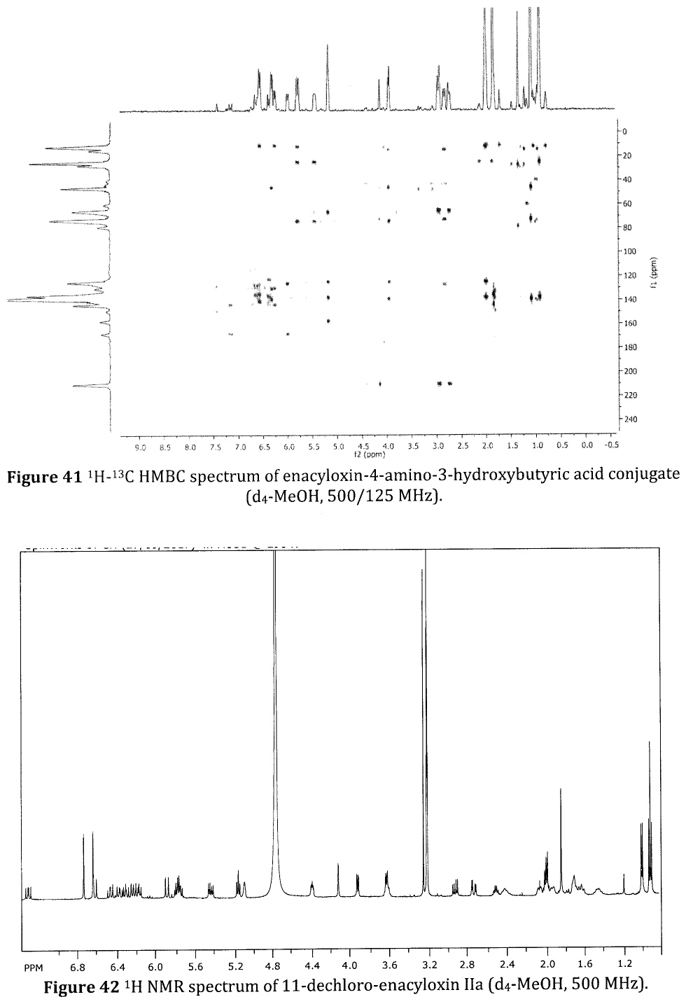

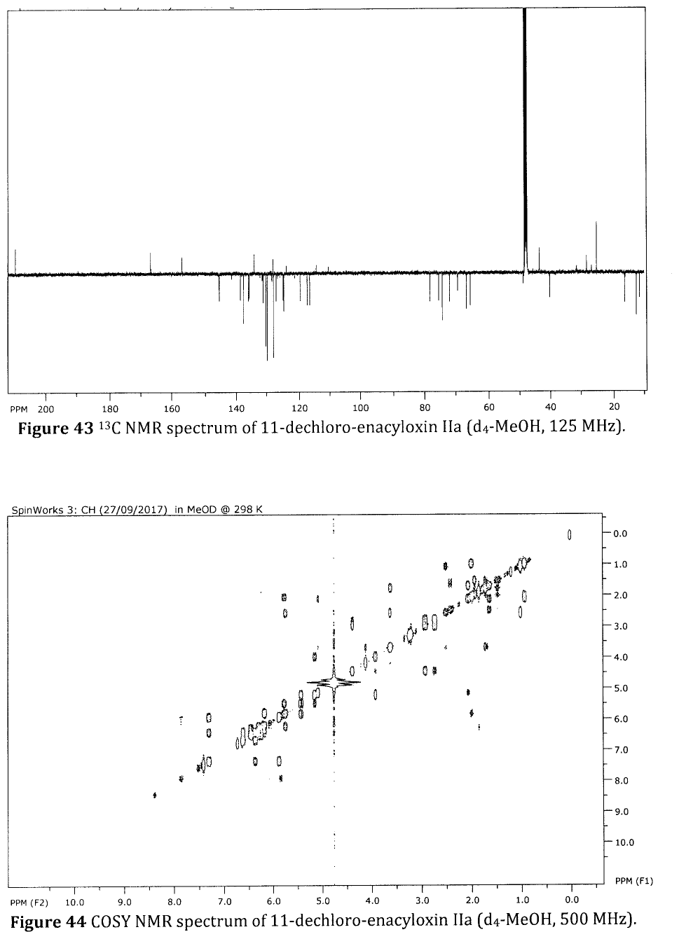

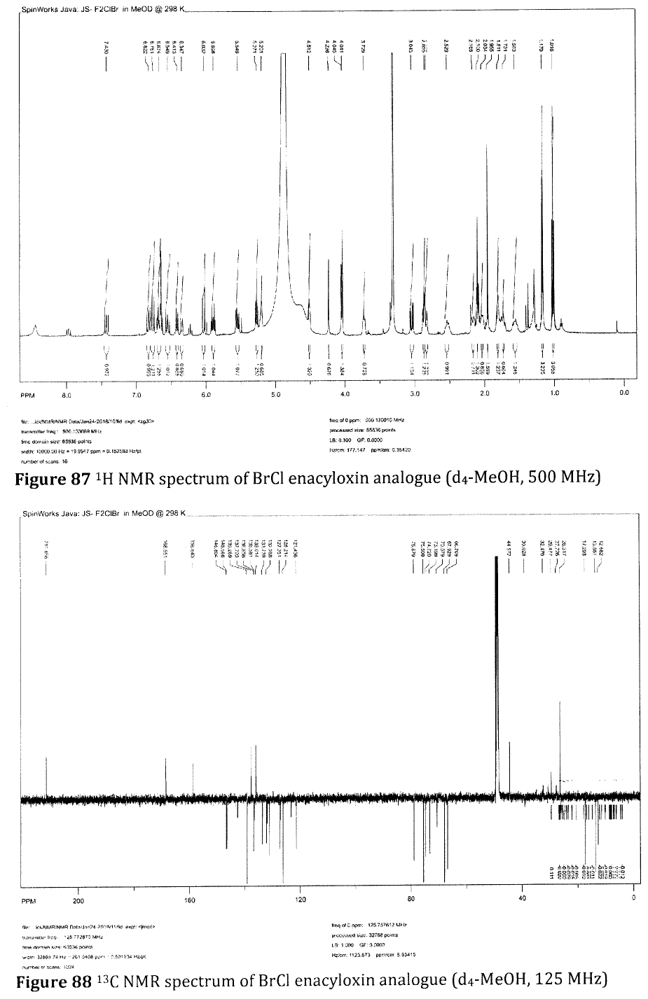

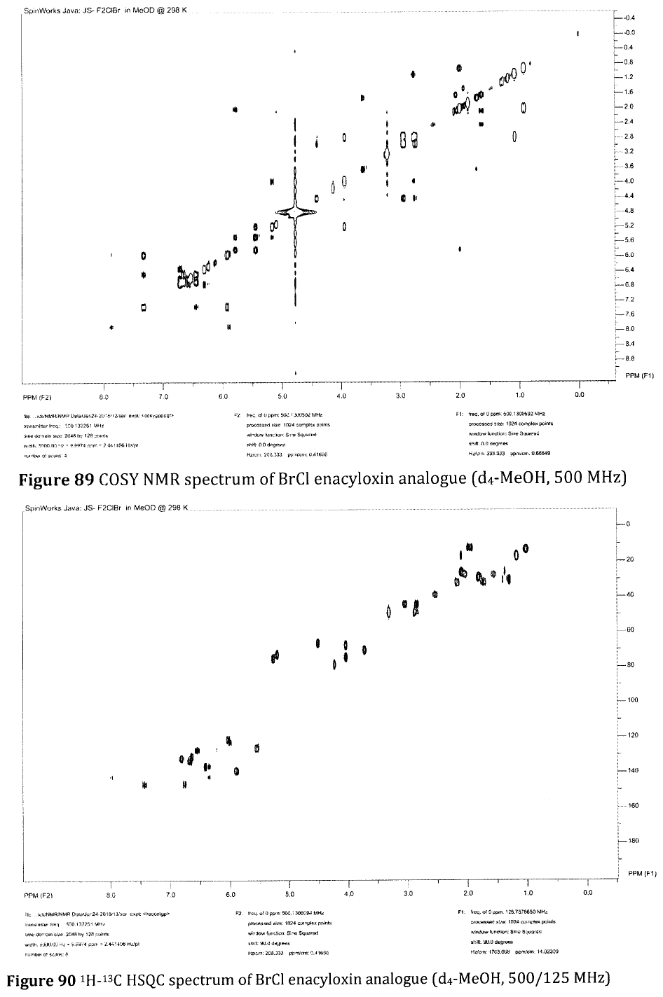

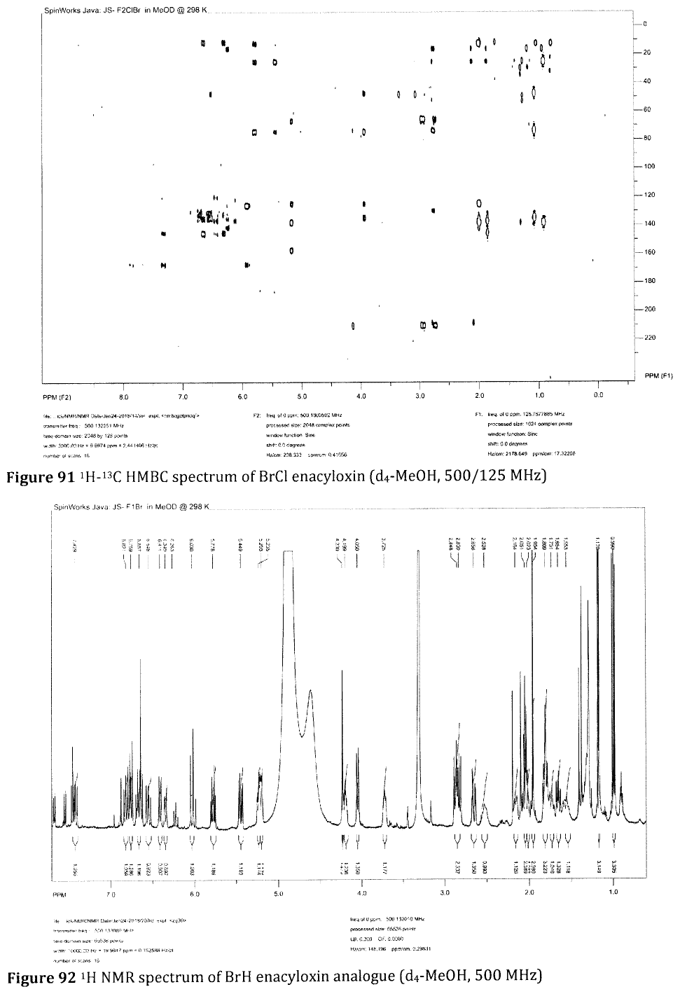

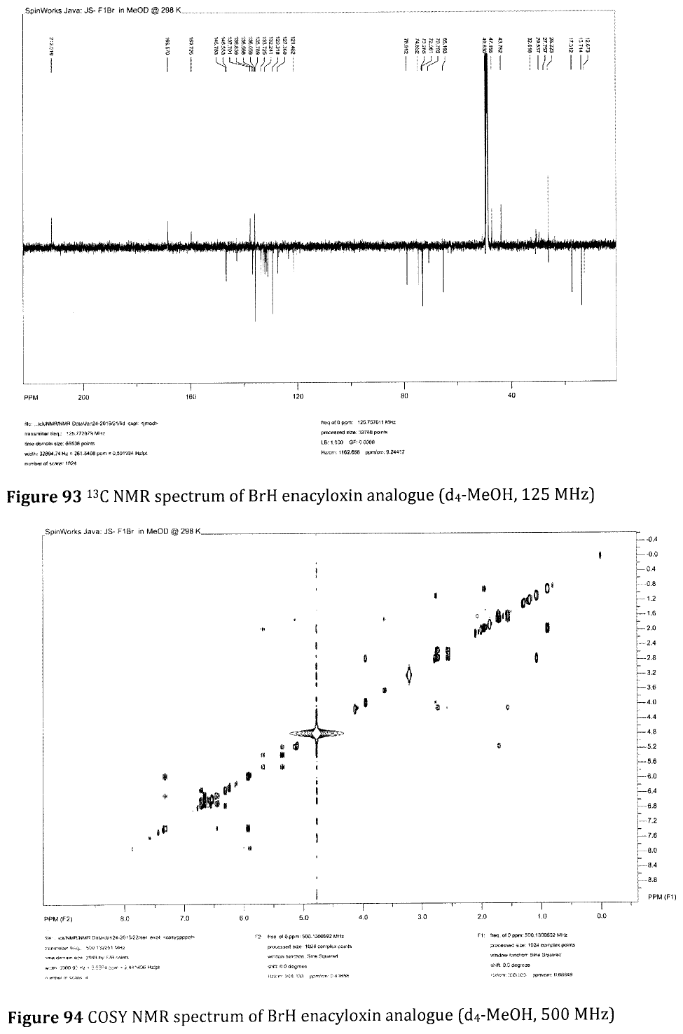

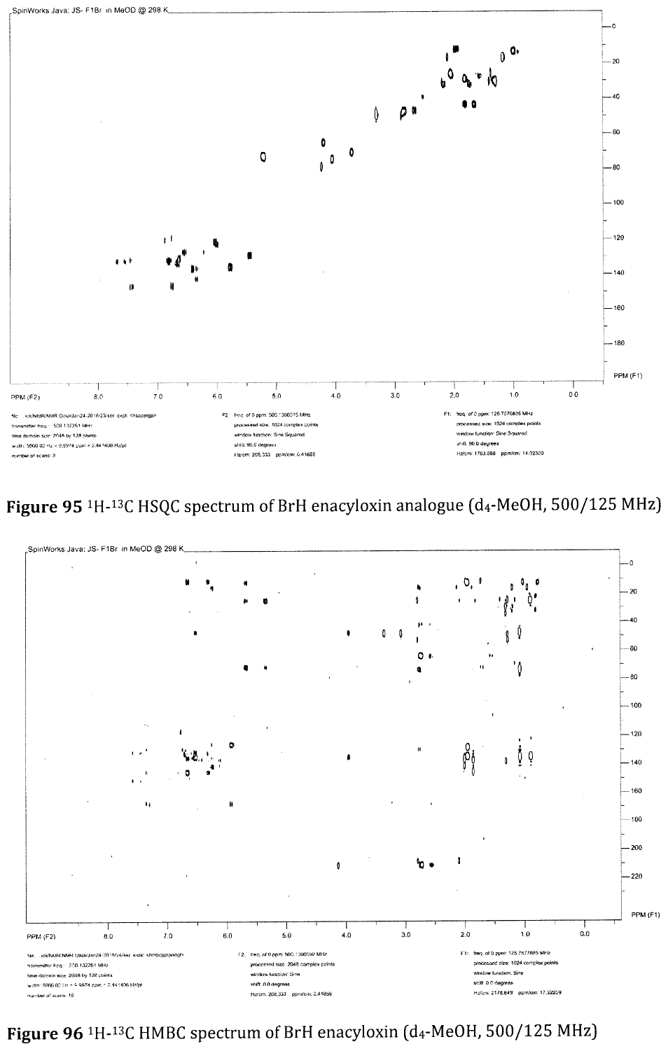

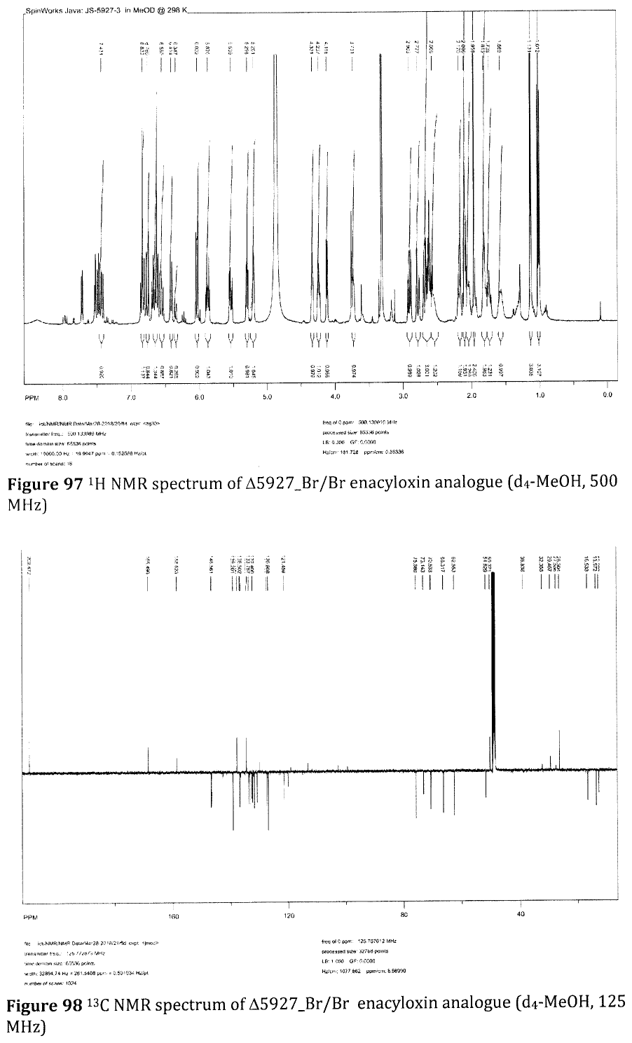

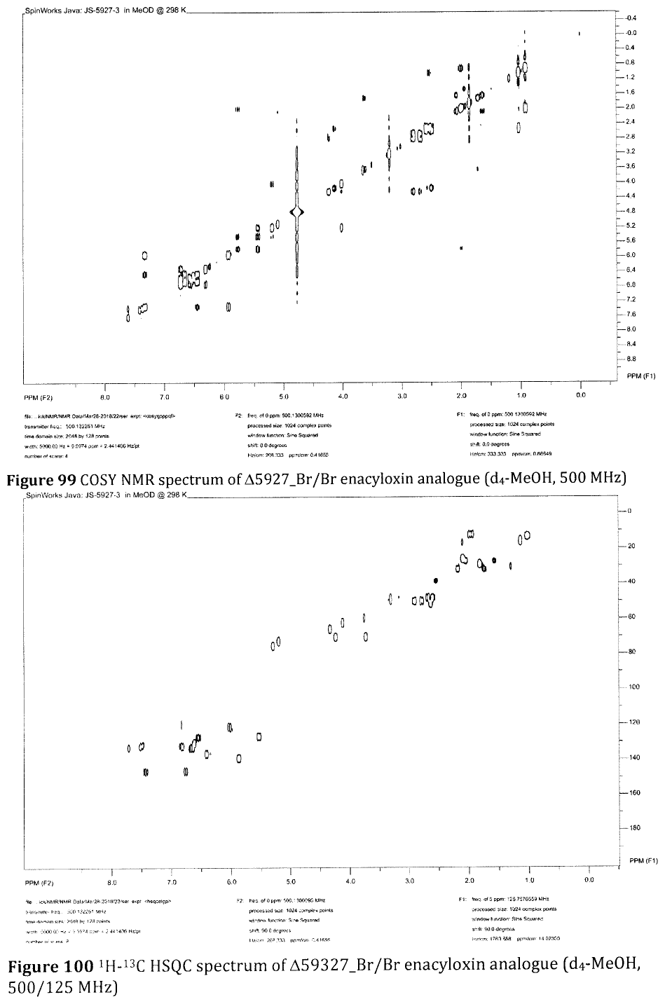

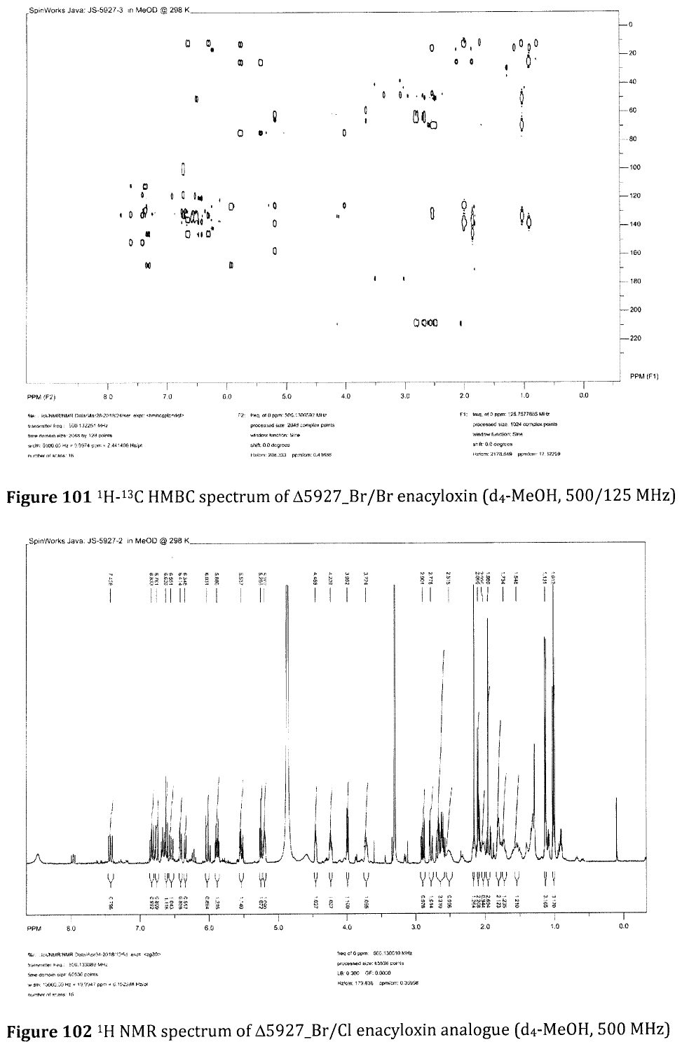

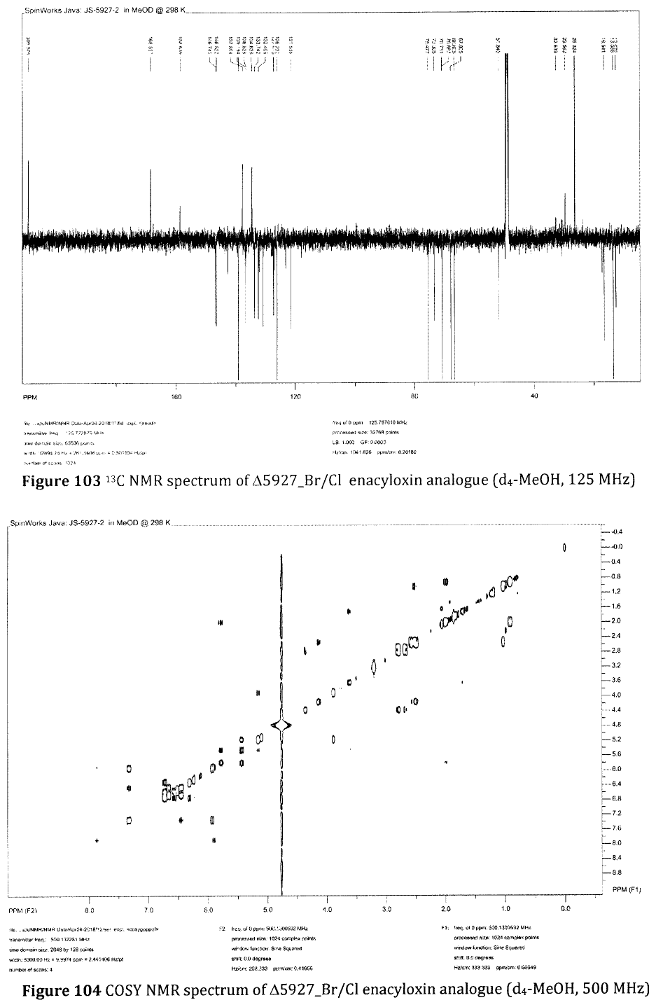

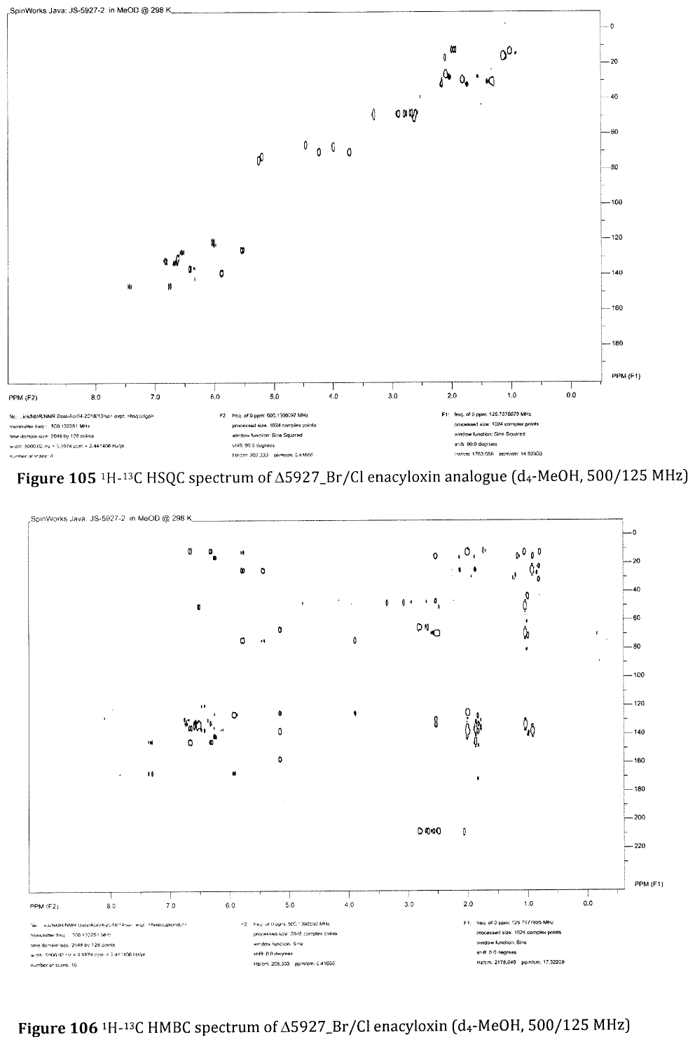

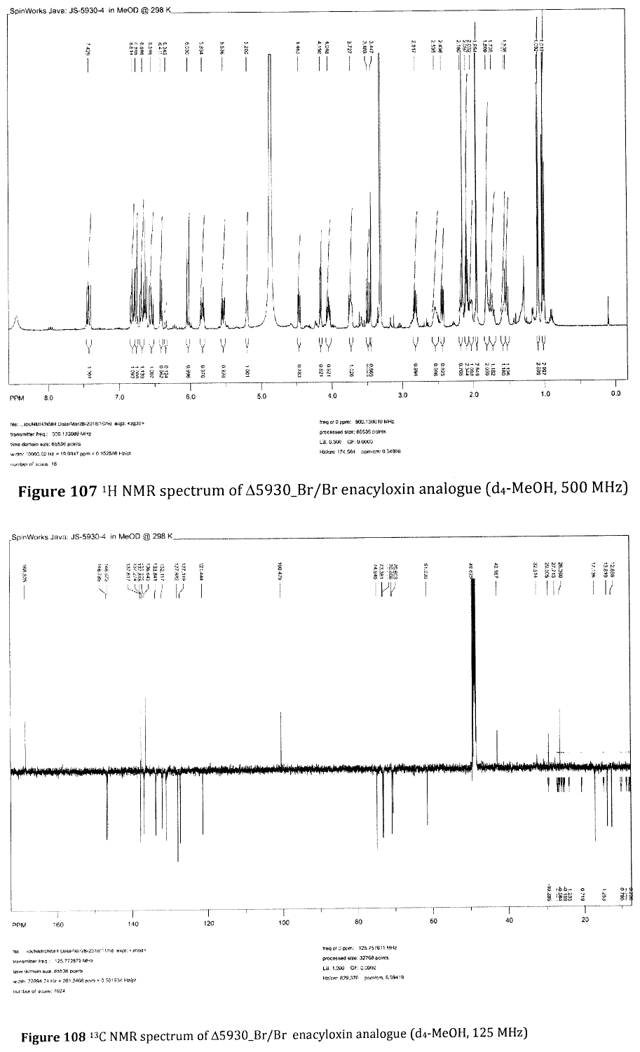

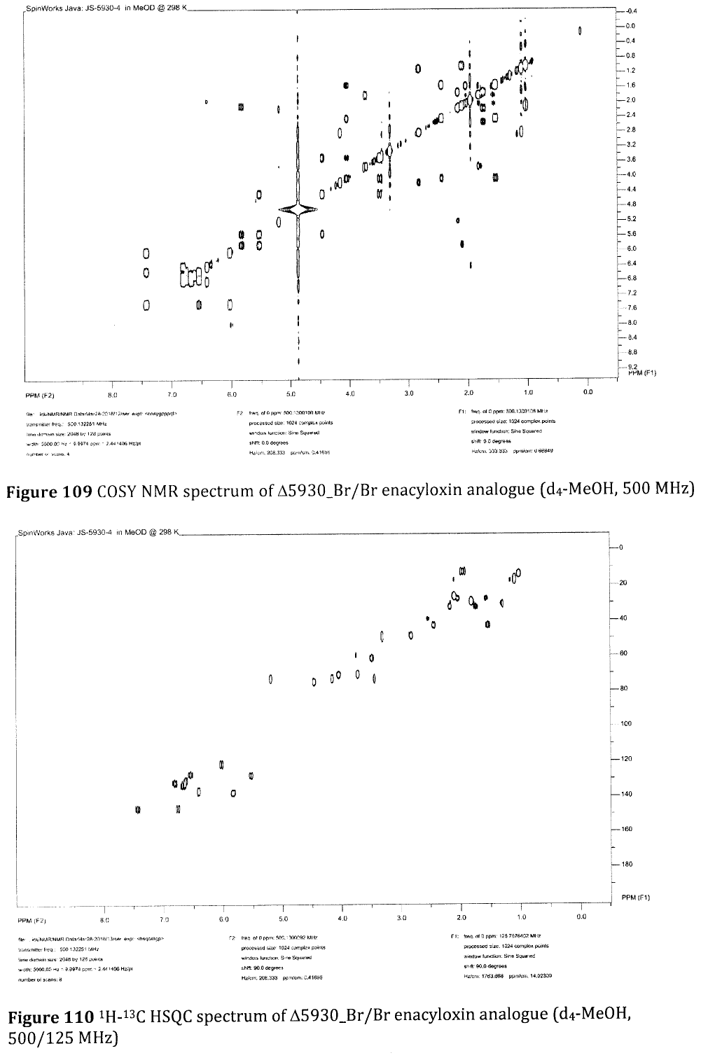

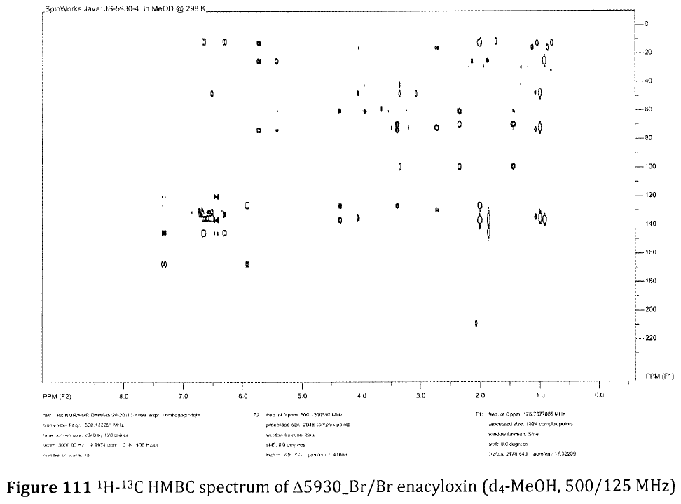

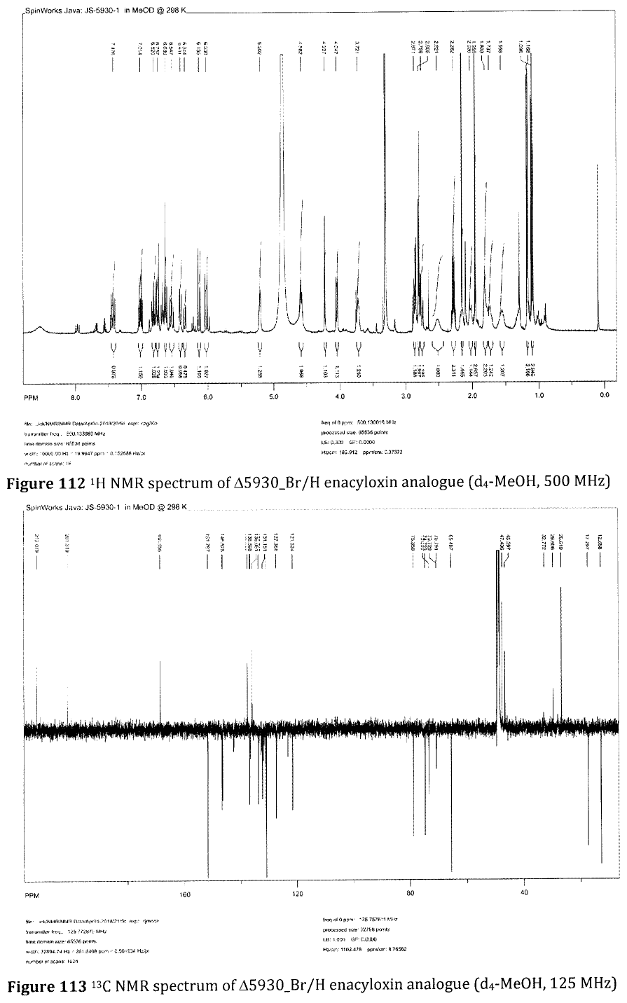

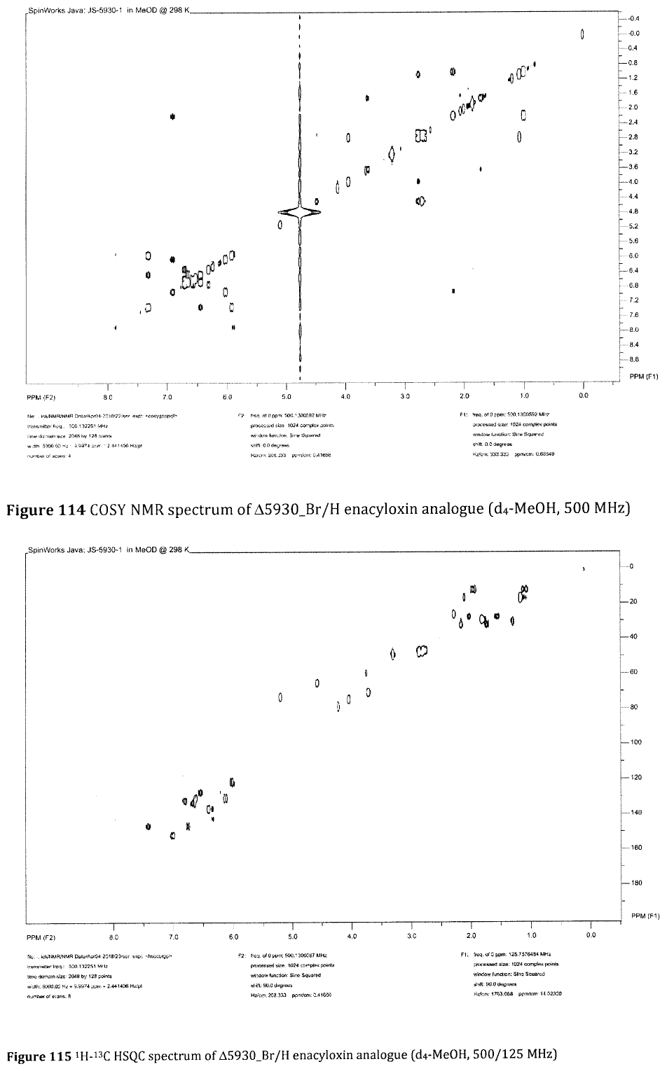

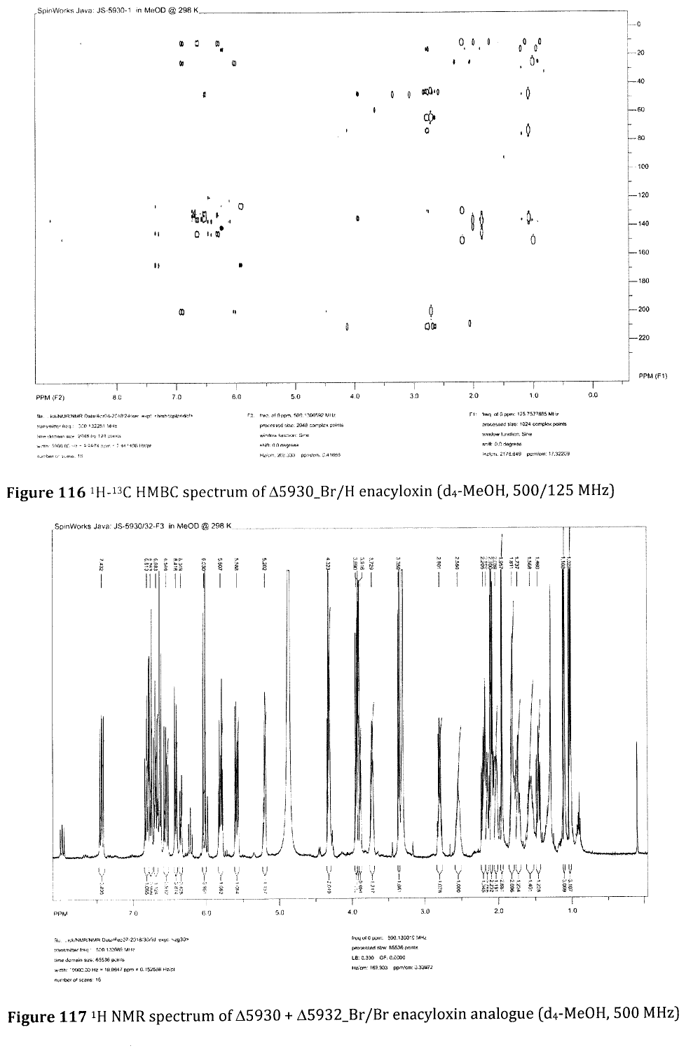

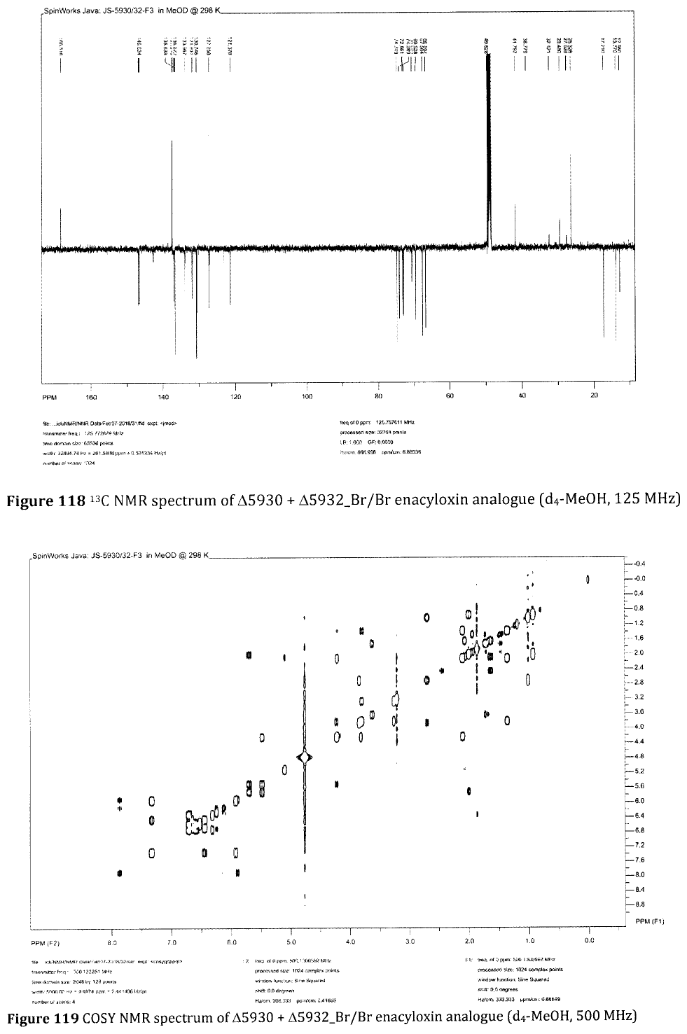

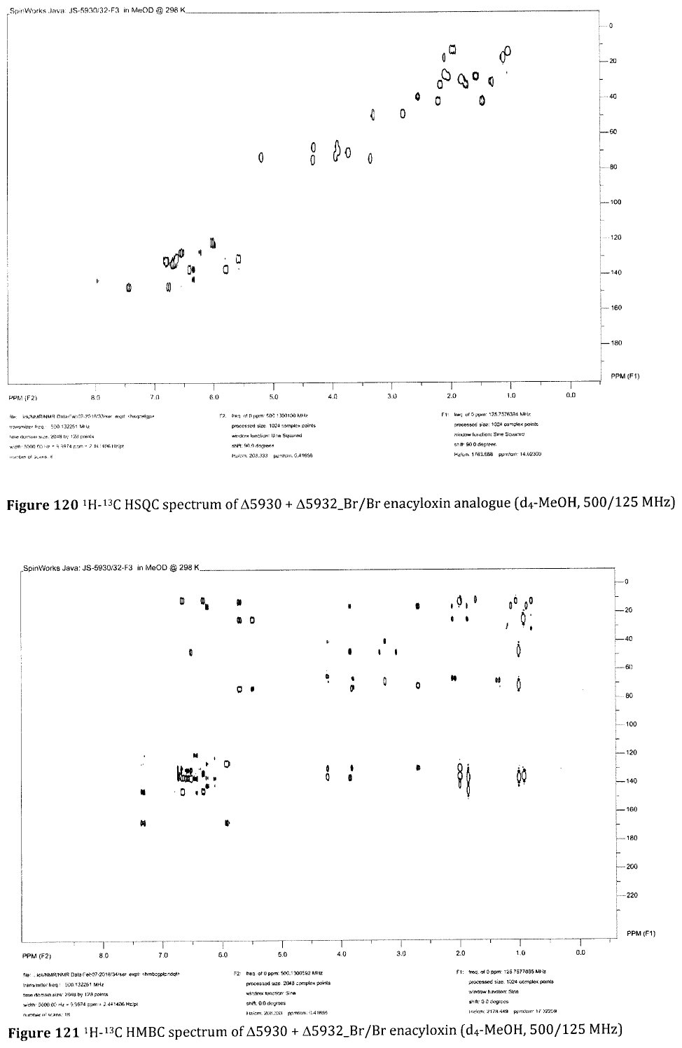

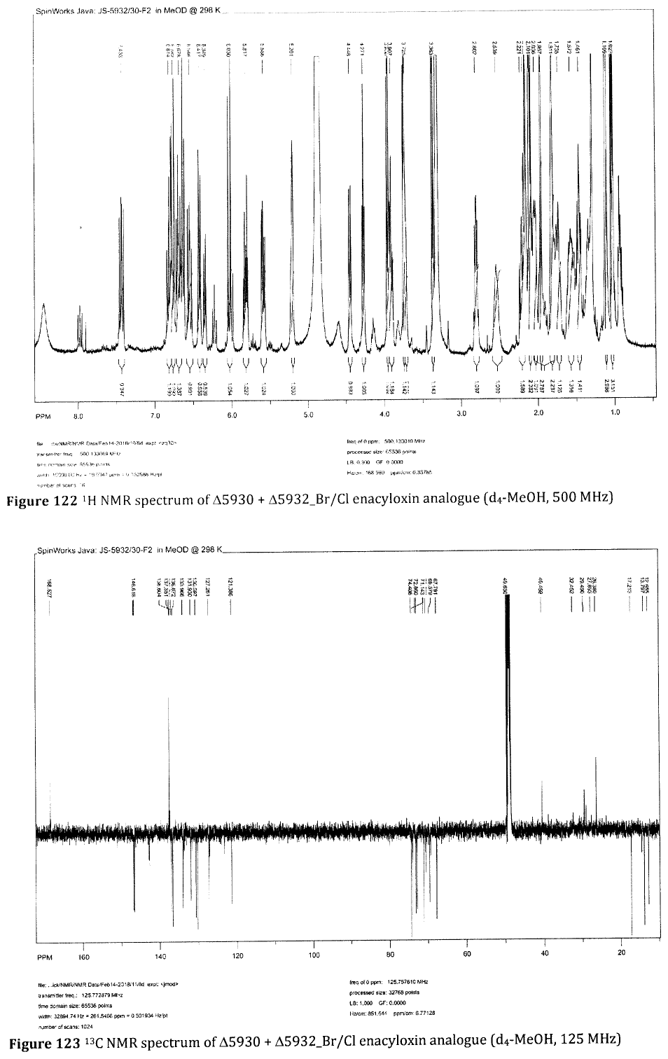

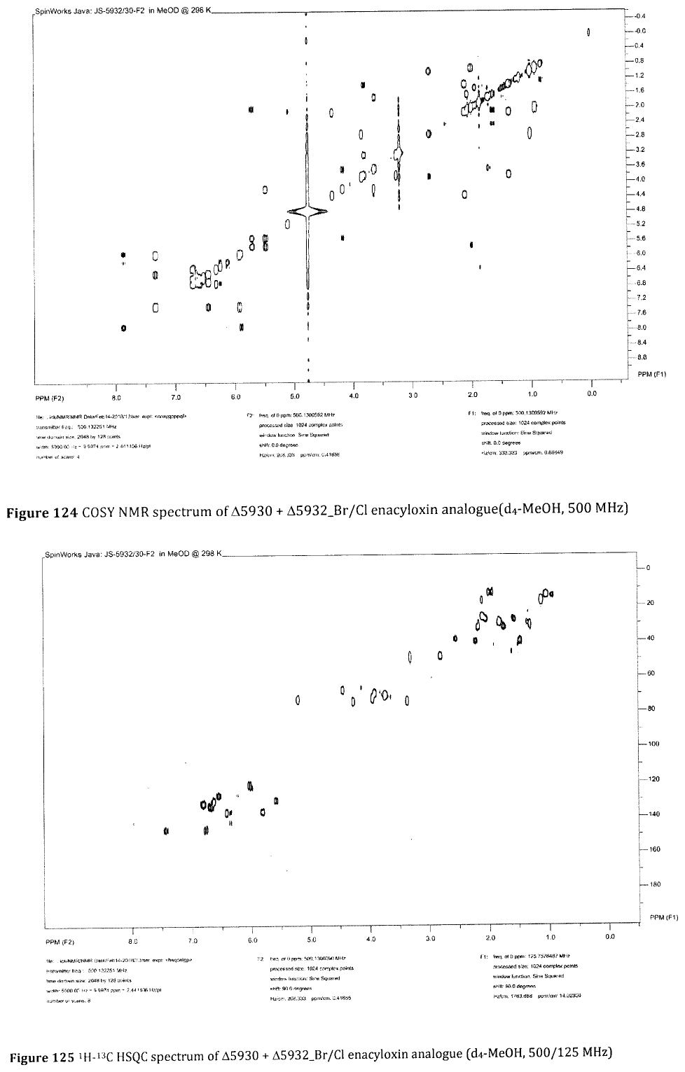

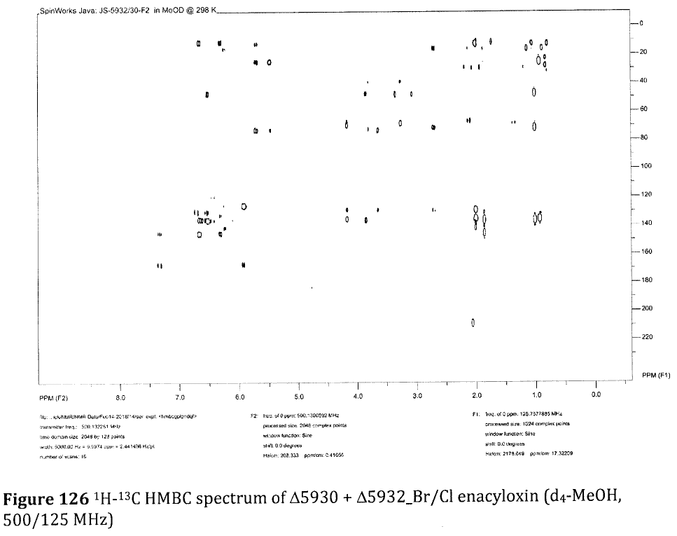

FIGS. 1 to 126 show ¹H NMR, ¹³C NMR, COSY NMR, ¹H-¹³C HSQC and ¹H-¹³C HMBC spectra of compounds according to the invention.

### EXAMPLES

### Example 1—Production and Purification of Enacyloxin Derivatives

### Gene Deletion

In-frame deletions in genes were introduced via double homologous recombination using the suicide plasmid pGPI and the I-SceI expression plasmid pDAI (Flannagan et al., Environ. Microbiol. 10: 1652-1660, 2008). The sequences (500-1000 bp) flanking the gene regions targeted for deletion were amplified from B. ambifaria BCC0203 genomic DNA using Q5 DNA polymerase (NEB). Restriction sites were introduced at the 5′-end of the primers to allow for directional cloning of the PCR products into pGPI. Constructs were mobilized into E. coli SY327 by electroporation and transformants were selected on LB agar plates supplemented with trimethoprim (50 μg/mL). Plasmids were purified from trimethoprim-resistant colonies using the GeneJET Plasmid Miniprep kit (Thermo Scientific) and correct assembly of the mutagenesis constructs was confirmed by Sanger sequencing (GATC Biotech). Validated constructs were transferred into B. ambifaria BCC0203 via triparental mating (Agnoli et al., Mol. Microbiol. 83: 362-378, 2012) and transconjugants were selected using trimethoprim (200 μg/ml) and gentamicin (50 μg/ml). Single B. ambifaria mutants were selected and correct integration of the mutagenesis plasmids into the genome was confirmed by colony PCR. Next, the pDAI plasmid was introduced into the B. ambifaria single crossover mutants by triparental mating using E. coli SY327 (pDAI) and E. coli HB101 (pRK2013) as the donor and helper strain, respectively (Agnoli et al., Mol. Microbiol. 83: 362-378, 2012). Transconjugants were selected on LB agar plates containing tetracycline (200 μg/ml) and gentamicin (50 μg/ml). Single B. ambifaria mutants were selected and correct gene deletion was confirmed by colony PCR and Sanger sequencing. To examine the effect of the gene deletions on enacyloxin biosynthesis, mutant strains were grown at 30° C. on solid minimal medium containing glycerol as a sole carbon source (BSM-G) (O'Sullivan et al., Environ. Microbiol. 9: 1017-1034, 2007). Following incubation for 3 days, the cells were scraped off and ethyl acetate extracts of the agar were analysed by UHPLC-ESI-Q-TOF-MS.

### Production and Purification of the Enacyloxin Derivatives

For production of enacyloxin derivatives, the B. ambifaria BCC0203 deletion mutants were grown in the dark at 30° C. on solid minimal medium containing glycerol as a sole carbon source (BSM-G) (O'Sullivan et al., Environ. Microbiol. 9: 1017-1034, 2007). Following incubation for 3 days, the cells were scraped off and the agar extracted twice using ethyl acetate (1:1). Enacyloxin derivative-containing extracts were concentrated by rotary evaporation in vacuo and the resulting residue was re-dissolved in acetonitrile for purification by preparative HPLC. For the mutasynthetic production of enacyloxin derivatives, B. ambifaria BCC0203 mutant Δ5912-5914 was grown in the dark at 30° C. on BSM-G agar supplemented with 10 mM of a relevant DHCCA analogue (described below). Bromine derivatives of enacyloxin were generated using the same procedure, however the ammonium chloride in the BSM-G was replaced with two equivalents of ammonium bromide.

Enacyloxin derivative-containing ethyl acetate extracts of the agar were fractionated by preparative HPLC using an Agilent 1260 instrument equipped with a Zorbax SB-C₁₈ column (21.2×100 mm, 5 μm), monitoring absorbance at 360 nm. Mobile phases consisted of water and acetonitrile, each supplemented with 0.1% formic acid. A gradient of 50% B to 100% B over 40 minutes was employed at a flow rate of 10 mL/min. Enacyloxin derivative-containing fractions were pooled, concentrated in vacuo, and subsequently lyophilized. To avoid photo-induced isomerization of the compounds, exposure to light was minimized throughout the entire purification process.

### Synthesis of DHCCA Derivatives—General Procedures

Room temperature refers to ambient temperature (20-22° C.), 5° C. refers to a cold water bath and 00° C. refers to an ice slush bath. Heated experiments were conducted using thermostatically controlled oil baths. All commercially available solvents and chemicals were used without any further purification. NMR spectra were recorded on Bruker Advance AV-300 and HD-500 MHz spectrometers at room temperature (298 K). Chemical shifts are reported in parts per million (ppm) referenced from CDCl3 (δH: 7.26 ppm and δC: 77.0 ppm). Coupling constants (J) are rounded to the nearest 0.5 Hertz (Hz). Multiplicities are given as multiplet (m), singlet (s), doublet (d), triplet (t), quartet (q), quintet (quin.), sextet (sext.), septet (sept.), octet (oct.) and nonet (non.). 1H and 13C assignments were established on the basis of COSY, DEPT, HMQC and HMBC correlations. Infra-red spectra were recorded using either a Perkin Elmer Spectrum 100 FT-IR spectrometer or an Alpha Bruker Platunium ATR single reflection diamond ATR module. Optical rotations were measured using an Optical Activity Ltd AA-1000 millidegree auto-ranging polarimeter (589 nm). Specific rotations are given in units of 10-1 deg cm²g⁻¹. Melting points were recorded on a Stuart scientific melting point apparatus and are uncorrected. Silica column chromatography was performed on 40-60 Å silica gel. Thin layer chromatography (TLC) was carried out aluminum sheets coated with 0.2 mm silica gel 60 F254. Visualisation was effected by UV light (254 nm) or by potassium permanganate solution followed by heating. Low resolution mass spectra (LRMS) were recorded using an Agilent 6130B single Quad (ESI). High resolution mass spectra (HRMS) were obtained using a Bruker micro-TOF ESI spectrometer.

### Synthesis of Cyclopentane DHCCA Derivative—(1S,3R,4S)-3,4-dihydroxycyclopentane-1-carboxylic acid (2)

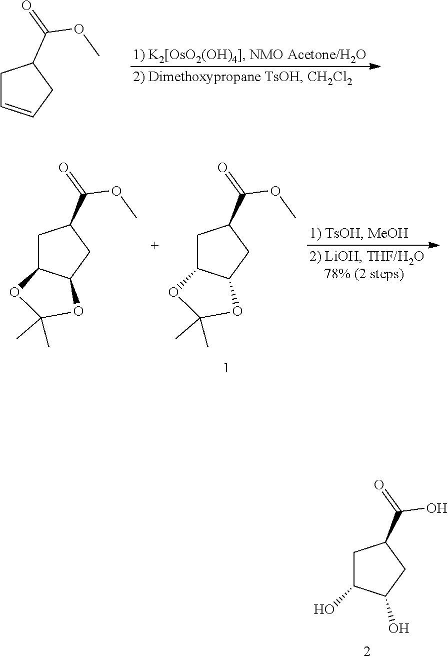

Acetonide (1) was synthesized according to literature procedure (WO 2013/170030). To a solution of acetonide (1) (328 mg, 1.64 mmol) in MeOH (10 mL) was added TsOH (35 mg, 0.20 mmol) and the reaction was stirred at room temperature for 1 hour. The mixture was then concentrated in vacuo and partitioned between EtOAc (20 mL) and saturated NaHCO₃ (20 mL). The layers were separated and the aqueous phase further extracted with EtOAc (2×20 mL), the combined organics were then washed with brine, dried (MgSO₄), filtered and concentrated in vacuo to afford the crude diol as a viscous oil. The diol was then dissolved in THF (10 mL) and H₂O (5 mL), and LiOH was added (40 mg, 2 mmol). The reaction was then stirred at room temperature overnight and concentrated in vacuo to afford the product as a viscous oil (162 mg, 78%).

δ_{H} (500 MHz, MeOD) 4.09-4.02 (2H, br. m, CHOH), 3.06 (1H, tt, J 9.5 and 3, CHCOOH), 2.09-1.88 (4H, m, CH₂); δ_{C} (125 MHz, MeOD) 183.2 (CO₂H), 73.2 (CHOH), 40.1 (CHCOOH), 35.1 (CH₂); HRMS (ESI) cald. for C₆H₁₀NaO₄ (M+Na⁺) requires 169.0477, found 169.0475.

### Synthesis of (1R,3R,4R)-3,4-dihydroxycyclohexane-1-carboxylic acid (3)

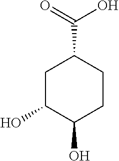 [SMILES: O=C(O)[C@@H]1CC[C@@H](O)[C@H](O)C1]

(1R,3R,4R)-3,4-dihydroxycyclohexane-1-carboxylic acid (3) was synthesized and used as a racemic mixture by following literature procedure (WO 2015/4455).

### Synthesis of Unsaturated DHCCA Derivative—(4S,5R)-4,5-dihydroxycyclohex-1-ene-1-carboxylic acid (7)

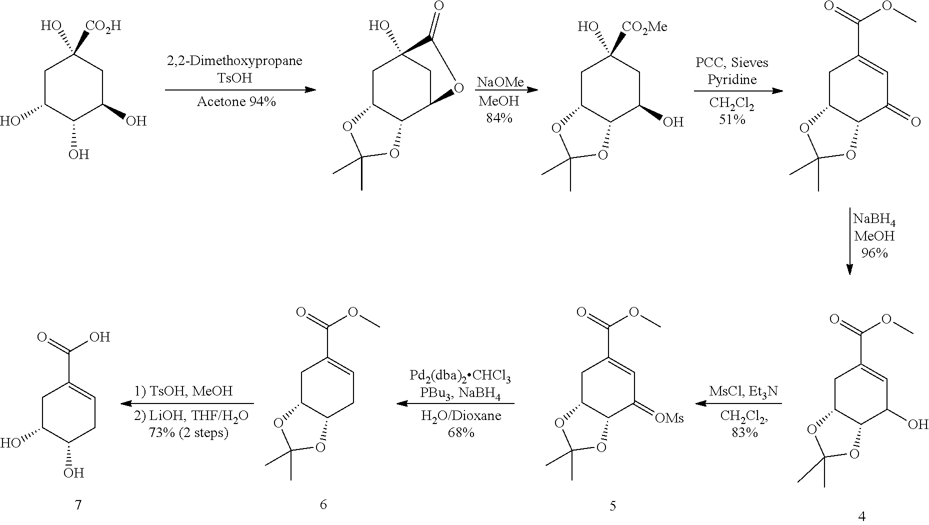

Alcohol (4) was synthesized according to literature procedure (Z. Wang et al., J. Org. Chem., 1997, 62, 8622-8623). To a stirred solution of alcohol (4) (370 mg, 1.62 mmol) in CH₂Cl₂ (10 mL) was added Et₃N (2.25 mL, 16.2 mmol) and methanesulfonyl chloride (0.63 mL, 8.11 mmol) at 000° C. and the mixture was stirred overnight at room temperature. The reaction was quenched with 1M HCl (10 mL) and the mixture extracted with CH₂Cl₂ (3×10 mL), the combined organics were then washed with brine, dried (MgSO₄), filtered and concentrated in vacuo to afford the crude product as an orange solid. The crude product was purified by silica chromatography (EtOAc:Petroleum ether, 60:40) to afford methyl (3aR,7aR)-2,2-dimethyl-7-((methylsulfonyl)oxy)-3a,4,7,7a-tetrahydrobenzo[d][1,3]dioxole-5-carboxylate (5) as a white solid (411 mg, 83%).

δ_{H} (500 MHz, CDCl₃) 6.95 (1H, br. s, CHCHOS), 5.06 (1H, br. s, CHOS), 4.75-4.71 (1H, m, CH₂CHOCHO), 4.67-4.63 (1H, m, CH₂CHO), 3.78 (3H, s, OCH₃), 3.17 (3H, s SCH₃), 3.07 (1H, d, J 16.5, CH₂), 2.00 (1H, d, J 16.5, CH₂), 1.34 (3H, s, CCH₃), 1.32 (3H, s, CCH₃); δ_{C} (125 MHz, CDCl₃) 165.5 (CO₂Me), 136.2 (CHCOS), 130.8 (CCOOMe), 109.9 (C(CH₃)₂), 75.7 (CHOS), 75.4 (CH₂CHOCHO), 72.5 (CH₂CHO), 52.4 (COOCH₃), 39.2 (SCH₃), 27.2 (CH₂), 25.8 (CH₃), 24.4 (CH₃); HRMS (ESI) cald. for C₁₂H₁₈NaO₇S (M+Na⁺) requires 329.0671, found 329.0672.

To a stirred solution of Pd₂(dba)₃.CHCl₃ (60 mg, 0.06 mmol), tributylphosphine (15 μl. 0.06 mmol) and mesylate (5) (330 mg, 1.1 mmol) in dioxane (10 mL) under and argon atmosphere was added a suspension of NaBH₄ (40 mg, 1.1 mmol) in H₂O (1 mL) and the reaction was stirred for 2 hours at room temperature. The reaction mixture was then diluted with saturated brine (10 mL) and extracted with Et₂O (3×20 mL), the combined organics were then washed with brine, dried (MgSO₄), filtered and concentrated in vacuo to afford the crude product as a black oil. The crude product was purified by silica chromatography (EtOAc:Petroleum ether, 10:90) to afford methyl (3aR,7aS)-2,2-dimethyl-3a,4,7,7a-tetrahydrobenzo[d][1,3]dioxole-5-carboxylate (6) as a colourless oil (159 mg, 68%).

δ_{H} (500 MHz, MeOD) 7.02 (1H, dt, J 6 and 3, CHCH₂CHO), 4.52 (1H, ddd, J 7, 4 and 3, CH₂CHO), 4.48 (1H, ddd, J 7, 5 and 2.5, CHCH₂CHO), 3.74 (3H, s, OCH₃), 2.74 (1H, dd, J 16.5 and 3, CH₂C), 2.50 (1H, dd, J 17, 6 and 2, CHCH₂), 2.29-2.20 (2H, m, CH₂C, CHCH₂), 1.29 (3H, s, CCH₃), 1.28 (3H, s, CCH₃); δ_{C} (125 MHz, MeOD) 168.4 (CO₂Me), 139.5 (CHCH₂), 130.7 (CCOOMe), 108.9 (C(CH₃)₂), 74.7 (CH₂CHO), 74.0 (CHCH₂CHO), 52.2 (COOCH₃), 30.0 (CH₂C), 28.0 (CHCH₂) 26.7 (CH₃), 24.6 (CH₃); HRMS (ESI) cald. for C₁₁H₁₆NaO₄ (M+Na⁺) requires 235.0946, found 235.0949.

To a solution of acetonide (6) (150 mg, 0.7 mmol) in MeOH (10 mL) was added TsOH (17 mg, 0.1 mmol) and the reaction was stirred at room temperature for 1 hour. The mixture was then concentrated in vacuo and partitioned between EtOAc (20 mL) and saturated NaHCO₃ (20 mL). The layers were separated and the aqueous phase further extracted with EtOAc (2×20 mL), the combined organics were then washed with brine, dried (MgSO₄), filtered and concentrated in vacuo to afford the crude diol as a viscous oil. The diol was then dissolved in THF (10 mL) and H₂O (5 mL), and LiOH was added (20 mg, 1 mmol). The reaction was then stirred at room temperature overnight and concentrated in vacuo to afford (4S,5R)-4,5-dihydroxycyclohex-1-ene-1-carboxylic acid (7) as a viscous oil (81 mg, 73%).

δ_{H} (500 MHz, MeOD) 6.87-6.84 (1H, m, OHCH₂CHO), 3.92-3.84 (2H, m, CHO), 2.55-2.35 (4H, m, CH₂), δ_{C} (125 MHz, MeOD) 170.25 (CO₂H), 137.8 (CHCH₂), 128.9 (CCOOH), 69.6 (CH₂CHO), 68.5 (CHCH₂CHO), 32.3 (CH₂C), 31.0 (CHCH₂) 26.7 (CH₃); HRMS (ESI) cald. for C₇H₁₀NaO₄ (M+Na⁺) requires 181.0477, found 181.0476.

### Structure Elucidation of the Enacyloxin Derivatives

Structure elucidation of the compounds was achieved using a combination of UHPLC-ESI-Q-TOF-MSIMS and 1- and 2-D NMR experiments. UHPLC-ESI-Q-TOF-MS analyses were performed on a Zorbax Eclipse Plus C₁₈ column (1.8 m, 2.1×100 mm, Agilent) coupled to a Bruker MaXis Impact mass spectrometer. Mobile phases consisted of water and acetonitrile (ACN), each supplemented with 0.1% formic acid. A gradient of 30% ACN to 100% ACN over 34 minutes was employed at a flow rate of 0.2 mL/min. The mass spectrometer was operated in positive ion mode with a scan range of 50-3000 m/z. UV absorbance was monitored at 360 nm.

For NMR analysis, purified compounds were dissolved in d4-MeOH and ¹H, ¹³C, COSY, HSQC and HMBC spectra were recorded on a Bruker Avance 500 MHz spectrometer equipped with a DCH cryoprobe at 25° C.

Knock-out mutagenesis or mutasynthesis methods as described above were used to prepare the compounds in Table 2.

TABLE 2
Polyketide compounds of the invention

| Compound | | |
|---|---|---|
| No. | Gene manipulation method | Structure of compound |
| 1 | B. ambifaria BCCO203 Δbamb_5927 | 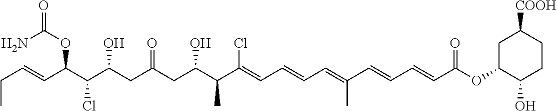 [SMILES: CC/C=C/[C@@H](OC(N)=O)[C@@H](Cl)[C@H](O)CC(=O)C[C@H](O)[C@H](C)/C(Cl)=C/C=C/C=C(C)/C=C/C=C/C(=O)O[C@@H]1C[C@@H](C(=O)O)CC[C@@H]1O] |
| 2 | B. ambifaria BCCO203 Δbamb_5931 | 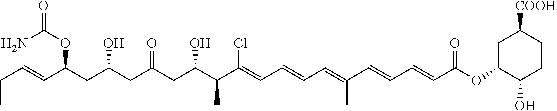 [SMILES: CC/C=C/[C@H](C[C@H](O)CC(=O)C[C@H](O)[C@H](C)/C(Cl)=C/C=C/C=C(C)/C=C/C=C/C(=O)O[C@@H]1C[C@@H](C(=O)O)CC[C@@H]1O)OC(N)=O] |
| 3 | B. ambifaria BCCO203 Δbamb_5930 | 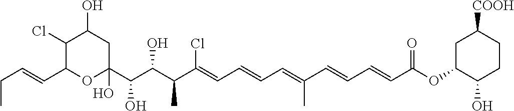 [SMILES: CC/C=C/C1OC(O)([C@@H](O)[C@H](O)[C@H](C)/C(Cl)=C/C=C/C=C(C)/C=C/C=C/C(=O)O[C@@H]2C[C@@H](C(=O)O)CC[C@@H]2O)CC(O)C1Cl] |
| 4 | B. ambifaria BCCO203 Δbamb_5912 | 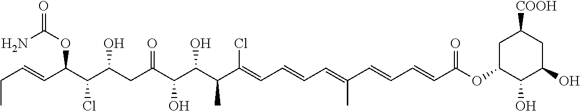 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@@H](C(=O)O)C[C@@H](O)[C@@H]1O] |
| 5 | B. ambifaria BCCO203 Δbamb_5927, Δbamb_5930 | 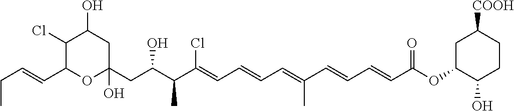 [SMILES: CC/C=C/C1OC(O)(C[C@H](O)[C@H](C)/C(Cl)=C/C=C/C=C(C)/C=C/C=C/C(=O)O[C@@H]2C[C@@H](C(=O)O)CC[C@@H]2O)CC(O)C1Cl] |
| 6 | B. ambifaria BCCO203 Δbamb_5927, Δbamb_5931 | 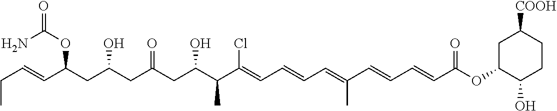 [SMILES: CC/C=C/[C@H](C[C@H](O)CC(=O)C[C@H](O)[C@H](C)/C(Cl)=C/C=C/C=C(C)/C=C/C=C/C(=O)O[C@@H]1C[C@@H](C(=O)O)CC[C@@H]1O)OC(N)=O] |
| 7 | B. ambifaria BCCO203 Δbamb_5928 | 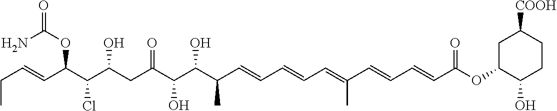 [SMILES: CC/C=C/[C@@H](OC(N)=O)[C@@H](Cl)[C@H](O)CC(=O)[C@@H](O)[C@H](O)[C@H](C)/C=C/C=C/C=C(C)/C=C/C=C/C(=O)O[C@@H]1C[C@@H](C(=O)O)CC[C@@H]1O] |
| 8 | B. ambifaria BCCO203 Δbamb_5932 | 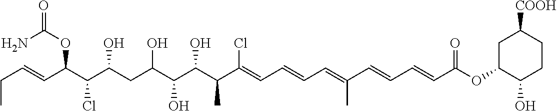 [SMILES: CC/C=C/[C@@H](OC(N)=O)[C@@H](Cl)[C@H](O)CC(O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O] |
| 9 | B. ambifaria BCCO203 Δbamb_5930, Δbamb_5932 | 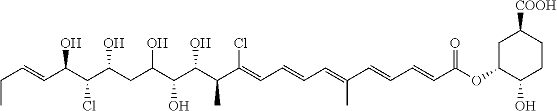 [SMILES: CC/C=C/[C@@H](O)[C@@H](Cl)[C@H](O)CC(O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O] |
| 10 | B. ambifaria BCCO203 Δbamb_5930, Δbamb_5931 | 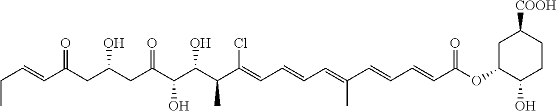 [SMILES: CC/C=C/C(=O)C[C@H](O)CC(=O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O] |
| 11 | B. ambifaria BCCO203 Δbamb_5930-5932 | 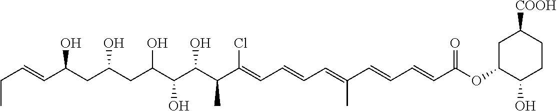 [SMILES: CC/C=C/[C@@H](O)C[C@H](O)CC(O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O] |
| 12 | B. ambifaria BCCO203 Δbamb_5927-5932 | 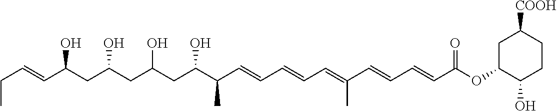 [SMILES: CC/C=C/[C@@H](O)C[C@H](O)CC(O)C[C@H](O)[C@H](C)/C=C/C=C/C=C(C)/C=C/C=C/C(=O)O[C@@H]1C[C@@H](C(=O)O)CC[C@@H]1O] |
| 13 | B. ambifaria BCC0203 Δ5912-5914 + 3-amino-4- hydroxycyclohexane carboxylic acid | 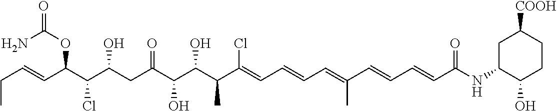 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)N[C@@H]1C[C@@H](C(=O)O)CC[C@@H]1O] |
| 14 | B. ambifaria BCC0203 Δ5912-5914 + 3-amino-4- hydroxycyclohexane carboxylic acid | 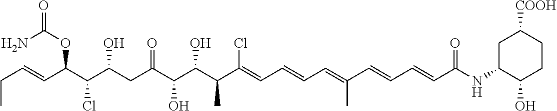 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)N[C@@H]1C[C@H](C(=O)O)CC[C@@H]1O] |
| 15 | B. ambifaria BCC0203 Δ5912-5914 + 3-amino-4- hydroxycyclohexane carboxylic acid | 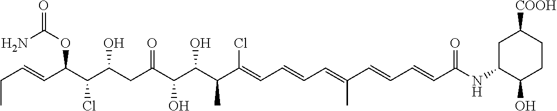 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)N[C@@H]1C[C@@H](C(=O)O)CC[C@H]1O] |
| 16 | B. ambifaria BCC0203 Δ5912-5914 + 3,4- dihydroxycyclohexane carboxylic acid | 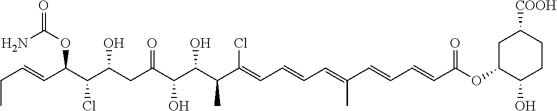 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@H](C(=O)O)CC[C@@H]1O] |
| 17 | B. ambifaria BCC0203 Δ5912-5914 + 3,4- dihydroxycyclohexane carboxylic acid | 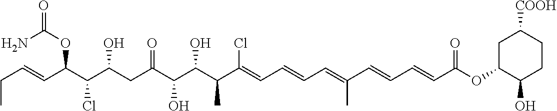 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@H](C(=O)O)CC[C@H]1O] |
| 18 | B. ambifaria BCC0203 Δ5912-5914 + 3- hydroxycyclopentane carboxylic acid | 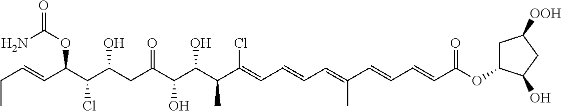 |
| 19 | B. ambifaria BCC0203 Δ5912-5914 + 3,4- diaminocyclohexane carboxylic acid | 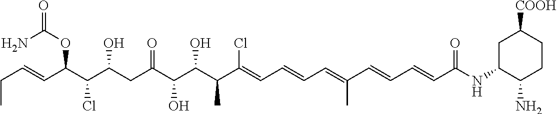 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)N[C@@H]1C[C@@H](C(=O)O)CC[C@@H]1N] |
| 20 | B. ambifaria BCC0203 Δ5912-5914 + syn-3- aminocyclopentane carboxylic acid | 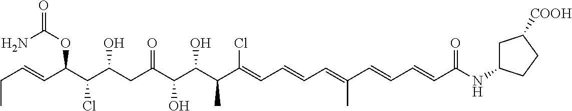 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)N[C@H]1CC[C@@H](C(=O)O)C1] |
| 21 | B. ambifaria BCC0203 Δ5912-5914 + 3- aminocyclohexane carboxylic acid | 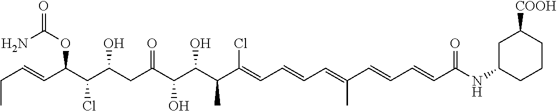 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)N[C@H]1CCC[C@H](C(=O)O)C1] |
| 22 | B. ambifaria BCC0203 Δ5912-5914 + 3- aminocyclohexane carboxylic acid | 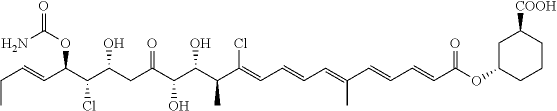 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@H]1CCC[C@H](C(=O)O)C1] |
| 23 | B. ambifaria BCC0203 Δ5912-5914 + 4-amino-3- hydroxybutyric acid | 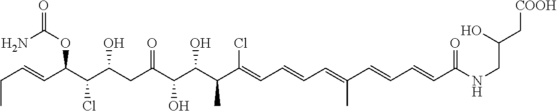 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)NCC(O)CC(=O)O] |
| 24 | B. ambifaria BCC0203 Δ5912-5914 + 4- aminobutyric acid | 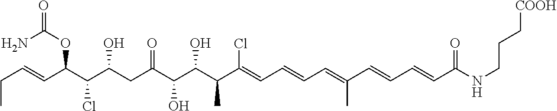 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)NCCCC(=O)O] |
| 25 | B. ambifaria BCC0203 Δ5912-5914 + shikimate | 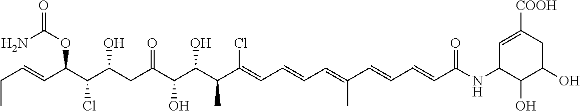 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)NC1C=C(C(=O)O)CC(O)C1O] |
| 26 | B. ambifaria BCC0203 Δ5912-5914 + shikimate | 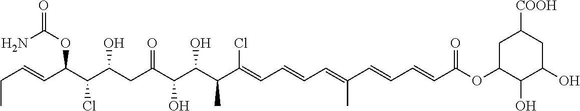 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)OC1CC(C(=O)O)CC(O)C1O] |
| 27 | B. ambifaria BCC0203 Δ5912-5914 + shikimate | 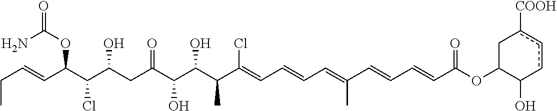 |
| 28 | B. ambifaria BCC0203 Δ5912-5914 + DHCCA derivative (7) | 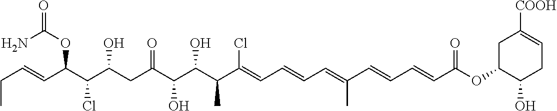 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1CC(C(=O)O)=CC[C@@H]1O] |
| 29 | B. ambifaria BCC0203 Δ5912-5914 + Δ5930 + Δ5932 + DHCCA derivative (7) | 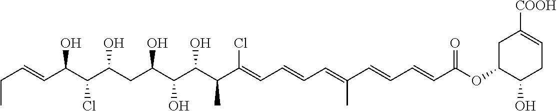 [SMILES: CC/C=C/[C@@H](O)[C@@H](Cl)[C@H](O)C[C@@H](O)[C@@H](O)[C@H](O)[C@H](C)/C(Cl)=C/C=C/C=C(C)/C=C/C=C/C(=O)O[C@@H]1CC(C(=O)O)=CC[C@@H]1O] |
| 30 | B. ambifaria BCC0203 | 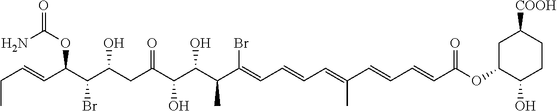 [SMILES: CC/C=C/[C@@H](OC(N)=O)[C@@H](Br)[C@H](O)CC(=O)[C@@H](O)[C@H](O)[C@H](C)/C(Br)=C/C=C/C=C(C)/C=C/C=C/C(=O)O[C@@H]1C[C@@H](C(=O)O)CC[C@@H]1O] |
| 31 | B. ambifaria BCC0203 | 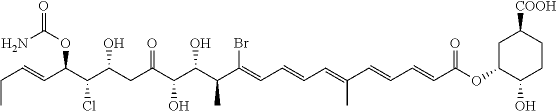 [SMILES: CC/C=C/[C@@H](OC(N)=O)[C@@H](Cl)[C@H](O)CC(=O)[C@@H](O)[C@H](O)[C@H](C)/C(Br)=C/C=C/C=C(C)/C=C/C=C/C(=O)O[C@@H]1C[C@@H](C(=O)O)CC[C@@H]1O] |
| 32 | B. ambifaria BCC0203 | 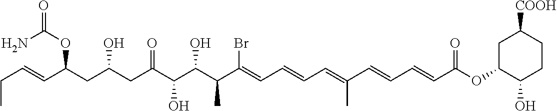 [SMILES: CC/C=C/[C@H](C[C@H](O)CC(=O)[C@@H](O)[C@H](O)[C@H](C)/C(Br)=C/C=C/C=C(C)/C=C/C=C/C(=O)O[C@@H]1C[C@@H](C(=O)O)CC[C@@H]1O)OC(N)=O] |
| 33 | B. ambifaria BCC0203 Δbamb_5930, Δbamb_5932 | 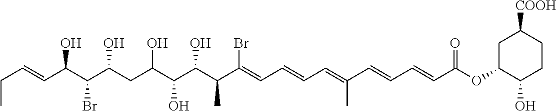 [SMILES: CC/C=C/[C@@H](O)[C@@H](Br)[C@H](O)CC(O)[C@@H](O)[C@H](O)[C@H](C)/C(Br)=C/C=C/C=C(C)/C=C/C=C/C(=O)O[C@@H]1C[C@@H](C(=O)O)CC[C@@H]1O] |
| 34 | B. ambifaria BCC0203 Δbamb_5930, Δbamb_5932 | 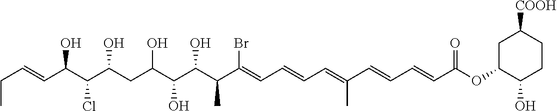 [SMILES: CC/C=C/[C@@H](O)[C@@H](Cl)[C@H](O)CC(O)[C@@H](O)[C@H](O)[C@H](C)/C(Br)=C/C=C/C=C(C)/C=C/C=C/C(=O)O[C@@H]1C[C@@H](C(=O)O)CC[C@@H]1O] |
| 35 | B. ambifaria BCC0203 Δbamb_5930, Δbamb_5932 | 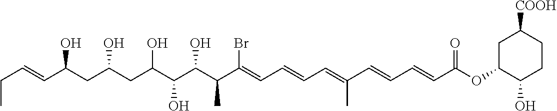 [SMILES: CC/C=C/[C@@H](O)C[C@H](O)CC(O)[C@@H](O)[C@H](O)[C@H](C)/C(Br)=C/C=C/C=C(C)/C=C/C=C/C(=O)O[C@@H]1C[C@@H](C(=O)O)CC[C@@H]1O] |
| 36 | B. ambifaria BCC0203 Δbamb_5927 | 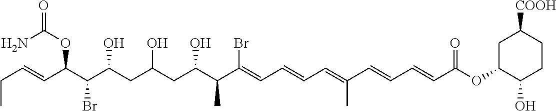 [SMILES: CC/C=C/[C@@H](OC(N)=O)[C@@H](Br)[C@H](O)CC(O)C[C@H](O)[C@H](C)/C(Br)=C/C=C/C=C(C)/C=C/C=C/C(=O)O[C@@H]1C[C@@H](C(=O)O)CC[C@@H]1O] |
| 37 | B. ambifaria BCC0203 Δbamb_5927 | 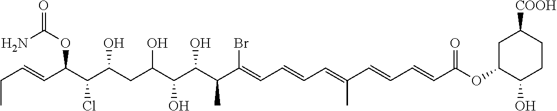 [SMILES: CC/C=C/[C@@H](OC(N)=O)[C@@H](Cl)[C@H](O)CC(O)[C@@H](O)[C@H](O)[C@H](C)/C(Br)=C/C=C/C=C(C)/C=C/C=C/C(=O)O[C@@H]1C[C@@H](C(=O)O)CC[C@@H]1O] |
| 38 | B. ambifaria BCC0203 Δbamb_5927 | 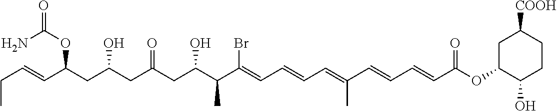 [SMILES: CC/C=C/[C@H](C[C@H](O)CC(=O)C[C@H](O)[C@H](C)/C(Br)=C/C=C/C=C(C)/C=C/C=C/C(=O)O[C@@H]1C[C@@H](C(=O)O)CC[C@@H]1O)OC(N)=O] |
| 39 | B. ambifaria BCC0203 Δbamb_5930 | 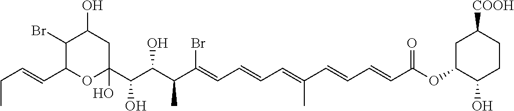 [SMILES: CC/C=C/C1OC(O)([C@@H](O)[C@H](O)[C@H](C)/C(Br)=C/C=C/C=C(C)/C=C/C=C/C(=O)O[C@@H]2C[C@@H](C(=O)O)CC[C@@H]2O)CC(O)C1Br] |
| 40 | B. ambifaria BCC0203 Δbamb_5930 | 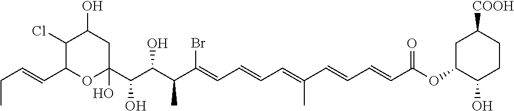 [SMILES: CC/C=C/C1OC(O)([C@@H](O)[C@H](O)[C@H](C)/C(Br)=C/C=C/C=C(C)/C=C/C=C/C(=O)O[C@@H]2C[C@@H](C(=O)O)CC[C@@H]2O)CC(O)C1Cl] |
| 41 | B. ambifaria BCC0203 Δbamb_5930 | 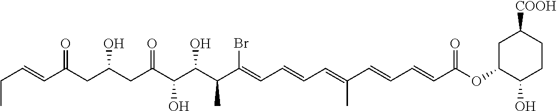 [SMILES: CC/C=C/C(=O)C[C@H](O)CC(=O)[C@@H](O)[C@H](O)[C@H](C)/C(Br)=C/C=C/C=C(C)/C=C/C=C/C(=O)O[C@@H]1C[C@@H](C(=O)O)CC[C@@H]1O] |

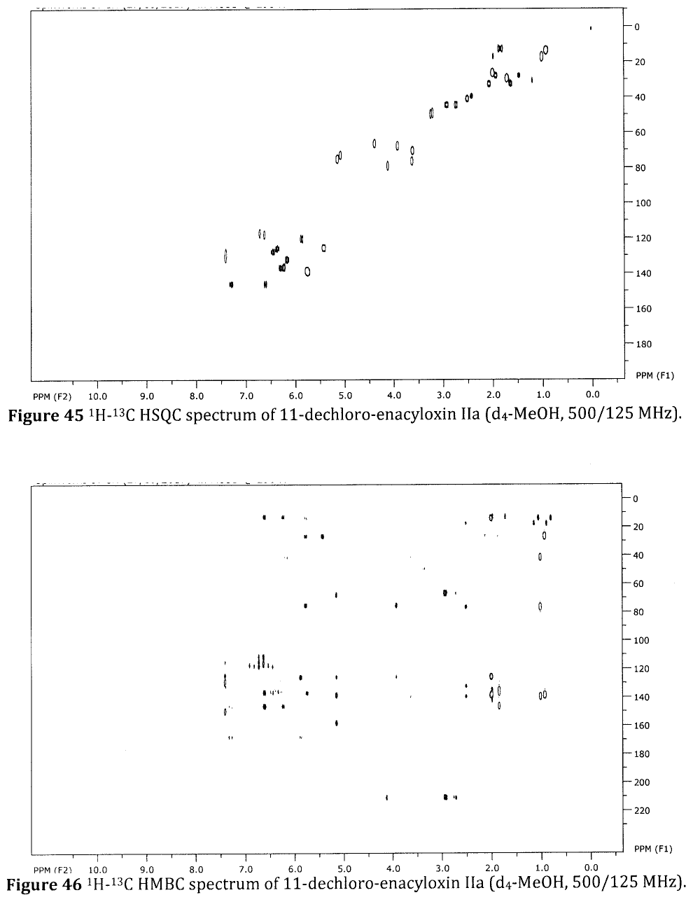

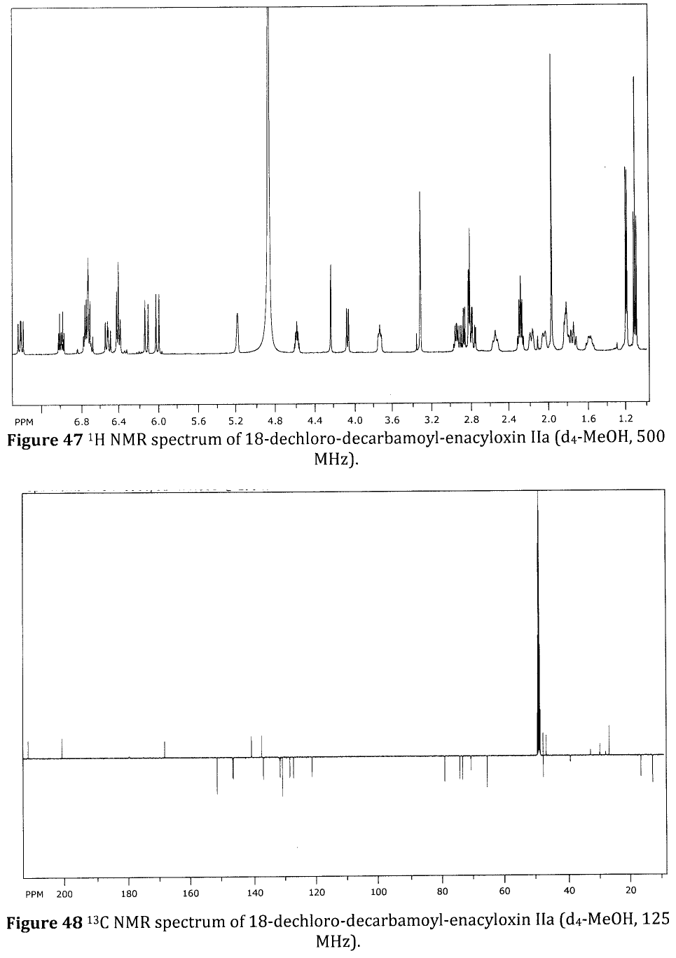

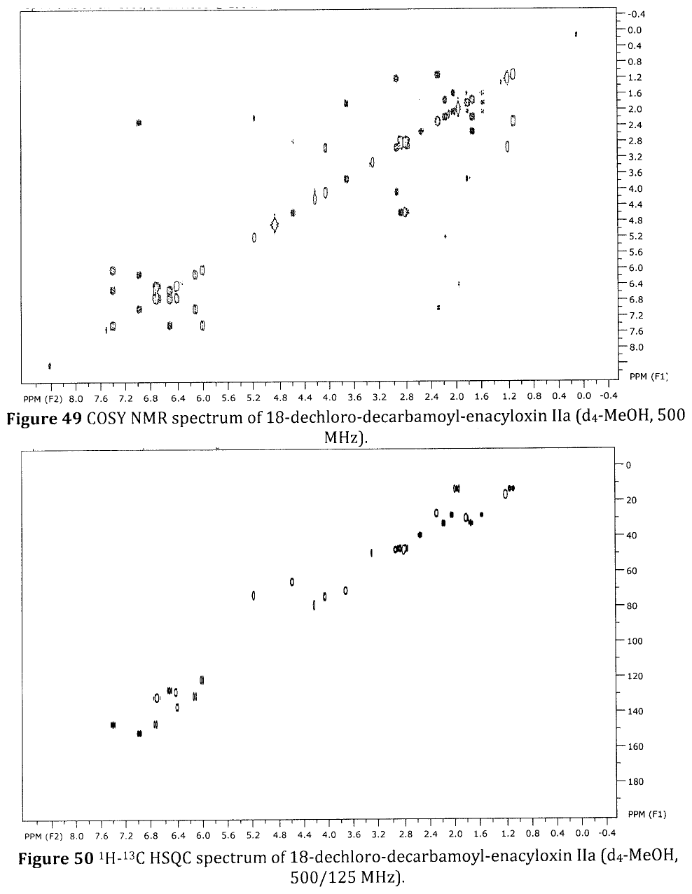

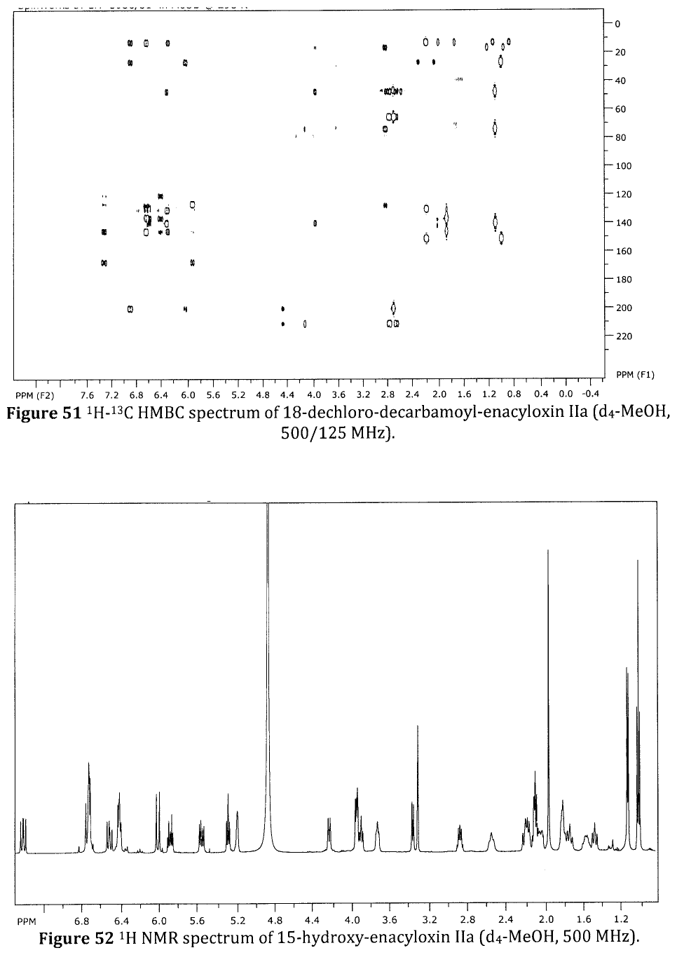

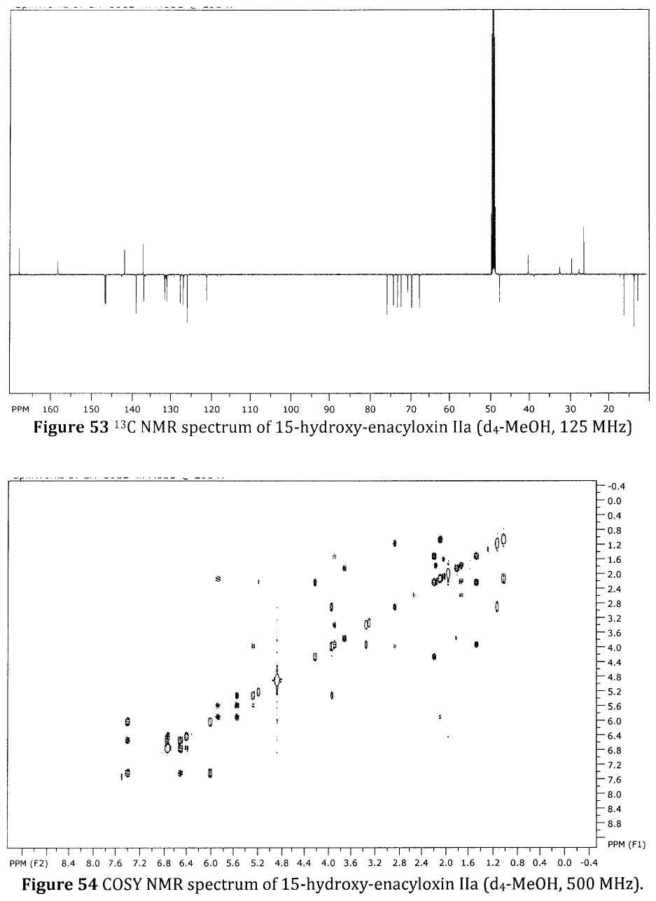

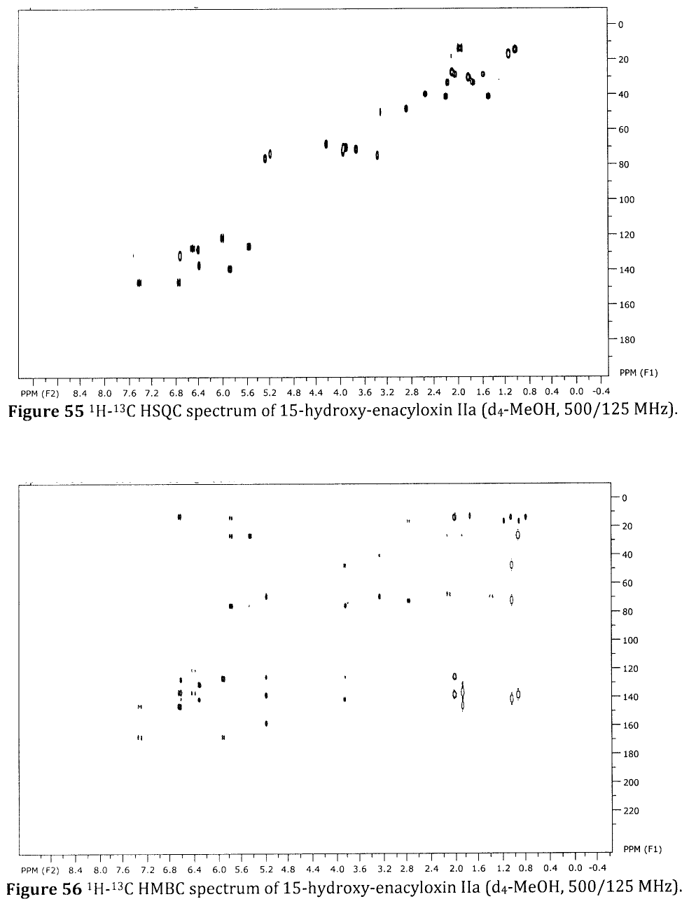

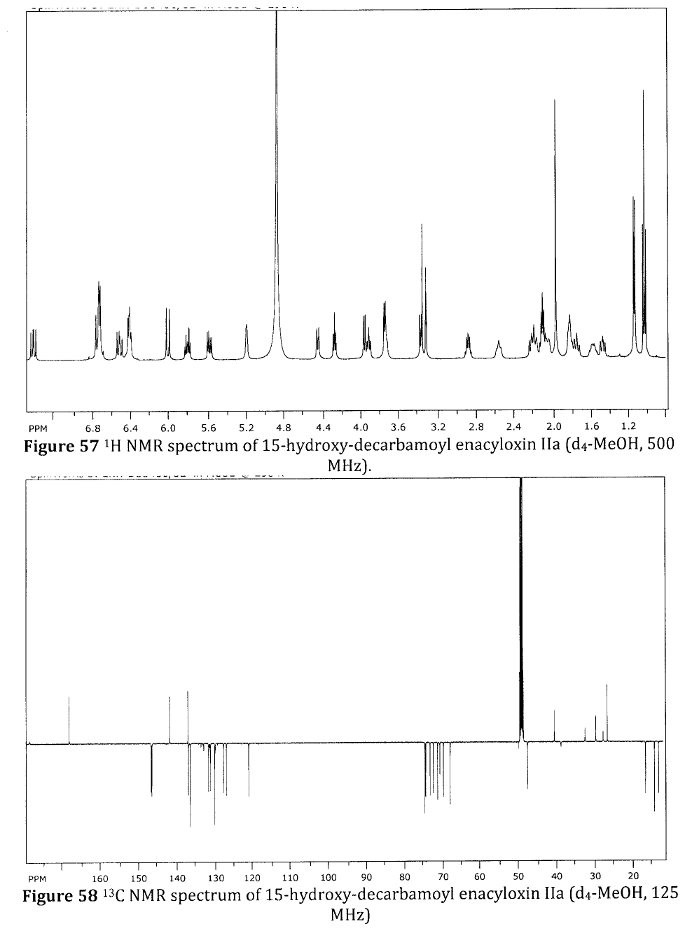

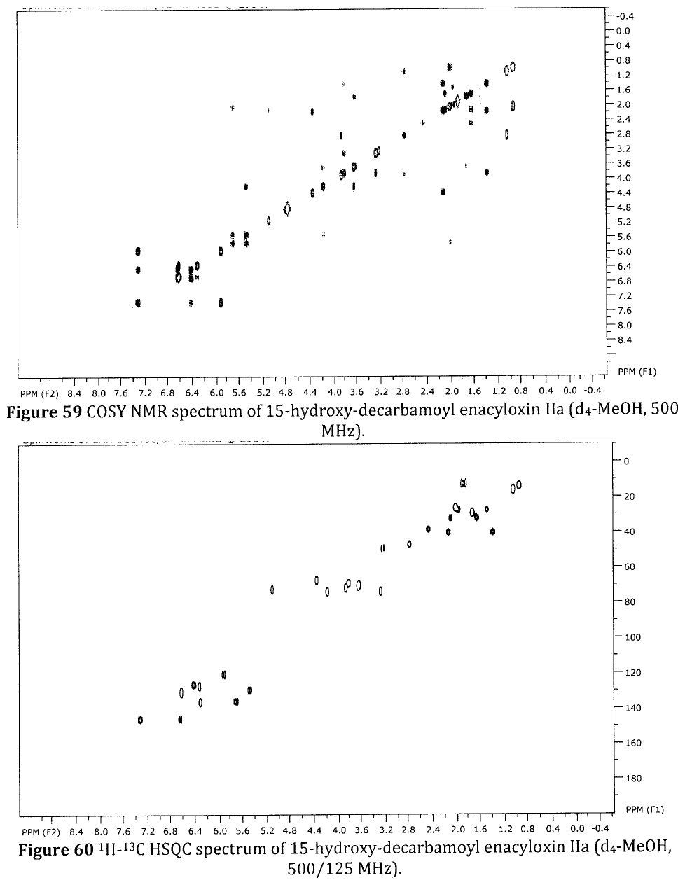

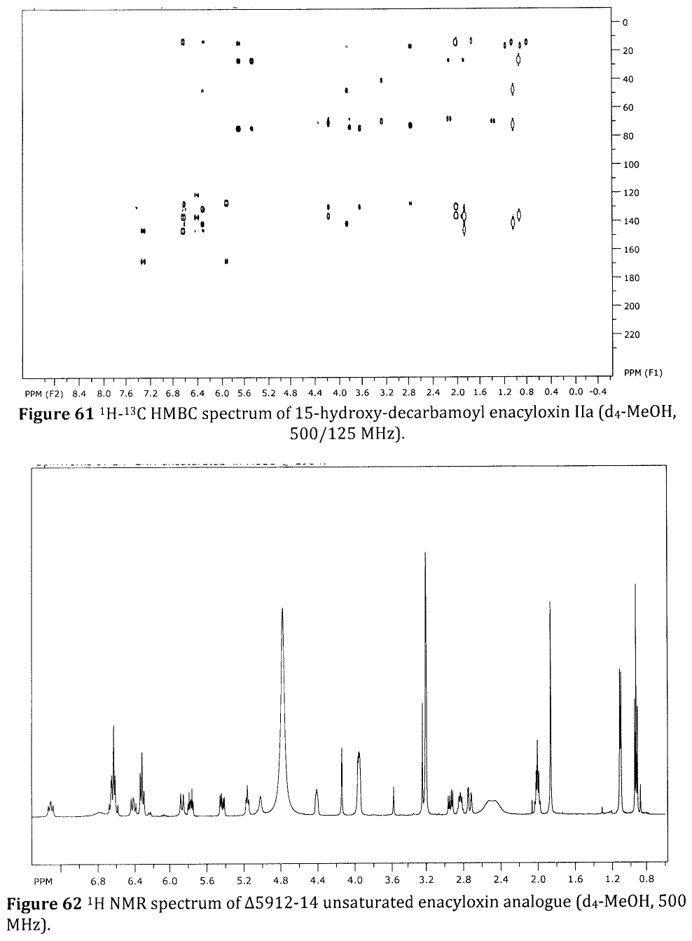

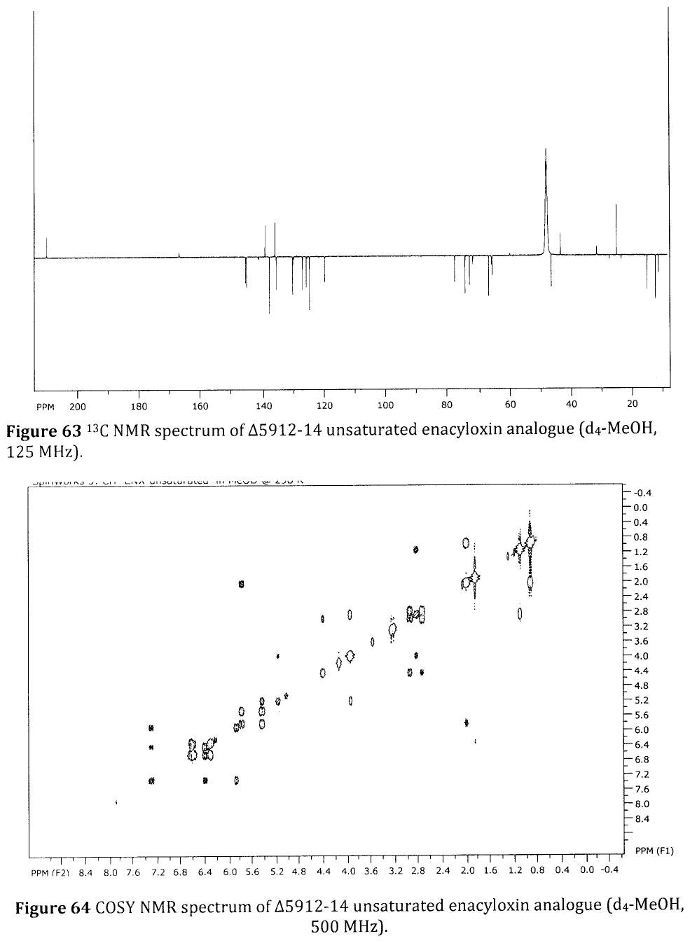

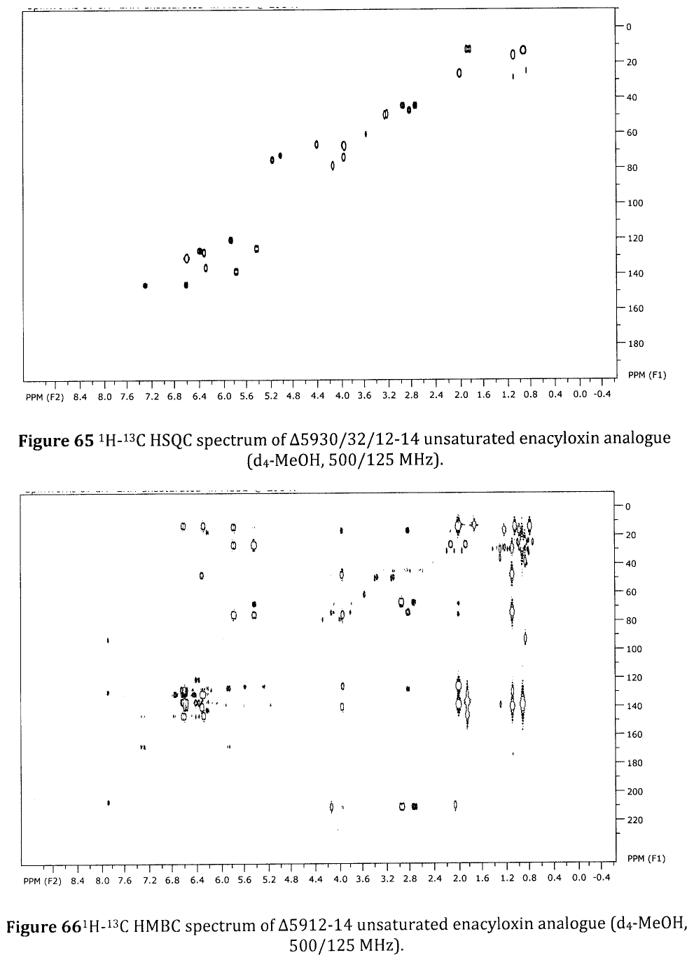

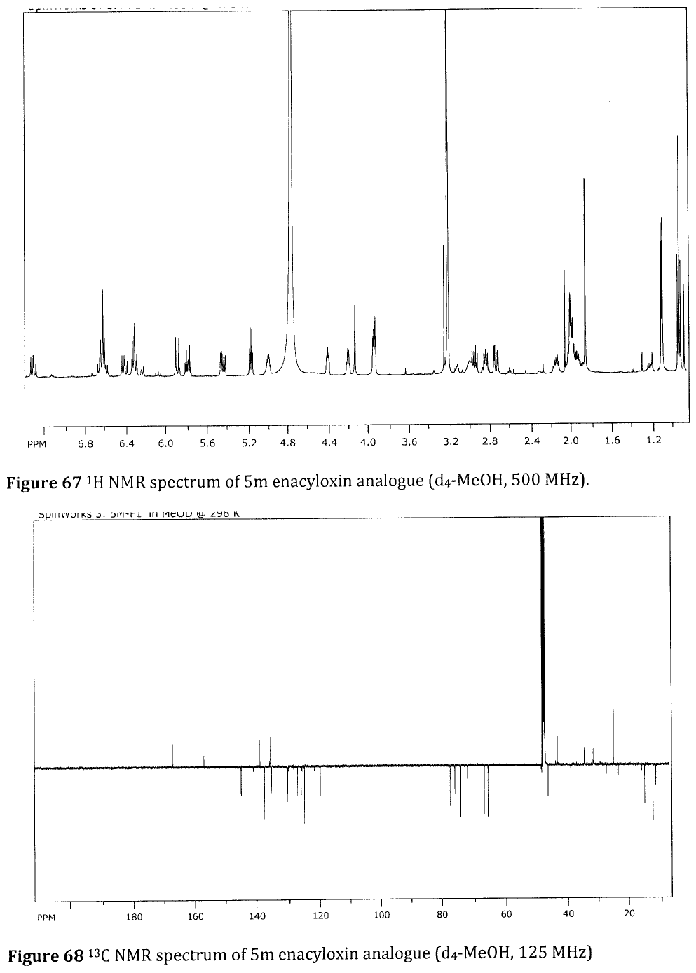

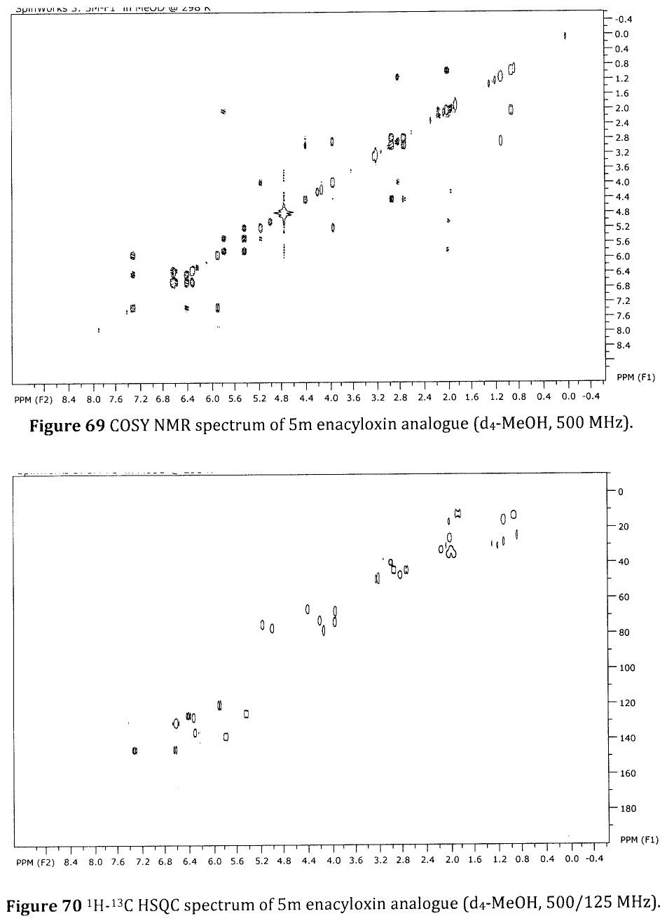

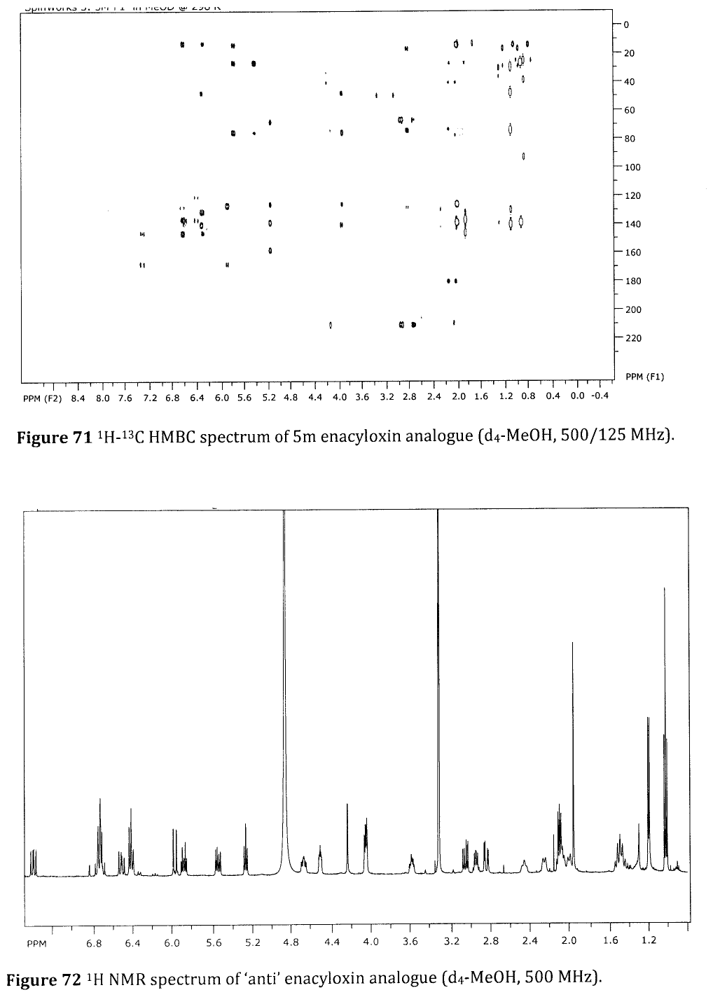

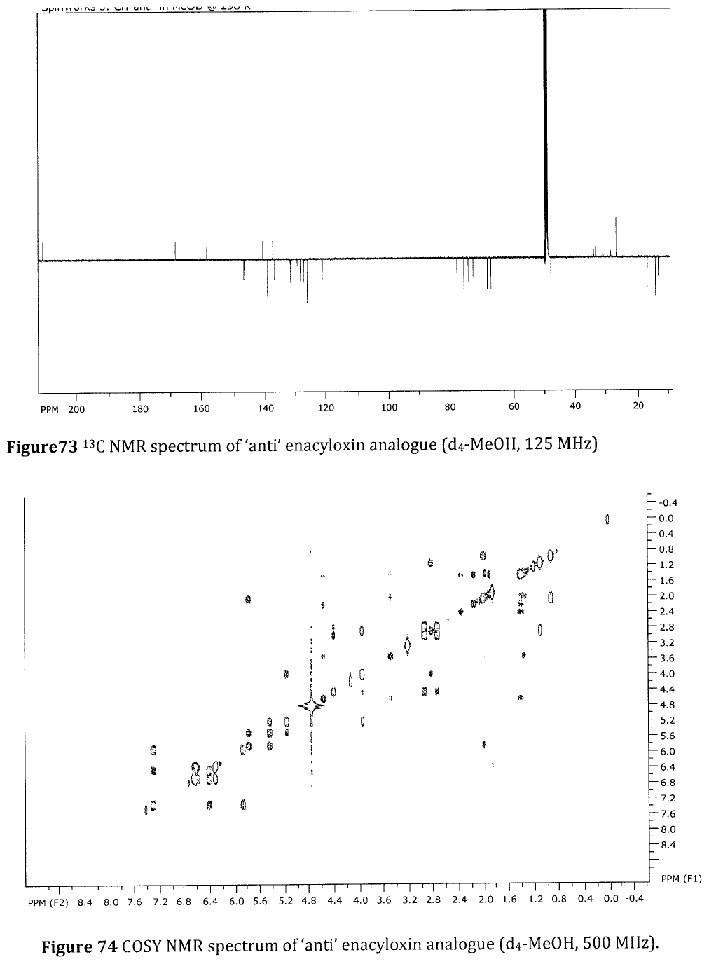

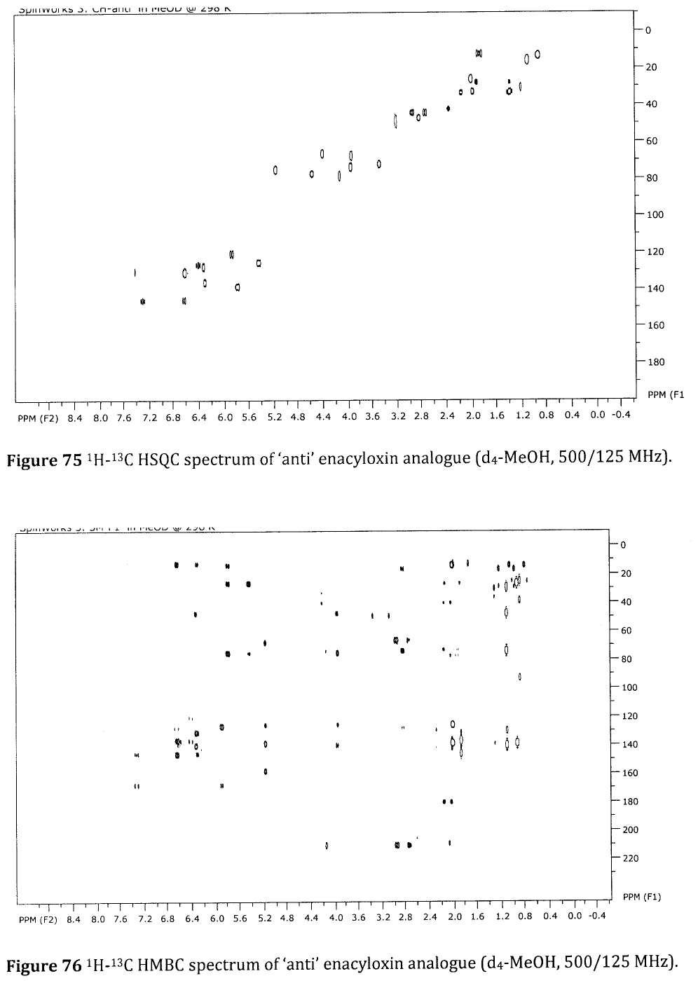

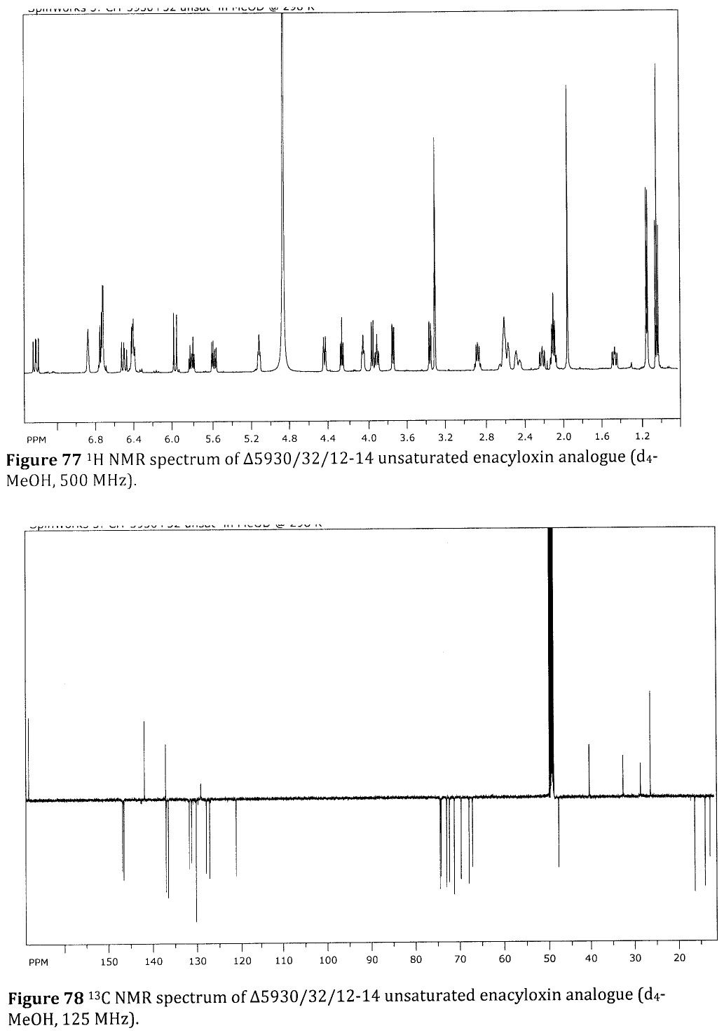

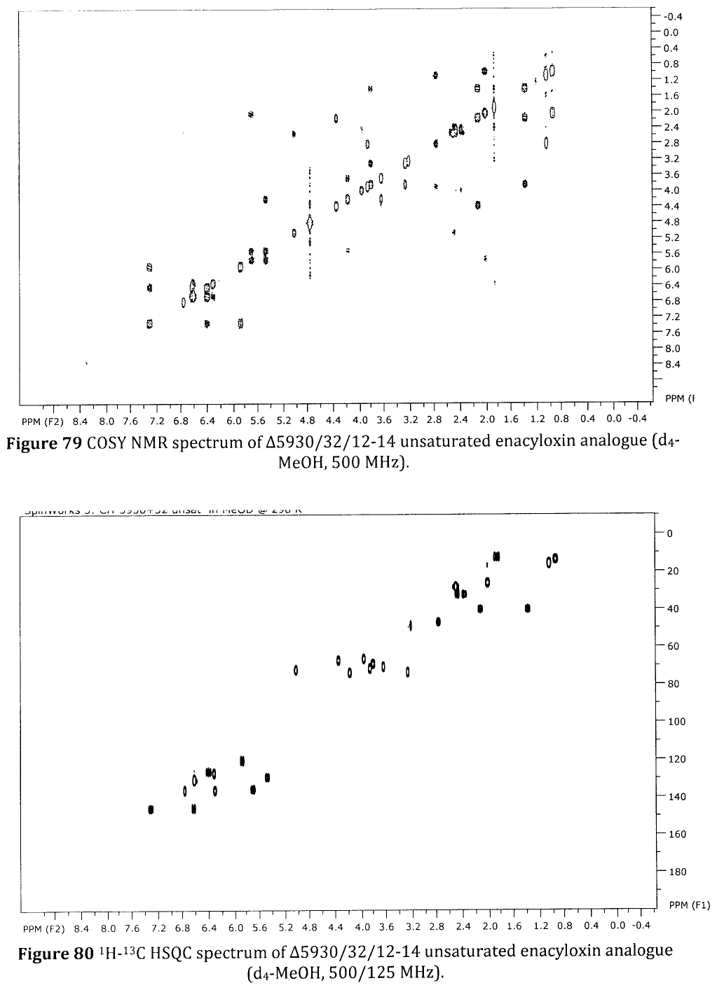

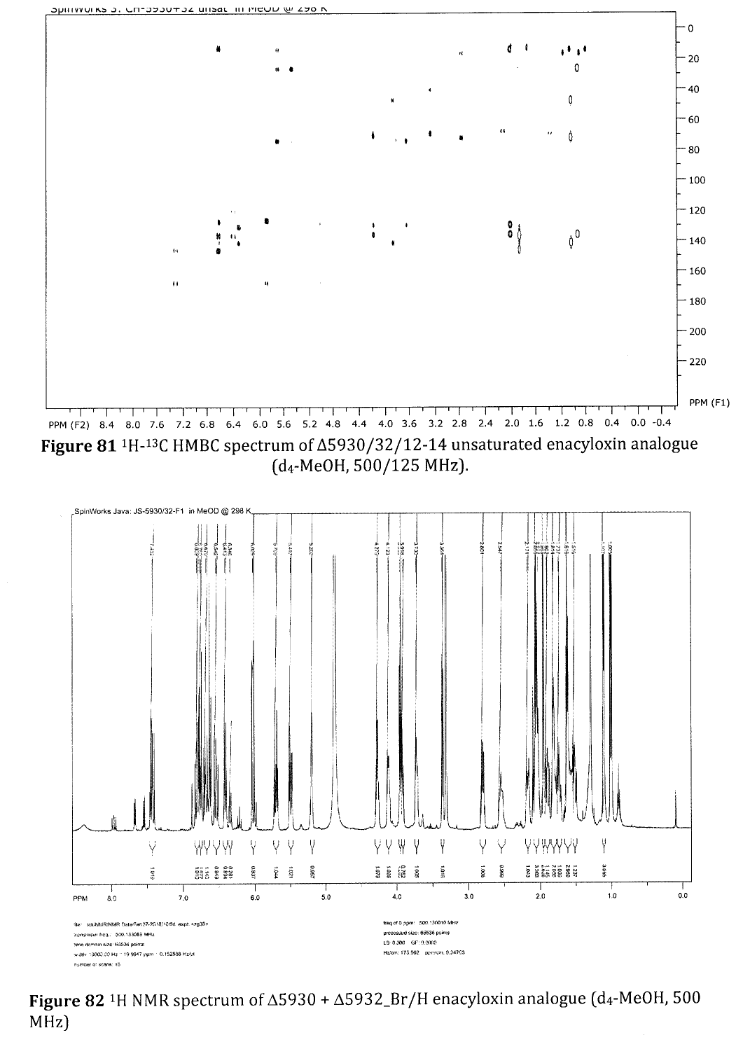

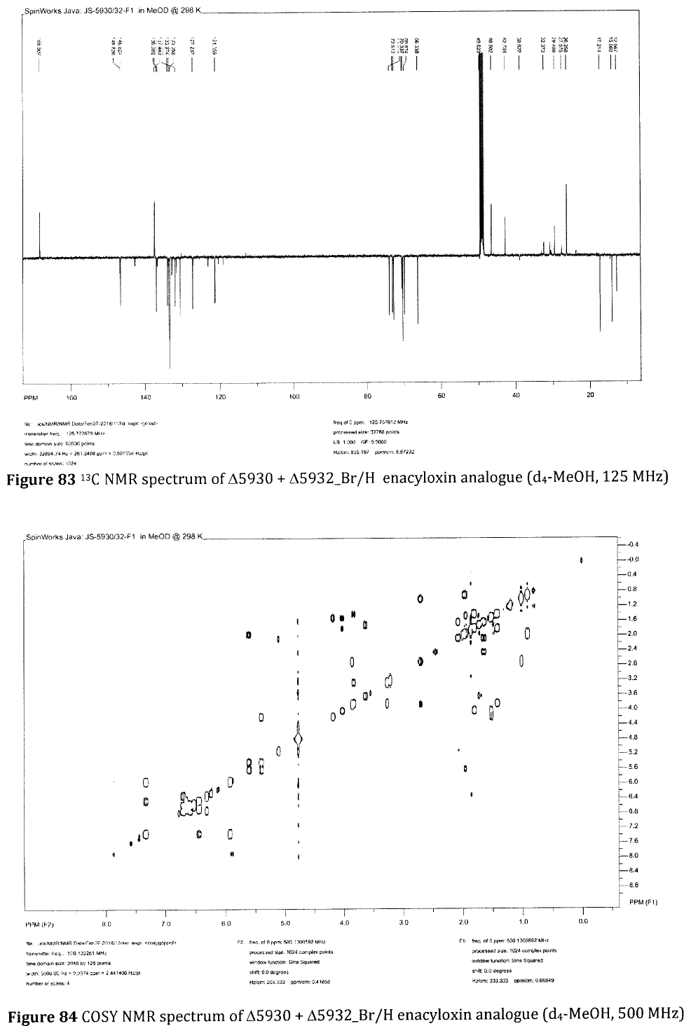

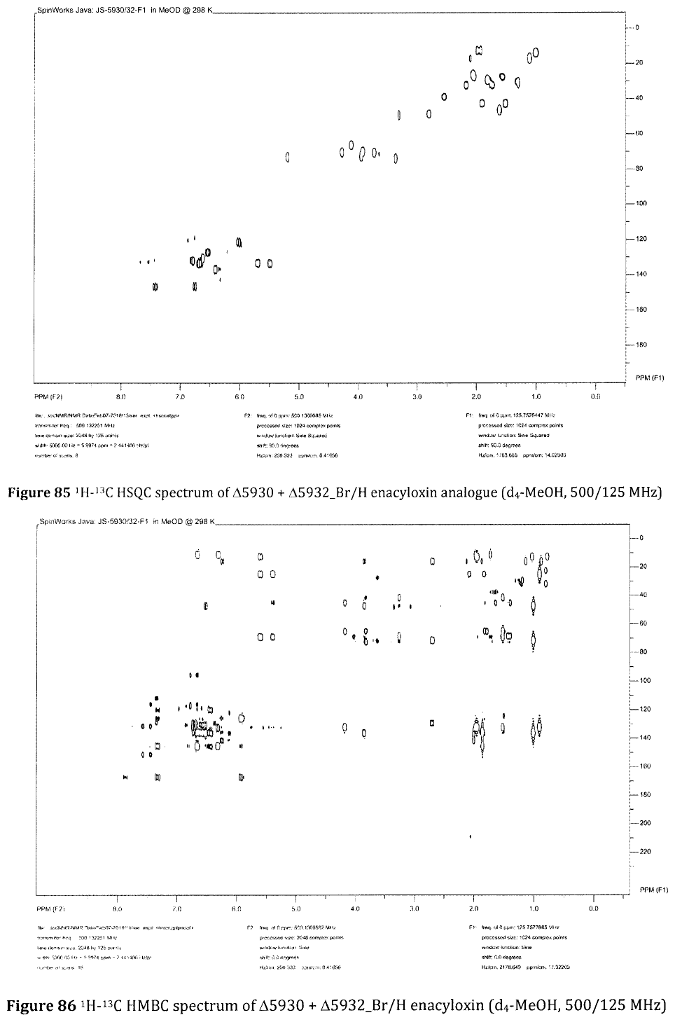

Structures were confirmed by ¹H NMR, ¹³C NMR, COSY NMR, ¹H-¹³C HSQC and ¹H-¹³C HMBC and spectra are shown in FIGS. 1 to 61. The resulting chemical shift assignments for the compounds are listed in Tables 3 to 27.

TABLE 3
NMR assignments for 18-dechloro-enacyloxin IIa (d₄-MeOH, ¹H 500 MHz, ¹³C 125 MHz).

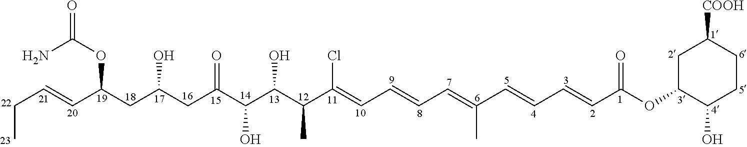 [SMILES: CC/C=C/[C@H](C[C@H](O)CC(=O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O)OC(N)=O]

| Position | ¹H (ppm) | ¹³C (ppm) |
|---|---|---|
| 1′-COOH | | 181.5 |
| 1′ | 2.53 (m) | 39.6 |
| 2′ | 1.73/2.17 (m/dm, 14.2) | 32.8 |
| 3′ | 5.19 (m) | 73.8 |
| 4′ | 3.72 (m) | 70.7 |
| 5′ | 1.80 (m) | 29.6 |
| 6′ | 1.56/2.02 (m/m) | 28.1 |
| 1 | | 168.6 |
| 2 | 6.01 (d, 15.0) | 121.7 |
| 3 | 7.43 (dd, 15.0, 11.0) | 147.0 |
| 4 | 6.52 (dd, 15.0, 11.0) | 127.3 |
| 5 | 6.76 (d, 15.0) | 147.0 |
| 6 | | 137.1 |
| 6-Me | 1.95 (s) | 12.9 |
| 7 | 6.41 (br, d, 10.0) | 137.4 |
| 8 | 6.78 (dd, 14.9, 10.0) | 132.1 |
| 9 | 6.71 (dd, 14.9, 9.9) | 131.9 |
| 10 | 6.45 (d, 9.8) | 128.6 |
| 11 | | 140.8 |
| 12 | 2.93 (dq, 9.5, 6.7) | 47.7 |
| 12-Me | 1.18 (d, 6.5) | 13.1 |
| 13 | 4.05 (dd, 9.5, 1.5) | 74.3 |
| 14 | 4.22 (d, 1.7) | 79.2 |
| 15 | | 211.1 |
| 16 | 2.64/2.82 (dd, 16.0, | 47.3 |
| | 4.0/dd, 16.0, 8.0) | |
| 17 | 4.19 (m) | 65.3 |
| 18 | 1.80/1.64 (ddd, 14.5, 10, | 43.9 |
| | 3/ddd, 14.5, 9.5, 3.5) | |
| 19 | 5.22 (ddd, 9.5, 7, 3.5) | 73.0 |
| 19-carbamate | | 158.0 |
| 20 | 5.43 (ddt, 15.5, 7.0, 1.5) | 126.5 |
| 21 | 5.76 (dt, 15.5, 6.5) | 139.7 |
| 22 | 2.03 (qdd, 7.9, 6.4, 1.5) | 26.8 |
| 23 | 1.00 (t, 7.4) | 14.0 |

TABLE 4
NMR assignments for 14-dehydroxy-enacyloxin IIa (d₄-MeOH, ¹H 500 MHz, ¹³C 125 MHz).

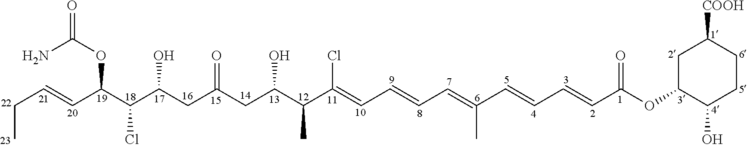 [SMILES: CC/C=C/[C@@H](OC(N)=O)[C@@H](Cl)[C@H](O)CC(=O)C[C@H](O)[C@H](C)/C(Cl)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | 181.5 |
| 1′ | 2.53 (m) | 39.8 |
| 2′ | 1.73/2.17 (m/dm, 14.2) | 32.9 |
| 3′ | 5.21 (m) | 73.5 |
| 4′ | 3.73 (m) | 70.8 |
| 5′ | 1.81 (m) | 29.5 |
| 6′ | 1.56/2.02 (m/m) | 28.3 |
| 1 | | 168.5 |
| 2 | 6.03 (d, 15.0) | 121.6 |
| 3 | 7.44 (dd, 15.0, 11.0) | 147.0 |
| 4 | 6.54 (dd, 15.0, 11.0) | 127.4 |
| 5 | 6.76 (d, 15.0) | 146.8 |
| 6 | | 136.6 |
| 6-Me | 1.97 (br, s) | 12.9 |
| 7 | 6.42 (br, d, 10.0) | 137.0 |
| 8 | 6.77 (dd, 14.8, 10.0) | 131.8 |
| 9 | 6.71 (dd, 14.8, 9.9) | 131.5 |
| 10 | 6.45 (d, 9.9) | 128.4 |
| 11 | | 140.2 |
| 12 | 2.66 (dq, 9.6, 6.7) | 48.5 |
| 12-Me | 1.16 (d, 7.0) | 13.1 |
| 13 | 4.24 (ddd, 9, 7.5, 3.5) | 67.8 |
| 14 | 2.61 (d, 9) | 49.5 |
| 15 | | 209.6 |
| 16 | 2.78/2.90 (dd, 17.0, 5.0/dd, | 44.9 |
| | 17.0, 8.0) | |
| 17 | 4.46 (m) | 67.3 |
| 18 | 4.00 (dd, 8.5, 2.5) | 68.0 |
| 19 | 5.26 (t, 7.5) | 75.8 |
| 19-carbamate | | 158.1 |
| 20 | 5.54 (ddt, 15.4, 7.4, 1.4) | 126.3 |
| 21 | 5.89 (dt, 15.2, 6.4) | 139.7 |
| 22 | 2.10 (qdd, 7.9, 6.4, 1.4) | 26.8 |
| 23 | 1.01 (t, 7.4) | 14.0 |

TABLE 5
NMR assignments for decarbamoyl-enacyloxin IIa (d₄-MeOH, ¹H 500 MHz, ¹³C 125 MHz).

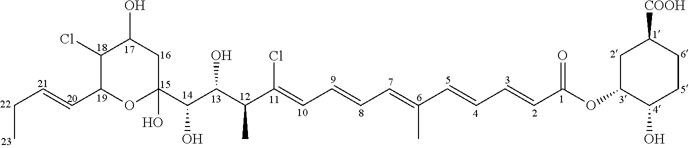 [SMILES: CC/C=C/C1OC(O)([C@@H](O)[C@H](O)[C@H](C)/C(Cl)=C/C=C/C=C(C)/C=C/C=C/C(=O)O[C@@H]2C[C@@H](C(=O)O)CC[C@@H]2O)CC(O)C1Cl]

| Position | ¹H (ppm) | ¹³C (ppm) |
|---|---|---|
| 1′-COOH | | 179.2 |
| 1′ | 2.54 (m) | 39.1 |
| 2′ | 1.72/2.18 (m/dm, 14.2) | 32.8 |
| 3′ | 5.20 (m) | 73.5 |
| 4′ | 3.73 (m) | 70.6 |
| 5′ | 1.81 (m) | 29.6 |
| 6′ | 1.56/2.01 (m/m) | 28.1 |
| 1 | | 168.5 |
| 2 | 6.03 (d, 15.0) | 121.6 |
| 3 | 7.43 (dd, 15.0, 11.0) | 146.8 |
| 4 | 6.54 (dd, 15.0, 11.0) | 127.4 |
| 5 | 6.75 (d, 15.0) | 146.6 |
| 6 | | 137.0 |
| 6-Me | 1.96 (br, s) | 12.6 |
| 7 | 6.41 (br, d, 10.0) | 137.0 |
| 8 | 6.77 (dd, 14.9, 10.0) | 131.8 |
| 9 | 6.73 (dd, 14.8, 9.9) | 131.5 |
| 10 | 6.43 (d, 9.9) | 128.4 |
| 11 | | 141.0 |
| 12 | 2.90 (dq, 9.6, 6.7) | 47.9 |
| 12-Me | 1.12 (d, 6.5) | 16.0 |
| 13 | 4.17 (dd, 9.5, 0.5) | 72.7 |
| 14 | 3.45 (d, 0.5) | 73.2 |
| 15 | | 100.5 |
| 16 | 1.53/2.45 (dd, 13, 12/dd, | 42.2 |
| | 13, 5.0) | |
| 17 | 3.97 (ddd, 11.5, 9.5, 5) | 70.8 |
| 18 | 3.36 (t, 9.5) | 67.5 |
| 19 | 5.26 (dd, 7.8, 7.6) | 74.9 |
| 20 | 5.54 (ddt, 15.5, 7, 1.5) | 127.2 |
| 21 | 5.84 (dt, 15.2, 6.4) | 137.7 |
| 22 | 2.10 (qdd, 7.9, 6.4, 1.5) | 26.7 |
| 23 | 1.01 (t, 7.4) | 13.8 |

TABLE 6
NMR assignments for 5′-hydroxy-enacyloxin IIa (d₄-MeOH, ¹H 500 MHz, ¹³C 125 MHz).

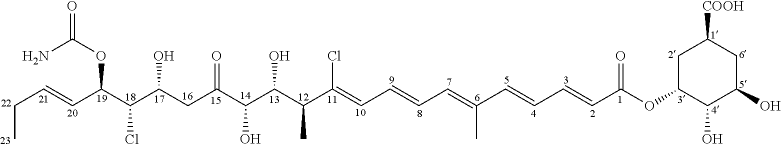 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@@H](C(=O)O)C[C@@H](O)[C@@H]1O]

| Position | ¹H (ppm) | ¹³C (ppm) |
|---|---|---|
| 1′-COOH | | 178.5 |
| 1′ | 2.66 (m) | 38.8 |
| 2′ | 1.70/2.09 (m/dm, 14.2) | 32.9 |
| 3′ | 5.30 (q, 3) | 73.7 |
| 4′ | 3.45 (dd, 9.5, 3) | 74.4 |
| 5′ | 3.82 (ddd, 11.5, 9.5, 5) | 69.9 |
| 6′ | 1.48/2.24 (m/m) | 35.7 |
| 1 | | 168.4 |
| 2 | 6.00 (d, 15.0) | 121.2 |
| 3 | 7.40 (dd, 15.0, 11.0) | 147.0 |
| 4 | 6.52 (dd, 15.0, 11.0) | 127.4 |
| 5 | 6.74 (d, 15.0) | 146.6 |
| 6 | | 137.5 |
| 6-Me | 1.95 (br, s) | 12.6 |
| 7 | 6.41 (br, d, 10.0) | 137.5 |
| 8 | 6.75 (dd, 14.8, 10.0) | 132.2 |
| 9 | 6.72 (dd, 14.8, 9.9) | 131.5 |
| 10 | 6.43 (d, 9.9) | 128.4 |
| 11 | | 140.7 |
| 12 | 2.93 (dq, 9.6, 6.7) | 47.6 |
| 12-Me | 1.19 (d, 6.5) | 16.3 |
| 13 | 4.04 (br, d, 9.4) | 74.2 |
| 14 | 4.22 (d, 1.7) | 79.0 |
| 15 | | 211.5 |
| 16 | 2.82/3.03 (dd, 17.0, 4.5/dd, | 44.6 |
| | 16.9, 8.0) | |
| 17 | 4.50 (m) | 66.8 |
| 18 | 4.04 (m) | 68.0 |
| 19 | 5.26 (dd, 7.8, 7.6) | 75.9 |
| 19-carbamate | | 158.7 |
| 20 | 5.54 (ddt, 15.4, 7.4, 1.4) | 126.2 |
| 21 | 5.88 (dt, 15.2, 6.4) | 139.2 |
| 22 | 2.09 (qdd, 7.9, 6.4, 1.4) | 26.4 |
| 23 | 1.01 (t, 7.4) | 13.7 |

TABLE 7
NMR assignments for 14-dehydroxy-18-decarbamoyl-enacyloxin IIa (d₄-MeOH, ¹H 500 MHz, ¹³C 125 MHz).

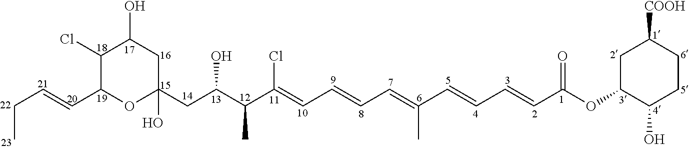 [SMILES: CC/C=C/C1OC(O)(C[C@H](O)[C@H](C)/C(Cl)=C/C=C/C=C(C)/C=C/C=C/C(=O)O[C@@H]2C[C@@H](C(=O)O)CC[C@@H]2O)CC(O)C1Cl]

| Position | ¹H (ppm) | ¹³C (ppm) |
|---|---|---|
| 1′-COOH | | 181.5 |
| 1′ | 2.55 (m) | 40.1 |
| 2′ | 1.80/2.19 (m/dm, 14.2) | 32.6 |
| 3′ | 5.21 (m) | 73.2 |
| 4′ | 3.73 (m) | 70.6 |
| 5′ | 1.83 (m) | 29.5 |
| 6′ | 1.55/2.01 (m/m) | 27.8 |
| 1 | | 168.5 |
| 2 | 6.03 (d, 15.0) | 121.5 |
| 3 | 7.43 (dd, 15.0, 11.0) | 146.8 |
| 4 | 6.54 (dd, 15.0, 11.0) | 127.3 |
| 5 | 6.75 (d, 15.0) | 146.6 |
| 6 | | 137.0 |
| 6-Me | 1.97 (br, s) | 12.6 |
| 7 | 6.41 (br, d, 10.0) | 137.5 |
| 8 | 6.78 (dd, 14.8, 10.0) | 131.6 |
| 9 | 6.72 (dd, 14.8, 9.9) | 130.1 |
| 10 | 6.43 (d, 10.0) | 128.2 |
| 11 | | 140.1 |
| 12 | 2.58 (m) | 51.2 |
| 12-Me | 1.14 (d, 6.5) | 15.3 |
| 13 | 4.25 (dt, 9, 1.5) | 70.5 |
| 14 | 1.7 (d, 10.5) | 32.6 |
| 15 | | 98.9 |
| 16 | 1.55/2.21 (dd, 12.5, | 45.3 |
| | 11.5/dd, 7.5, 5) | |
| 17 | 3.97 (ddd, 11.5, 9.5, 5) | 70.7 |
| 18 | 3.34 (d, 10) | 67.5 |
| 19 | 4.31 (dd, 10, 6.5) | 75.9 |
| 20 | 5.52 (ddt, 15.5, 8.5, 1.5) | 127.3 |
| 21 | 5.84 (dt, 15.5, 6.5) | 136.9 |
| 22 | 2.09 (qdd, 7.9, 6.4, 1.4) | 26.4 |
| 23 | 1.02 (t, 7.4) | 13.8 |

TABLE 8
NMR assignments for 14-dehydroxy-18-dechloro-enacyloxin IIa (d₄-MeOH, ¹H 500 MHz, ¹³C 125 MHz).

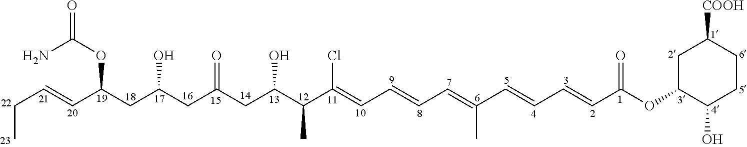 [SMILES: CC/C=C/[C@H](C[C@H](O)CC(=O)C[C@H](O)[C@H](C)/C(Cl)=C/C=C/C=C(C)/C=C/C=C/C(=O)O[C@@H]1C[C@@H](C(=O)O)CC[C@@H]1O)OC(N)=O]

| Position | ¹H (ppm) | ¹³C (ppm) |
|---|---|---|
| 1′-COOH | | 181.5 |
| 1′ | 2.53 (m) | 39.1 |
| 2′ | 1.74/2.17 (m/dm, 14.5) | 32.4 |
| 3′ | 5.20 (m) | 73.3 |
| 4′ | 3.75 (m) | 70.5 |
| 5′ | 1.83 (m) | 29.3 |
| 6′ | 1.43/2.02 (m/m) | 27.8 |
| 1 | | 168.9 |
| 2 | 6.04 (d, 15.5) | 121.4 |
| 3 | 7.44 (dd, 15.0, 11.0) | 147.1 |
| 4 | 6.55 (dd, 15.0, 11.0) | 127.1 |
| 5 | 6.75 (d, 15.0) | 146.8 |
| 6 | | 137.1 |
| 6-Me | 1.96 (s) | 12.7 |
| 7 | 6.41 (br, d, 10.0) | 137.4 |
| 8 | 6.78 (dd, 14.9, 10.0) | 132.7 |
| 9 | 6.71 (dd, 14.9, 9.9) | 131.4 |
| 10 | 6.42 (d, 9.8) | 127.6 |
| 11 | | 141.0 |
| 12 | 2.59 (m) | 51.0 |
| 12-Me | 1.14 (d, 6.5) | 13.9 |
| 13 | 3.98 (m) | 70.2 |
| 14 | 2.59 (m) | 51.0 |
| 15 | | 210.9 |
| 16 | 1.64/1.54 (m/m) | 43.3 |
| 17 | 4.10 (m) | 65.9 |
| 18 | 1.74/ 1.64 (m, m) | 43.9 |
| 19 | 5.24 (ddd, 9.5, 7, 3.5) | 73.3 |
| 19-carbamate | | 159.9 |
| 20 | 5.46 (ddt, 15.5, 7.0, 1.5) | 129.2 |
| 21 | 5.74 (dt, 15.5, 6.5) | 135.8 |
| 22 | 2.05 (qdd, 7.9, 6.4, 1.5) | 26.2 |
| 23 | 0.99 (t, 7.4) | 13.8 |

TABLE 9
NMR assignments for 2′-dehydroxy-enacyloxin IIa (d₄-MeOH, ¹H 500 MHz, ¹³C 125 MHz).

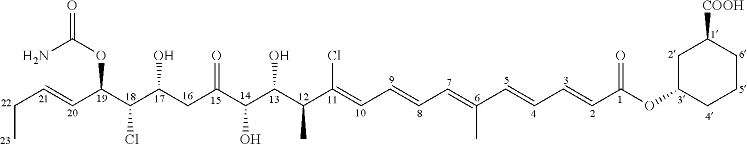 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@H]1CCC[C@H](C(=O)O)C1]

| Position | ¹H (ppm) | ¹³C (ppm) |
|---|---|---|
| 1′-COOH | | 181.5 |
| 1′ | 2.68 (m) | 39.9 |
| 2′ | 1.79/2.03 (m/dm, 12.5) | 33.8 |
| 3′ | 5.15 (m) | 70.8 |
| 4′ | 1.63 (m) | 30.8 |
| 5′ | 1.93 (m) | 29.4 |
| 6′ | 1.57 (m) | 21.5 |
| 1 | | 168.4 |
| 2 | 5.98 (d, 15.0) | 121.4 |
| 3 | 7.39 (dd, 15.0, 11.0) | 146.7 |
| 4 | 6.53 (dd, 15.0, 11.0) | 127.2 |
| 5 | 6.77 (d, 15.0) | 147.6 |
| 6 | | 137.0 |
| 6-Me | 1.96 (ar, s) | 12.6 |
| 7 | 6.42 (br, d, 10.0) | 137.4 |
| 8 | 6.77 (dd, 14.8, 10.0) | 131.6 |
| 9 | 6.72 (dd, 14.8, 9.9) | 131.5 |
| 10 | 6.44 (d, 9.9) | 128.4 |
| 11 | | 140.7 |
| 12 | 2.94 (dq, 9.6, 6.7) | 47.6 |
| 12-Me | 1.19 (d, 6.5) | 16.25 |
| 13 | 4.05 (br, d, 9.4) | 74.0 |
| 14 | 4.25 (d, 1.7) | 78.9 |
| 15 | | 211.5 |
| 16 | 2.84/3.05 (dd, 16.9, 4.5/dd, | 44.6 |
| | 16.9, 8.0) | |
| 17 | 4.51 (m) | 66.8 |
| 18 | 4.05 (m) | 68.0 |
| 19 | 5.28 (dd, 7.8, 7.6) | 75.5 |
| 19-carbamate | | 158.7 |
| 20 | 5.56 (ddt, 15.4, 7.4, 1.4) | 126.2 |
| 21 | 5.90 (dt, 15.2, 6.4) | 139.2 |
| 22 | 2.10 (qdd, 7.9, 6.4, 1.4) | 26.3 |
| 23 | 1.02 (t, 7.4) | 13.6 |

TABLE 10
NMR assignments for enacyloxin-4-amino-3-hydroxybutyric acid conjugate (d₄-MeOH, ¹H 500 MHz, ¹³C 125 MHz).

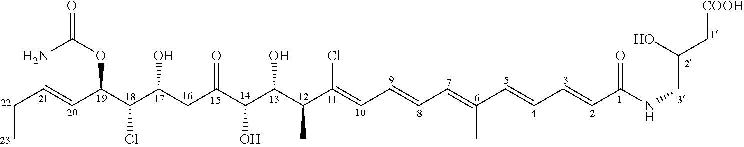

| Position | ¹H (ppm) | ¹³C (ppm) |
|---|---|---|
| 1′-COOH | | 181.5 |
| 1′ | 2.48, 2.39 (m) | 41.9 |
| 2′ | 4.11 (m) | 69.5 |
| 3′ | 3.40 (m) | 46.9 |
| 1 | | 169.5 |
| 2 | 6.10 (d, 15.0) | 124.2 |
| 3 | 7.26 (dd, 14.5, 11.0) | 145.0 |
| 4 | 6.48 (dd, 15.0, 11.0) | 127.5 |
| 5 | 6.76 (d, 15.0) | 142.5 |
| 6 | | 136.2 |
| 6-Me | 1.95 (br, s) | 12.7 |
| 7 | 6.42 (br, d, 10.0) | 137.5 |
| 8 | 6.75 (dd, 14.8, 10.0) | 131.7 |
| 9 | 6.69 (dd, 14.8, 9.9) | 131.1 |
| 10 | 6.45 (d, 9.9) | 128.5 |
| 11 | | 139.2 |
| 12 | 2.94 (dq, 9.6, 6.7) | 48.2 |
| 12-Me | 1.19 (d, 6.5) | 16.3 |
| 13 | 4.04 (br, d, 9.4) | 74.1 |
| 14 | 4.24 (d, 1.7) | 78.9 |
| 15 | | 211.5 |
| 16 | 2.84/3.05 (dd, 16.9, 4.5/dd, | 44.6 |
| | 16.9, 8.0) | |
| 17 | 4.52 (m) | 66.8 |
| 18 | 4.05 (m) | 67.9 |
| 19 | 5.28 (dd, 7.8, 7.6) | 75.5 |
| 19-carbamate | | 158.7 |
| 20 | 5.55 (ddt, 15.4, 7.4, 1.4) | 126.2 |
| 21 | 5.90 (dt, 15.2, 6.4) | 140.4 |
| 22 | 2.10 (qdd, 7.9, 6.4, 1.4) | 26.3 |
| 23 | 1.02 (t, 7.4) | 13.6 |

TABLE 11
NMR assignments for 11-dechloro-enacyloxin IIa (d₄-MeOH, ¹H 500 MHz, ¹³C 125 MHz).

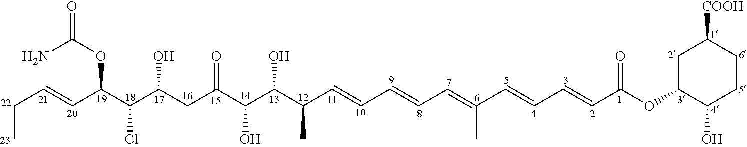 [SMILES: CC/C=C/[C@@H](OC(N)=O)[C@@H](Cl)[C@H](O)CC(=O)[C@@H](O)[C@H](O)[C@H](C)/C=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | 179.4 |
| 1′ | 2.51 (br. t, 11.1) | 39.7 |
| 2′ | 1.72/2.19-2.13 (br. t | 32.7 |
| | 11.9/m) | |
| 3′ | 5.22-5.18 (m) | 73.3 |
| 4′ | 3.75-3.69 (m) | 70.7 |
| 5′ | 1.84-1.77 (2H) (m) | 29.6 |
| 6′ | 1.60-51/2.05-1.99 (m/m) | 27.9 |
| 1 | | 168.7 |
| 2 | 5.99 (d, 15.3) | 120.9 |
| 3 | 7.42 (dd, 15.0, 11.2) | 147.0 |
| 4 | 6.48 (dd, 15.2, 10.8) | 128.6 |
| 5 | 6.73 (d, 15.2) | 146.9 |
| 6 | | 135.7 |
| 6-Me | 1.94 (s) | 12.6 |
| 7 | 6.35 (d, 11.0) | 137.6 |
| 8 | 6.41 (dd, 14.8, 10.5) | 137.4 |
| 9 | 6.48 (dd, 11.3, 15.1) | 126.4 |
| 10 | 6.29 (dd, 15.0, 10.8) | 132.8 |
| 11 | 5.86 (dd, 15.0, 8.4) | 140.2 |
| 12 | 2.60 (tq, 8.5, 7.4) | 41.4 |
| 12-Me | 1.10 (d, 6.8) | 17.3 |
| 13 | 3.73 (dd, 8.4, 2.3) | 76.7 |
| 14 | 4.22 (d, 2.6) | 79.4 |
| 15 | | 211.7 |
| 16 | 3.02/2.83 (dd 17.1, 8.0/dd, | 44.7 |
| | 17.2, 4.8) | |
| 17 | 4.49 (ddd, 7.5, 4.5, 2.4) | 66.7 |
| 18 | 4.02 (dd, 8.0, 2.5) | 68.0 |
| 19 | 5.26 (t, 7.8) | 75.5 |
| 19-carbamate | | 158.6 |
| 20 | 5.54 (ddt, 15.4, 7.4, 1.5) | 126.1 |
| 21 | 5.89 (dt, 15.4, 6.5) | 139.2 |
| 22 | 2.09 (qdd, 7.6, 6.3, 1.4) | 26.3 |
| 23 | 1.01 (t, 7.4) | 13.6 |

TABLE 12
NMR assignments for 18-dechloro-decarbamoyl-enacyloxin IIa (d₄-MeOH, ¹H 500 MHz, ¹³C 125 MHz).

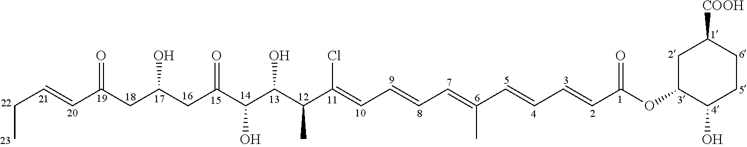 [SMILES: CC/C=C/C(=O)C[C@H](O)CC(=O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | 179.8 |
| 1′ | 2.54 (tt, 11.5, 3.4) | 39.9 |
| 2′ | 1.73/2.20-2.13 (ddd 14.0, | 32.4 |
| | 11.5) 2.0/m) | |
| 3′ | 5.22-5.18 (m) | 73.2 |
| 4′ | 3.73 (ddd 9.4, 5.4, 2.9) | 70.6 |
| 5′ | 1.85-1.77 (2H) (m) | 29.5 |
| 6′ | 1.56/2.07-2.00 (dtd 13.0, | 27.7 |
| | 11.2, 6.0/m) | |
| 1 | | 168.5 |
| 2 | 6.02 (d, 15.1) | 121.4 |
| 3 | 7.43 (dd, 15.0, 11.0) | 146.8 |
| 4 | 6.53 (dd, 15.0, 11.2) | 127.2 |
| 5 | 6.79-6.71 (m) | 146.6 |
| 6 | | 137.4 |
| 6-Me | 1.96 (s) | 12.5 |
| 7 | 6.41 (d, 10.0) | 136.9 |
| 8 | 6.79-6.71 (m) | 131.5 |
| 9 | 6.79-6.71 (m) | 131.5 |
| 10 | 6.43 (d, 9.6) | 128.4 |
| 11 | | 140.7 |
| 12 | 2.94 (dq, 9.5, 6.6) | 47.5 |
| 12-Me | 1.18 (d, 7.0) | 16.2 |
| 13 | 4.06 (dd, 9.7, 1.4) | 74.1 |
| 14 | 4.23 (d, 1.4) | 78.9 |
| 15 | | 212.1 |
| 16 | 2.87/2.76 (dd 16.6, 7.6/dd, | 46.6 |
| | 16.6, 4.8) | |
| 17 | 4.58 (tt, 7.1, 5.6) | 66.5 |
| 18 | 2.79-2.82 (2H) (m) | 47.6 |
| 19 | | 201.3 |
| 20 | 6.14 (dt, 15.9, 1.5) | 130.8 |
| 21 | 7.01 (dt, 15.9, 6.3) | 151.8 |
| 22 | 2.28 (qdd, 7.5, 6.4, 1.5) | 26.6 |
| 23 | 1.09 (t, 7.5) | 12.6 |

TABLE 13
NMR assignments for 15-hydroxy-enacyloxin IIa (d₄-MeOH, ¹H 500 MHz, ¹³C 125 MHz).

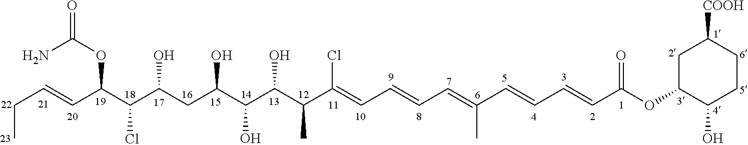 [SMILES: CC/C=C/[C@@H](OC(N)=O)[C@@H](Cl)[C@H](O)C[C@@H](O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | 179.5 |
| 1′ | 2.55 (br. t, 11.4) | 38.8 |
| 2′ | 1.74/2.20-2.14 (br. t 7.9/m) | 32.4 |
| 3′ | 5.22-5.18 (m) | 73.1 |
| 4′ | 3.74-3.70 (m) | 70.5 |
| 5′ | 1.85-1.77 (2H) (m) | 26.3 |
| 6′ | 1.61-52/2.07-2.01 (m/m) | 27.6 |
| 1 | | 168.5 |
| 2 | 6.02 (d, 15.2) | 121.3 |
| 3 | 7.43 (dd, 15.2, 11.2) | 146.8 |
| 4 | 6.52 (dd, 15.1, 11.3) | 127.8 |
| 5 | 6.76 (d, 15.0) | 146.6 |
| 6 | | 137.2 |
| 6-Me | 1.96 (s) | 12.7 |
| 7 | 6.45-6.40 (m) | 137.1 |
| 8 | 6.76-6.72 (m) | 131.7 |
| 9 | 6.76-6.72 (m) | 131.3 |
| 10 | 6.45-6.40 (m) | 127.1 |
| 11 | | 142.0 |
| 12 | 2.87 (dq, 9.5, 6.6) | 47.4 |
| 12-Me | 1.13 (d, 6.6) | 16.2 |
| 13 | 3.97-3.93 (m) | 72.2 |
| 14 | 3.36 (d, 8.2) | 74.1 |
| 15 | 3.90 (ddd 10.1, 8.2, 1.8) | 69.6 |
| 16 | 2.20/1.48 (ddd 14.3, 10.4, 1.8/ddd, 14.1, 10.2, 2.2) | 40.3 |
| 17 | 4.24 (dt, 10.1, 2.5) | 67.6 |
| 18 | 3.97-93 (m) | 72.2 |
| 19 | 5.29 (t, 7.6) | 75.7 |
| 19-carbamate | | 158.7 |
| 20 | 5.57 (ddt, 15.3, 7.3, 1.4) | 126.1 |
| 21 | 5.89 (dt, 15.4, 6.5) | 139.0 |
| 22 | 2.10 (qdd, 7.6, 6.4, 1.4) | 26.3 |
| 23 | 1.01 (t, 7.5) | 13.6 |

TABLE 14
NMR assignments for 15-hydroxy-decarbamoyl enacyloxin IIa (d₄-MeOH, ¹H 500 MHz, ¹³C 125 MHz).

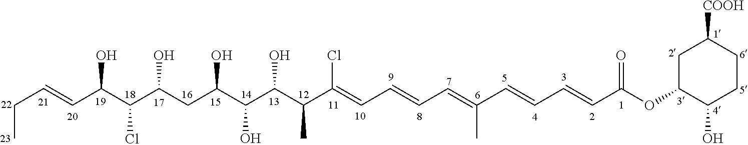 [SMILES: CC/C=C/[C@@H](O)[C@@H](Cl)[C@H](O)C[C@@H](O)[C@@H](O)[C@H](O)[C@H](C)/C(Cl)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | 179.1 |
| 1′ | 2.55 (tt, 11.2 and 3.4) | 38.6 |
| 2′ | 1.74/2.19-2.14 (ddd 14.0, 11.5, 2.1/m) | 32.3 |
| 3′ | 5.22-5.18 (m) | 73.1 |
| 4′ | 3.74-3.70 (m) | 71.1 |
| 5′ | 1.85-1.77 (2H) (m) | 26.3 |
| 6′ | 1.61-52/2.07-2.01 (m/m) | 27.5 |
| 1 | | 168.5 |
| 2 | 6.02 (d, 15.2) | 121.2 |
| 3 | 7.43 (dd, 15.1, 11.2) | 146.9 |
| 4 | 6.52 (dd, 15.1, 11.1) | 127.4 |
| 5 | 6.76 (d, 15.0) | 147.0 |
| 6 | | 137.2 |
| 6-Me | 1.96 (s) | 12.7 |
| 7 | 6.44-6.40 (m) | 137.0 |
| 8 | 6.75-6.72 (m) | 131.7 |
| 9 | 6.75-6.72 (m) | 131.2 |
| 10 | 6.44-6.40 (m) | 127.1 |
| 11 | | 142.0 |
| 12 | 2.87 (dq, 9.5, 6.7) | 47.5 |
| 12-Me | 1.13 (d, 6.7) | 16.1 |
| 13 | 3.96 (d, 9.7) | 72.2 |
| 14 | 3.37 (d, 8.2) | 74.1 |
| 15 | 3.91 (ddd 10.1, 8.2, 2.2) | 69.5 |
| 16 | 2.21/1.47 (ddd 14.2, 10.1, 2.1/ddd, 14.1, 10.1, 2.5) | 40.4 |
| 17 | 4.45 (dt, 10.2, 2.3) | 67.7 |
| 18 | 3.75 (dd, 7.6, 2.2) | 70.5 |
| 19 | 4.27 (t, 7.3) | 74.4 |
| 20 | 5.59 (ddt, 15.3, 7, 1.4) | 126.5 |
| 21 | 5.82 (dtd, 15.4, 6.4, 0.7) | 136.6 |
| 22 | 2.10 (qdd, 7.5, 6.5, 1.3) | 26.3 |
| 23 | 1.02 (t, 7.4) | 13.8 |

TABLE 15
NMR assignments for Δ5912-14 unsaturated enacyloxin analogue (d₄-MeOH, ¹H 500 MHz, ¹³C 125 MHz).

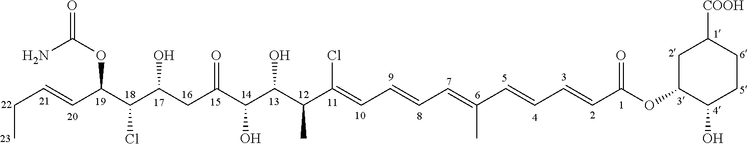 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1CC(C(=O)O)CC[C@@H]1O]

| Position | ¹H (ppm) | ¹³C (ppm) |
|---|---|---|
| 1′-COOH | | 171.2 |
| 1′ | | 130.1 |
| 2′ | 2.74-2.55 (2H) (m) | 32.6 |
| 3′ | 5.15-5.09 (m) | 72.8 |
| 4′ | 4.08-4.02 (m) | 66.9 |
| 5′ | 2.74-2.55 (2H) (m) | 28.6 |
| 6′ | 6.88 (br. s) | 136.9 |
| 1 | | 168.5 |
| 2 | 5.99 (d, 15.9) | 121.0 |
| 3 | 7.42 (dd, 14.9, 11.2) | 146.6 |
| 4 | 6.51 (dd, 14.8, 11.4) | 128.3 |
| 5 | 6.77-6.67 (1H) (m) | 146.9 |
| 6 | | 137.3 |
| 6-Me | 1.95 (s) | 12.6 |
| 7 | 6.45-6.48 (1H) (m) | 136.9 |
| 8 | 6.77-6.67 (1H) (m) | 131.5 |
| 9 | 6.77-6.67 (1H) (m) | 131.5 |
| 10 | 6.45-6.48 (1H) (m) | 127.0 |
| 11 | | 140.6 |
| 12 | 2.93 (dq, 9.6, 6.4) | 47.4 |
| 12-Me | 1.18 (d, 6.0) | 16.2 |
| 13 | 4.08-4.02 (m) | 74.0 |
| 14 | 4.23 (br. s) | 78.8 |
| 15 | | 211.4 |
| 16 | 3.04/2.83 (dd 16.9, 8.0, 2.1/dd, 17.4, 4.6) | 44.4 |
| 17 | 4.53-4.48 (m) | 66.7 |
| 18 | 4.05 (dd, 4.5, 1.5) | 67.8 |
| 19 | 5.27 (t, 7.7) | 75.4 |
| 20 | 5.55 (dd, 15.4, 7.4) | 126.0 |
| 21 | 5.89 (dt, 15.2, 6.4) | 139.1 |
| 22 | 2.09 (dt, 13.9, 7.1) | 26.2 |
| 23 | 1.02 (t, 7.4) | 13.5 |

TABLE 16
NMR assignments for 5m enacyloxin analogue (d₄-MeOH, ¹H 500 MHz, ¹³C 125 MHz).

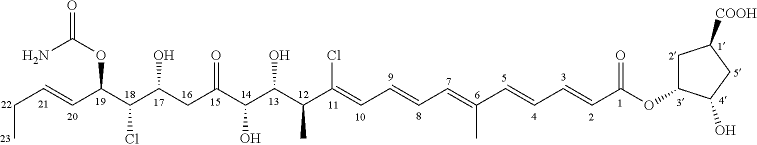 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@@H](C(=O)O)C[C@@H]1O]

| Position | ¹H (ppm) | ¹³C (ppm) |
|---|---|---|
| 1′-COOH | | 180.8 |
| 1′ | 2.25 (dt, 12.8, 5.9) | 40.2 |
| 2′ | 2.14-2.01 (2H) (m) | 35.8 |
| 3′ | 5.13-5.08 (m) | 74.1 |
| 4′ | 4.30 (q, 5.1) | 73.2 |
| 5′ | 2.14-2.01 (m/m) | 33.0 |
| 1 | | 168.5 |
| 2 | 6.00 (d, 15.2) | 121.1 |
| 3 | 7.43 (dd, 15.1, 11.1) | 146.6 |
| 4 | 6.52 (dd, 15.2, 11.4) | 128.4 |
| 5 | 6.79-6.70 (1H) (m) | 146.9 |
| 6 | | 137.4 |
| 6-Me | 1.95 (s) | 12.6 |
| 7 | 6.45-6.39 (1H) (m) | 137.0 |
| 8 | 6.79-6.70 (1H) (m) | 131.6 |
| 9 | 6.79-6.70 (1H) (m) | 131.6 |
| 10 | 6.45-6.39 (1H) (m) | 127.2 |
| 11 | | 140.7 |
| 12 | 2.94 (dq, 9.4, 6.9) | 47.5 |
| 12-Me | 1.19 (d, 6.7) | 16.2 |
| 13 | 4.06-4.02 (1H) (m) | 77.4 |
| 14 | 4.24 (d, 1.5) | 78.9 |
| 15 | | 211.5 |
| 16 | 3.05/2.83 (dd 17.0, 8.0/dd, 17.0, 4.5) | 44.5 |
| 17 | 4.51 (ddd, 7.3, 4.4, 2.5) | 66.8 |
| 18 | 4.07-4.03 (1H) (m) | 67.9 |
| 19 | 5.27 (t, 7.7) | 75.5 |
| 19-carbamate | | 158.6 |
| 20 | 5.55 (ddt, 15.4, 7.3, 1.4) | 126.2 |
| 21 | 5.89 (dt, 15.2, 6.3) | 139.2 |
| 22 | 2.14-2.05 (m) | 26.3 |
| 23 | 1.01 (t, 7.5) | 13.6 |

TABLE 17
NMR assignments for ‘anti’ enacyloxin analogue (d₄-MeOH, ¹H 500 MHz, ¹³C 125 MHz).

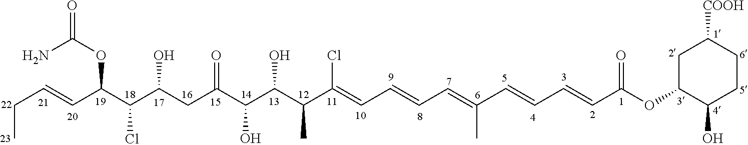 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@H](C(=O)O)CC[C@H]1O]

| Position | ¹H (ppm) | ¹³C (ppm) |
|---|---|---|
| 1′-COOH | | 179.4 |
| 1′ | 2.45 (br. t, 11.3) | 42.5 |
| 2′ | 1.54-1.42 (2H) (m) | 33.8 |
| 3′ | 4.68 (ddd, 11.2, 9.2, 4.5) | 74.1 |
| 4′ | 3.58 (td, 10.1, 4.7) | 72.5 |
| 5′ | 2.28-2.22/1.54-1.42 (m/m) | 33.1 |
| 6′ | 2.02-1.98/1.54-1.42 (m/m) | 28.1 |
| 1 | | 168.6 |
| 2 | 5.98 (d, 15.4) | 121.3 |
| 3 | 7.41 (dd, 15.0, 11.0) | 146.6 |
| 4 | 6.52 (dd, 15.2, 11.3) | 128.4 |
| 5 | 6.78-6.70 (1H) (m) | 146.8 |
| 6 | | 137.4 |
| 6-Me | 1.95 (s) | 12.6 |
| 7 | 6.45-6.39 (1H) (m) | 136.9 |
| 8 | 6.78-6.70 (1H) (m) | 131.6 |
| 9 | 6.78-6.70 (1H) (m) | 131.5 |
| 10 | 6.45-6.39 (1H) (m) | 127.2 |
| 11 | | 140.7 |
| 12 | 2.94 (dq, 9.7, 7.0) | 47.6 |
| 12-Me | 1.19 (d, 6.7) | 16.2 |
| 13 | 4.06-4.02 (1H) (m) | 77.7 |
| 14 | 4.23 (d, 1.6) | 78.9 |
| 15 | | 211.4 |
| 16 | 3.04/2.83 (dd 17.2, 8.0/dd, 17.1, 4.8) | 44.5 |
| 17 | 4.51 (ddd, 7.3, 4.4, 2.5) | 66.8 |
| 18 | 4.06-4.02 (1H) (m) | 67.9 |
| 19 | 5.27 (t, 7.8) | 75.5 |
| 19-carbamate | | 158.6 |
| 20 | 5.55 (ddt, 15.4, 7.4, 1.5) | 126.2 |
| 21 | 5.89 (dt, 15.3, 6.4) | 139.2 |
| 22 | 2.13-2.06 (m) | 26.3 |
| 23 | 1.01 (t, 7.5) | 13.6 |

TABLE 18
NMR assignments for Δ5930/32/12-14 unsaturated enacyloxin analogue (d₄-MeOH, ¹H 500 MHz, ¹³C 125 MHz).

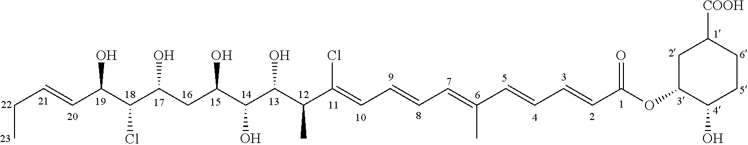 [SMILES: CC/C=C/[C@@H](O)[C@@H](Cl)[C@H](O)C[C@@H](O)[C@@H](O)[C@H](O)[C@H](C)/C(Cl)=C/C=C/C=C(C)/C=C/C=C/C(=O)O[C@@H]1CC(C(=O)O)CC[C@@H]1O]

| Position | ¹H (ppm) | ¹³C (ppm) |
|---|---|---|
| 1′-COOH | | 170.4 |
| 1′ | | 129.1 |
| 2′ | 2.65-2.53 (2H) (m) | 32.6 |
| 3′ | 5.14-5.10 (m) | 72.9 |
| 4′ | 4.07-4.03 (m) | 66.9 |
| 5′ | 2.49-2.42 (1H) (m), 2.65-2.53 (1H) (m) | 28.6 |
| 6′ | 6.88 (br. s) | 137.1 |
| 1 | | 168.6 |
| 2 | 5.98 (d, 15.2) | 121.1 |
| 3 | 7.42 (dd, 15.2, 11.1) | 146.7 |
| 4 | 6.51 (dd, 15.6, 11.5) | 127.8 |
| 5 | 6.77-6.71 (1H) (m) | 147.0 |
| 6 | | 137.2 |
| 6-Me | 1.95 (s) | 12.6 |
| 7 | 6.45-6.40 (1H) (m) | 137.1 |
| 8 | 6.77-6.71 (1H) (m) | 131.8 |
| 9 | 6.77-6.71 (1H) (m) | 131.2 |
| 10 | 6.45-6.40 (1H) (m) | 127.1 |
| 11 | | 142.1 |
| 12 | 2.87 (dq, 9.5, 6.7) | 47.5 |
| 12-Me | 1.12 (d, 6.7) | 16.1 |
| 13 | 3.96 (dd, 9.6, 0.7) | 72.2 |
| 14 | 3.36 (dd, 8.1, 0.7) | 74.1 |
| 15 | 3.90 (ddd, 10.1, 8.1, 2.1) | 69.5 |
| 16 | 2.21/1.46 (ddd 14.1, 10.1, 2.1/ddd, 14.1, 10.0, 2.3) | 40.4 |
| 17 | 4.44 (dt, 10.1, 2.3) | 67.7 |
| 18 | 3.74 (dd, 7.6, 2.2) | 70.1 |
| 19 | 4.27 (t, 7.3) | 74.4 |
| 20 | 5.58 (ddt, 15.3, 7.1, 1.4) | 130.1 |
| 21 | 5.81 (dtd, 15.3, 6.4, 0.6) | 136.6 |
| 22 | 2.13-2.06 (m) | 26.4 |
| 23 | 1.02 (t, 7.5) | 13.8 |

TABLE 19
NMR assignments for Δ5930 + Δ5932_Br/H enacyloxin analogue (d₄-MeOH, ¹H 500 MHz, ¹³C 125 MHz)

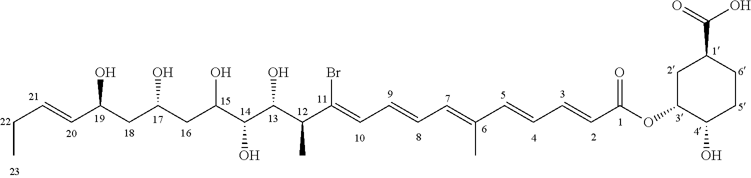 [SMILES: CC/C=C/[C@@H](O)C[C@H](O)CC(O)[C@@H](O)[C@H](O)[C@H](C)/C(Br)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | |
| 1′ | 2.48-2.59 (m) | 38.8 |
| 2′ | 1.70-1.76, 2.13-2.20 (m, m) | 32.4 |
| 3′ | 5.18-5.22 (m) | 73.1 |
| 4′ | 3.70-3.75 (m) | 70.5 |
| 5′ | 1.79-1.84 (m) | 29.5 |
| 6′ | 1.57-1.63, 2.00-2.03 (m, m) | 27.6 |
| 1 | | 168.5 |
| 2 | 6.03 (d, 15.2) | 121.4 |
| 3 | 7.43 (dd, 15.1, 11.0) | 146.8 |
| 4 | 6.54 (dd, 15.1, 11.2) | 127.2 |
| 5 | 6.76 (d, 15.2) | 146.6 |
| 6 | | 137.5 |
| 6-Me | 1.96 (s) | 12.7 |
| 7 | 6.41 (d, 11.4) | 137.0 |
| 8 | 6.81 (dd, 14.2, 11.5) | 131.9 |
| 9 | 6.63-6.72 (m) | 133.6 |
| 10 | 6.35 (d, 11.3) | 136.7 |
| 11 | | 137.5 |
| 12 | 2.80 (dq, 9.8, 6.7) | 49.6 |
| 12-Me | 1.11 (d, 6.7) | 17.2 |
| 13 | 3.95 (d, 9.7) | 72.8 |
| 14 | 3.36 (d, 8.1) | 74.0 |
| 15 | 3.89-3.93 (m) | 69.9 |
| 16 | 1.52, 1.90 (ddd, 13.9, 9.6, | 42.7 |
| | 2.7, ddd, 13.9, 9.8, 2.5) | |
| 17 | 4.12 (tt, 9.8, 2.8) | 66.3 |
| 18 | 1.57-1.66 | 46.5 |
| 19 | 4.28 (dt, 6.5, 6.0) | 70.3 |
| 20 | 5.50 (ddt, 15.5, 6.8, 1.3) | 133.4 |
| 21 | 5.71 (dt, 15.4, 6.3) | 134.0 |
| 22 | 2.06 (dq, 7.4, 1.2) | 26.3 |
| 23 | 1.01 (t, 7.4) | 14.0 |

TABLE 20
NMR assignments for BrCl enacyloxin analogue (d₄-MeOH, ¹H 500 MHz, ¹³C 125 MHz)

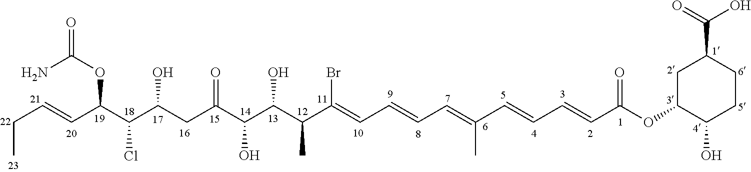 [SMILES: CC/C=C/[C@@H](OC(N)=O)[C@@H](Cl)[C@H](O)CC(=O)[C@@H](O)[C@H](O)[C@H](C)/C(Br)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | |
| 1′ | 2.48-2.58 (m) | 39.0 |
| 2′ | 1.70-1.76, 2.14-2.19 (m, m) | 32.5 |
| 3′ | 5.18-5.23 (m) | 73.2 |
| 4′ | 3.70-3.76 (m) | 70.6 |
| 5′ | 1.79-1.84 (m) | 29.5 |
| 6′ | 1.50-1.62, 2.01-2.06 (m, m) | 27.7 |
| 1 | | 168.6 |
| 2 | 6.03 (d, 15.5) | 121.4 |
| 3 | 7.43 (dd, 15.1, 11.2) | 146.8 |
| 4 | 6.55 (dd, 15.1, 11.1) | 127.4 |
| 5 | 6.76 (d, 15.0) | 146.6 |
| 6 | | 137.7 |
| 6-Me | 1.96 (s) | 12.7 |
| 7 | 6.41 (d, 11.4) | 136.8 |
| 8 | 6.82 (dd, 13.7, 11, 7) | 132.3 |
| 9 | 6.65-6.70 (m) | 133.7 |
| 10 | 6.34 (d, 11.5) | 136.6 |
| 11 | | 136.0 |
| 12 | 2.84-2.90 (m) | 47.7 |
| 12-Me | 1.17 (d, 6.5) | 17.3 |
| 13 | 4.04 (dd, 2.1, 8.1) | 74.7 |
| 14 | 4.24 (d, 1.4) | 78.9 |
| 15 | | 211.5 |
| 16 | 2.84, 3.04 (dd, 4.2, 17.2, dd, 16.9, 8.6) | 44.6 |
| 17 | 4.51 (ddd, 2.4, 4.3, 7.2) | 66.8 |
| 18 | 4.02-4.07 (m) | 67.9 |
| 19 | 5.27 (t, 8.0) | 75.5 |
| 19-carbamate | | 158.6 |
| 20 | 5.55 (ddt, 15.0, 7.6, 1.4) | 126.2 |
| 21 | 5.90 (dt, 15.5, 6.4) | 139.3 |
| 22 | 2.06-2.13 (m) | 26.3 |
| 23 | 1.02 (t, 7.5) | 13.6 |

TABLE 21
NMR assignments for BrH enacyloxin analogue (d₄-MeOH, ¹H 500 MHz, ¹³C 125 MHz)

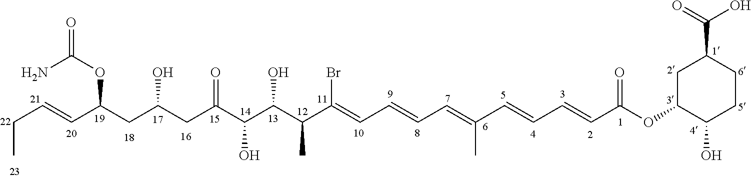 [SMILES: CC/C=C/[C@H](C[C@H](O)CC(=O)[C@@H](O)[C@H](O)[C@H](C)/C(Br)=C/C=C/C=C(C)/C=C/C=C/C(=O)O[C@@H]1C[C@@H](C(=O)O)CC[C@@H]1O)OC(N)=O]

| Position | ¹H (ppm) | ¹³C (ppm) |
|---|---|---|
| 1′COOH | | |
| 1′ | 2.47-2.58 (m) | 39.1 |
| 2′ | 1.71-1.75, 2.13-2.19 (m, m) | 32.6 |
| 3′ | 5.19-5.22 (m) | 73.3 |
| 4′ | 3.73 (ddd, 8.6, 6.2, 2.8) | 70.7 |
| 5′ | 1.79-1.84 (m) | 29.5 |
| 6′ | 1.50-1.57, 2.00-2.03 (m, m) | 27.8 |
| 1 | | 168.6 |
| 2 | 6.03 (d, 15.5) | 121.5 |
| 3 | 7.43 (dd, 15.2, 11.3) | 146.8 |
| 4 | 6.55 (dd, 15.2, 11.2) | 127.4 |
| 5 | 6.76 (d, 15.1) | 146.6 |
| 6 | | 137.7 |
| 6-Me | 1.95 (s) | 12.7 |
| 7 | 6.41 (d, 11.8) | 136.8 |
| 8 | 6.82 (dd, 13.6, 11.3) | 132.2 |
| 9 | 6.63-6.70 (m) | 133.7 |
| 10 | 6.35 (d, 11.3) | 136.6 |
| 11 | | 136.1 |
| 12 | 2.84-2.89 (m) | 49.6 |
| 12-Me | 1.17 (d, 6.9) | 17.3 |
| 13 | 4.05 (dd, 10.0, 1.05) | 74.6 |
| 14 | 4.23 (d, 1.4) | 78.9 |
| 15 | | 212.0 |
| 16 | 2.83, 2.66 (dd, 16.4, 8.4, dd, 16.6, 4.0) | 47.2 |
| 17 | 4.20 (tt, 8.3, 3.5) | 65.2 |
| 18 | 1.80, 1.65 (ddd, 13.8, 9.8, | 43.8 |
| | 3.23, ddd, 13.9, 9.3, 3.5) | |
| 19 | 5.23 (ddd, 9.9, 7.0, 3.3) | 73.0 |
| 19-carbamate | | 159.7 |
| 20 | 5.45 (ddt, 15.5, 7.4, 1.4) | 129.3 |
| 21 | 5.78 (dt, 15.5, 6.5) | 135.8 |
| 22 | 2.05 (dq, 7.4, 1.2) | 26.2 |
| 23 | 0.99 (t, 7.5) | 13.7 |

TABLE 22
NMR assignments for Δ5927_Br/Br enacyloxin analogue (d₄-MeOH, ¹H 500 MHz, ¹³C 125 MHz)

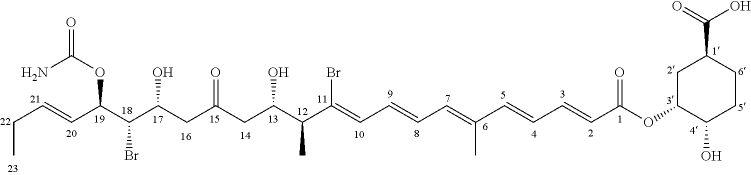 [SMILES: CC/C=C/[C@@H](OC(N)=O)[C@@H](Br)[C@H](O)CC(=O)C[C@H](O)[C@H](C)/C(Br)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | |
| 1′ | 2.49-2.58 | 38.8 |
| 2′ | 1.69-1.77, 2.13-2.20 (m, m) | 32.4 |
| 3′ | 5.17-5.24 (m) | 73.1 |
| 4′ | 3.73 (ddd, 8.6, 6.0, 2.8) | 70.7 |
| 5′ | 1.78-1.85 (m) | 29.5 |
| 6′ | 1.51-1.62, 2.00-2.07 (m, m) | 27.6 |
| 1 | | 168.5 |
| 2 | 6.03 (d, 15.2) | 121.5 |
| 3 | 7.43 (dd, 15.2, 11.2) | 146.9 |
| 4 | 6.55 (dd, 15.2, 11.2) | 127.4 |
| 5 | 6.76 (d, 15.2) | 146.6 |
| 6 | | 137.8 |
| 6-Me | 1.96 (s) | 12.7 |
| 7 | 6.41 (d, 11.6) | 136.8 |
| 8 | 6.83 (dd, 13.7, 11.7) | 132.4 |
| 9 | 6.61-6.68 (m) | 133.8 |
| 10 | 6.35 (d, 11.3) | 136.5 |
| 11 | | 134.7 |
| 12 | 2.58-2.74 (m) | 51.8 |
| 12-Me | 1.13 (d, 6.8) | 16.5 |
| 13 | 4.24 (td, 7.9, 3.3) | 70.5 |
| 14 | 2.58-2.71 (m) | Solvent |
| 15 | | 209.5 |
| 16 | 2.90, 2.78 (dd, 17.0, 8.0, dd, 17.0, 4.7) | 50.4 |
| 17 | 4.31-4.35 (m) | 66.3 |
| 18 | 4.12 (dd, 8.1, 2.4) | 62.6 |
| 19 | 5.30 (t, 7.8) | 75.7 |
| 19-carbamate | | 158.6 |
| 20 | 5.54 (ddt, 15.4, 7.5, 1.5) | 126.9 |
| 21 | 5.89 (dt, 15.4, 6.4) | 139.2 |
| 22 | 2.10 (dq, 7.4, 1.2) | 26.3 |
| 23 | 1.01 (t, 7.5) | 13.6 |

TABLE 23
NMR assignments for Δ5927_Br/Cl enacyloxin analogue (d₄-MeOH, ¹H 500 MHz, ¹³C 125 MHz)

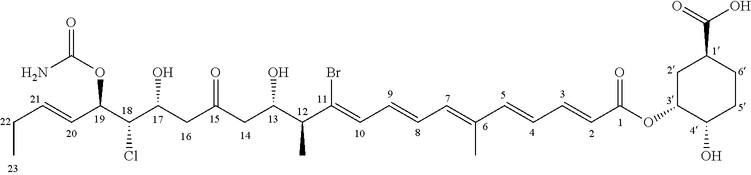 [SMILES: CC/C=C/[C@@H](OC(N)=O)[C@@H](Cl)[C@H](O)CC(=O)C[C@H](O)[C@H](C)/C(Br)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | |
| 1′ | 2.46-2.55 (m) | |
| 2′ | 1.69-1.76, 2.14-2.19 (m, m) | 32.6 |
| 3′ | 5.18-5.23 (m) | 73.3 |
| 4′ | 3.73 (ddd, 8.6, 6.0, 2.8) | 70.8 |
| 5′ | 1.79-1.83 (m) | 29.6 |
| 6′ | 1.47-1.64, 1.99-2.06 (m, m) | 26.3 |
| 1 | | 168.5 |
| 2 | 6.03 (d, 15.3) | 121.5 |
| 3 | 7.43 (dd, 15.2 11.2) | 146.7 |
| 4 | 6.55 (dd, 15.1, 11.2) | 127.4 |
| 5 | 6.76 (d, 15.2) | 146.5 |
| 6 | | 137.8 |
| 6-Me | 1.96 (s) | 12.7 |
| 7 | 6.41 (d, 11.6) | 136.8 |
| 8 | 6.83 (dd, 14.0, 11.6) | 132.4 |
| 9 | 6.60-6.65 (m) | 133.7 |
| 10 | 6.35 (d, 11.4) | 136.5 |
| 11 | | 134.6 |
| 12 | 2.58-2.70 (m) | 51.8 |
| 12-Me | 1.13 (d, 6.8) | 16.5 |
| 13 | 4.24 (td, 8.0, 3.3) | 70.7 |
| 14 | 2.58-2.70 (m) | Solvent |
| 15 | | 209.6 |
| 16 | 2.90, 2.78 (dd, 17.0, 7.9, dd, 17.0, 4.8) | Solvent |
| 17 | 4.44-4.48 (m) | 66.6 |
| 18 | 3.99 (dd, 8.1, 2.4) | 67.8 |
| 19 | 5.26 (t, 7.8) | 75.5 |
| 19-carbamate | | 158.6 |
| 20 | 5.54 (ddt, 15.4, 7.5, 1.5) | 126.2 |
| 21 | 5.89 (dt, 15.4, 6.4) | 139.2 |
| 22 | 2.10 (dq, 7.4, 1.2) | 26.3 |
| 23 | 1.01 (t, 7.5) | 13.6 |

TABLE 24
NMR assignments for Δ5930_Br/Br enacyloxin analogue (d₄-MeOH, ¹H 500 MHz, ¹³C 125 MHz)

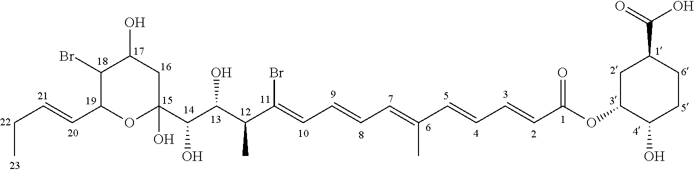 [SMILES: CC/C=C/C1OC(O)([C@@H](O)[C@H](O)[C@H](C)/C(Br)=C/C=C/C=C(C)/C=C/C=C/C(=O)O[C@@H]2C[C@@H](C(=O)O)CC[C@@H]2O)CC(O)C1Br]

| Position | ¹H (ppm) | ¹³C (ppm) |
|---|---|---|
| 1′-COOH | | |
| 1′ | 2.49-2.58 (m) | |
| 2′ | 1.69-1.77, 2.16-2.20 (m, m) | 32.5 |
| 3′ | 5.18-5.22 (m) | 73.2 |
| 4′ | 3.73 (ddd, 8.6, 6.1, 2.8) | 70.6 |
| 5′ | 1.78-1.85 (m) | 29.5 |
| 6′ | 1.52-1.61, 2.00-2.06 (m, m) | 27.7 |
| 1 | | 168.5 |
| 2 | 6.03 (d, 15.2) | 121.4 |
| 3 | 7.43 (dd, 15.2, 11.1) | 146.8 |
| 4 | 6.55 (dd, 15.1, 11.2) | 127.3 |
| 5 | 6.76 (d, 15.1) | 146.6 |
| 6 | | 137.6 |
| 6-Me | 1.95 (s) | 12.7 |
| 7 | 6.41 (d, 11.5) | 136.9 |
| 8 | 6.81 (dd, 14.2, 11.5) | 132.1 |
| 9 | 6.64-6.71 (m) | 133.8 |
| 10 | 6.35 (d, 11.4) | 136.6) |
| 11 | | 137.3 |
| 12 | 2.82 (dq, 9.6, 6.7) | 49.6 |
| 12-Me | 1.10 (d, 6.7) | 17.1 |
| 13 | 4.16 (d, 9.6) | 73.3 |
| 14 | 3.45 (d, 0.7) | 73.4 |
| 15 | | 100.5 |
| 16 | 2.44, 1.54 (dd, 13.0, 5.0, dd, 12.9, 1.3) | 43.0 |
| 17 | 3.49 (t, 10.1) | 61.5 |
| 18 | 4.01-4.08 (m) | 70.9 |
| 19 | 4.46 (dd, 10.2, 6.9) | 74.9 |
| 20 | 5.54 (ddt, 15.4, 7.0, 1.4) | 128.0 |
| 21 | 5.83 (dt, 15.3, 6.4) | 137.8 |
| 22 | 2.10 (dq, 7.5, 1.3) | 26.4 |
| 23 | 1.02 (t, 7.5) | 13.8 |

TABLE 25
NMR assignments for Δ5930_Br/H enacyloxin analogue (d₄-MeOH, ¹H 500 MHz, ¹³C 125 MHz)

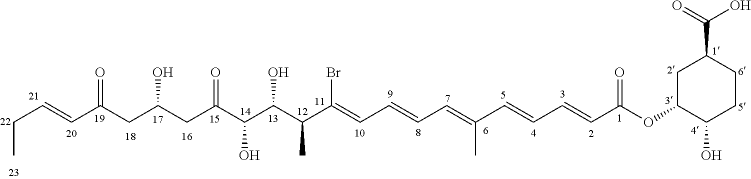 [SMILES: CC/C=C/C(=O)C[C@H](O)CC(=O)[C@@H](O)[C@H](O)[C@H](C)/C(Br)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | |
| 1′ | 2.46-2.58 (m) | |
| 2′ | 1.69-1.79, 2.13-2.19 (m, m) | 32.8 |
| 3′ | 5.18-5.23 (m) | 73.4 |
| 4′ | 3.72 (ddd, 8.6, 6.0, 2.8) | 70.8 |
| 5′ | 1.78-1.83 (m) | 29.6 |
| 6′ | 1.49-1.63, 1.99-2.06 (m, m) | 26.6 |
| 1 | | 168.6 |
| 2 | 6.03 (d, 15.3) | 121.5 |
| 3 | 7.43 (dd, 15.2, 11.2) | 146.7 |
| 4 | 6.55 (dd, 15.1, 11.2) | 127.4 |
| 5 | 6.76 (d, 15.2) | 146.5 |
| 6 | | 137.7 |
| 6-Me | 1.96 (s) | 12.6 |
| 7 | 6.41 (d, 11.6) | 136.8 |
| 8 | 6.82 (dd, 14.0, 11.6) | 132.2 |
| 9 | 6.62-6.65 (m) | 133.7 |
| 10 | 6.35 (d, 11.4) | 136.6 |
| 11 | | 136.0 |
| 12 | 2.79-2.81 (m) | 46.6 |
| 12-Me | 1.17 (d, 6.7) | 17.3 |
| 13 | 4.05 (dd, 10.0, 2.0) | 74.7 |
| 14 | 4.23 (d, 1.7) | 78.9 |
| 15 | | 212.1 |
| 16 | 2.87 (dd, 16.6, 7.0) | 47.6 |
| 17 | 2.79-2.82 (m) | 47.5 |
| 18 | 4.55-4.61 (m) | 65.5 |
| 19 | | 201.3 |
| 20 | 6.14 (dt, 15.9, 1.6) | 131.2 |
| 21 | 7.01 (dt, 15.9, 6.5) | 151.8 |
| 22 | 2.28 (dq, 7.6, 1.5) | 26.6 |
| 23 | 1.10 (t, 7.5) | 12.7 |

TABLE 26
NMR assignments for Δ5930 + Δ5932_Br/Br enacyloxin analogue (d₄-MeOH, ¹H 500 MHz, ¹³C 125 MHz)

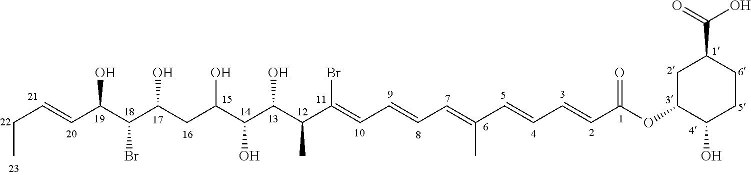 [SMILES: CC/C=C/[C@@H](O)[C@@H](Br)[C@H](O)CC(O)[C@@H](O)[C@H](O)[C@H](C)/C(Br)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | |
| 1′ | 2.48-2.61 (m) | 38.8 |
| 2′ | 1.69-1.76, 2.14-2.17 (m, m) | 32.4 |
| 3′ | 5.18-5.24 (m) | 73.2 |
| 4′ | 3.73 (ddd, 8.6, 6.1, 2.8) | 70.6 |
| 5′ | 1.78-1.85 (m) | 29.5 |
| 6′ | 1.52-1.62, 2.01-2.06 (m, m) | 27.6 |
| 1 | | 168.5 |
| 2 | 6.03 (d, 15.3) | 121.4 |
| 3 | 7.43 (dd, 15.2, 11.1) | 146.8 |
| 4 | 6.55 (dd, 15.1, 11.2) | 127.3 |
| 5 | 6.76 (d, 15.1) | 146.6 |
| 6 | | 137.5 |
| 6-Me | 1.96 (s) | 12.7 |
| 7 | 6.42 (d, 11.6) | 137.0 |
| 8 | 6.81 (dd, 14.3, 11.4) | 131.9 |
| 9 | 6.65-6.72 (m) | 134.0 |
| 10 | 6.35 (d, 11.4) | 136.7 |
| 11 | | 137.3 |
| 12 | 2.80 (dq, 9.6, 6.6) | 49.6 |
| 12-Me | 1.11 (d, 6.7) | 17.2 |
| 13 | 3.95 (d, 9.5) | 72.9 |
| 14 | 3.36 (d, 8.1) | 74.1 |
| 15 | 3.88-3.90 (m) | 69.5 |
| 16 | 1.46, 2.21 (ddd, 14.0, 9.9, 2.4, ddd, 13.9, 9.9, 2.4) | 41.8 |
| 17 | 4.29-4.35 (m) | 67.6 |
| 18 | 3.92 (dd, 7.6, 2.3) | 66.7 |
| 19 | 4.29-4.35 (m) | 74.7 |
| 20 | 5.59 (ddt, 15.5, 7.1, 1.4) | 130.7 |
| 21 | 5.81 (dt, 15.3, 6.4) | 136.6 |
| 22 | 2.10 (dq, 7.5, 1.3) | 26.3 |
| 23 | 1.03 (t, 7.5) | 13.8 |

TABLE 27
NMR assignments for Δ5930 + 5932_Br/Cl enacyloxin analogue (d₄-MeOH, ¹H 500 MHz, ¹³C 125 MHz)

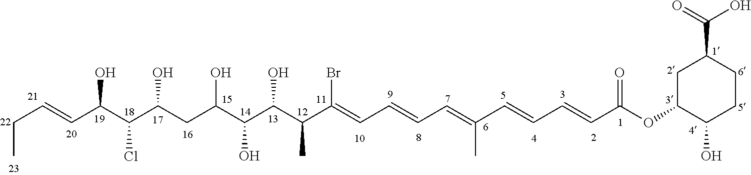 [SMILES: CC/C=C/[C@@H](O)[C@@H](Cl)[C@H](O)CC(O)[C@@H](O)[C@H](O)[C@H](C)/C(Br)=C/C=C/C=C(C)/C=C/C=C/C(=O)O[C@@H]1C[C@@H](C(=O)O)CC[C@@H]1O]

| Position | ¹H (ppm) | ¹³C (ppm) |
|---|---|---|
| 1′-COOH | | |
| 1′ | 2.47-2.59 (m) | |
| 2′ | 1.70-1.76, 2.17-2.19 (m, m) | 32.5 |
| 3′ | 5.18-5.22 (m) | 73.2 |
| 4′ | 3.70-3.74 (m) | 70.6 |
| 5′ | 1.79-1.85 (m) | 29.5 |
| 6′ | 1.54-1.61, 2.01-2.05 (m, m) | 27.7 |
| 1 | | 168.5 |
| 2 | 6.03 (d, 15.2) | 121.4 |
| 3 | 7.43 (dd, 15.2, 11.2) | 146.8 |
| 4 | 6.55 (dd, 14.9, 11.3) | 127.3 |
| 5 | 6.76 (d, 15.2) | 146.6 |
| 6 | | 137.5 |
| 6-Me | 1.96 (s) | 12.7 |
| 7 | 6.42 (d, 11.4) | 137.0 |
| 8 | 6.81 (dd, 14.3, 11.6) | 131.9 |
| 9 | 6.65-6.73 (m) | 134.0 |
| 10 | 6.35 (d, 11.5) | 136.7 |
| 11 | | 137.4 |
| 12 | 2.80 (dq, 9.4, 6.7) | 49.6 |
| 12-Me | 1.11 (d, 6.7) | 17.2 |
| 13 | 3.95 (d, 9.5) | 72.9 |
| 14 | 3.36 (d, 8.3) | 74.1 |
| 15 | 3.88-3.93 (m) | 69.6 |
| 16 | 1.46, 2.20-2.24 (ddd, 14.1, 9.9, 2.4, m) | 40.5 |
| 17 | 4.27 (t, 7.5) | 74.4 |
| 18 | 3.74 (dd, 7.6, 2.3) | 71.1 |
| 19 | 4.43-4.47 (m) | 67.8 |
| 20 | 5.59 (ddt, 15.5, 7.0, 1.3) | 130.6 |
| 21 | 5.82 (dt, 15.3, 6.4) | 136.6 |
| 22 | 2.10 (dq, 7.5, 1.3) | 26.4 |
| 23 | 1.03 (t, 7.5) | 13.8 |

### Example 2—Minimum Inhibitory Concentration (MIC) and Minimal Bactericidal Concentration (MBC) Measurements

MIC values for the compounds were determined using the broth microdilution method according to the official CLSI guidelines. Acinetobacter baumannii test strains were grown overnight in Mueller-Hinton (MH) broth at 37° C. In a 96-well microtiter plate, 50 μl of serial two-fold dilutions of an enacyloxin derivative in MH broth were mixed with 50 μl of bacterial suspension, diluted to a concentration of 10⁶ CFU/ml in MH broth. The desired inoculum density was achieved using a 0.5 McFarland turbidity standard. Following incubation for 18 h at 37° C., MICs were determined (defined as the lowest concentrations that visibly inhibited bacterial growth).

Cell suspensions without visible growth were then plated out on LB agar plates to determine the minimal bactericidal concentration (MBC). The MBC was set as the lowest concentration required to kill 99.9% of the originally inoculated 5·10⁵ CFU/ml. All MIC and MBC determinations were performed in triplicate.

TABLE 28
MIC values for enacyloxin derivatives against multi-drug resistant
A. baumannii strains

| | MIC against A. baumannii |
|---|---|
| Compound No. | (μg/ml) |
| 1 | 2 |
| 2 | 4 |
| 3 | 4 |
| 4 | 16 |
| 5 | 8 |
| 6 | 8 |
| 7 | 4 |
| 8 | 8 |
| 9 | 1 |
| 10 | 32 |
| 11 | 8 |
| 12 | >64 |
| 17 | 2 |
| 18 | 2-4 |
| 21 | 8 |
| 22 | 4 |
| 23 | 64 |
| 28 | 0.5 |
| 29 | 1 |
| 30 | 1 |
| 31 | 2 |
| 32 | 16 |
| 33 | 1 |
| 34 | 2 |
| 35 | 32 |

1. A compound of formula (A), or a pharmaceutically acceptable salt, metabolite, isomer (e.g. stereoisomer) or prodrug thereof: 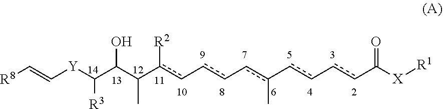
wherein:
X is O or NR^{x} (where R^{x} is either H or C₁₋₃ alkyl, e.g. CH₃);
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁸ is a straight-chained or branched C₁₋₈ alkyl group (e.g. a C₁₋₆ alkyl group);
Y is one of the following groups: 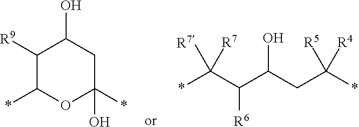
(where each * denotes the point of attachment of the group to the remainder of the molecule);
R⁹ is H, F, Cl, Br or I;
R⁴ and R⁵ are independently selected from H and OH, or R⁴ and R⁵ together are ═O, preferably R⁴ is H and R⁵ is OH;
R⁶ is H, F, Cl, Br, I or CH₃;
R⁷ is H and R^{7′} is OH, or R⁷ and R^{7′} together are ═O, preferably R⁷ is H and R⁷ is OH; and
each — independently represents an optional bond (i.e. each of C₂-C₃, C₄-C₅, C₆-C₇, C₈-C₉ and C₁₀-C₁₁ are independently either C—C (single) or C═C (double) bonds).

2. A compound as claimed in claim 1 of formula (IV), or a pharmaceutically acceptable salt, metabolite, isomer (e.g. stereoisomer) or prodrug thereof: 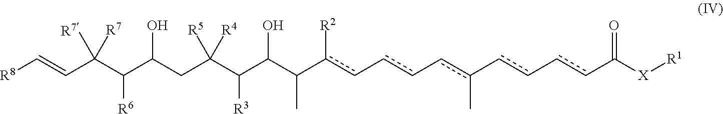
wherein:
X is O or NR^{x} (where R^{x} is either H or C₁₋₃ alkyl, e.g. CH₃);
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁴ and R⁵ are independently selected from H and OH, or R⁴ and R⁵ together are ═O, preferably R⁴ is H and R⁵ is OH;
R⁶ is H, F, Cl, Br, I or CH₃;
R⁷ is H and R^{7′} is OH, or R⁷ and R^{7′} together are ═O, preferably R⁷ is H and R⁷ is OH;
R⁸ is a straight-chained or branched C₁₋₈ alkyl group (e.g. C₁₋₆ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds).

3. A compound as claimed in claim 2, wherein R¹ is an optionally substituted cyclohexyl or cyclopentyl ring, an optionally substituted cyclohexenyl ring, or an optionally substituted straight-chained C₁₋₆ alkyl group.

4. A compound as claimed in claim 2 or claim 3, wherein R¹ is substituted by one or more of the following groups: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof).

5. A compound as claimed in any one of claims 2 to 4, wherein R⁸ is a straight-chained or branched C₁₋₅ alkyl, preferably a straight-chained or branched C₁₋₄ alkyl, e.g. methyl, ethyl, isopropyl, or tert. butyl.

6. A compound as claimed in claim 5, wherein R⁸ is ethyl.

7. A compound as claimed in claim 2 of formula (IVb) or a pharmaceutically acceptable salt, metabolite, isomer (e.g. stereoisomer) or prodrug thereof: 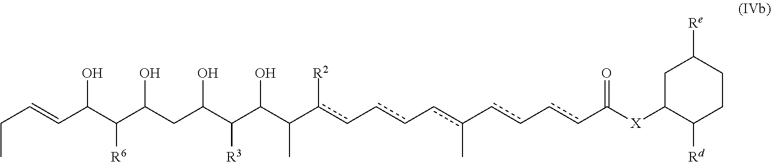
wherein:
X is as defined in claim 2;
R^{d} is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or
C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R², R³ and R⁶ are as defined in claim 2; and
each — independently represents an optional bond (i.e. each of C₂-C₃, C₄-C₅, C₆-C₇, C₈-C₉ and C₁₀-C₁₁ are independently either C—C (single) or C═C (double) bonds).

8. A compound as claimed in claim 7, wherein:
X is O or NH, preferably O;
R^{d} is H, OH, NH₂, SH, F, Cl, Br, I, or CH₃, preferably OH;
R^{e} is H, CO₂H, PO₃H₂, or SO₃H₂, preferably CO₂H;
R² is H, F, Cl, Br, I or CH₃, preferably H, Cl or Br, e.g. H or Cl;
R³ is H or OH;
R⁶ is H, Cl or Br, e.g. R⁶ is H or Cl; and
each of C₂-C₃, C₄-C₀, C₆-C₇, C₈-C₉ and C₁₀-C₁₁ are independently either C—C (single) or C═C (double) bonds.

9. A compound as claimed in any one of claims 2 to 8, wherein X is O.

10. A compound as claimed in any one of claims 2 to 9, wherein R² is Cl or H, preferably Cl, or wherein R² is Br.

11. A compound as claimed in any one of claims 2 to 10, wherein R³ is OH.

12. A compound as claimed in any one of claims 2 to 11, wherein R⁴ is H and R⁵ is OH.

13. A compound as claimed in any one of claims 2 to 12, wherein R⁶ is H or Cl, preferably H.

14. A compound as claimed in any one of claims 2 to 13, wherein R⁷ is H and R^{7′} is OH.

15. A compound as claimed in any one of claims 2 to 14, wherein each of C₂-C₃, C₄-C₅, C₆-C₇, C₈-C₉ and C₁₀-C₁₁ are C═C (double) bonds.

16. A compound as claimed in claim 2 of formula (IVc), or a pharmaceutically acceptable salt, metabolite, isomer (e.g. stereoisomer) or prodrug thereof: 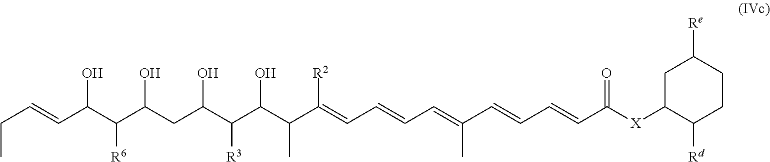
wherein X, R^{d}, R^{e}, R², R³ and R⁶ are as defined in any one of claims 2, 7 to 11 and 13.

17. A compound as claimed in claim 2 of formula (IVd), or a pharmaceutically acceptable salt, metabolite, or prodrug thereof: 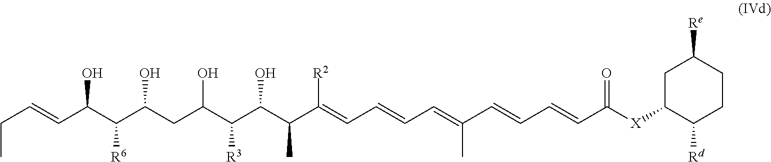
wherein X, R^{d}, R^{e}, R², R³ and R⁶ are as defined in any one of claims 2, 7 to 11 and 13.

18. A compound as claimed in claim 2 selected from any of the following compounds, and their pharmaceutically acceptable salts, metabolites, isomers (e.g. stereoisomers) and prodrugs:

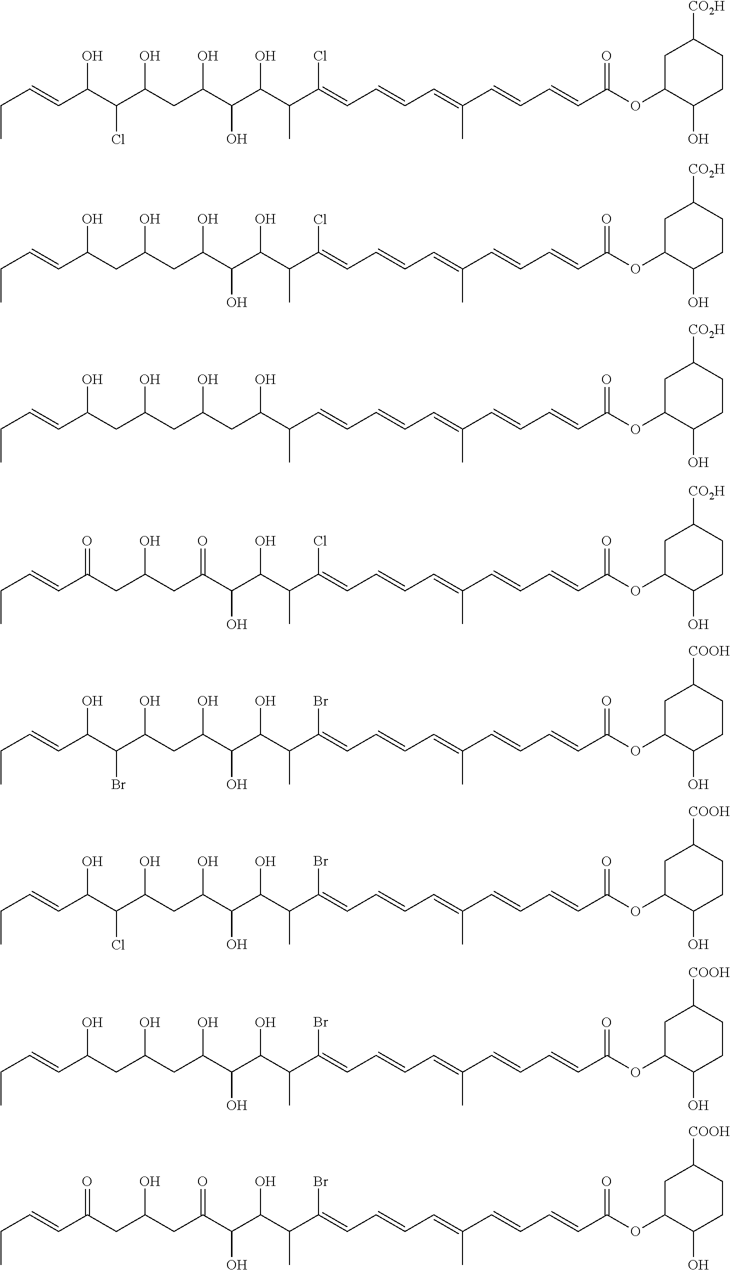

19. A compound as claimed in claim 2 selected from any of the following compounds, and their pharmaceutically acceptable salts, metabolites, and prodrugs:

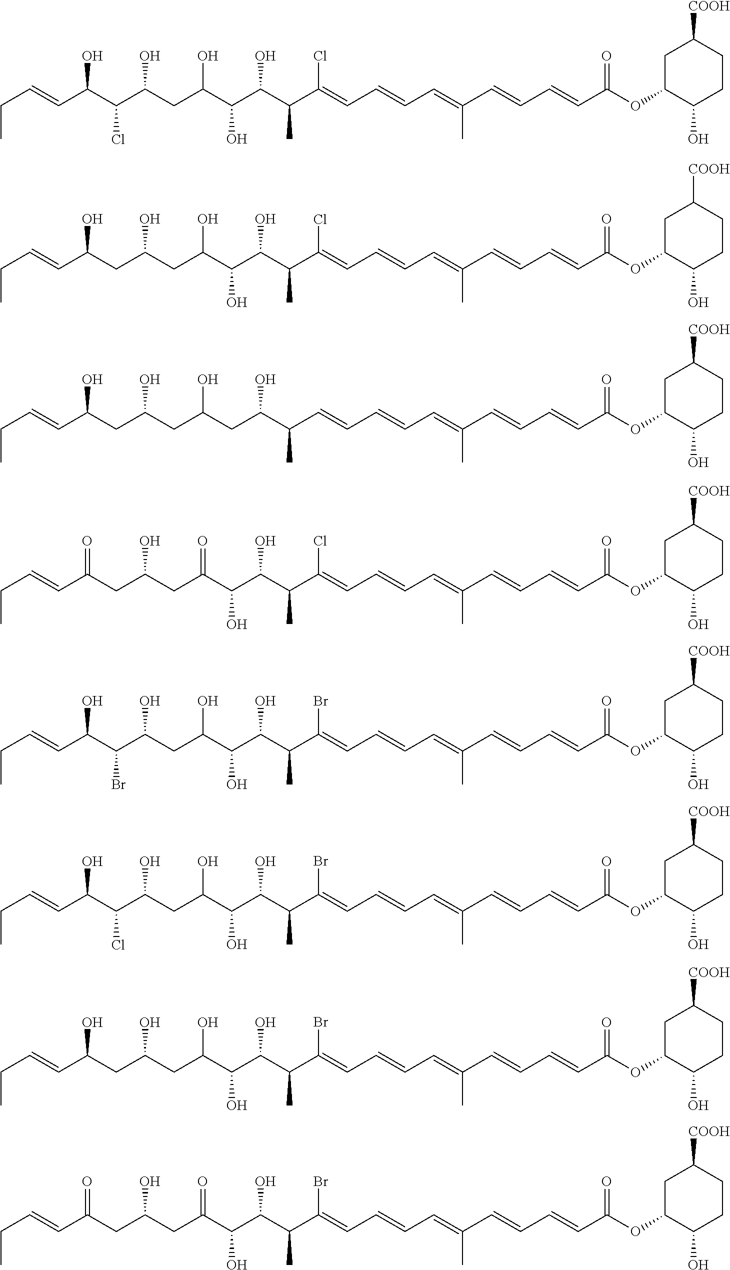

20. A compound as claimed in claim 1 of formula (I), or a pharmaceutically acceptable salt, metabolite, isomer (e.g. stereoisomer) or prodrug thereof: 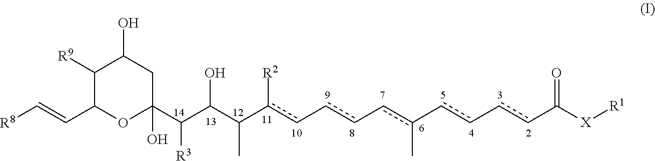
wherein:
X is O or NR^{x} (where R^{x} is either H or C₁₋₃ alkyl, e.g. CH₃);
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁸ is a straight-chained or branched C₁₈ alkyl group (e.g. a C₁₋₆ alkyl group);
R⁹ is H, F, Cl, Br or I, preferably H or Cl; and
each — independently represents an optional bond (i.e. each of C₂-C₃, C₄-C₅, C₆-C₇, C₈-C₉ and C₁₀-C₁₁ are independently either C—C (single) or C═C (double) bonds).

21. A compound as claimed in claim 20, wherein R¹ is an optionally substituted cyclohexyl or cyclopentyl ring, an optionally substituted cyclohexenyl ring, or an optionally substituted straight-chained C₁₋₆ alkyl group.

22. A compound as claimed in claim 20 or claim 21, wherein R¹ is substituted by one or more of the following groups: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof).

23. A compound as claimed in any one of claims 20 to 22, wherein R⁸ is a straight-chained or branched C₁₋₅ alkyl, preferably a straight-chained or branched C₁₋₄ alkyl, e.g. methyl, ethyl, isopropyl, or tert. butyl.

24. A compound as claimed in claim 23, wherein R⁸ is ethyl.

25. A compound as claimed in claim 20 of formula (Ia), or a pharmaceutically acceptable, salt, metabolite, isomer (e.g. stereoisomer) or prodrug thereof: 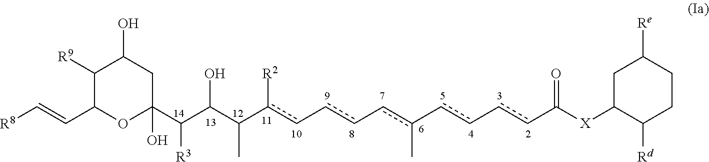
wherein:
X is as defined in claim 20;
R^{d} is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or
C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R², R³ and R⁹ are as defined in claim 20; and
each — independently represents an optional bond (i.e. each of C₂-C₃, C₄-C₅, C₆-C₇, C₈-C₉ and C₁₀-C₁₁ are independently either C—C (single) or C═C (double) bonds).

26. A compound as claimed in claim 25, wherein:
X=O or NH;
R^{d} is H, OH, NH₂, SH, F, Cl, Br, I, or CH₃, preferably OH;
R^{e} is H, CO₂H, PO₃H₂, or SO₃H₂, preferably CO₂H;
R² is H, F, Cl, Br, I or CH₃;
R³ is H or OH;
R⁹ is H, Cl or Br, e.g. R⁹ is H or Cl; and
each of C₂-C₃, C₄-C₅, C₆-C₇, C₈-C₉ and C₁₀-C₁₁ are independently either C—C (single) or C═C (double) bonds.

27. A compound as claimed in any one of claims 20 to 26, wherein X is O.

28. A compound as claimed in any one of claims 20 to 27, wherein R² is Cl, or wherein R² is Br.

29. A compound as claimed in any one of claims 20 to 28, wherein R³ is OH.

30. A compound as claimed in any one of claims 20 to 29, wherein each of C₂-C₃, C₄-C₅, C₆-C₇, C₈-C₉ and C₁₀-C₁₁ are C═C (double) bonds.

31. A compound as claimed in claim 20 of formula (Ib), or a pharmaceutically acceptable salt, metabolite, isomer (e.g. stereoisomer) or prodrug thereof: 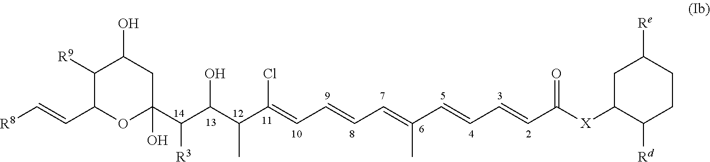
wherein X, R^{d}, R^{e}, R³ and R⁹ are as defined in any one of claims 20, 25 to 27 and 29.

32. A compound as claimed in claim 20 of formula (Ic), or a pharmaceutically acceptable salt, metabolite, or prodrug thereof: 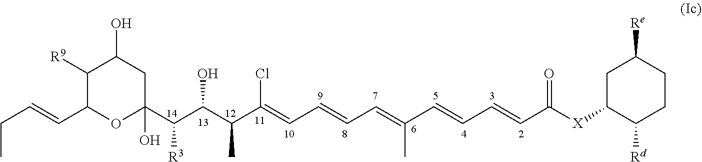
wherein X, R^{d}, R^{e}, R³ and R⁹ are as defined in any one of claims 20, 25 to 27 and 29.

33. A compound as claimed in claim 20 selected from any of the following compounds, or a pharmaceutically acceptable salt, metabolite, isomer (e.g. stereoisomer) or prodrug thereof:

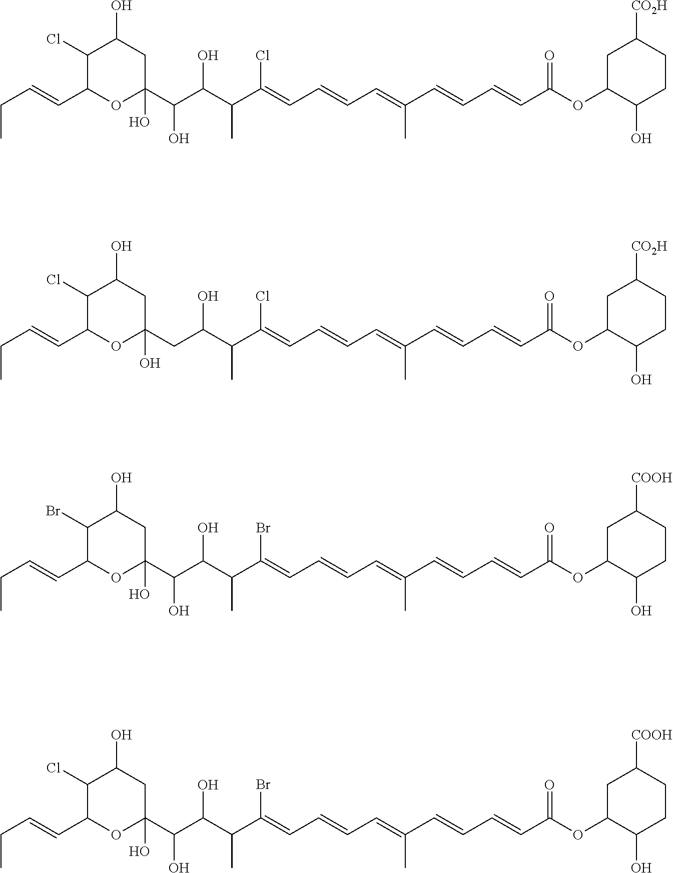 [SMILES: CC/C=C/C1OC(O)(C(O)C(O)C(C)/C(Cl)=C/C=C/C=C(C)/C=C/C=C/C(=O)OC2CC(C(=O)O)CCC2O)CC(O)C1Cl ; CC/C=C/C1OC(O)(CC(O)C(C)/C(Cl)=C/C=C/C=C(C)/C=C/C=C/C(=O)OC2CC(C(=O)O)CCC2O)CC(O)C1Cl ; CC/C=C/C1OC(O)(C(O)C(O)C(C)/C(Br)=C/C=C/C=C(C)/C=C/C=C/C(=O)OC2CC(C(=O)O)CCC2O)CC(O)C1Br ; CC/C=C/C1OC(O)(C(O)C(O)C(C)/C(Br)=C/C=C/C=C(C)/C=C/C=C/C(=O)OC2CC(C(=O)O)CCC2O)CC(O)C1Cl]

34. A compound as claimed in claim 20 selected from any of the following compounds, or a pharmaceutically acceptable salt, metabolite, or prodrug thereof:

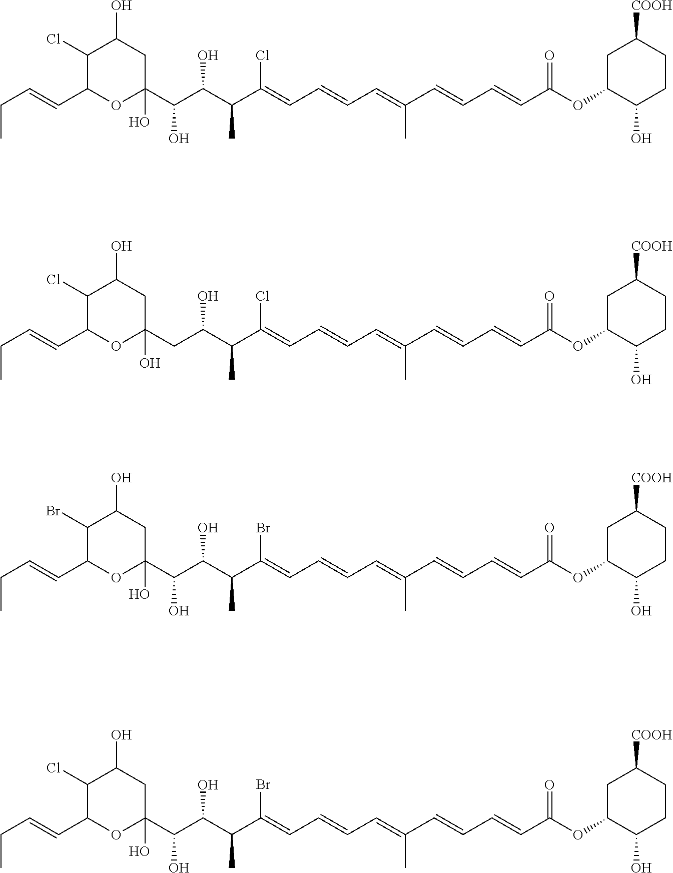 [SMILES: CC/C=C/C1OC(O)([C@@H](O)[C@H](O)[C@H](C)/C(Cl)=C/C=C/C=C(C)/C=C/C=C/C(=O)O[C@@H]2C[C@@H](C(=O)O)CC[C@@H]2O)CC(O)C1Cl ; CC/C=C/C1OC(O)(C[C@H](O)[C@H](C)/C(Cl)=C/C=C/C=C(C)/C=C/C=C/C(=O)O[C@@H]2C[C@@H](C(=O)O)CC[C@@H]2O)CC(O)C1Cl ; CC/C=C/C1OC(O)([C@@H](O)[C@H](O)[C@H](C)/C(Br)=C/C=C/C=C(C)/C=C/C=C/C(=O)O[C@@H]2C[C@@H](C(=O)O)CC[C@@H]2O)CC(O)C1Br ; CC/C=C/C1OC(O)([C@@H](O)[C@H](O)[C@H](C)/C(Br)=C/C=C/C=C(C)/C=C/C=C/C(=O)O[C@@H]2C[C@@H](C(=O)O)CC[C@@H]2O)CC(O)C1Cl]

35. A compound of formula (II), or a pharmaceutically acceptable salt, metabolite, isomer (e.g. stereoisomer) or prodrug thereof: 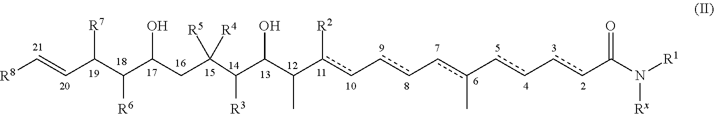
wherein:
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁷ is H, OH, or —OC(O)NR′₂ (where each R′ is independently H or C₁₋₃ alkyl, e.g. CH₃), preferably R⁷ is H, OH or —OC(O)NH₂;
R⁸ is a straight-chained or branched C₁₋₃ alkyl group (e.g. C₁₋₆ alkyl group);
R^{x} is either H or C₁₋₃ alkyl, e.g. CH₃; and
each — independently represents an optional bond (i.e. each of C₂-C₃, C₄-C₅, C₆-C₇, C₈-C₉ and C₁₀-C₁₁ are independently either C—C (single) or C═C (double) bonds).

36. A compound as claimed in claim 35, wherein R¹ is an optionally substituted cyclohexyl or cyclopentyl ring, an optionally substituted cyclohexenyl ring, or an optionally substituted straight-chained C₁₋₆ alkyl group.

37. A compound as claimed in claim 35 or claim 36, wherein R¹ is substituted by one or more of the following groups: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof).

38. A compound as claimed in any one of claims 35 to 37, wherein R⁸ is a straight-chained or branched C₁₋₅ alkyl, preferably a straight-chained or branched C₁₋₄ alkyl, e.g. methyl, ethyl, isopropyl, or tert. butyl.

39. A compound as claimed in claim 38, wherein R⁸ is ethyl.

40. A compound as claimed in claim 35 of formula (IIa) or a pharmaceutically acceptable salt, metabolite, isomer (e.g. stereoisomer) or prodrug thereof: 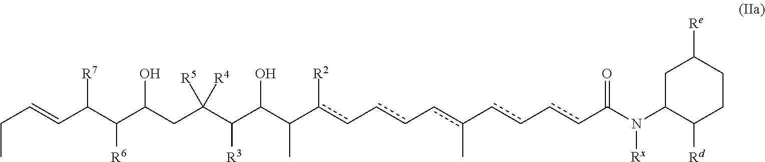
wherein:
R^{d} is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or
C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R² to R⁷, and R^{x} are as defined in claim 35; and
each — independently represents an optional bond (i.e. each of C₂-C₃, C₄-C₅, C₆-C₇, C₈-C₉ and C₁₀-C₁₁ are independently either C—C (single) or C═C (double) bonds).

41. A compound as claimed in claim 40, wherein
R^{d} is H, OH, NH₂, SH, F, Cl, Br, I, or CH₃, preferably H, OH or NH₂;
R^{e} is H, CO₂H, PO₃H₂, or SO₃H₂, preferably CO₂H;
R² is F, Cl, Br or I, preferably Cl;
R³ is OH;
R⁴ and R⁵ together are ═O;
R⁶ is F, Cl, Br or I, preferably Cl;
R⁷ is —OC(O)NH₂;
R^{x} is H or CH₃, preferably H; and
C₂-C₃, C₄-C₅, C₆-C₇, C₈-C₉ and C₁₀-C₁₁ are independently either C—C (single) or C═C (double) bonds.

42. A compound as claimed in any one of claims 35 to 41, wherein R² is Cl.

43. A compound as claimed in any one of claims 35 to 42, wherein R³ is OH.

44. A compound as claimed in any one of claims 35 to 43, wherein R⁴ and R⁵ together are ═O.

45. A compound as claimed in any one of claims 35 to 44, wherein R⁶ is Cl.

46. A compound as claimed in any one of claims 35 to 45, wherein R⁷ is —OC(O)NH₂.

47. A compound as claimed in any one of claims 35 to 46, wherein each of C₂-C₃, C₄-C₅, C₆-C₇, C₈-C₉ and C₁₀-C₁₁ are C═C (double) bonds.

48. A compound as claimed in claim 35 of formula (lib), or a pharmaceutically acceptable salt, metabolite, isomer (e.g. stereoisomer) or prodrug thereof: 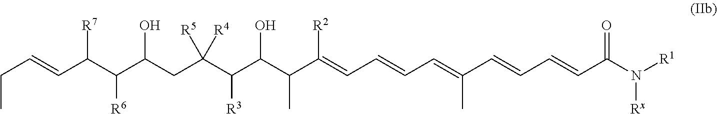
wherein R¹ to R⁷, and R^{x} are as defined in any one of claims 35 to 37 and 40 to 46.

49. A compound as claimed in claim 25 of formula (IIc), or a pharmaceutically acceptable salt, metabolite, or prodrug thereof: 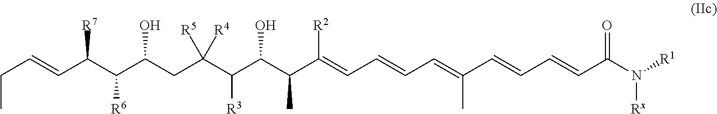
wherein R¹ to R⁷, and R^{x} are as defined in any one of claims 35 to 37 and 40 to 46.

50. A compound as claimed in claim 35 selected from any of the following compounds, or a pharmaceutically acceptable salt, metabolite, isomer (e.g. stereoisomer) or prodrug thereof:

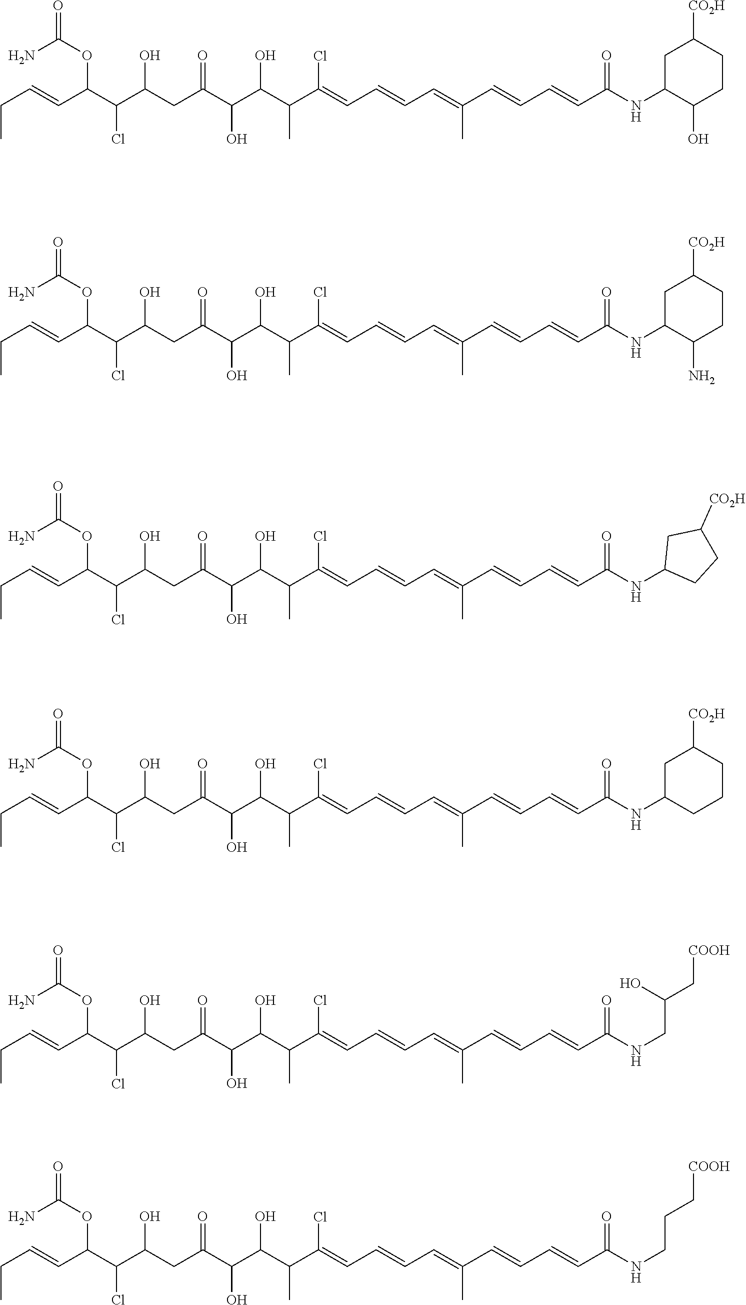

51. A compound as claimed in claim 35 selected from any of the following compounds, or a pharmaceutically acceptable salt, metabolite, or prodrug thereof:

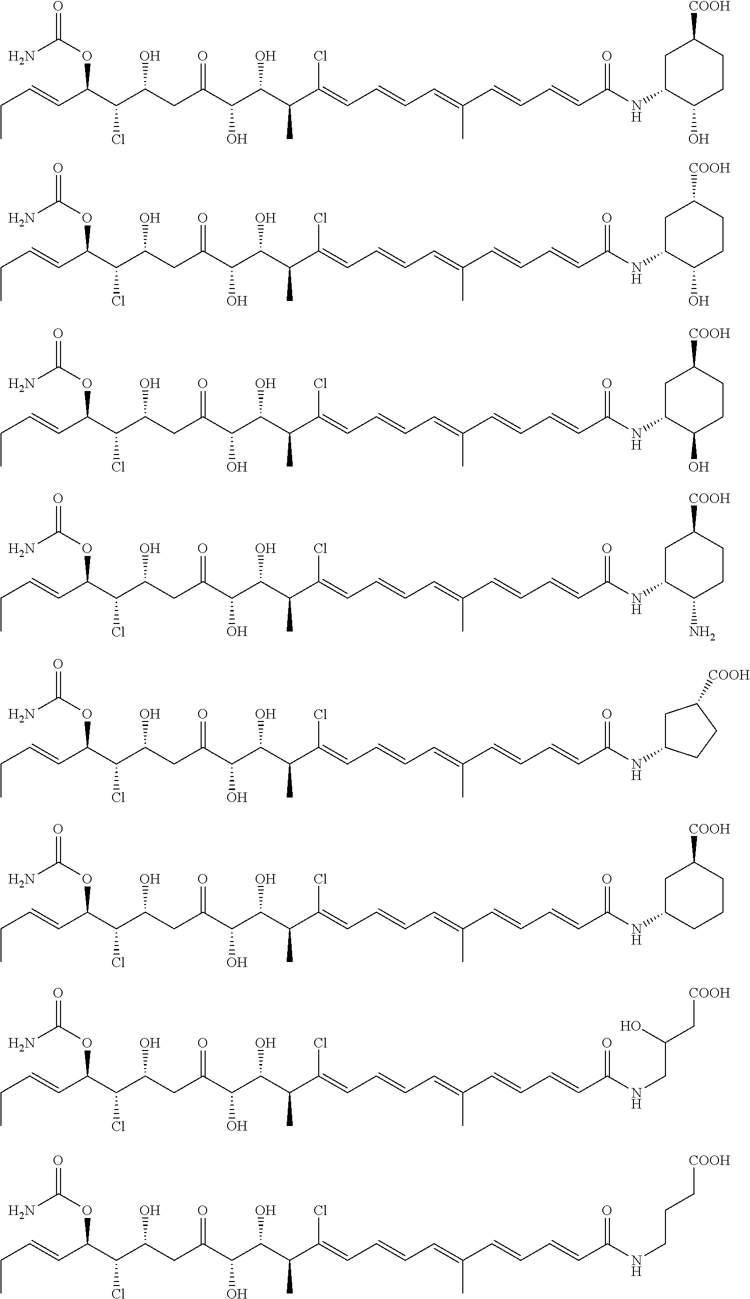

52. A compound of formula (IIIa) or (IIIb), or a pharmaceutically acceptable salt, metabolite, or prodrug thereof, or a stereoisomeric form thereof at one or more of positions C6, C11 to C15, and C17 to C19 in the polyketide chain: 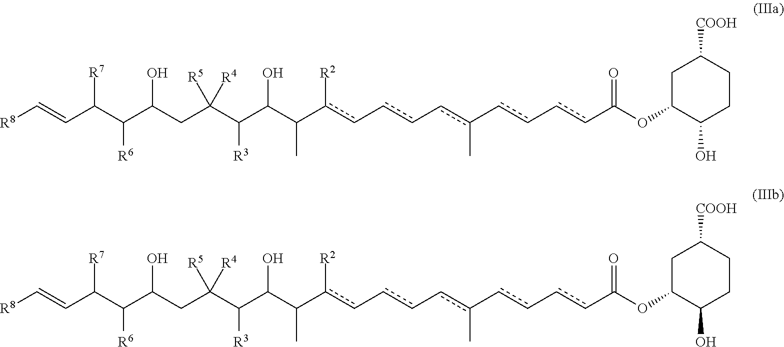
wherein:
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁷ is H, OH, or —OC(O)NR′₂ (where each R′ is independently H or C₁₋₃ alkyl, e.g. CH₃), preferably R⁷ is H, OH or —OC(O)NH₂;
R⁸ is a straight-chained or branched C₁₋₈ alkyl group (e.g. C₁₋₆ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds).

53. A compound as claimed in claim 52, wherein R⁸ is a straight-chained or branched C₁₋₅ alkyl, preferably a straight-chained or branched C₁₋₄ alkyl, e.g. methyl, ethyl, isopropyl, or tert. butyl.

54. A compound as claimed in claim 53, wherein R⁸ is ethyl.

55. A compound as claimed in any one of claims 52 to 54, wherein R² is F, Cl, Br or I, preferably Cl.

56. A compound as claimed in any one of claims 52 to 55, wherein R³ is OH.

57. A compound as claimed in any one of claims 52 to 56, wherein R⁴ and R⁵ together are ═O.

58. A compound as claimed in any one of claims 52 to 57, wherein R⁶ is Cl.

59. A compound as claimed in any one of claims 52 to 58, wherein R⁷ is —OC(O)NH₂.

60. A compound as claimed in any one of claims 52 to 59, wherein each of C₂-C₃, C₃-C₄, C₄-C₅, C₆-C₇, C₈-C₉ and C₁₀-C₁₁ are C═C (double) bonds.

61. A compound as claimed in claim 52 of formula (IIIc) or (IIId), or a pharmaceutically acceptable salt, metabolite, isomer (e.g. stereoisomer) or prodrug thereof: 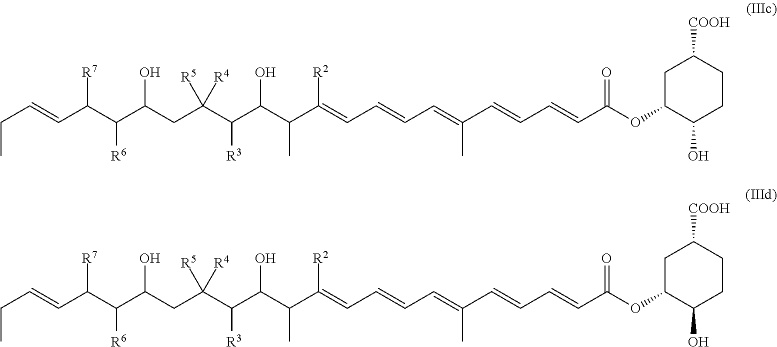
wherein R² to R⁷ are as defined in any one of claims 52 and 55 to 59.

62. A compound as claimed in claim 52 of formula (IIIe) or (IIIf), or a pharmaceutically acceptable salt, metabolite or prodrug thereof: 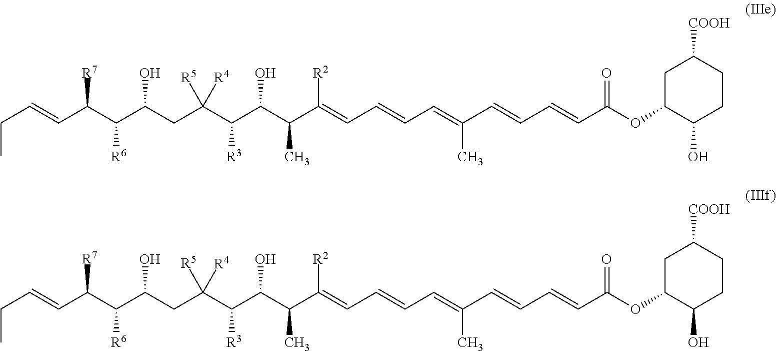
wherein R² to R⁷ are as defined in any one of claims 52 and 55 to 59.

63. A compound as claimed in claim 52 selected from any of the following compounds, or a pharmaceutically acceptable salt, metabolite, isomer (e.g. stereoisomer) or prodrug thereof:

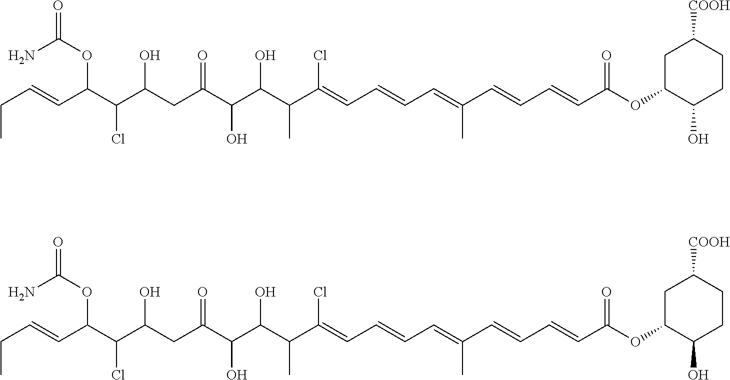 [SMILES: CC/C=C/C(OC(N)=O)C(Cl)C(O)CC(=O)C(O)C(O)C(C)/C(Cl)=C/C=C/C=C(C)/C=C/C=C/C(=O)O[C@@H]1C[C@H](C(=O)O)CC[C@@H]1O ; CC/C=C/C(OC(N)=O)C(Cl)C(O)CC(=O)C(O)C(O)C(C)/C(Cl)=C/C=C/C=C(C)/C=C/C=C/C(=O)O[C@@H]1C[C@H](C(=O)O)CC[C@H]1O]

64. A compound as claimed in claim 52 selected from any of the following compounds, or a pharmaceutically acceptable salt, metabolite, or prodrug thereof:

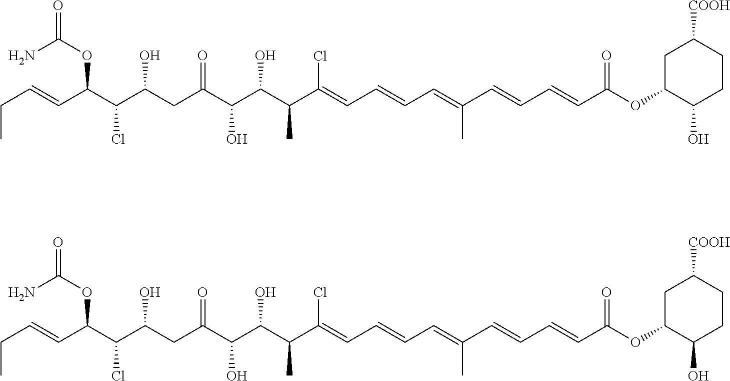 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@H](C(=O)O)CC[C@@H]1O ; CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1C[C@H](C(=O)O)CC[C@H]1O]

65. A compound of formula (V), or a pharmaceutically acceptable salt, metabolite, isomer (e.g. stereoisomer) or prodrug thereof: 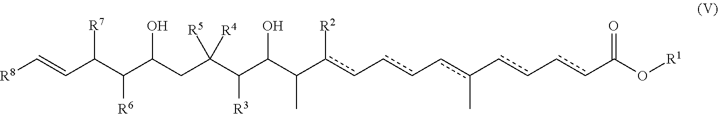
wherein:
R¹ is a 5-membered, saturated or unsaturated, carbocyclic ring substituted by one or more substituents, or
R¹ is a 6-membered, unsaturated, carbocyclic ring substituted by one or more substituents, or
R¹ is a 6-membered, saturated, carbocyclic ring substituted by one or three substituents;
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁷ is H, OH, or —OC(O)NR′₂ (where each R′ is independently H or C₁₋₃ alkyl, e.g. CH₃), preferably R⁷ is H, OH or —OC(O)NH₂;
R⁸ is a straight-chained or branched C₁₋₈ alkyl group (e.g. C₁₋₆ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds).

66. A compound as claimed in claim 65, wherein R¹ is a cyclohexyl ring substituted by one or three substituents, or a cyclopentyl ring substituted by one or more substituents, for example one or two substituents.

67. A compound as claimed in claim 65, wherein R¹ is a cyclohexenyl ring which is optionally substituted by one or more substituents.

68. A compound as claimed in any one of claims 65 to 67, wherein R¹ is substituted by one or more of the following groups: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof).

69. A compound as claimed in claim 65, wherein R¹ is selected from any of the following groups (in which * denotes the point of attachment of the substituent to the remainder of the molecule):

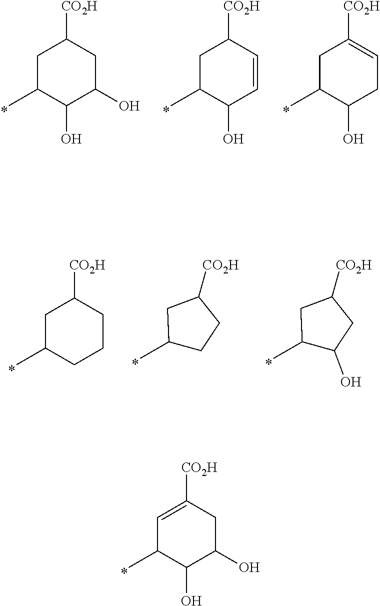

70. A compound as claimed in any one of claims 65 to 69, wherein R⁸ is a straight-chained or branched C₁₋₅ alkyl, preferably a straight-chained or branched C₁₋₄ alkyl, e.g. methyl, ethyl, isopropyl, or tert. butyl.

71. A compound as claimed in claim 70, wherein R⁸ is ethyl.

72. A compound as claimed in any one of claims 65 to 71, wherein each of C₂-C₃, C₄-C₅, C₆-C₇, C₈-C₉ and C₁₀-C₁₁ are C═C (double) bonds.

73. A compound as claimed in claim 65 of formula (Va) or a pharmaceutically acceptable, salt, metabolite, isomer (e.g. stereoisomer) or prodrug thereof: 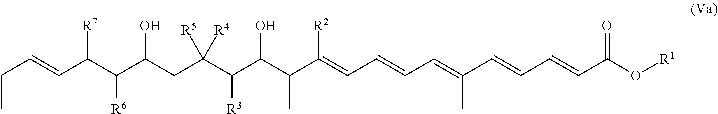
wherein R¹ to R⁷ are as defined in any one of claims 65 to 69.

74. A compound as claimed in claim 65 of formula (Vb), or a pharmaceutically acceptable salt, metabolite, or prodrug thereof: 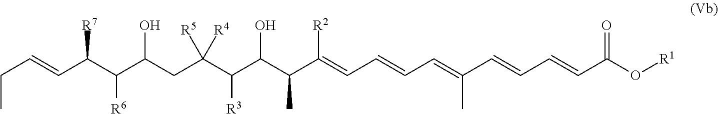
wherein R¹ to R⁷ are as defined in any one of claims 65 to 69.

75. A compound as claimed in any one of claims 65 to 74, wherein R² is Cl.

76. A compound as claimed in any one of claims 65 to 75, wherein R³ is OH.

77. A compound as claimed in any one of claims 65 to 76, wherein R⁴ and R⁵ together are ═O, or wherein R⁴ is H and R⁵ is OH.

78. A compound as claimed in any one of claims 65 to 77, wherein R⁶ is Cl.

79. A compound as claimed in claim 65 selected from any of the following compounds, their pharmaceutically acceptable salts, metabolites, isomers (e.g. stereoisomers) and prodrugs:

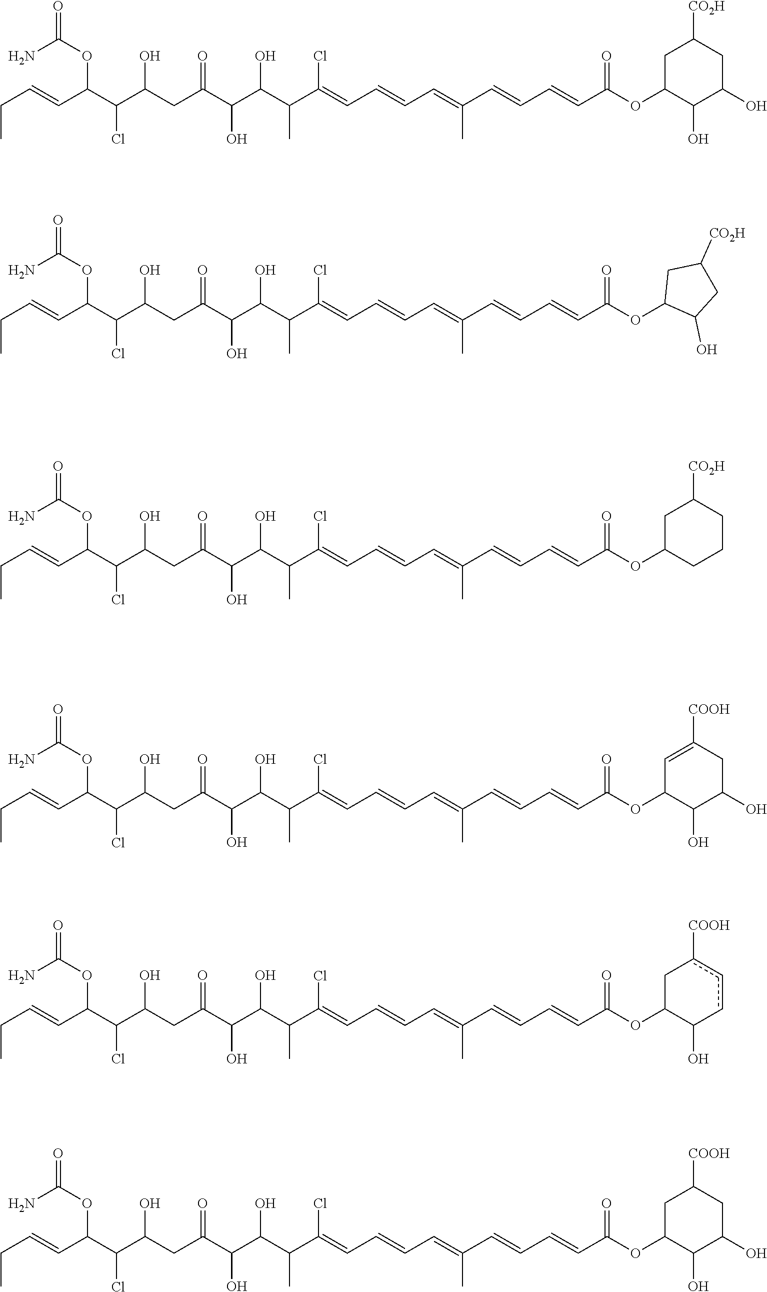

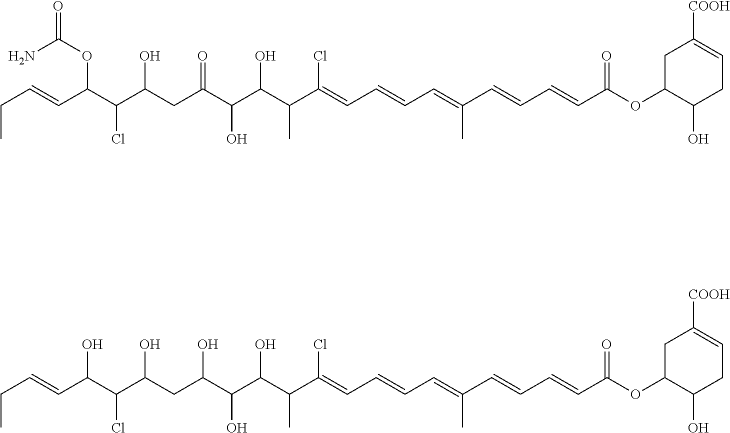 [SMILES: CC/C=C/C(OC(N)=O)C(Cl)C(O)CC(=O)C(O)C(O)C(C)/C(Cl)=C/C=C/C=C(C)/C=C/C=C/C(=O)OC1CC(C(=O)O)=CCC1O ; CC/C=C/C(O)C(Cl)C(O)CC(O)C(O)C(O)C(C)/C(Cl)=C/C=C/C=C(C)/C=C/C=C/C(=O)OC1CC(C(=O)O)=CCC1O]

80. A compound as claimed in claim 65 selected from any of the following compounds, their pharmaceutically acceptable salts, metabolites, and prodrugs:

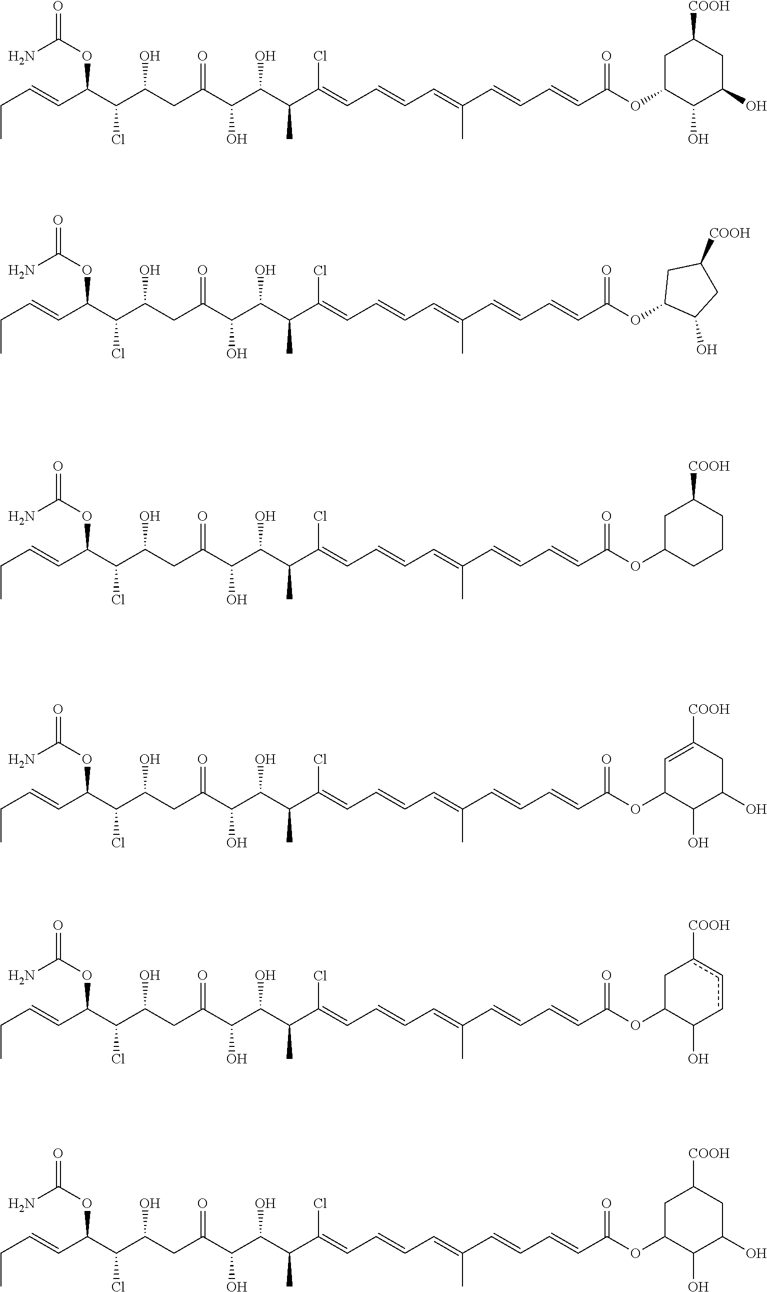

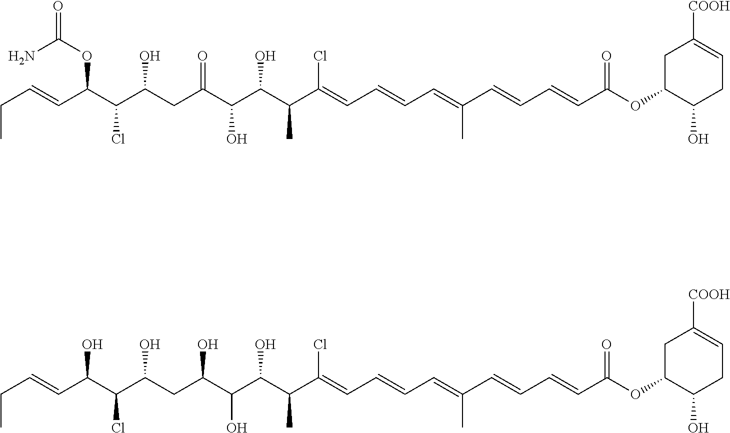 [SMILES: CC/C=C/[C@@H](OC(N)=O)[C@@H](Cl)[C@H](O)CC(=O)[C@@H](O)[C@H](O)[C@H](C)/C(Cl)=C/C=C/C=C(C)/C=C/C=C/C(=O)O[C@@H]1CC(C(=O)O)=CC[C@@H]1O ; CC/C=C/[C@@H](O)[C@H](Cl)[C@H](O)C[C@@H](O)C(O)[C@H](O)[C@H](C)/C(Cl)=C/C=C/C=C(C)/C=C/C=C/C(=O)O[C@@H]1CC(C(=O)O)=CC[C@@H]1O]

81. A compound of formula (VI), or a pharmaceutically acceptable salt, metabolite, isomer (e.g. stereoisomer) or prodrug thereof: 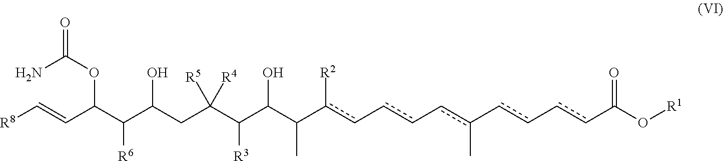
wherein:
R¹ is a 5- or 6-membered, saturated or unsaturated, carbocyclic ring optionally substituted by one or more substituents, or R¹ is an optionally substituted straight-chained or branched C₁₋₆ alkyl group (e.g. C₁₋₃ alkyl group);
R² is H, F, Cl, Br, I or CH₃;
R³ is H or OH;
R⁴ and R⁵ are independently selected from H and OH, or R⁴ and R⁵ together are ═O;
R⁶ is H, F, Cl, Br, I or CH₃;
R⁸ is a straight-chained or branched C₁₋₈ alkyl group (e.g. C₁₋₆ alkyl group); and
each — independently represents an optional bond (i.e. each of C₂-C₃, C₄-C₅, C₆-C₇, C₈-C₉ and C₁₀-C₁₁ are independently either C—C (single) or C═C (double) bonds;
with the proviso that either at least one of R², R³ and R⁶ is H, or R⁴ is H and R⁵ is OH).

82. A compound as claimed in claim 81, wherein R¹ is an optionally substituted cyclohexyl or cyclopentyl ring, an optionally substituted cyclohexenyl ring, or an optionally substituted straight-chained C₁₋₆ alkyl group.

83. A compound as claimed in claim 81 or claim 82, wherein R¹ is substituted by one or more of the following groups: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof).

84. A compound as claimed in any one of claims 81 to 83, wherein R⁸ is a straight-chained or branched C₁₋₅ alkyl, preferably a straight-chained or branched C₁₋₄ alkyl, e.g. methyl, ethyl, isopropyl, or tert. butyl.

85. A compound as claimed in claim 84, wherein R⁸ is ethyl.

86. A compound as claimed in claim 81 of formula (VIa), or a pharmaceutically acceptable, salt, metabolite, isomer (e.g. stereoisomer) or prodrug thereof: 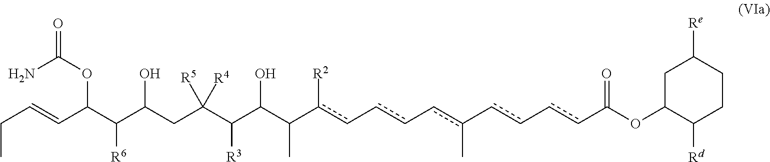
wherein:
R^{d} is H, OH, NR^{a}₂ (where each R^{a} is independently H or C₁₋₃ alkyl, e.g. CH₃), SR^{b} (where R^{b} is H or C₁-3 alkyl, e.g. CH₃), halogen (e.g. F, Cl, Br, or I), or
C₁₋₃ alkyl (e.g. CH₃), preferably R^{d} is H, OH, NH₂, SH, F, Cl, Br, I, or CH₃;
R^{e} is H, CO₂H (or an ester thereof), PO₃H₂ (or an ester thereof) or SO₃H₂ (or an ester thereof), preferably R^{e} is H, CO₂H, PO₃H₂, or SO₃H₂;
R² to R⁶ are as defined in claim 81; and
each — independently represents an optional bond (i.e. each of C₂-C₃, C₄-C₅, C₆-C₇, C₈-C₉ and C₁₀-C₁₁ are independently either C—C (single) or C═C (double) bonds).

87. A compound as claimed in claim 86, wherein:
R^{d} is H, OH, NH₂, SH, F, Cl, Br, I, or CH₃, preferably OH;
R^{e} is H, CO₂H, PO₃H₂, or SO₃H₂, preferably CO₂H;
each of C₂-C₃, C₄-C₅, C₆-C₇, C₈-C₉, and C₁₀-C₁₁ are independently either C—C (single) or C═C (double) bonds;
R² is H, Cl or Br, e.g. R² is H or Cl;
R³ is H or OH;
R⁴ is H and R⁵ is OH; and
R⁶ is H, Cl or Br, e.g. R⁶ is H or Cl.

88. A compound as claimed in any one of claims 81 to 87, wherein R² is H or Cl, preferably H, or wherein R² is Br.

89. A compound as claimed in any one of claims 81 to 88, wherein R³ is H.

90. A compound as claimed in any one of claims 81 to 89, wherein R⁴ is H and R⁵ is OH.

91. A compound as claimed in any one of claims 81 to 90, wherein R⁶ is H or Cl, preferably H, or wherein R⁶ is Br.

92. A compound as claimed in any one of claims 81 to 91, wherein each of C₂-C₃, C₄-C₅, C₆-C₇, C₈-C₉ and C₁₀-C₁₁ are C═C (double) bonds.

93. A compound as claimed in claim 81 of formula (VIb), or a pharmaceutically acceptable salt, metabolite, isomer (e.g. stereoisomer) or prodrug thereof: 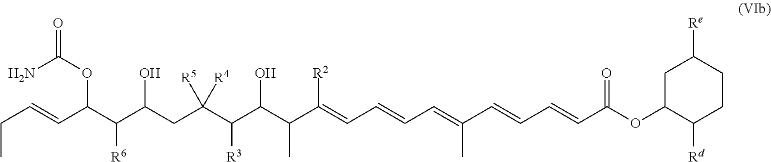
wherein R^{d}, R^{e}, and R² to R⁶ are as defined in any one of claims 81 and 86 to 91.

94. A compound as claimed in claim 81 of formula (VIc), or a pharmaceutically acceptable salt, metabolite, or prodrug thereof: 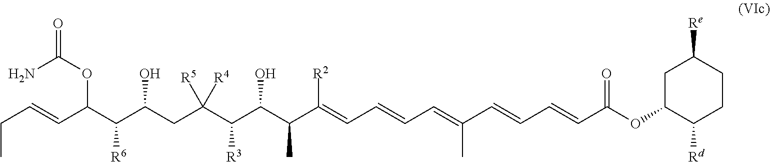
wherein R^{d}, R^{e}, and R² to R⁶ are as defined in any one of claims 81 and 86 to 91.

95. A compound as claimed in claim 81 selected from any one of the following compounds, their pharmaceutically acceptable salts, metabolites, isomers (e.g. stereoisomers) and prodrugs:

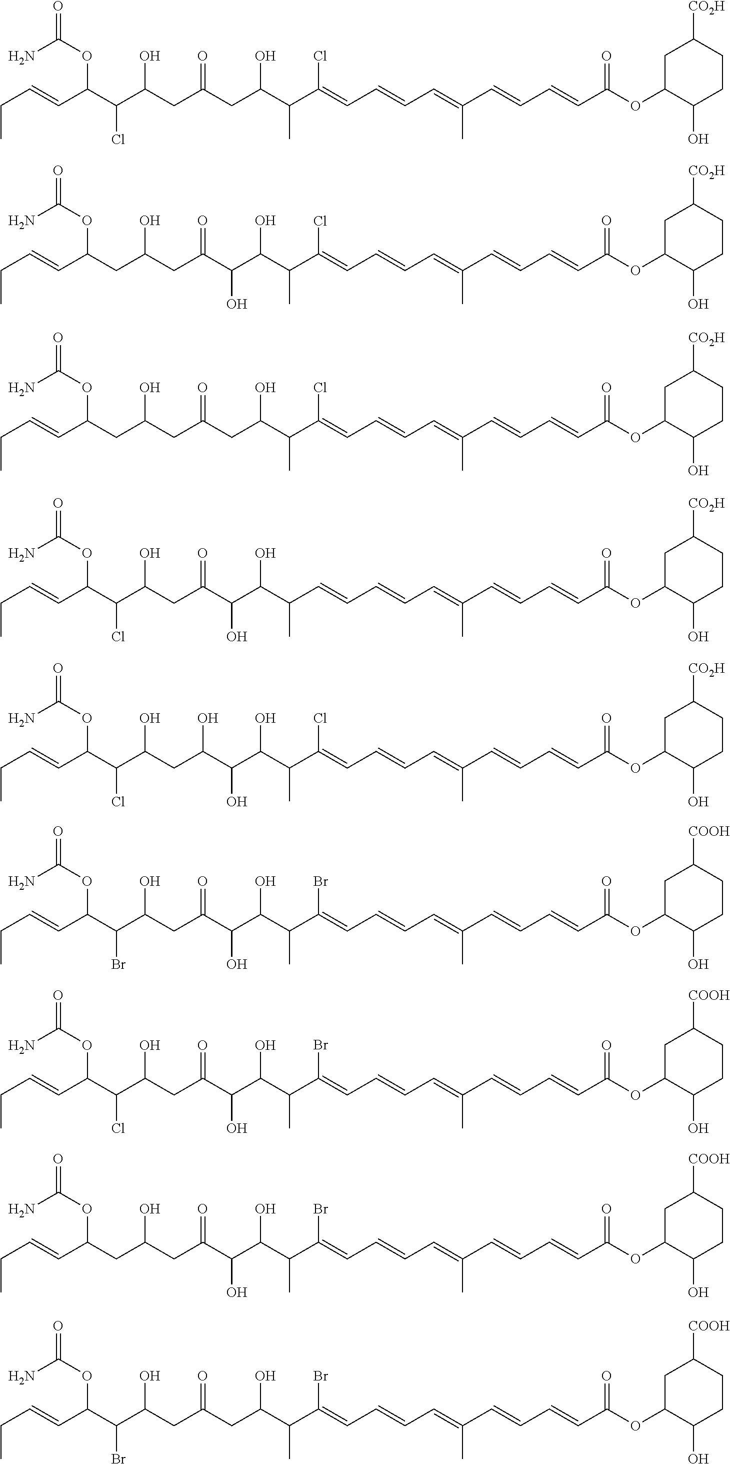

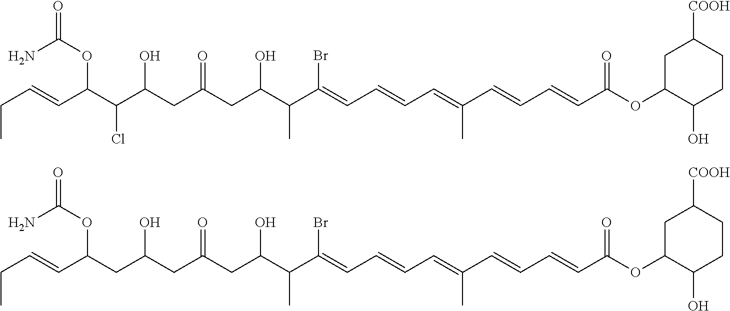

96. A compound as claimed in claim 81 selected from any one of the following compounds, their pharmaceutically acceptable salts, metabolites, and prodrugs:

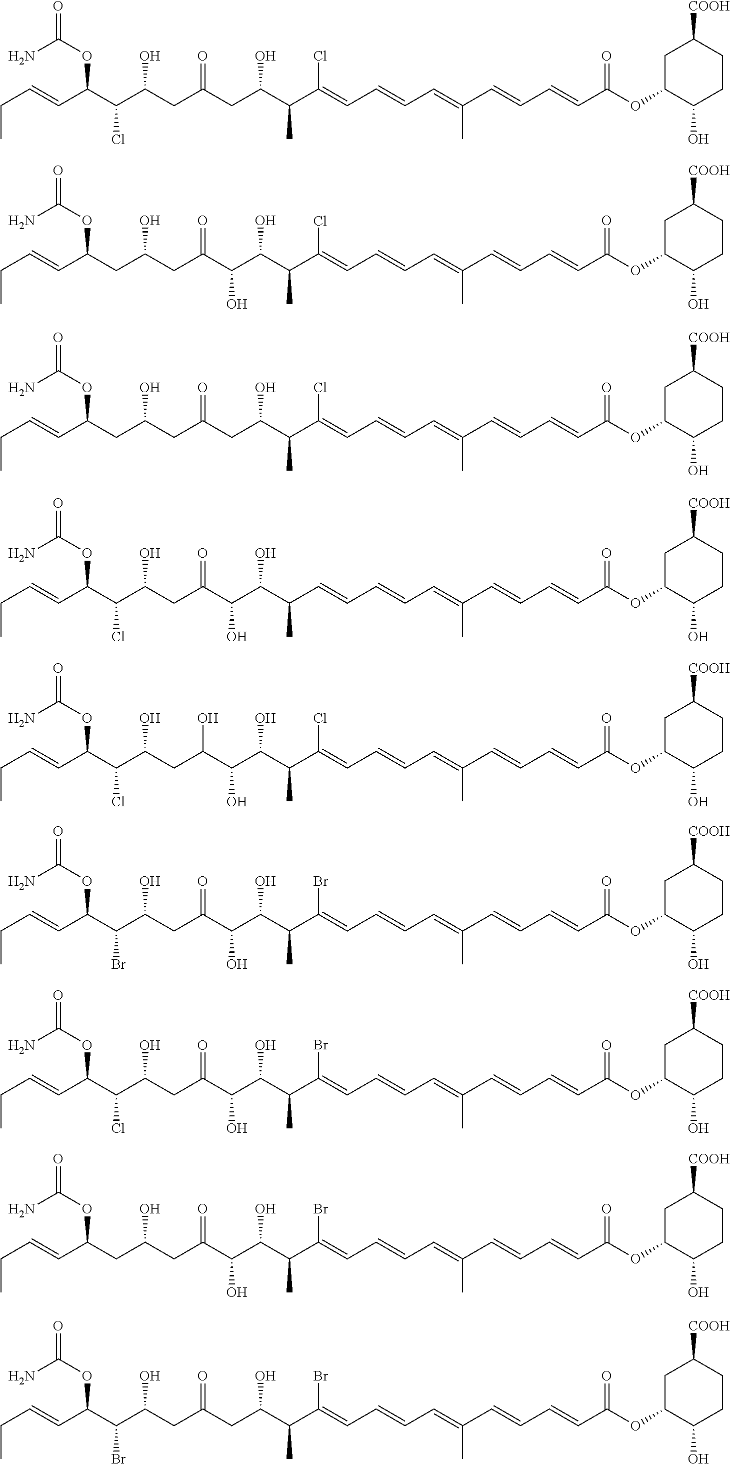

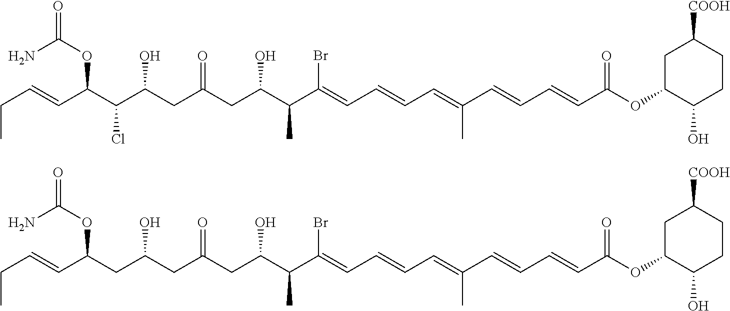

97. A compound as claimed in any one of claims 1 to 96 for use as a medicament.

98. A compound as claimed in any one of claims 1 to 96 for use as an antimicrobial agent.

99. A compound as claimed in any one of claims 1 to 96 for use in the treatment of an infection caused by a microbe which is a bacterium.

100. A compound for use as claimed in claim 99 in the treatment of an infection caused by a microbe which is a Gram-negative bacterium, e.g. selected from Acinetobacter species, Burkholderia species, Ralstonia species and Stenotrophomonas species.

101. A compound as claimed in any one of claims 1 to 96 for use in the treatment of an infection caused by at least one microbe which is resistant to at least one antimicrobial drug.

102. A compound for use as claimed in claim 101 in the treatment of an infection, wherein the antimicrobial drug is selected from drugs of the carbapenem family, drugs of the penicillin family, drugs of the vancomycin family, drugs of the aminoglycoside family, drugs of the quinolone family, drugs of the daptomycin family, drugs of the cephalosporin family, drugs of the macrolide family, and combinations thereof.

103. A compound for use as claimed in claim 102 in the treatment of an infection, wherein the antimicrobial drug is selected from penicillin, ampicillin, methicillin, vancomycin, gentamycin, ofloxacin, ciprofloxacin, daptomycin, cefdimir, erythromycin, equivalents thereof, and combinations thereof.

104. Use of a compound as claimed in any one of claims 1 to 96 in the manufacture of a medicament for use in treating an infection caused by at least one microbe as defined in any one of claims 99 to 103.

105. A compound for use as claimed in any one of claims 99 to 103 in the treatment of infection, or a use as claimed in claim 104, wherein the infection is an infection of the respiratory system, digestive system, urinary system, nervous system, a blood infection, a soft tissue infection, a skin infection, a nasal canal infection, or combinations thereof.

106. A pharmaceutical composition comprising a compound as claimed in any one of claims 1 to 96 and a pharmaceutically acceptable carrier.

107. A pharmaceutical composition as claimed in claim 106, further comprising at least one other therapeutically active agent.

108. A pharmaceutical composition as claimed in claim 107, wherein the compound according to any one of claims 1 to 96 and the other therapeutically active agent are adapted for sequential, separate or simultaneous administration.

109. An active agent, especially an antimicrobial agent, having mass spectral and/or NMR spectroscopic properties substantially according to one or more of FIGS. 1 to 126 and/or any one of Tables 3 to 27.

110. A process for the preparation of a compound as claimed in any one of claims 1 to 96, comprising cultivating a microorganism capable of producing said compound, in a culture medium comprising a source of assimilable carbon, nitrogen, and inorganic salts and, optionally, recovering said compound from the culture medium and, optionally, further converting the compound into a pharmaceutically acceptable salt thereof.

111. A process as claimed in claim 110, wherein the microorganism is Burkholderia ambifaria, e.g. one or more strains selected from BCCC0203 (also known as LMG P-24640; BCF), BCC0118 (also known as LMG P-24636; JLO), BCC 1248 (also known as LMG P-24641; KWO-1), BCC0250 (also known as LMG P-24637; WM2), BCC1241 (also known as LMG P-24639; KC311-6), BCC0207 (also known as LMG P-19182; AMMD; ATCC BAA-244; CCUG 44356; KCTC 12943; FC768; J2742 Vandamme R-696FC0768), and BCC0267 (also known as LMG P-19467; CEP0996; Coenye R-9935), or a mutant or variant thereof.

112. A process as claimed in claim 110 or claim 111, further comprising converting the compound into another compound of any one of formula (I) to (VI) by chemical synthesis and, optionally, further converting the resultant compound into a pharmaceutically acceptable salt thereof.

113. A method for the treatment of an infection, the method comprising administering to a subject in need thereof a compound as claimed in any one of claims 1 to 96, wherein the infection is caused by at least one microbe, optionally wherein the microbe is resistant to an antimicrobial drug.
